(19) United States
(12) Patent Application Publication
(10) Pub. No.: US 2026/0092121 A1
(43) Pub. Date: Apr. 2, 2026

(54) ANTI-TRKB/CD3 ANTIBODIES AND USES THEREOF

(71) Applicant: Talem Therapeutics LLC, Fargo, ND (US)

(72) Inventors: Ilse ROODINK, Loenen (NL); Jennifer L. BATH, Plymouth, MN (US)

(21) Appl. No.: 19/120,202

(22) PCT Filed: Oct. 13, 2023
(86) PCT No.: PCT/US2023/076894

§ 371 Date: Apr. 10, 2025

Related U.S. Application Data
(60) Provisional application No. 63/379,637, filed on Oct. 14, 2022.
(60) Provisional application No. 63/379,642, filed on Oct. 14, 2022.
(60) Provisional application No. 63/495,751, filed on Apr. 12, 2023.

(51) Int. Cl. C07K 16/28

(52) U.S. Cl. CPC C07K 16/2863; C07K 16/2809; C07K 2317/24; C07K 2317/31

(57) ABSTRACT

The present application relates to antibodies or anti-gen-binding fragments thereof that specifically bind to TrkB and/or CD3, and to nucleic acids encoding such antibodies or antigen-binding fragments thereof, as well as to pharmaceutical compositions comprising the antibodies, antigen-binding fragments thereof or nucleic acids. Bispecific antibodies specifically binding to TrkB and CD3 are also described. The antibodies, antigen-binding fragments thereof, nucleic acids or pharmaceutical compositions may be useful for modulating the activity of CD3 and/or TrkB-expressing cells such as neural cell and T lymphocytes, and for the treatment of diseases or conditions in which stimulation or inhibition of CD3 and/or TrkB is beneficial, such as neurodegenerative diseases, optic neuropathy and metabolic diseases, mental disorders as well as for the treatment of autoimmune disorders and cancers.

### CROSS REFERENCE TO RELATED APPLICATIONS

The present application claims the benefit of U.S. provisional Patent applications Nos. 63/379,637 and 63/379,642, filed on Oct. 14, 2022, and U.S. provisional Patent application No. 63/495,571, filed on Apr. 12, 2023. The entire content of these applications is incorporated herein by reference.

### SEQUENCE LISTING

A sequence listing is submitted herewith as an XML file named 140018-00119-SSS_TALM-004-02WO_Seq listing.xml, that was created on Oct. 13, 2023, and having a size of 637,174 bytes. The content of the aforementioned file is hereby incorporated by reference in its entirety.

### TECHNICAL FIELD

The present disclosure generally relates to the field of mental, neurodegenerative and immune disorders and oncology, and more particularly to new products and uses thereof such as for the treatment of neurodegenerative disorders and cancers associated with TrkB expression and/or activity.

### BACKGROUND ART

### TrkB

The tropomyosin receptor kinase (Trk) family is a family of neurotrophin receptors and consists of three members, namely TrkA, TrkB, and TrkC [Bertrand et al., 2012]. These receptors are single transmembrane proteins that are located at the cellular membrane and possess an intracellular (cytoplasmic) tyrosine kinase domain. Binding of a neurotrophin to the extracellular binding site of the receptor stimulates this intracellular domain, upon which it becomes catalytically active [Huang & Reichardt, 2003].

Neurotrophins are a type of signaling molecules that are functionally similar to the class of molecules that we refer to as growth factors [Reichardt, 2006]. They are essential for neuronal development (growth and differentiation) and normal functioning of the central and peripheral nervous system. Some well-characterized neurotrophins include nerve growth factor (NGF), brain-derived neurotropic factor (BDNF), neurotrophin-3 (NT-3), and neurotrophin-4 and 5 (NT-4/5). Each of these has the potential to activate one or several members of the Trk family. NGF is primarily a ligand for TrkA, whereas BDNF, and NT-4/5 are predominantly ligands for TrkB. TrkC is a receptor for NT-3. [Nakagawara, 2001; Chao & Lee, 2004].

When a neurotrophic growth-factor binds to its respective Trk, homodimerization is induced and catalytic activity of the intracellular tyrosine kinase domain is initiated [Chao, 2003]. Ligand-induced tyrosine autophosphorylation results in activation of the receptor and, subsequently, in the induction of several different intracellular signaling cascades through phosphorylation of target proteins [Gupta et al., 2013]. These signaling cascades, which include the Ras/MAP kinase (MAPK) pathway, the phosphoinositide 3 kinase (PI3-kinase) pathway, and the phospholipase Cŷ (PLCŷ) pathway, can alter the function of proteins or bring about changes in gene expression in the ligand-induced cells [Kaplan et al., 1991; Kaplan & Miller, 2000]. These alterations or changes influence cellular responses that encompass neurite outgrowth, activity-dependent synapse plasticity, and the prevention or promotion of cell death (apoptosis) [Huang & Reichardt, 2003].

TrkB plays a role in neuronal survival, differentiation, and function. TrkB agonists may have therapeutic potential for treating a number of neurodegenerative, psychiatric and metabolic disorders.

TrkB, in combination with one of its predominant ligands, BDNF, has been found to possess tumor formation- and metastasis-promoting properties [Desmet & Peeper, 2006]. Overexpression of the TrkB protein has been reported in various types of tumors [Meldolesi, 2018]. Gene fusions and overexpression of the gene encoding TrkB (the NTRK2 gene) have been identified in relation to increased tumor cell survival, protection from anti-tumor agent-induced apoptosis, and stimulation of invasion of surrounding tissue in a variety of cancer types [Desmet & Peeper, 2006; Gupta, 2013]. Although well-identified, gene fusions for the NTRK2 gene involved in cancer are relatively rare [Vaishnavi et al., 2015; Khotskaya et al., 2017]. In contrast, excessive expression of the TrkB protein is related to tumor proliferation in myelomas and breast, lung, colon-rectum, pancreas, prostate, liver, and lymphoid cancer [Meldolesi, 2018].

There is thus a need for the identification and development of TrkB-specific molecules, such as TrkB modulators that can provide improved specificity in addition to exhibiting neuronal survival and neuroprotective properties, or that can target tumor cells.

### CD3 and the TCR-CD3 Complex

The immune system works through soluble factors (antibodies) and through effector cells in humoral and cellular, respectively, responses to injury, infection and cancer. The cellular response involves macrophages and T-lymphocytes (T-cells). The latter cells are essential for the secretion of antibodies by B lymphocytic cells. In addition, once activated, T-cells are also known for their production and release of cytolytic factors and their ability to kill cells, including tumor cells. Activation of T-cells is through several signals, such as the recognition of an antigen on an MHC molecule, which can go through the T-cell receptor (TCR) complex.

A cluster of differentiation 3 (CD3) polypeptides are associated with the TCR complex to form a higher order complex with multiple extracellularly accessory chains. The TCR recognizes antigen-peptides bound to a major histocompatibility complex (MHC) molecule, but it is not able to signal binding without the associated CD3 complex. The TCR complex is a heterodimer of an α- and β-chain (most T-cells), or of a γ- and δ-chain (minority of the T-cells) covalently linked through a disulfide bond. The CD3 polypeptides include four subunits, viz. the glycoproteins CD3γ and CD3δ, non-glycosylated CD3ε and an intracytoplasmic CD3ζ. The δ-, ε- and γ-chains are structurally closely related having an external immunoglobulin-like C-domain, whereas the sequences of the Z-subunit are unique compared to those of the other chains. The CD3 chains are organized as heterodimers CD3γ-CD3ε, CD3δ-CD3ε and as homodimer CD3ζ-CD3ζ. The stoichiometry of the CD3-TCR complex is (α-β)₂γδε₂ζ₂. The opposite charges of the transmembrane parts of CD3 chains (negative) and TCR (positive) allow their association in a working complex.

Following a TCR-antigen binding, a cascade is activated through the CD3 employing its cytoplasmic tails, which each contain one (δ, ε and γ) or three (Z) immunoreceptor tyrosine activation motifs (ITAMs), to recruit adaptors, i.e., phosphorylation by specific protein kinases. The presence of ten ITAMs in the CD3-TCR complex makes it very sensitive for antigen binding. This is the first step in T-cell activation resulting finally in gene transcription in the nucleus of the T-cell. The CD3ε plays a central role in the CD3 core and full complex formation. Activation may also occur through the binding of antibodies to the CD3 cluster with an antagonistic or agonistic effect on the T-cell depending on the region to which the antibody binds [Ellerman, 2019, Methods 154:102-117]. The spatial arrangement of the CD3 subunits plays a role in the activation and type of activation [Ellerman, 2019].

Each CD3 chain has its own unique biological function [Deng et al., 2022, Drug Discovery Today, Volume 27, Number 8]. The CD3ε subunit is involved in cerebellar development, synaptic growth, and thymic T-cell negative selection. It is a key molecule in numerous signaling pathways, including those involving the G-protein receptor signaling, apoptosis signaling and transmembrane receptor protein tyrosine kinase signaling [Deng et al., 2022, supra]. It promotes calcium-mediated signal transmission, cell-matrix adhesion, and integrin-mediated cell adhesion. Furthermore, the CD3E chain stimulates T-cell proliferation, increases interferon-gamma, interleukin 2 (IL-2) and 4 (IL-4) production.

The CD3ζ can form a complex with CD16 (an antibody Fc receptor) which is involved in the activation of macrophages and natural killer cells. It also promotes the production of IL-2 and regulates its localization. This chain is also required for the defense response to viruses by T-cells.

Besides CD3ζ, the CD3γ chain is also involved in the Fcγ signaling pathway of ADCP [Deng et al., 2022, supra]. The CD3γ chain maintains the polarity of the cell and transportation of proteins in the cell. Without the involvement of the CD3γ, lymphocyte apoptosis is not accomplished. The CD3γε heterodimer mediates T-cell activation.

### Autoimmunity and Immune Over-Reactivity

Autoimmunity arises when the immune system of the patient reacts against its own normal tissues. In humans, the autoimmune diseases commonly involve both B cells and T-cells. Although T-cells play important roles in various autoimmune diseases including those mediated primarily via autoimmune antibodies or immune complexes, there are diseases that are primarily T-cell mediated including sympathetic ophthalmia, multiple sclerosis, and type-1 diabetes mellitus. The treatment of autoimmune diseases is mainly based on immunosuppression with either corticosteroids or T-cell activation pathway antagonists [Arevalo et al., Middle East Afr J Ophthalmol 19 (1): 13-21 (2012); Galea et al., BMJ 350: h1765 (2015)].

Anti-CD3 antibodies may be used to modulate immune over-reactivity in order to re-establish immune homeostasis by i) induction of apoptosis of activated autoreactive T-cells by a process called ‘activation-induced cell death’, ii) functional inactivation (clonal anergy) by (over) stimulation in the absence of co-stimulatory signals, and/or iii) through influencing the TCR-CD3 complex to become ‘blind’ to the self-antigen [Chatenoud & Bleustone, 2007, Nat Rev Immunol, 7 (8): 622-32].

The first approved anti-CD3ε mAb (OKT3; muromonab-CD3) was a strong immunosuppressive agent due to its broad reactivity with all T-cells. The use of OKT3 was limited by its potent agonistic activity inducing severe cytokine release syndrome (CRS). In addition, its murine origin gave rise to human anti-mouse antibodies (HAMA, or anti-drug antibodies, ADA). Since then, engineered anti-CD3 mAbs have been discovered with improved tolerability and safety properties. These engineered anti-CD3 mAbs include for example foralumab, oteliximumab (also referred to as ChAglyCD3), teplizumab, and visilizumab [Kuhn & Weiner, 2016, Immunotherapy 8 (8), 889-906; Gogesch et al., 2021, Int. J. Mol. Sci., 22, 8947].

### Antitumor T Cell Response

Various attempts have been made to help the immune system to fight tumors. One early approach, in the late 19th century, involved a general stimulation of the immune system, e.g., through the administration of bacteria (live or killed) to elicit a general immune response which would also be directed against the tumor.

Recent approaches aimed at helping the immune system specifically to recognize tumor-specific antigens (TSAs) (or tumor associated antigens, TAAs) involve administration of tumor-specific antigens, typically combined with an adjuvant to the subject. However, a lack of a powerful immune response to TAAs is often observed in cancer. One of the factors responsible for the weak response to TAAs is the induction of inhibitory pathways/signals that suppress the immune response (often referred to as “immune checkpoints”). Whereas such inhibitory signals are important for maintenance of self-tolerance and to protect tissues from damage when the immune system is responding to pathogenic infection, they may also reduce what could otherwise be a helpful response by the body to the development of tumors.

A novel therapeutic era has come of age with immune checkpoint inhibitors or blockers (ICB) targeting inhibitory T-cell receptors such as CTLA-4, PD-L1 and PD-1 [Marabelle et al. Oncoimmunology vol. 5 (3): e1068494]. This burgeoning field has even been awarded by the 2018 Nobel Prize in Medicine. These immunotherapeutic agents provide good clinical results in several advanced cancers including lung [Reck et al., N Engl J Med 2016; 375:1823-1833], melanoma [Robert et al., N Engl J Med 2011; 364:2517-2526], genitourinary [Motzer et al., N Engl J Med 2018; 378:1277-1290] as well as head and neck (Ferris et al., N Engl J Med 2016; 375:1856-1867). However, primary resistance rates range from 35-44% in patients with non-small cell lung cancer (NSCLC) while secondary resistance rates approach 100% (Reck, 2016, supra).

CD3-related bispecific antibodies (bsAb) have attracted much attention in the field of cancer immunotherapy. In such bsAb constructs, one of the arms binds and activates the T-cell while the other binds a tumor-specific antigen (TAA) to guide the activated immune cell to the tumor cell, which it then can attack by releasing cytotoxic granules. Cytotoxic granules cause tumor cell membrane perforation followed by lysis and apoptosis. Most of the developed CD3×TAA bsAbs target CD3ε. Most of the first-generation CD3-engaging bsAbs bind to a similar domain as they were derived from only a few antibodies, such as OKT3, UCHT1 or TR66 [Trinklein et al., 2019, MABS, Vol. 11, No. 4, 639-652]. The resulting bsAbs with a high affinity for CD3 showed potent tumor cell killing but are associated with the release of high levels of cytokines causing CRS resulting in a narrower therapeutic window.

Given the tremendous therapeutic potential of anti-CD3 antibodies in autoimmune diseases and cancer, there is a need for novel CD3 antibodies with optimized properties.

The present description refers to a number of documents, the content of which is herein incorporated by reference in their entirety.

### SUMMARY

In various aspects and embodiments, the present disclosure provides the following items 1 to 105:

1. An antibody comprising a combination of heavy chain complementarity determining regions (CDR-H1, CDR-H2 and CDR-H3) and light chain complementarity determining regions (CDR-L1, CDR-L2 and CDR-L3), wherein said CDR-H1, CDR-H2, CDR-H3, CDR-L1, CDR-L2 and CDR-L3 comprise or consist of amino acid sequences having at least 70% identity with the sequences depicted in Table 1:

TABLE 1

| CDR-H1 | CDR-H2 | CDR-H3 | CDR-L1 | | CDR-L3 |
|---|---|---|---|---|---|
| SEQ ID | SEQ ID | SEQ ID | (SEQ ID | | (SEQ ID |
| NO: ) | NO: ) | NO: ) | NO: ) | CDR-L2 | NO: ) |
| 23 | 39 | 72 | KSY | DNT | 134 |
| 1 | 24 | 40 | 73 | QDT | 99 |
| 2 | 25 | 41 | 74 | DNI | 100 |
| 3 | 26 | 42 | 75 | DVS | 101 |
| 4 | 27 | 43 | 76 | GKN | 102 |
| 3 | 26 | 42 | 77 | EVT | 103 |
| 5 | 24 | 44 | 78 | QDN | 104 |
| 2 | 25 | 41 | 79 | DDI | 105 |
| 6 | 29 | 45 | 80 | GNN | 106 |
| 7 | 30 | 46 | 81 | QDT | 107 |
| 8 | 29 | 47 | 82 | QDS | 108 |
| 9 | 31 | 48 | 83 | KVS | 109 |
| 10 | 29 | 49 | 84 | QDT | 99 |
| 10 | 29 | 50 | 85 | QDS | 110 |
| 10 | 29 | 51 | 86 | QDT | 111 |
| 10 | 29 | 52 | 78 | QDS | 112 |
| 11 | 29 | 53 | 87 | RNN | 106 |
| 8 | 31 | 47 | 78 | QDT | 113 |
| 10 | 29 | 54 | 88 | QDS | 114 |
| 12 | 29 | 55 | 78 | QDN | 115 |
| 10 | 29 | 56 | 78 | QDN | 116 |
| 13 | 29 | 57 | 89 | RNN | 117 |
| 10 | 29 | 58 | 90 | STF | 118 |
| 10 | 29 | 59 | 73 | QDN | 119 |
| 10 | 29 | 59 | 91 | TNN | 117 |
| 10 | 29 | 60 | 92 | GNS | 120 |
| 14 | 29 | 61 | 85 | QDD | 121 |
| 10 | 29 | 62 | 78 | QDK | 122 |
| 15 | 26 | 63 | 93 | RND | 123 |
| 16 | 32 | 64 | 94 | KVS | 124 |
| 10 | 29 | 65 | 73 | EDN | 125 |
| 17 | 33 | 66 | SSD | YSD | 126 |
| 17 | 33 | 66 | SSD | YSD | 126 |
| 17 | 33 | 66 | SSD | YSD | 133 |
| 18 | 34 | 67 | TYD | YND | 127 |
| 19 | 35 | 68 | 95 | SND | 128 |
| 710 | 711 | 712 | 96 | NND | 129 |
| 20 | 36 | 69 | 97 | NNN | 130 |
| 21 | 37 | 70 | INN | QND | 131 |
| 22 | 38 | 71 | 98 | SND | 132 |
| 28 | 26 | 696 | 697 | RNN | 698 |

or an antigen-binding fragment thereof.
   2. The antibody or antigen-binding fragment thereof of item 1, wherein the CDR-H1, CDR-H2, CDR-H3, CDR-L1, CDR-L2 and CDR-L3 comprise or consist of amino acid sequences having at least 80% identity with the sequences depicted in Table 1.
   3. The antibody or antigen-binding fragment thereof of item 1, wherein the CDR-H1, CDR-H2, CDR-H3, CDR-L1, CDR-L2 and CDR-L3 comprise or consist of amino acid sequences having at least 90% identity with the sequences depicted in Table 1.
   4. The antibody or antigen-binding fragment thereof of item 1, wherein the CDR-H1, CDR-H2, CDR-H3, CDR-L1, CDR-L2 and CDR-L3 comprise or consist of the sequences depicted in Table 1.
   5. The antibody or antigen binding fragment thereof according to any one of items 1 to 4, which further comprises a heavy chain framework region 1 (VH FR1) comprising an amino acid sequence having at least 60% identity with one of the following sequences:

(SEQ ID NO: 392)
QVQLLQSAAEVKKPGESLKISCKGS,
(SEQ ID NO: 135)
QVQLVQSGAEVKKPGESLRISCKGS,
(SEQ ID NO: 136)
QVQLQQSGPGLVKPAQTLSLTCDIS,
(SEQ ID NO: 137)
QVQLQQSGGGVVQPGRSLRLSCAAS,
(SEQ ID NO: 138)
QVQLQQSGPGLVKPSQTLSVTCVIS,
(SEQ ID NO: 139)
QVQLVQSGAEVKKPGESLKISCTGS,
(SEQ ID NO: 140)
EVOLVESGAEVKKPGESLRISCKGS,
(SEQ ID NO: 141)
QVQLVESGAEVKKPGESLKISCKGS,
(SEQ ID NO: 142)
EVQLVQSGGGLVKPGGSLRLSCAAS,
(SEQ ID NO: 143)
QVQLVQSGAEVKKPGEPLKISCKGS,
(SEQ ID NO: 144)
EVQLVQSGAEVKEPGASVKVSCKAS,
(SEQ ID NO: 145)
EVQLVQSGAEVKKPGESLKISCKGS,
(SEQ ID NO: 146)
QVQLVQSGAEVKKPGESLKISCKGS,
(SEQ ID NO: 147)
QVQLVQSGAEVKKPGESLKISCKGS,
(SEQ ID NO: 148)
EVQLVQSGAEVKKPGESLKISCKGS,
(SEQ ID NO: 149)
EVQLVQSGAEVKKPGESLKISCQGS,
(SEQ ID NO: 150)
QVQLVESGAEVKKPGEPLKISCKGS,
(SEQ ID NO: 151)
EVQLVQSGAEVKKPGESLKISCKGS,
(SEQ ID NO: 152)
QMQLVQSGAEVKKPGESLKISCKGS,
(SEQ ID NO: 153)
EVQLVQSGAEVKKPGESLKISCKGS,
(SEQ ID NO: 154)
EVQLVQSGAEVKKPGESLKISCKGS,
(SEQ ID NO: 155)
EVQLVQSGAEVKKPGESLKISCKGS,
(SEQ ID NO: 156)
EVQLVQSGAEVKKPGESLKISCKGS,
(SEQ ID NO: 157)
QVQLVQSGAEVKKPGESLKISCKGS,
(SEQ ID NO: 158)
QMQLVQSGAEVKKPGESLKISCKGS,
(SEQ ID NO: 159)
QVQLVQSGAEVKKPGESLKISCKGF,
(SEQ ID NO: 160)
QVQLVQSGAEVKKPGESLKISCKGS,
(SEQ ID NO: 161)
QVQLQQSGAGLVKASQTLALTCAIS,
(SEQ ID NO: 162)
QVQLVQSGGGVVQPGRSLRLSCAAS,
(SEQ ID NO: 163)
EVQLVQSGAEVKKPGESLKISCKGS,
(SEQ ID NO: 164)
AVTLDESGGGLQTPGGALSLVCKAS,
(SEQ ID NO: 165)
AVTLDESGGGLQTPGGGLSLVCKAS,
or
(SEQ ID NO: 166)
AVTLDESGGGLQTPGGPLSLVCEGS.

6. The antibody or antigen binding fragment thereof according to any one of items 1 to 5, which further comprises a heavy chain framework region 2 (VH FR2) comprising an amino acid sequence having at least 60% identity with one of the following sequences:

(SEQ ID NO: 167)
IGWVRQMPGKGLEWVGI,
(SEQ ID NO: 168)
INWVRQMPGKGLEWMGR,
(SEQ ID NO: 169)
WHWIRQSPSRGLEWLGR,
(SEQ ID NO: 170)
MHWVRQAPGKGLEWVAV,
(SEQ ID NO: 171)
IGWVRQMPGKGLEWMGM,
(SEQ ID NO: 172)
INWVRQMPGKGLEWMGR,
(SEQ ID NO: 173)
IAWVRQMPGKGLEWMGI,
(SEQ ID NO: 174)
MSWVRQAPGKGLEWVGR,
(SEQ ID NO: 175)
MHWVRQAPGQRLEWMGW,
(SEQ ID NO: 176)
IGWVRQMPGKGLEWMGI,
(SEQ ID NO: 177)
IGWVRQMPGKGLEWLGI,
(SEQ ID NO: 178)
WNWIRQSPSRGLEWLGR,
(SEQ ID NO: 179)
MVWVRQAPGKGLEFVAG,
(SEQ ID NO: 180)
MGWVRQAPGKGLEFIAS,
(SEQ ID NO: 181)
MAWVRQAPGKGLEYVGL,
(SEQ ID NO: 182)
MFWVRQAPGKGLEFVAH,
(SEQ ID NO: 183)
MQWVRQAPGKGLEWVAG,
(SEQ ID NO: 184)
MAWVRQAPGKRLEWVAS,
(SEQ ID NO: 185)
MAWVRQAPGKGLEFVAG,
or
(SEQ ID NO: 186)
MFWVRQAPGKGLEYVAY.

7. The antibody or antigen binding fragment thereof according to any one of items 1 to 6, which further comprises a heavy chain framework region 3 (VH FR3) comprising an amino acid sequence having at least 60% identity with one of the following sequences:

(SEQ ID NO: 187)
RYSPSFQGQVTISADTSISTTYLQWSSLKASDTAIYYC,
(SEQ ID NO: 188)
NYSPSFQGHVIFSADKSSNTVYLQWSSLEASDTAMYYC,
(SEQ ID NO: 189)
YSDYGESVKSRVVINADTSNNQISLQLSSVTPEDTAAYYC,
(SEQ ID NO: 190)
YYADSVKGRFTISRDNSRNTLDLQMNSLRAEDTAVYYC,
(SEQ ID NO: 191)
RYSPSFQGQVTISADKSINTAYLQWTSLRASDSAIYYC,
(SEQ ID NO: 192)
NYSPSFQGHVIFSADKSSNTVYLQWSSLEASDTAMYYC,
(SEQ ID NO: 193)
RYSPSFQGQVTISADKSISTAYLQWSSLKASDTAMYYC,
(SEQ ID NO: 194)
DYAAPVKGRFTISRDDSKNTLYLQMNSLKTEDTAVYYC,
(SEQ ID NO: 195)
RYSPSFQGQVTISADKSISTAYLQWGSLRASDTAMYYC,
(SEQ ID NO: 196)
NYAQKFQGRVTMTRDTSISTAYMELSSLRSDDTAVYYC,
(SEQ ID NO: 197)
RYSPSFQGQVTISADKSISTAYLQWGSLRASDTAMYYC,
(SEQ ID NO: 198)
RYSPSFQGQVTISADKSITTAYLQWSSLKASDTAMYYC,
(SEQ ID NO: 199)
RYSPSFQGQVTISADKSISTAYLQWSSLKASDTAIYYC,
(SEQ ID NO: 200)
KYGAAVTGRATISRDNGQSTVRLQLNNLRAEDTGTYFC,
(SEQ ID NO: 201)
YYGTAVKGRATISRDNGQSTVRLQLNNLRAEDTATYYC,
(SEQ ID NO: 202)
DYGAAVKGRATISRDDGQSTVRLQLNNLRAEDTATYFC,
(SEQ ID NO: 203)
NYGSAVKGRATISRDNGQSTLRLQLNNLRAEDTGIYFC,
(SEQ ID NO: 204)
YYGAAVKGRATISRDNGQSTVRLQLNNLRAEDTGIYYC,
(SEQ ID NO: 205)
TYGAAVKGRATISRDDGQSTLRLQLNNLRAEDTATYYC,
(SEQ ID NO: 206)
YYAPAVKGRATISRDNGQSTVRLQLNNLRAADTGTYFC,
(SEQ ID NO: 207)
FYGAAVKGRATISRDNGQSTVRLQLNNLRAEDTATYFC,
or
(SEQ ID NO: 699)
YTDFAVSVKSRITINPDTSKNQFSLQLNSVTPEDTAVYYC.

8. The antibody or antigen binding fragment thereof according to any one of items 1 to 7, which further comprises a heavy chain framework region 4 (VH FR4) comprising an amino acid sequence having at least 60% identity with one of the following sequences: WGQGTTVTVSS (SEQ ID NO:208), WGQGTLVTVSS (SEQ ID NO:209), WGQGTMVTVSS (SEQ ID NO:210), or WGHGTEVIVSS (SEQ ID NO:211).
9. The antibody or antigen binding fragment thereof according to any one of items 1 to 8, which further comprises a light chain framework region 1 (VL FR1) comprising or consisting of an amino acid sequence having at least 60% identity with one of the following sequences:

(SEQ ID NO: 212)
QSVLTQPPSLSVSPRQTASITCSGD,
(SEQ ID NO: 213)
QSVLTQPPSVSGAPGQRVTISCTGS,
(SEQ ID NO: 214)
QSVVTQPASVSGSPGQSITISCTGT,
(SEQ ID NO: 215)
LSELTQDPAVSVALGQTVRITCQGD,
(SEQ ID NO: 216)
QSVLTQPPSASGSPGQSVTISCTGT,
(SEQ ID NO: 217)
SYVLTQPPSVSVSPGQTARITCSGD,
(SEQ ID NO: 218)
QSVLTQPSSVSGAPGQRVTISCTGS,
(SEQ ID NO: 219)
QAGLTQPPSASGTPGQRVTISCSGS,
(SEQ ID NO: 220)
QSVLTQPPSVSVSPGQTASITCSGD,
(SEQ ID NO: 221)
SYELMQPPSVSVSPGQTASITCSGD,
(SEQ ID NO: 222)
DIVMTQTPLSLPVTLGQPASISCRSS,
(SEQ ID NO: 223)
QAGLTQPPSVSVSPGQTVTITCSAD,
(SEQ ID NO: 224)
LPVLTQPPSVSVSPGQTASITCSGD,
(SEQ ID NO: 225)
QPVLTQPPSVSVSPGQTASITCSGD,
(SEQ ID NO: 226)
QPVLTQPPSASGTPGQRVSISCSGS,
(SEQ ID NO: 227)
QAGLTQPPSVSVSPGQTAIIPCSGE,
(SEQ ID NO: 228)
SYVLTQPPSVSVSPGQTATITCSGD,
(SEQ ID NO: 229)
QAVLTQPPSASGTPGQRVTISCSGS,
(SEQ ID NO: 230)
QPVLTQPPSASGTPGQRVTISCSGS,
(SEQ ID NO: 231)
LPVLTQPPSASGTPGQRVTISCSGS,
(SEQ ID NO: 232)
QSVVTQPPSVSGTPGQRVSISCSGS,
(SEQ ID NO: 233)
QAGLTQPPSVSVSPGQTASITCSGE,
(SEQ ID NO: 234)
SYELMQPPSLSVSPGQTASITCSGD,
(SEQ ID NO: 235)
QSVLTQPPSASGTPGQRVTISCSGS,
(SEQ ID NO: 236)
DIQMTQTPLSLSVTLGQPASISCRSS,
(SEQ ID NO: 237)
QSVLTQPPSVSVSPGQTASITCSGN,
(SEQ ID NO: 238)
QAALTQPASVSANPGETVKITCSGG,
(SEQ ID NO: 239)
QAALTQPSSVSANPGETVKITCSGP,
(SEQ ID NO: 240)
QAALTQPASVSANLGGTVEITCSGG,
(SEQ ID NO: 241)
QAALTQPSSVSANPGETVKITCSGS,
(SEQ ID NO: 242)
QAALTQPASVSANPGETVKITCSGY,
or
(SEQ ID NO: 243)
QAALTQPASVSSNPGETVKITCSGG.

10. The antibody or antigen binding fragment thereof according to any one of items 1 to 9, which further comprises a light chain framework region 2 (VL FR2) comprising or consisting of an amino acid sequence having at least 60% identity with one of the following sequences:

(SEQ ID NO: 244)
VSWYQQKPGQSPVLVIY,
(SEQ ID NO: 245)
VHWYQQVAGLAPKLLIH,
(SEQ ID NO: 246)
VSWYQQHPGKAPKLMIF,
(SEQ ID NO: 247)
ASWYQQKPGQAPVLVIY,
(SEQ ID NO: 248)
VSWYQQHPGKAPKLILY,
(SEQ ID NO: 249)
VSWYQLKPGQSPVVVIY,
(SEQ ID NO: 250)
VHWYQQLPGTAPKLLIY,
(SEQ ID NO: 251)
VSWYRQIPGTAPKFLLY,
(SEQ ID NO: 252)
ASWYRQKPGQSPVLVIY,
(SEQ ID NO: 253)
LNWFRQRPGQSPRRLIY,
(SEQ ID NO: 254)
TSWYQQRPGQSPVLVIY,
(SEQ ID NO: 255)
ASWYQQKPGQSPVLVIY,
(SEQ ID NO: 256)
VSWFQQKPGQSPVLVIY,
(SEQ ID NO: 257)
VYWYQQLPGTAPKLLIY,
(SEQ ID NO: 258)
VSWYQQKPGESPLSIIY,
(SEQ ID NO: 259)
ASWYQQKPGQSPVLVIF,
(SEQ ID NO: 260)
ASWYQQKPGQSPVLIIY,
(SEQ ID NO: 261)
VHWYQQLPGAAPKVLIY,
(SEQ ID NO: 262)
VNWYQQFPGTAPKLLMY,
(SEQ ID NO: 263)
VSWYQQLPGSAPKLLIY,
(SEQ ID NO: 264)
VTWFQQLPGTAPKLLIY,
(SEQ ID NO: 265)
VSWYQQRPGQSPVLVMY,
(SEQ ID NO: 266)
VSWYQQLPGTAPKLLIY,
(SEQ ID NO: 267)
LNWFQQRPGQSPRRLIY,
(SEQ ID NO: 268)
YGWFQQKSPGSAPVTVIY,
(SEQ ID NO: 269)
YGWYQQKSPGSAPVTVIY,
(SEQ ID NO: 270)
YGWYQQKSPGSAPVTLIY,
or
(SEQ ID NO: 700)
VYWYQQLPGAAPKLLIY.

11. The antibody or antigen binding fragment thereof according to any one of items 1 to 10, which further comprises a light chain framework region 3 (VL FR3) comprising or consisting of an amino acid sequence having at least 60% identity with one of the following sequences:

(SEQ ID NO: 271)
KRPSGIPERFSGSNSGNTATLTISGTQAMDEADYYC,
(SEQ ID NO: 272)
NRPSGVPDRFSGSKSGASASLTITGLQAEDEADYYC,
(SEQ ID NO: 273)
NRPSGVSNRFSGSKSGNTASLTISGLQADDEADYYC,
(SEQ ID NO: 274)
NRPSGIPDRFSGSDSGKTASLTITGAQAEDEADYYC,
(SEQ ID NO: 275)
KRPSGVPDRFSGSKSGNTASLTVSGLQAEDEADYYC,
(SEQ ID NO: 276)
KRPSGIPERFSASNSENTGTLTISGTQAMDEADYYC,
(SEQ ID NO: 277)
NRPSGVPDRFSGSKSGTSASLAITGLRAEDEADYYC,
(SEQ ID NO: 278)
QRPSGVPARFSGSKSGTSASLAVDGLQSDDEADYYC,
(SEQ ID NO: 279)
KRPSGIPERFSGSNSGNTATLTISGTQAMDEGDYYC,
(SEQ ID NO: 280)
NRDSGVPGTFSGSGSGTDFTLKISRVEADDVGVYFC,
(SEQ ID NO: 281)
KRPSGIPARFSGSNSGNTATLTISGTQAMDEADYYC,
(SEQ ID NO: 282)
KRPSGIPERFSGSNSGSTATLTITGTQAMDDADYYC,
(SEQ ID NO: 283)
KRPSGIPERFSGSNSGSTATLTISGTQALDEADYYC,
(SEQ ID NO: 284)
QRPSGVPDRFSGSKSGTSASLAISGLRSEDEADYYC,
(SEQ ID NO: 285)
ERPSGIPERFSGSNSGNTATLTIRGTQAMDEADYYC,
(SEQ ID NO: 286)
KRPSGIPERFSGSNSGNTATLTISGAQAVDEADYYC,
(SEQ ID NO: 287)
QRPSGVPDRFSGSKSGTSASLAISGLRSEDETDYFC,
(SEQ ID NO: 288)
QRPSGVPDRFSGSKSGTSASLAISGLQSEDEADYYC,
(SEQ ID NO: 289)
KRPSRVPERFSGSNSGNTATLTISETQAMDEADYYC,
(SEQ ID NO: 290)
KRPSGVPERFSGSNSGNTATLNISGTQPMDEADYYC,
(SEQ ID NO: 291)
KRPSGIPKRFSGSNSGNTATLTISGTQAMDEADYYC,
(SEQ ID NO: 292)
KRPSGVPERFSGSKSGTSASLAISGLQSEDEADYYC,
(SEQ ID NO: 293)
NRDSGVPDRFSGSGSGTDFTLKISRVEAEDVGVYYC,
(SEQ ID NO: 294)
KRPSDIPSRFSGSASGSMGALIITGVQAEDEAVYFC,
(SEQ ID NO: 295)
NRPSDIPSRFSGSKSGSTATLTITGVQAEDEAVYYC,
(SEQ ID NO: 296)
KRPSDIPSRFSGSASGSTATLTITGVQADDEAVYYC,
(SEQ ID NO: 297)
KRPSDIPSRFSGSLSGSTNTLTITGVQVEDESVYFC,
(SEQ ID NO: 298)
KRPSDIPSRFSGSTSGSMGALIITGVQAEDEAVYFC,
(SEQ ID NO: 299)
NRPSDIPSRFSGSLSDSTHTLTITGVRAEDEAVYFC,
(SEQ ID NO: 300)
KRPSDIPSRFSGSKSGSTGTLTITGVQAEDEAVYFC,
(SEQ ID NO: 301)
KRPSDIPSRFSGSTSGSTNTLTITGVQVEDEAVYFC,
or
(SEQ ID NO: 302)
NRPSNIPSRFSGSTSGSTATLTITGVQADDEAVYYC.

12. The antibody or antigen binding fragment thereof according to any one of items 1 to 11, which further comprises a light chain framework region 4 (VL FR4) comprising or consisting of an amino acid sequence having at least 60% identity with one of the following sequences: FGGGTKLTVL (SEQ ID NO:303), FGTGTKVTVL (SEQ ID NO:304), FGGGTKVTVL (SEQ ID NO: 305), FGTGTKLTVL (SEQ ID NO:306), FGSGTKLTVL (SEQ ID NO:307), FGQGTKVEIK (SEQ ID NO:308), FGTGTQLTVL (SEQ ID NO:309), FGAGTKVTVL (SEQ ID NO:310), FGGGTQLTVL (SEQ ID NO:311), FGAGTTLTVL (SEQ ID NO:312), or YGAGTTLTVL (SEQ ID NO: 313).
13. The antibody or antigen binding fragment thereof according to any one of items 1 to 12, wherein said antibody comprises one of the following combinations of heavy chain variable region (HCVR) and light chain variable region (LCVR):

| HCVR | LCVR |
|---|---|
| AVTLDESGGGLQTPGGALSLVCKASGFTFSS | QAALTQPASVSANPGETVKITCSGYKSY |
| SGMAWVRQAPGKGLEFVAGITSSAGNTYYAP | YGWYQQKSPGSAPVTVIYDNTNRPSNIP |
| AVKGRATISRDNGQSTVRLQLNNLRAADTGTY | SRFSGSTSGSTATLTITGVQADDEAVYY |
| FCAKDSNSGSPDASEIDAWGHGTEVIVSS | CGNADHSYVGVFGAGTTLTVL (SEQ ID |
| (SEQ ID NO: 390) | NO: 391) |
| QVQLLQSAAEVKKPGESLKISCKGSGYRFTSY | QSVLTQPPSLSVSPRQTASITCSGDKLG |
| WIGWVRQMPGKGLEWVGIIYPDDSDTRYSPS | DKFVSWYQQKPGQSPVLVIYQDTKRPS |
| FQGQVTISADTSISTTYLQWSSLKASDTAIYYC | GIPERFSGSNSGNTATLTISGTQAMDEA |
| ASGEVAFDVWGQGTTVTVSS (SEQ ID | DYYCQAWDSSTGVFGGGTKLTVL (SEQ |
| NO: 314) | ID NO: 315) |
| QVQLVQSGAEVKKPGESLRISCKGSGYSFTN | QSVLTQPPSVSGAPGQRVTISCTGSSSN |
| NWINWVRQMPGKGLEWMGRIDPDDSYTNYS | IGAGFNVHWYQQVAGLAPKLLIHDNINRP |
| PSFQGHVIFSADKSSNTVYLQWSSLEASDTA | SGVPDRFSGSKSGASASLTITGLQAEDE |
| MYYCARRAQGGGGYDYWGQGTLVTVSS | ADYYCQSYDSSLSGWFAFGTGTKVTVL |
| (SEQ ID NO: 316) | (SEQ ID NO: 317) |
| QVQLQQSGPGLVKPAQTLSLTCDISGDSVSSK | QSVVTQPASVSGSPGQSITISCTGTSSDI |
| SAAWHWIRQSPSRGLEWLGRTYYRSKWYSD | GGYDYVSWYQQHPGKAPKLMIFDVSNR |
| YGESVKSRVVINADTSNNQISLQLSSVTPEDT | PSGVSNRFSGSKSGNTASLTISGLQADD |
| AAYYCARGIGSLNFDIWGQGTMVTVSS (SEQ | EADYYCSSYRAGSTLNLLFGGGTKLTVL |
| ID NO: 318) | (SEQ ID NO: 319) |
| QVQLQQSGGGVVQPGRSLRLSCAASGFTFS | LSELTQDPAVSVALGQTVRITCQGDSLR |
| NYAMHWVRQAPGKGLEWVAVISYDGSNEYY | SYYASWYQQKPGQAPVLVIYGKNNRPS |
| ADSVKGRFTISRDNSRNTLDLQMNSLRAEDTA | GIPDRFSGSDSGKTASLTITGAQAEDEA |
| VYYCARGTNYGMDVWGQGTMVTVSS (SEQ | DYYCQSRDTTGNYLLFGGGTKLTVL |
| ID NO: 320) | (SEQ ID NO: 321) |
| QVQLQQSGPGLVKPSQTLSVTCVISGDSVSS | QSVLTQPPSASGSPGQSVTISCTGTSGD |
| KSAAWHWIRQSPSRGLEWLGRTYYRSKWYS | VGGYNYVSWYQQHPGKAPKLILYEVTKR |
| DYGESVKSRVVINADTSNNQISLQLSSVTPED | PSGVPDRFSGSKSGNTASLTVSGLQAE |
| TAAYYCARGIGSLNFDIWGQGTMVTVSS (SEQ | DEADYYCSSYGGPNNLVLFGGGTKVTVL |
| ID NO: 322) | (SEQ ID NO: 323) |
| QVQLVQSGAEVKKPGESLKISCTGSGYRFSH | SYVLTQPPSVSVSPGQTARITCSGDKLG |
| YWIGWVRQMPGKGLEWMGMIYPDDSDTRYS | DKYVSWYQLKPGQSPVVVIYQDNKRPS |
| PSFQGQVTISADKSINTAYLQWTSLRASDSAIY | GIPERFSASNSENTGTLTISGTQAMDEA |
| YCATGVISGTDYWGQGTLVTVSS (SEQ ID | DYYCQTWDSGTGVFGTGTKLTVL (SEQ |
| NO: 324) | ID NO: 325) |
| EVQLVESGAEVKKPGESLRISCKGSGYSFTNN | QSVLTQPSSVSGAPGQRVTISCTGSSSN |
| WINWVRQMPGKGLEWMGRIDPDDSYTNYSP | IGAGYGVHWYQQLPGTAPKLLIYDDINRP |
| SFQGHVIFSADKSSNTVYLQWSSLEASDTAM | SGVPDRFSGSKSGTSASLAITGLRAEDE |
| YYCARRAQGGGGYDYWGQGTLVTVSS (SEQ | ADYYCQSYDTSRGGLYVFGSGTKLTVL |
| ID NO: 326) | (SEQ ID NO: 327) |
| QVQLVESGAEVKKPGESLKISCKGSGYSFTTY | QAGLTQPPSASGTPGQRVTISCSGSSSN |
| WIAWVRQMPGKGLEWMGIIYPGDSDTRYSPS | IGSNTVSWYRQIPGTAPKFLLYGNNQRP |
| FQGQVTISADKSISTAYLQWSSLKASDTAMYY | SGVPARFSGSKSGTSASLAVDGLQSDD |
| CARLGGGMDVWGQGTMVTVSS (SEQ ID | EADYYCAAWDDSLSGYVFGTGTKLTVL |
| NO: 328) | (SEQ ID NO: 329) |
| EVQLVQSGGGLVKPGGSLRLSCAASGFTFSN | QSVLTQPPSVSVSPGQTASITCSGDKLG |
| AWMSWVRQAPGKGLEWVGRIKSKTDGGTTD | NKYASWYRQKPGQSPVLVIYQDTKRPS |
| YAAPVKGRFTISRDDSKNTLYLQMNSLKTEDT | GIPERFSGSNSGNTATLTISGTQAMDEG |
| AVYYCTTENEVWPSLDVWGQGTTVTVSS | DYYCQAWDSSTAVVFGGGTKLTVL |
| (SEQ ID NO: 330) | (SEQ ID NO: 331) |
| QVQLVQSGAEVKKPGEPLKISCKGSGYSFPTY | SYELMQPPSVSVSPGQTASITCSGDKLG |
| WIAWVRQMPGKGLEWMGIIYPGDSDTRYSPS | TKYVSWYQQKPGQSPVLVIYQDSKRPS |
| FQGQVTISADKSISTAYLQWGSLRASDTAMYY | GIPERFSGSNSGNTATLTISGTQAMDEA |
| CARGGSGLDYWGQGTLVTVSS (SEQ ID | DYYCQAWDSSTVVFGGGTKLTVL (SEQ |
| NO: 332) | ID NO: 333) |
| EVQLVQSGAEVKEPGASVKVSCKASGYTFTN | DIVMTQTPLSLPVTLGQPASISCRSSQSL |
| YAMHWVRQAPGQRLEWMGWINPNTGGTNY | VDRDGNTYLNWFRQRPGQSPRRLIYKV |
| AQKFQGRVTMTRDTSISTAYMELSSLRSDDTA | SNRDSGVPGTFSGSGSGTDFTLKISRVE |
| VYYCARGGGGLVFWGQGTLVTVSS (SEQ ID | ADDVGVYFCMQATHWPPTFGQGTKVEI |
| NO: 334) | K (SEQ ID NO: 335) |
| EVQLVQSGAEVKKPGESLKISCKGSGYSFTSY | QAGLTQPPSVSVSPGQTVTITCSADNLG |
| WIGWVRQMPGKGLEWMGIIYPGDSDTRYSPS | DKYTSWYQQRPGQSPVLVIYQDTKRPS |
| FQGQVTISADKSISTAYLQWSSLKASDTAMYY | GIPARFSGSNSGNTATLTISGTQAMDEA |
| CATPMEDGMDVWGQGTTVTVSS (SEQ ID | DYYCQAWDSSTGVFGTGTKVTVL (SEQ |
| NO: 336) | ID NO: 337) |
| QVQLVQSGAEVKKPGESLKISCKGSGYSFTSY | LPVLTQPPSVSVSPGQTASITCSGDRLG |
| WIGWVRQMPGKGLEWMGIIYPGDSDTRYSPS | DKYASWYQQKPGQSPVLVIYQDSKRPS |
| FQGQVTISADKSISTAYLQWSSLKASDTAMYY | GIPERFSGSNSGNTATLTISGTQAMDEA |
| CARGGPPDYWGQGTLVTVSS (SEQ ID | DYYCQAWDSSTAVFGGGTKLTVL (SEQ |
| NO: 338) | ID NO: 339) |
| QVQLVQSGAEVKKPGESLKISCKGSGYSFTSY | QPVLTQPPSVSVSPGQTASITCSGDKLG |
| WIGWVRQMPGKGLEWMGIIYPGDSDTRYSPS | NKFVSWFQQKPGQSPVLVIYQDTKRPS |
| FQGQVTISADKSISTAYLQWSSLKASDTAMYY | GIPERFSGSNSGSTATLTITGTQAMDDA |
| CTRSSFNDHWGQGTLVTVSS (SEQ ID | DYYCQAWNTNTALFGGGTKLTVL (SEQ |
| NO: 340) | ID NO: 341) |
| EVQLVQSGAEVKKPGESLKISCKGSGYSFTSY | QSVLTQPPSVSVSPGQTASITCSGDKLG |
| WIGWVRQMPGKGLEWMGIIYPGDSDTRYSPS | DKYVSWYQQKPGQSPVLVIYQDSKRPS |
| FQGQVTISADKSISTAYLQWSSLKASDTAMYY | GIPERFSGSNSGSTATLTISGTQALDEAD |
| CARLGGSLPIDYWGQGTLVTVSS (SEQ ID | YYCQAWDNNIGVFGGGTKLTVL (SEQ ID |
| NO: 342) | NO: 343) |
| EVQLVQSGAEVKKPGESLKISCQGSGYTFTNY | QPVLTQPPSASGTPGQRVSISCSGSNSN |
| WIAWVRQMPGKGLEWMGIIYPGDSDTRYSPS | IGSNSVYWYQQLPGTAPKLLIYRNNQRP |
| FQGQVTISADKSISTAYLQWSSLKASDTAMYY | SGVPDRFSGSKSGTSASLAISGLRSEDE |
| CAIGYDYGAFDIWGQGTMVTVSS (SEQ ID | ADYYCAAWDDSLSGYVFGTGTQLTVL |
| NO: 344) | (SEQ ID NO: 345) |
| QVQLVESGAEVKKPGEPLKISCKGSGYSFPTY | QAGLTQPPSVSVSPGQTAIIPCSGEKLG |
| WIAWVRQMPGKGLEWMGIIYPADSDTRYSPS | DKYVSWYQQKPGESPLSIIYQDTERPSGI |
| FQGQVTISADKSISTAYLQWGSLRASDTAMYY | PERFSGSNSGNTATLTIRGTQAMDEADY |
| CARGGSGLDYWGQGTLVTVSS (SEQ ID | YCQAWDSNTMVFGGGTKLTVL (SEQ ID |
| NO: 346) | NO: 347) |
| EVQLVQSGAEVKKPGESLKISCKGSGYSFTSY | SYVLTQPPSVSVSPGQTATITCSGDKLG |
| WIGWVRQMPGKGLEWMGIIYPGDSDTRYSPS | DEYVSWYQQKPGQSPVLVIYQDSKRPS |
| FQGQVTISADKSISTAYLQWSSLKASDTAMYY | GIPERFSGSNSGNTATLTISGAQAVDEA |
| CASRLLFGDDAFDIWGQGTMVTVSS (SEQ ID | DYYCQAWDSNAGAFGTGTKLTVL (SEQ |
| NO: 348) | ID NO: 349) |
| QMQLVQSGAEVKKPGESLKISCKGSGYSFST | QSVLTQPPSVSVSPGQTASITCSGDKLG |
| NWIGWVRQMPGKGLEWMGIIYPGDSDTRYSP | DKYASWYQQKPGQSPVLVIFQDNKRPS |
| SFQGQVTISADKSISTAYLQWSSLKASDTAMY | GIPERFSGSNSGNTATLTISGTQAMDEA |
| YCARFYDSSGNDYWGQGTLVTVSS (SEQ ID | DYYCQTWDSSTAIFGGGTKLTVL (SEQ |
| NO: 350) | ID NO: 351) |
| EVQLVQSGAEVKKPGESLKISCKGSGYSFTSY | QSVLTQPPSVSVSPGQTASITCSGDKLG |
| WIGWVRQMPGKGLEWMGIIYPGDSDTRYSPS | DKYASWYQQKPGQSPVLIIYQDNKRPSG |
| FQGQVTISADKSISTAYLQWSSLKASDTAMYY | IPERFSGSNSGNTATLTISGTQAMDEAD |
| CVVRPAAAGTRIDYWGQGTLVTVSS (SEQ ID | YYCQAWDSSTYVFGTGTKLTVL (SEQ ID |
| NO: 352) | NO: 353) |
| EVQLVQSGAEVKKPGESLKISCKGSRYSFTSY | QAVLTQPPSASGTPGQRVTISCSGSSSN |
| WIGWVRQMPGKGLEWMGIIYPGDSDTRYSPS | IEGNYVHWYQQLPGAAPKVLIYRNNQRP |
| FQGQVTISADKSISTAYLQWSSLKASDTAMYY | SGVPDRFSGSKSGTSASLAISGLRSEDE |
| CARLKDGAFDIWGQGTMVTVSS (SEQ ID | TDYFCAAWDDSLNGYVFGAGTKVTVL |
| NO: 354) | (SEQ ID NO: 355) |
| EVQLVQSGAEVKKPGESLKISCKGSGYSFTSY | QPVLTQPPSASGTPGQRVTISCSGSSSN |
| WIGWRQMPGKGLEWMGIIYPGDSDTRYSPS | IGTHTVNWYQQFPGTAPKLLMYSTFQRP |
| FQGQVTISADKSISTAYLQWSSLKASDTAMYY | SGVPDRFSGSKSGTSASLAISGLQSEDE |
| CASWGMDVWGQGTLVTVSS (SEQ ID | ADYYCAAWDDSLNGPVFGGGTQLTVL |
| NO: 356) | (SEQ ID NO: 357) |
| EVQLVQSGAEVKKPGESLKISCKGSGYSFTSY | QSVLTQPPSVSVSPGQTASITCSGDKLG |
| WIGWVRQMPGKGLEWMGIIYPGDSDTRYSPS | DKFASWYQQKPGQSPVLVIYQDNKRPS |
| FQGQVTISADKSISTAYLQWSSLKASDTAMYY | RVPERFSGSNSGNTATLTISETQAMDEA |
| CARHDYGMDVWGQGTTVTVSS (SEQ ID | DYYCQAWVNNTVVFGGGTKVTVL (SEQ |
| NO: 358) | ID NO: 359) |
| QVQLVQSGAEVKKPGESLKISCKGSGYSFTSY | LPVLTQPPSASGTPGQRVTISCSGSRSN |
| WIGWVRQMPGKGLEWMGIIYPGDSDTRYSPS | VGGNTVSWYQQLPGSAPKLLIYTNNQRP |
| FQGQVTISADKSISTAYLQWSSLKASDTAMYY | SGVPDRFSGSKSGTSASLAISGLQSEDE |
| CARHDYGMDVWGQGTMVTVSS (SEQ ID | ADYYCAAWDDSLNGYVFGTGTQLTVL |
| NO: 360) | (SEQ ID NO: 361) |
| QMQLVQSGAEVKKPGESLKISCKGSGYSFTS | QSVVTQPPSVSGTPGQRVSISCSGSSSN |
| YWIGWVRQMPGKGLEWLGIIYPGDSDTRYSP | IGSHSVTWFQQLPGTAPKLLIYGNSQRP |
| SFQGQVTISADKSISTAYLQWSSLKASDTAMY | SGVPDRFSGSKSGTSASLAISGLQSEDE |
| YCARLGQGSGPFDYWGQGTLVTVSS (SEQ ID | ADYYCASWDDSLNGYVFGTGTKLTVL |
| NO: 362) | (SEQ ID NO: 363) |
| QVQLVQSGAEVKKPGESLKISCKGFGYSFTNY | QAGLTQPPSVSVSPGQTASITCSGERLG |
| WIGWVRQMPGKGLEWMGIIYPGDSDTRYSPS | DKYVSWYQQKPGQSPVLVIYQDDKRPS |
| FQGQVTISADKSITTAYLQWSSLKASDTAMYY | GVPERFSGSNSGNTATLNISGTQPMDEA |
| CASHTKDGTWDIWGQGTMVTVSS (SEQ ID | DYYCQAWDSSTSLYVFGTGTKLTVL |
| NO: 364) | (SEQ ID NO: 365) |
| QVQLVQSGAEVKKPGESLKISCKGSGYSFTSY | SYELMQPPSLSVSPGQTASITCSGDKLG |
| WIGWRQMPGKGLEWMGIIYPGDSDTRYSPS | DKYVSWYQQRPGQSPVLVMYQDKKRP |
| FQGQVTISADKSISTAYLQWSSLKASDTAIYYC | SGIPKRFSGSNSGNTATLTISGTQAMDE |
| ARLGGAMDVWGQGTLVTVSS (SEQ ID | ADYYCAAWDDSLNGYYVFGTGTQLTVL |
| NO: 366) | (SEQ ID NO: 367) |
| QVQLQQSGAGLVKASQTLALTCAISGDSVSSN | QSVLTQPPSASGTPGQRVTISCSGSSSN |
| SAAWNWIRQSPSRGLEWLGRTYYRSKWYND | IGSYFVSWYQQLPGTAPKLLIYRNDKRP |
| YAVSVKSRITINPDTSTNQFSLQLNSVTPEDTA | SGVPERFSGSKSGTSASLAISGLQSEDE |
| VYYCTRGTGDKRAFDYWGQGTLVTVSS (SEQ | ADYYCAAWDDSLSGRIFGGGTKLTVL |
| ID NO: 368) | (SEQ ID NO: 369) |
| QVQLVQSGGGVVQPGRSLRLSCAASGFTFSS | DIQMTQTPLSLSVTLGQPASISCRSSQGL |
| YAMHWVRQAPGKGLEWVAVISYDGSNKYYA | VYSDGNTYLNWFQQRPGQSPRRLIYKV |
| DSVKGRFTISRDNSKNTLYLQMNSLRAEDTAV | SNRDSGVPDRFSGSGSGTDFTLKISRVE |
| YYCARAFRRYDAFDIWGQGTLVTVSS (SEQ ID | AEDVGVYYCMQGTHWPPAFGQGTKVEI |
| NO: 370) | K (SEQ ID NO: 371) |
| EVQLVQSGAEVKKPGESLKISCKGSGYSFTSY | QSVLTQPPSVSVSPGQTASITCSGNKLG |
| WIGWVRQMPGKGLEWMGIIYPGDSDTRYSPS | DKFVSWYHQRPGQSPVLVIYEDNKRPS |
| FQGQVTISADKSISTAYLQWSSLKASDTAMYY | GIPERFSGSNSGNTATLTISGTQAMDEG |
| CARGSGGYDYWGQGTLVTVSS (SEQ ID | DYYCQTWDVSSYVFGTGTKVTVL (SEQ |
| NO: 372) | ID NO: 373) |
| AVTLDESGGGLQTPGGALSLVCKASGFTFSSF | QAALTQPASVSANPGETVKITCSGGSSD |
| PMVWVRQAPGKGLEFVAGIVRTSTPKYGAAV | YGWFQQKSPGSAPVTVIYYSDKRPSDIP |
| TGRATISRDNGQSTVRLQLNNLRAEDTGTYFC | SRFSGSASGSMGALIITGVQAEDEAVYF |
| AKSATICGNCPEDIDAWGHGTEVIVSS (SEQ | CGSRDSSNVAIFGAGTTLTVL (SEQ ID |
| ID NO: 374) | NO: 375) |
| AVTLDESGGGLQTPGGALSLVCKASGFTFSSF | QAALTQPASVSANPGETVKITCSGGSSD |
| PMVWVRQAPGKGLEFVAGIVRTSTPKYGAAV | YGWFQQKSPGSAPVTVIYYSDKRPSDIP |
| TGRATISRDNGQSTVRLQLNNLRAEDTGTYFC | SRFSGSTSGSMGALIITGVQAEDEAVYF |
| AKSATICGNCPEDIDAWGHGTEVIVSS (SEQ | CGSRDSSNVAIFGAGTTLTVL (SEQ ID |
| ID NO: 376) | NO: 377) |
| AVTLDESGGGLQTPGGGLSLVCKASGFTFSN | QAALTQPSSVSANPGETVKITCSGPTYD |
| HQMAWVRQAPGKGLEYVGLINAAGSWTYYG | YGWFQQKSPGSAPVTVIYYNDNRPSDIP |
| TAVKGRATISRDNGQSTVRLQLNNLRAEDTAT | SRFSGSKSGSTATLTITGVQAEDEAVYY |
| YYCAKDVYGYCSTCGATIDTWGHGTEVIVSS | CGTYEDNNGASFGAGTTLTVL (SEQ ID |
| (SEQ ID NO: 378) | NO: 379) |
| AVTLDESGGGLQTPGGALSLVCKASGFTFSSF | QAALTQPASVSANPGETVKITCSGGGR |
| NMFWVRQAPGKGLEFVAHIKRNGGETDYGAA | WYGTYYYGWYQQKSPGSAPVTVIYSND |
| VKGRATISRDDGQSTVRLQLNNLRAEDTATYF | KRPSDIPSRFSGSASGSTATLTITGVQAD |
| CAKDAGDNCRWNYCTTNIDAWGHGTEVIVSS | DEAVYYCGSWDSTDTTAFGAGTTLTVL |
| (SEQ ID NO: 380) | (SEQ ID NO: 381) |
| AVTLDESGGGLQTPGGGLSLVCKASGFTFSS | QAALTQPASVSANPGETVKITCSGGGSY |
| YTMQWVRQAPGKGLEWVAGICNDGTTTNYG | YGSYYYGWYQQKSPGSAPVTVIYNNDK |
| SAVKGRATISRDNGQSTLRLQLNNLRAEDTGI | RPSDIPSRFSGSLSGSTNTLTITGVQVED |
| YFCTRGVDICWNSGNNIDAWGHGTEVIVSS | ESVYFCGSKDSSGAGIYGAGTTLTVL |
| (SEQ ID NO: 713) | (SEQ ID NO: 714) |
| AVTLDESGGGLQTPGGPLSLVCEGSGFTFSS | QAALTQPASVSANLGGTVEITCSGGSSG |
| YGMAWVRQAPGKRLEWVASISSTGINAYYGA | YGYGWYQQKSPGSAPVTVIYNNNNRPS |
| AVKGRATISRDNGQSTVRLQLNNLRAEDTGIY | DIPSRFSGSLSDSTHTLTITGVRAEDEAV |
| YCSKCAYSGSCSTSIDAWGHGTEVIVSS (SEQ | YFCGAWESSATDIFGAGTTLTVL (SEQ ID |
| ID NO: 382) | NO: 383) |
| AVTLDESGGGLQTPGGALSLVCKASGFDFSS | QAALTQPSSVSANPGETVKITCSGSINNY |
| NAMGWVRQAPGKGLEFIASITTDGSYTTYGAA | GWYQQKSPGSAPVTVIYQNDKRPSDIPS |
| VKGRATISRDDGQSTLRLQLNNLRAEDTATYY | RFSGSKSGSTGTLTITGVQAEDEAVYFC |
| CAKDSSIHWNGYDIDAWGHGTEVIVSS (SEQ | GSEDSRTYLGIFGAGTTLTVL (SEQ ID |
| ID NO: 384) | NO: 385) |
| AVTLDESGGGLQTPGGGLSLVCKASGFTFSS | QAALTQPASVSSNPGETVKITCSGGSSG |
| HAMFWVRQAPGKGLEYVAYINYDGITFYGAA | YAYGWYQQKSPGSAPVTLIYSNDKRPS |
| VKGRATISRDNGQSTVRLQLNNLRAEDTATYF | DIPSRFSGSKSGSTGTLTITGVQAEDEAV |
| CAKTTCSGCWDAAALIDAWGHGTEVIVSS | YYCGSFDSSSYIFGAGTTLTVL (SEQ ID |
| (SEQ ID NO: 386) | NO: 387) |
| AVTLDESGGGLQTPGGALSLVCKASGFTFSSF | QAALTQPASVSANPGETVKITCSGGSSD |
| PMVWVRQAPGKGLEFVAGIVRTSTPKYGAAV | YGWFQQKSPGSAPVTVIYYSDKRPSDIP |
| TGRATISRDNGQSTVRLQLNNLRAEDTGTYFC | SRFSGSTSGSTNTLTITGVQVEDEAVYF |
| AKSATICGNCPEDIDAWGHGTEVIVSS (SEQ | CGSGDNSYVGFGAGTTLTVL (SEQ ID |
| ID NO: 388) | NO: 389) |
| QVQLQQSGPGLVKPSQTLSLTCAISGDSVSSN | LPVLTQPPSASGTPGQRVTISCSGSSSNI |
| SAVWNWIRQSPSRGLEWLGRTYYRSKWYTD | GHKYVYWYQQLPGAAPKLLIYRNNQRP |
| FAVSVKSRITINPDTSKNQFSLQLNSVTPEDTA | SGVPDRFSASKSGTSASLAISGLRSEDE |
| VYYCARGRNGMDVWGQGTMVTVST (SEQ ID | ADYYCATWDDSLNGVVFGGGTKVTVL |
| NO: 701) | (SEQ ID NO: 702) |

14. The antibody or antigen binding fragment thereof according to any one of items 1 to 13, wherein said antigen binding fragment is a Fab fragment, a F(ab′)₂ fragment, a Fd fragment, an Fv fragment, a single-chain Fv (scFv) molecule, or a single-domain antibody (dAb).
15. The antibody or antigen binding fragment thereof according to any one of items 1 to 14, which is a multispecific antibody.
16. The antibody or antigen binding fragment thereof according to item 15, wherein said multispecific antibody is a bispecific antibody.
17. The antibody or antigen binding fragment thereof of item 15 or 16, wherein the multispecific antibody further comprises a second antibody or antigen binding fragment or binding domain thereof that specifically binds to a tumor antigen.
18. The antibody or antigen binding fragment thereof of item 15 or 16, wherein the multispecific antibody further comprises a second antibody or antigen binding fragment thereof or binding domain that specifically binds to a protein involved in immune cell activation.
19. The antibody or antigen binding fragment thereof of item 18, wherein the protein involved in immune cell activation is CD3.
20. The antibody or antigen binding fragment thereof of item 19, wherein the second antibody or antigen binding fragment thereof or binding domain comprises a combination of CDR-H1, CDR-H2, CDR-H3, CDR-L1, CDR-L2 and CDR-L3 comprising or consisting of the following amino acid sequences:

1)
(SEQ ID NO: 414)
CDR-H1: GFTFSSYS;
(SEQ ID NO: 433)
CDR-H2: ISSSSSYI;
(SEQ ID NO: 449)
CDR-H3: ARVKDYDSSLDY;
(SEQ ID NO: 78)
CDR-L1: KLGDKY;
CDR-L2: QDS;
and
(SEQ ID NO: 108)
CDR-L3: QAWDSSTVV;
2)
(SEQ ID NO: 415)
CDR-H1: GFSFGSSA;
(SEQ ID NO: 434)
CDR-H2: ISHSGSTT;
(SEQ ID NO: 451)
CDR-H3: AKIGAYGYYFHY;
(SEQ ID NO: 475)
CDR-L1: GGSIASNY;
CDR-L2: DDN;
and
(SEQ ID NO: 494)
CDR-L3: QSSDNINHVI;
or
3)
(SEQ ID NO: 415)
CDR-H1: GFSFGSSA;
(SEQ ID NO: 434)
CDR-H2: ISHSGSTT;
(SEQ ID NO: 451)
CDR-H3: AKIGAYGYYFHY;
(SEQ ID NO: 476)
CDR-L1: SGSIASKY;
CDR-L2: DDK;
and
(SEQ ID NO: 495)
CDR-L3: QSFDSTNQAVV.

21. The antibody or antigen binding fragment thereof of item 19, wherein the second antibody or antigen binding fragment thereof or binding domain comprises one of the following combinations of HCVR and LCVR:

1)
HCVR:
(SEQ ID NO: 638)
EVQLVQSGGGLVKPGGSLRLSCAASGFTFSSYSMNWVRQAPGKGLEWVSS
ISSSSSYIYYADSVKGRFTISRDNSKNTLYLQMNSLRAEDTAVYYCARVK
DYDSSLDYWGQGTLVTVSS
LCVR:
(SEQ ID NO: 639)
SVSPGQTARITCSGFKLGDKYVSWYQQKPGQSPVLVIYQDSKRPSGIPER
FSGSNSGNTATLTISGTQAMDEADYYCQAWDSSTVVFGGGTKLTVL;
2)
HCVR:
(SEQ ID NO: 646)
QVQLQQSGGGLVQSGGSLRLSCAASGFSFGSSAMSWVRQAPGRGLEWVSG
ISHSGSTTYYADSVKGRFTISRDNSKNTLYLQMNSLRVEDTAVYYCAKIG
AYGYYFHYWGQGTLVTVSS
LCVR:
(SEQ ID NO: 647)
NFMLTQPHSVSESPGKTVTISCTRSGGSIASNYVQWYQQRPGSSPTTVIY
DDNQRPSGVPDRFSGSIDSSSNSASLTISGLKTEDEADYYCQSSDNINHV
IFGGGTKLTVL;
or
3)
HCVR:
(SEQ ID NO: 648)
QVQLQQSGGGLVQPGGSLRLSCAASGFSFGSSAMSWVRQAPGRGLEWVSG
ISHSGSTTYYADSVKGRFTISRDNSKNTLYLQMNSLRVEDTAVYYCAKIG
AYGYYFHYWGQGTLVTVSS
LCVR:
(SEQ ID NO: 649)
NFMLTQPHSLSESPGKTVTISCTRSSGSIASKYVQWYQQRPGSAPTTVIY
DDKQRPSGVPDRFSGSFDSSSNSASLTISGLKTEDEADYYCQSFDSTNQA
VVFGGGTKLTVL.

22. A conjugate comprising the antibody or antigen-binding fragment thereof of any one of items 1 to 21, and a therapeutic agent such as an antitumor agent.
23. A pharmaceutical composition comprising the antibody or antigen-binding fragment thereof of any one of items 1 to 21, or the conjugate of item 22, and a pharmaceutically acceptable excipient.
24. A method for treating a TrkB-related disease or disorder in a subject in need thereof, comprising administering to the subject an effective amount of the antibody or antigen-binding fragment thereof of any one of items 1 to 21, the conjugate of item 22, or the pharmaceutical composition of item 23.
25. The method of item 24, wherein the TrkB-related disease or disorder is nerve cell damage associated with nervous system injury or a neurodegenerative or motor neuron disease.
26. The method of item 25, wherein the neurodegenerative or motor neuron disease is Parkinson's Disease (PD), mild cognitive impairment (MCI), Alzheimer's disease (AD), Huntington's disease (HD), motor neuron disease, Tourette's syndrome, dementia, amyotrophic Lateral Sclerosis (ALS), idiopathic motor neuropathy, hereditary spastic paraplegia (HSP), primary lateral sclerosis (PLS), progressive muscular atrophy (PMA), progressive bulbar palsy (PBP), pseudobulbar palsy, Bell's palsy, or spinal muscular atrophy (SMA).
27. The method of item 24, wherein the TrkB-related disease or disorder is an optic neuropathy.
28. The method of item 27, wherein the optic neuropathy is glaucoma, anterior ischaemic optic neuropathy (AION), posterior ischemic optic neuropathy, diabetic retinopathy, retinopathy of prematurity, age-related macular degeneration, retinal artery or vein occlusion, radiation optic neuropathy, compressive optic neuropathy, infiltrative optic neuropathy, traumatic optic neuropathy, mitochondrial optic neuropathy, toxic optic neuropathies, hereditary optic neuropathies, Leber hereditary optic neuropathy, Rosenberg Chutorian syndrome, Wolfram syndrome, optic nerve hypoplasia, optic neuritis, photoreceptor degeneration, or retinitis pigmentosa.
29. The method of item 24, wherein the TrkB-related disease or disorder is a metabolic disease, such as obesity or diabetes.
30. The method of item 24, wherein the TrkB-related disease or disorder is a mental disorder, such as depression.
31. A method for treating a TrkB-expressing cancer in a subject in need thereof, comprising administering to the subject an effective amount of the antibody or antigen-binding fragment thereof of any one of items 1 to 21, the conjugate of item 22, or the pharmaceutical composition of item 23.
32. The method of item 31, wherein the cancer is breast cancer, cholangiocarcinoma, colorectal cancer, gastrointestinal cancer, head and neck neoplasms, lymphoma, melanoma, neuroendocrine tumors, sarcoma, non-small cell lung cancer, ovarian cancer, pancreatic cancer, papillary thyroid cancer, primary brain tumor, renal cell carcinoma, sarcoma, salivary gland cancer.
33. The method of item 31 or 32, wherein the antibody or antigen-binding fragment thereof is the multispecific antibody defined in any one of items 17 to 21.
34. Use of the antibody or antigen-binding fragment thereof of any one of items 1 to 21, the conjugate of item 22, or the pharmaceutical composition of item 23, for treating, or for the manufacture of a medicament for treating, a TrkB-related disease or disorder in a subject.
35. The use of item 34, wherein the TrkB-related disease or disorder is nerve cell damage associated with nervous system injury or a neurodegenerative or motor neuron disease.
36. The use of item 35, wherein the neurodegenerative or motor neuron disease is Parkinson's Disease (PD), mild cognitive impairment (MCI), Alzheimer's disease (AD), Huntington's disease (HD), motor neuron disease, Tourette's syndrome, dementia, amyotrophic Lateral Sclerosis (ALS), idiopathic motor neuropathy, hereditary spastic paraplegia (HSP), primary lateral sclerosis (PLS), progressive muscular atrophy (PMA), progressive bulbar palsy (PBP), pseudobulbar palsy, Bell's palsy, or spinal muscular atrophy (SMA).
37. The use of item 34, wherein the TrkB-related disease or disorder is an optic neuropathy.
38. The use of item 37, wherein the optic neuropathy is glaucoma, anterior ischaemic optic neuropathy (AION), posterior ischemic optic neuropathy, diabetic retinopathy, retinopathy of prematurity, age-related macular degeneration, retinal artery or vein occlusion, radiation optic neuropathy, compressive optic neuropathy, infiltrative optic neuropathy, traumatic optic neuropathy, mitochondrial optic neuropathy, toxic optic neuropathies, hereditary optic neuropathies, Leber hereditary optic neuropathy, Rosenberg Chutorian syndrome, Wolfram syndrome, optic nerve hypoplasia, optic neuritis, photoreceptor degeneration, or retinitis pigmentosa.
39. The use of item 34, wherein the TrkB-related disease or disorder is a metabolic disease.
40. The use of item 39, wherein the metabolic disease is obesity or diabetes.
41. The use of item 34, wherein the TrkB-related disease or disorder is a mental disorder.
42. The use of claim 41, wherein the mental disorder is depression.
43. Use of the antibody or antigen-binding fragment thereof of any one of items 1 to 21, the conjugate of item 22, or the pharmaceutical composition of item 23, for treating, or for the manufacture of a medicament for treating, a TrkB-expressing cancer in a subject.
44. The use of item 43, wherein the cancer is breast cancer, cholangiocarcinoma, colorectal cancer, gastrointestinal cancer, head and neck neoplasms, lymphoma, melanoma, neuroendocrine tumors, sarcoma, non-small cell lung cancer, ovarian cancer, pancreatic cancer, papillary thyroid cancer, primary brain tumor, renal cell carcinoma, sarcoma, salivary gland cancer.
45. The use of item 43 or 44, wherein the antibody or antigen-binding fragment thereof is the multispecific antibody defined in any one of items 17 to 21.
46. The antibody or antigen-binding fragment thereof of any one of items 1 to 21, the conjugate of item 22, or the pharmaceutical composition of item 23, for use in treating a TrkB-related disease or disorder in a subject.
47. The antibody or antigen-binding fragment thereof, conjugate, or composition for use of item 46, wherein the TrkB-related disease or disorder is nerve cell damage associated with nervous system injury or a neurodegenerative or motor neuron disease.
48. The antibody or antigen-binding fragment thereof, conjugate or composition for use of item 47, wherein the neurodegenerative or motor neuron disease is Parkinson's Disease (PD), mild cognitive impairment (MCI), Alzheimer's disease (AD), Huntington's disease (HD), motor neuron disease, Tourette's syndrome, dementia, amyotrophic Lateral Sclerosis (ALS), idiopathic motor neuropathy, hereditary spastic paraplegia (HSP), primary lateral sclerosis (PLS), progressive muscular atrophy (PMA), progressive bulbar palsy (PBP), pseudobulbar palsy, Bell's palsy, or spinal muscular atrophy (SMA).
49. The antibody or antigen-binding fragment thereof, conjugate or composition for use of item 46, wherein the TrkB-related disease or disorder is an optic neuropathy.
50. The antibody or antigen-binding fragment thereof, conjugate or composition for use of item 49, wherein the optic neuropathy is glaucoma, anterior ischaemic optic neuropathy (AION), posterior ischemic optic neuropathy, diabetic retinopathy, retinopathy of prematurity, age-related macular degeneration, retinal artery or vein occlusion, radiation optic neuropathy, compressive optic neuropathy, infiltrative optic neuropathy, traumatic optic neuropathy, mitochondrial optic neuropathy, toxic optic neuropathies, hereditary optic neuropathies, Leber hereditary optic neuropathy. Rosenberg Chutorian syndrome, Wolfram syndrome, optic nerve hypoplasia, optic neuritis, photoreceptor degeneration, or retinitis pigmentosa.
51. The antibody or antigen-binding fragment thereof, conjugate or composition for use of item 46, wherein the TrkB-related disease or disorder is a metabolic disease.
52. The antibody or antigen-binding fragment thereof, conjugate or composition for use of item 51, wherein the metabolic disease is obesity or diabetes.
53. The antibody or antigen-binding fragment thereof, conjugate or composition for use of item 46, wherein the TrkB-related disease or disorder is a mental disorder.
54. The antibody or antigen-binding fragment thereof, conjugate or composition for use of claim 53, wherein the mental disorder is depression.
55. The antibody or antigen-binding fragment thereof of any one of items 1 to 21, the conjugate of item 22, or the pharmaceutical composition of item 23, for use in treating a TrkB-expressing cancer in a subject.
56. The antibody or antigen-binding fragment thereof, conjugate or composition for use of item 55, wherein the cancer is breast cancer, cholangiocarcinoma, colorectal cancer, gastrointestinal cancer, head and neck neoplasms, lymphoma, melanoma, neuroendocrine tumors, sarcoma, non-small cell lung cancer, ovarian cancer, pancreatic cancer, papillary thyroid cancer, primary brain tumor, renal cell carcinoma, sarcoma, salivary gland cancer.
57. The antibody or antigen-binding fragment thereof, conjugate or composition for use of item 55 or 56, wherein the antibody or antigen-binding fragment thereof is the multispecific antibody defined in any one of items 17 to 21.
58. A method for detecting TrkB or a TrkB-expressing cell in a sample comprising contacting the sample with the antibody or antigen-binding fragment thereof of any one of items 1 to 21.
59. An antibody that binds to cluster of differentiation 3 (CD3) comprising a combination of heavy chain complementarity determining regions (CDR-H1, CDR-H2 and CDR-H3) and light chain complementarity determining regions (CDR-L1, CDR-L2 and CDR-L3), wherein said CDR-H1, CDR-H2, CDR-H3, CDR-L1, CDR-L2 and CDR-L3 comprise or consist of amino acid sequences having at least 70% identity with the sequences depicted in Table 2:

TABLE 2

| CDR-H1 | CDR-H2 | CDR-H3 | CDR-L1 | | CDR-L3 |
|---|---|---|---|---|---|
| SEQ ID | SEQ ID | SEQ ID | (SEQ ID | | (SEQ ID |
| NO: ) | NO: ) | NO: ) | NO: ) | CDR-L2 | NO: ) |
| 16 | 432 | 448 | 471 | DNN | 490 |
| 414 | 433 | 449 | 78 | QDS | 108 |
| 15 | 26 | 450 | 472 | NND | 491 |
| 415 | 434 | 451 | 473 | DDS | 492 |
| 703 | 434 | 451 | 474 | DDS | 493 |
| 415 | 434 | 451 | 475 | DDN | 494 |
| 415 | 434 | 451 | 476 | DDK | 495 |
| 415 | 434 | 451 | 477 | EDN | 496 |
| 416 | 435 | 452 | 80 | SNN | 497 |
| 416 | 435 | 452 | 80 | SNN | 497 |
| 15 | 26 | 453 | 478 | RNN | 498 |
| 15 | 26 | 453 | 478 | RNN | 118 |
| 15 | 26 | 453 | 478 | RNN | 499 |
| 417 | 436 | 454 | 479 | EDN | 500 |
| 418 | 437 | 455 | 480 | EDK | 501 |
| 419 | 438 | 456 | 481 | QDN | 502 |
| 420 | 439 | 457 | 482 | DNN | 503 |
| 421 | 437 | 458 | 476 | DDD | 504 |
| 422 | 432 | 459 | 471 | DNN | 490 |
| 423 | 440 | 460 | 483 | DNN | 505 |
| 424 | 441 | 461 | 484 | DDS | 506 |
| 425 | 442 | 462 | 477 | EDN | 507 |
| 20 | 432 | 463 | 477 | EDN | 508 |
| 426 | 441 | 464 | 485 | RNN | 509 |
| 427 | 443 | 465 | 486 | EDK | 510 |
| 20 | 444 | 466 | 487 | DDV | 511 |
| 428 | 445 | 467 | 488 | GAS | 512 |
| 429 | 446 | 468 | 489 | DNN | 513 |
| 430 | 433 | 469 | 477 | DDK | 514 |
| 431 | 447 | 470 | 78 | QDN | 515 |

or an antigen-binding fragment thereof.
   60. The antibody or antigen binding fragment thereof according to item 59, wherein the CDR-H1, CDR-H2, CDR-H3, CDR-L1, CDR-L2 and CDR-L3 comprise or consist of amino acid sequences having at least 80% identity with the sequences depicted in Table 2.
   61. The antibody or antigen binding fragment thereof according to item 59, wherein the CDR-H1, CDR-H2, CDR-H3, CDR-L1, CDR-L2 and CDR-L3 comprise or consist of the amino acid sequences depicted in Table 2.
   62. The antibody or antigen binding fragment thereof according to any one of items 59 to 61, which further comprises a heavy chain framework region 1 (VH FR1) comprising an amino acid sequence having at least 60% identity with one of the following sequences:

(SEQ ID NO: 516)
EVQLVESGGGVVQPGRSLRLSCAAS,
(SEQ ID NO: 142)
EVQLVQSGGGLVKPGGSLRLSCAAS,
(SEQ ID NO: 517)
QVQLQQSGPGLVKPSQTLSLTCAIS,
(SEQ ID NO: 518)
QVQLVQSEGGLVQPGGSLRLSCAAS,
(SEQ ID NO: 519)
QVQLLQSAGGLVQPGGSLRLSCAAS,
(SEQ ID NO: 520)
QVQLQQSGGGLVQSGGSLRLSCAAS,
(SEQ ID NO: 521)
QVQLQQSGGGLVQPGGSLRLSCAAS,
(SEQ ID NO: 522)
QVQLVQSGAEGKKPGSSVKVSCKAS,
(SEQ ID NO: 523)
QVQLVQSGAEVKKPGSSVKVSCKAS,
(SEQ ID NO: 524)
EVQLVESGGGLVQPGGSLRLSCAAS,
(SEQ ID NO: 525)
QVTLRESGGGLVKPGGSLRLSCSAS,
(SEQ ID NO: 526)
QVQLLQSAGGVVQPGRSLRLSCAAS,
(SEQ ID NO: 527)
HVQLVQSGGGSAQHGGSLRLSCAAS,
(SEQ ID NO: 528)
AVQLVESAGGVVQLRRSLSLSSAAS,
(SEQ ID NO: 529)
QVQLQQSGAEVKKPGASLKVSCKAS,
(SEQ ID NO: 530)
QVQLVQSGPEVKKPGASVNISCKTS,
(SEQ ID NO: 531)
QVQLVQSGAEVKKPGASVKVSCKAS,
(SEQ ID NO: 532)
QVQLVQSGGGVVQPARSLRLSSAAS,
(SEQ ID NO: 533)
EVQLVQSGGGLVKPGRSLRLSCTAS,
(SEQ ID NO: 534)
QVNLRESGGGVVQPGGSLRLSCAAS,
or
(SEQ ID NO: 535)
QVQLVQSGGGLVQPGGSLRLSCAAS.

63. The antibody or antigen binding fragment thereof according to any one of items 59 to 62, which further comprises a heavy chain framework region 2 (VH FR2) comprising an amino acid sequence having at least 60% identity with one of the following sequences:

(SEQ ID NO: 536)
MHWVRQAPGKGLEWVSA,
(SEQ ID NO: 537)
MNWVRQAPGKGLEWVSS,
(SEQ ID NO: 178)
WNWIRQSPSRGLEWLGR,
(SEQ ID NO: 538)
MSWVRQAPGRGLEWVSG,
(SEQ ID NO: 539)
ISWVRQAPGQGLEWMGG,
(SEQ ID NO: 540)
MHWVRQAPGQGLEWMGI,
(SEQ ID NO: 541)
MNWVRQAPGKGLEWIST,
(SEQ ID NO: 542)
MAWIRQAPGKGLEWVAY,
(SEQ ID NO: 543)
VHWVRQAPGKGLEWVAA,
(SEQ ID NO: 544)
MNWVRQAPGKRLEWIST,
(SEQ ID NO: 545)
MHWIRQAPVKGMEWVSA,
(SEQ ID NO: 546)
MHWVRQAPGKGLEWVSG,
(SEQ ID NO: 547)
FSWVRQAPGQGLEWMGW,
(SEQ ID NO: 548)
VHWVRQAPGQGFEYMGR,
(SEQ ID NO: 549)
IHWVRQAPGKGLEWVSA,
(SEQ ID NO: 550)
ISWVRQAPGQGLEWMGW,
(SEQ ID NO: 551)
MSWVRQAPGKGLEWVST,
(SEQ ID NO: 552)
MHWVRQAPGKRLEWVAV,
(SEQ ID NO: 553)
MSWFRQAPGKGLEWVGF,
(SEQ ID NO: 554)
MNWIRQAPGKGLEWVSS,
or
(SEQ ID NO: 555)
MSWVRQAPGKGLEWVSS.

64. The antibody or antigen binding fragment thereof according to any one of items 59 to 63, which further comprises a heavy chain framework region 3 (VH FR3) comprising an amino acid sequence having at least 60% identity with one of the following sequences:

(SEQ ID NO: 556)
YYADSVKGRFTISRDNSKNTLYLQMNSLRAEDTAVYYC,
(SEQ ID NO: 557)
YNDYAVSVKSRITINPDTSKNQFSLQLNSVTPEDTAVYYC,
(SEQ ID NO: 558)
YYADSVKGRFTISRDNSKNTLYLQMNSLRVEDTAVYYC,
(SEQ ID NO: 559)
NYAQKFQGRVTITADESTSTAYMELRSLRSDDTAVYYC,
(SEQ ID NO: 560)
NYAQKFQGRVTITADESTSTAYMELSSLRSEDTAVYYC,
(SEQ ID NO: 561)
YNDYAVSVKSRITINPDTSKNQFSLHLNSVTPEDTAVYYC,
(SEQ ID NO: 562)
SYAQKFQGRVTMTRDTSTSTVYMELSSLRSEDTAVYYC,
(SEQ ID NO: 563)
YYADSVKGRFTVSRDNAKNSLFLQMDSLRDEDTAVYYC,
(SEQ ID NO: 564)
YHADSVKGRFTVSRDNAKNLMYLEMNSLRADDTAVYYC,
(SEQ ID NO: 565)
YYGDSVRGRFTISRDDSKNTVYLQMNSLRAEDTAVYYC,
(SEQ ID NO: 566)
YYADSVKGRFTVSRDNTKNLTFLQMDSLRDEDTAVYYC,
(SEQ ID NO: 567)
YYADSVKGRFTISRHNSKNTLYLQMNSLRAEDTGVYWG,
(SEQ ID NO: 568)
IYADSVKGRFTISRDNAKNSLYLQMNSLRPEDTAVYYC,
(SEQ ID NO: 569)
NYAQKFQGRVTMTTDTSTSTAYMEMRSLRSDDTAVYYC,
(SEQ ID NO: 570)
RYAQKFQGRVAMTSDTSTSTAYMELSSLRSEDTAVYYC,
(SEQ ID NO: 571)
YYADSVKGRFTISRDNAKNSLYLQMNSLRDEDTAVYYC,
(SEQ ID NO: 572)
NYAQKLQGRVTMTTDTPTSTAYMEVRSLRSDDTAVYYC,
(SEQ ID NO: 573)
NYADSVKGRFTISRDSSKNTLYLQMNSLRAEDTAVYYC,
(SEQ ID NO: 574)
YYADSVKGRFSVSRGNSKNMLYLQMTGLTTEDTAVYYC,
(SEQ ID NO: 575)
EYAASVKGRFTISRDDSKSIAYLQMNSLKTEDTAVYYC,
(SEQ ID NO: 576)
YYADSVKGRFTISRDNAKNSLYLQMNSLRAEDTAMYYC,
or
(SEQ ID NO: 577)
YYADSVKGRFTISRDNSDGSVYLKMNSLAAGDTAAYYC.

65. The antibody or antigen binding fragment thereof according to any one of items 59 to 64, which further comprises a heavy chain framework region 4 (VH FR4) comprising an amino acid sequence having at least 60% identity with one of the following sequences: WGQGTTVTVSS (SEQ ID NO:208), WGQGTLVTVSS (SEQ ID NO:209), WGQGTMVTVSS (SEQ ID NO:210), or WGRGTLVTVSS (SEQ ID NO:578).
66. The antibody or antigen binding fragment thereof according to any one of items 59 to 65, which further comprises a light chain framework region 1 (VL FR1) comprising or consisting of an amino acid sequence having at least 60% identity with one of the following sequences:

(SEQ ID NO: 579)
QSVLTQPPSVSAAPGQKATISCSGG,
(SEQ ID NO: 580)
SYELMQPPSVSVSPGQTARITCSGF,
(SEQ ID NO: 235)
QSVLTQPPSASGTPGQRVTISCSGS,
(SEQ ID NO: 581)
NFMLTQPHSVSESPGKTVTISCTGS,
(SEQ ID NO: 582)
NFMLTQPHSVSESPGKTVTISCTGN,
(SEQ ID NO: 583)
NFMLTQPHSVSESPGKTVTISCTRS,
(SEQ ID NO: 584)
NFMLTQPHSLSESPGKTVTISCTRS,
(SEQ ID NO: 585)
NFMLTQPHSVSESPGRTVTISCTRS,
(SEQ ID NO: 586)
QPVLTQSPSASGTPGQRVTISCSGS,
(SEQ ID NO: 219)
QAGLTQPPSASGTPGQRVTISCSGS,
(SEQ ID NO: 230)
QPVLTQPPSASGTPGQRVTISCSGS,
(SEQ ID NO: 587)
QPVLTQPPSASGTPEQRVTISCSGS,
(SEQ ID NO: 588)
QSALTQPHSVSESPGKTVTISCTRT,
(SEQ ID NO: 589)
NFMLTQPHSVSESPGKTVAISCSRS,
(SEQ ID NO: 590)
QSVLTQPPSVSAAPGQKVTISCSGS,
(SEQ ID NO: 591)
NFMLTQPHSVSESPGKTVTLSCTRS,
(SEQ ID NO: 592)
QSVLTQPPSVSAAPGKKVTISCSGG,
(SEQ ID NO: 593)
SYVLTQPPSVSVAPGQTAKITCGGD,
(SEQ ID NO: 594)
NFMLTQPHSVAESPGKTVTISCTRS,
(SEQ ID NO: 595)
NFMLTQPHSVSESPGKTVTISCTRN,
(SEQ ID NO: 596)
SYVLTQPPSVSVAPGQTARITCGGD,
(SEQ ID NO: 597)
EIVLTQSPGTLSLSPGERATLSCRAS,
or
(SEQ ID NO: 598)
SYVLTQPPSVSASPGQTASITCSGH.

67. The antibody or antigen binding fragment thereof according to any one of items 59 to 66, which further comprises a light chain framework region 2 (VL FR2) comprising or consisting of an amino acid sequence having at least 60% identity with one of the following sequences:

(SEQ ID NO: 266)
VSWYQQLPGTAPKLLIY,
(SEQ ID NO: 244)
VSWYQQKPGQSPVLVIY,
(SEQ ID NO: 257)
VYWYQQLPGTAPKLLIY,
(SEQ ID NO: 599)
VQWYQQRPDSPPSTVIY,
(SEQ ID NO: 600)
VQWYQHRPDSAPIAVIY,
(SEQ ID NO: 601)
VQWYQQRPGSSPTTVIY,
(SEQ ID NO: 602)
VQWYQQRPGSAPTTVIY,
(SEQ ID NO: 603)
VNWYQQLPGTAPKLLIY,
(SEQ ID NO: 604)
VQWYQQRPGSAPTVVIY,
(SEQ ID NO: 605)
VQWYQQRPGSSPTTVIL,
(SEQ ID NO: 606)
VQWYQQRPGNVPTTIIY,
(SEQ ID NO: 607)
VQWYQQRPGSAPTIVIF,
(SEQ ID NO: 608)
VHWYQQKPGQAPVVVVY,
(SEQ ID NO: 609)
VHWYQQRPGSAPTTVIY,
(SEQ ID NO: 610)
VQWYHQRPGSAPTTVIY,
(SEQ ID NO: 611)
VHWYQQKPGQAPVLVVY,
(SEQ ID NO: 612)
LAWYQQKPGQAPRLLIY,
or
(SEQ ID NO: 613)
TSWYQQKPGQSPVLVIY.

68. The antibody or antigen binding fragment thereof according to any one of items 59 to 67, which further comprises a light chain framework region 3 (VL FR3) comprising or consisting of an amino acid sequence having at least 60% identity with one of the following sequences:

(SEQ ID NO: 614)
KRPSGIPDRFSGSKSGTSAALAITGLQTGDEADYYC,
(SEQ ID NO: 271)
KRPSGIPERFSGSNSGNTATLTISGTQAMDEADYYC,
(SEQ ID NO: 615)
QRPSGVPDRFSGSKSGTSGSLTISGLQAEDEADYYC,
(SEQ ID NO: 616)
QRPSGVPDRFSGSIDRSSNSASLTISGLRTEDEADYYC,
(SEQ ID NO: 617)
QRPSGIPDRFSGSIDRSSNSASLTISGLRPEDEADYYC,
(SEQ ID NO: 618)
QRPSGVPDRFSGSIDSSSNSASLTISGLKTEDEADYYC,
(SEQ ID NO: 619)
QRPSGVPDRFSGSFDSSSNSASLTISGLKTEDEADYYC,
(SEQ ID NO: 620)
QRLSGVPDRFSGSIDRSSNSASLTISGLKTEDEADYYC,
(SEQ ID NO: 288)
QRPSGVPDRFSGSKSGTSASLAISGLQSEDEADYYC,
(SEQ ID NO: 621)
QRPSGVPDRFSASKSGTSASLAISGLRSEDEADYYC,
(SEQ ID NO: 284)
QRPSGVPDRFSGSKSGTSASLAISGLRSEDEADYYC,
(SEQ ID NO: 622)
QRPSGVPDRFSGSIDSSTNSASLTISRLKTEDEADYYC,
(SEQ ID NO: 623)
QRPSGVPDRFSGSIDTSSNSATLTISGLKTEDEADYYC,
(SEQ ID NO: 624)
QRPSGVPDRFSGSIDKSSNSASLTISGLKTEDEADYYC,
(SEQ ID NO: 625)
QRPSGIPDRFSGSKSGTSATLGITGLQTGDEADYYC,
(SEQ ID NO: 626)
RRPSGVPDRFSGSIDRSSNSASLTIFGMKTEDEADYYC,
(SEQ ID NO: 627)
KRPSGIPDRFSGSKSGTSASLAITGLQTGDEADYYC,
(SEQ ID NO: 628)
KRPSGIPDRFSGSKSGTSATLGITGLQTGDEADYYC,
(SEQ ID NO: 629)
DRPSGIPDRFSGSNSGNTATLTITRVEAGDEADYYC,
(SEQ ID NO: 630)
QRPSGVPDRFSGSKSGTSASLVISGLRSEDEADYHC,
(SEQ ID NO: 631)
QRPSGVPGRFSGSIDGSSNSASLTISGLQAEDEGDYYC,
(SEQ ID NO: 632)
DRPSDIPERFSGSNSGNTATLTISRVEAGDEADYYC,
(SEQ ID NO: 633)
SRATGIPDRFSGSGSGTDFTLTISRLEPEDFAVYYC,
or
(SEQ ID NO: 634)
KRPSGVPDRFSGSIDSSTNSASLTISGLKTEDEADYYC.

69. The antibody or antigen binding fragment thereof according to any one of items 59 to 68, which further comprises a light chain framework region 4 (VL FR4) comprising or consisting of an amino acid sequence having at least 60% identity with one of the following sequences: FGGGTKLTVL (SEQ ID NO:303), FGGGTKVTVL (SEQ ID NO:305), FGQGTKVDIK (SEQ ID NO: 635) or FGTGTKLTVL (SEQ ID NO:306).
70. The antibody or antigen binding fragment thereof according to any one of items 59 to 69, wherein said antibody comprises one of the following combinations of heavy chain variable region (HCVR) and light chain variable region (LCVR):

| HCVR | LCVR |
|---|---|
| EVQLVESGGGVVQPGRSLRLSCAASGFTFSSY | QSVLTQPPSVSAAPGQKATISCSGGSS |
| AMHWVRQAPGKGLEWVSAISGSGGSTYYADS | NLGNNYVSWYQQLPGTAPKLLIYDNNK |
| VKGRFTISRDNSKNTLYLQMNSLRAEDTAVYY | RPSGIPDRFSGSKSGTSAALAITGLQTG |
| CARDAHDYWGQGTLVTVSS (SEQ ID NO: 636) | DEADYYCGTWDSSLSAGVFGGGTKLTV |
| | L (SEQ ID NO: 637) |
| EVQLVQSGGGLVKPGGSLRLSCAASGFTFSSY | SYELMQPPSVSVSPGQTARITCSGFKL |
| SMNWVRQAPGKGLEWVSSISSSSSYIYYADSV | GDKYVSWYQQKPGQSPVLVIYQDSKRP |
| KGRFTISRDNSKNTLYLQMNSLRAEDTAVYYC | SGIPERFSGSNSGNTATLTISGTQAMDE |
| ARVKDYDSSLDYWGQGTLVTVSS (SEQ ID | ADYYCQAWDSSTVVFGGGTKLTVL |
| NO: 638) | (SEQ ID NO: 639) |
| QVQLQQSGPGLVKPSQTLSLTCAISGDSVSSN | QSVLTQPPSASGTPGQRVTISCSGSSS |
| SAAWNWIRQSPSRGLEWLGRTYYRSKWYNDY | NIGRNHVYWYQQLPGTAPKLLIYNNDQ |
| AVSVKSRITINPDTSKNQFSLQLNSVTPEDTAV | RPSGVPDRFSGSKSGTSGSLTISGLQA |
| YYCARGITGTGYGMDVWGQGTLVTVSS (SEQ | EDEADYYCAAWDDSLSSGVFGGGTKV |
| ID NO: 640) | TVL (SEQ ID NO: 641) |
| QVQLVQSEGGLVQPGGSLRLSCAASGFSFGS | NFMLTQPHSVSESPGKTVTISCTGSGG |
| SAMSWVRQAPGRGLEWVSGISHSGSTTYYAD | NIGNNYVQWYQQRPDSPPSTVIYDDSQ |
| SVKGRFTISRDNSKNTLYLQMNSLRVEDTAVY | RPSGVPDRFSGSIDRSSNSASLTISGLR |
| YCAKIGAYGYYFHYWGQGTLVTVSS (SEQ ID | TEDEADYYCQSSDSGRVVFGGGTKLTV |
| NO: 642) | L (SEQ ID NO: 643) |
| QVQLLQSAGGLVQPGGSLRLSCAASGYSFGS | NFMLTQPHSVSESPGKTVTISCTGNGG |
| SAMSWVRQAPGRGLEWVSGISHSGSTTYYAD | NIASNYVQWYQHRPDSAPIAVIYDDSQR |
| SVKGRFTISRDNSKNTLYLQMNSLRAEDTAVY | PSGIPDRFSGSIDRSSNSASLTISGLRPE |
| YCAKIGAYGYYFHYWGQGTLVTVSS (SEQ ID | DEADYYCQSSDNANVVFGGGTKLTVL |
| NO: 644) | (SEQ ID NO: 645) |
| QVQLQQSGGGLVQSGGSLRLSCAASGFSFGS | NFMLTQPHSVSESPGKTVTISCTRSGG |
| SAMSWVRQAPGRGLEWVSGISHSGSTTYYAD | SIASNYVQWYQQRPGSSPTTVIYDDNQ |
| SVKGRFTISRDNSKNTLYLQMNSLRVEDTAVY | RPSGVPDRFSGSIDSSSNSASLTISGLK |
| YCAKIGAYGYYFHYWGQGTLVTVSS (SEQ ID | TEDEADYYCQSSDNINHVIFGGGTKLTV |
| NO: 646) | L (SEQ ID NO: 647) |
| QVQLQQSGGGLVQPGGSLRLSCAASGFSFGS | NFMLTQPHSLSESPGKTVTISCTRSSGS |
| SAMSWVRQAPGRGLEWVSGISHSGSTTYYAD | IASKYVQWYQQRPGSAPTTVIYDDKQR |
| SVKGRFTISRDNSKNTLYLQMNSLRVEDTAVY | PSGVPDRFSGSFDSSSNSASLTISGLKT |
| YCAKIGAYGYYFHYWGQGTLVTVSS (SEQ ID | EDEADYYCQSFDSTNQAVVFGGGTKLT |
| NO: 648) | VL (SEQ ID NO: 649) |
| QVQLQQSGGGLVQPGGSLRLSCAASGFSFGS | NFMLTQPHSVSESPGRTVTISCTRSSG |
| SAMSWVRQAPGRGLEWVSGISHSGSTTYYAD | SIASNYVQWYQQRPGSAPTTVIYEDNQ |
| SVKGRFTISRDNSKNTLYLQMNSLRVEDTAVY | RLSGVPDRFSGSIDRSSNSASLTISGLK |
| YCAKIGAYGYYFHYWGQGTLVTVSS (SEQ ID | TEDEADYYCQSSDDSNHLVFGGGTKLT |
| NO: 650) | VL (SEQ ID NO: 651) |
| QVQLVQSGAEGKKPGSSVKVSCKASGGTFSS | QPVLTQSPSASGTPGQRVTISCSGSSS |
| YAISWVRQAPGQGLEWMGGIIPIFGTANYAQK | NIGSNTVNWYQQLPGTAPKLLIYSNNQR |
| FQGRVTITADESTSTAYMELRSLRSDDTAVYYC | PSGVPDRFSGSKSGTSASLAISGLQSE |
| ARDAVVVADSGSMDVWGQGTTVTVSS (SEQ | DEADYYCAAWDDSLNGWVFGGGTKVT |
| ID NO: 652) | VL (SEQ ID NO: 653) |
| QVQLVQSGAEVKKPGSSVKVSCKASGGTFSS | QAGLTQPPSASGTPGQRVTISCSGSSS |
| YAISWVRQAPGQGLEWMGGIIPIFGTANYAQK | NIGSNTVNWYQQLPGTAPKLLIYSNNQR |
| FQGRVTITADESTSTAYMELSSLRSEDTAVYYC | PSGVPDRFSGSKSGTSASLAISGLQSE |
| ARDAVVVADSGSMDVWGQGTTVTVSS (SEQ | DEADYYCAAWDDSLNGWVFGGGTKLT |
| ID NO: 654) | VL (SEQ ID NO: 655) |
| QVQLQQSGPGLVKPSQTLSLTCAISGDSVSSN | QSVLTQPPSASGTPGQRVTISCSGSSS |
| SAAWNWIRQSPSRGLEWLGRTYYRSKWYNDY | NIGSNYVYWYQQLPGTAPKLLIYRNNQ |
| AVSVKSRITINPDTSKNQFSLHLNSVTPEDTAV | RPSGVPDRFSASKSGTSASLAISGLRSE |
| YYCATWGMDVWGQGTTVTVSS (SEQ ID | DEADYYCASWDDSLRGPVFGGGTKLT |
| NO: 656) | VL (SEQ ID NO: 657) |
| QVQLQQSGPGLVKPSQTLSLTCAISGDSVSSN | QPVLTQPPSASGTPGQRVTISCSGSSS |
| SAAWNWIRQSPSRGLEWLGRTYYRSKWYNDY | NIGSNYVYWYQQLPGTAPKLLIYRNNQ |
| AVSVKSRITINPDTSKNQFSLQLNSVTPEDTAV | RPSGVPDRFSGSKSGTSASLAISGLQS |
| YYCATWGMDVWGQGTTVTVSS (SEQ ID | EDEADYYCAAWDDSLNGPVFGGGTKL |
| NO: 658) | TVL (SEQ ID NO: 659) |
| QVQLQQSGPGLVKPSQTLSLTCAISGDSVSSN | QPVLTQPPSASGTPEQRVTISCSGSSS |
| SAAWNWIRQSPSRGLEWLGRTYYRSKWYNDY | NIGSNYVYWYQQLPGTAPKLLIYRNNQ |
| AVSVKSRITINPDTSKNQFSLHLNSVTPEDTAV | RPSGVPDRFSGSKSGTSASLAISGLRSE |
| YYCATWGMDVWGQGTMVTVSS (SEQ ID | DEADYYCAAWDDSLSGPVFGGGTKLTV |
| NO: 660) | L (SEQ ID NO: 661) |
| QVQLLQSAAEVKKPGASVKVPCKASGYTFTGH | QSALTQPHSVSESPGKTVTISCTRTSGR |
| YMHWVRQAPGQGLEWMGIINPRGGSTSYAQK | IGSNYVQWYQQRPGSAPTVVIYEDNQR |
| FQGRVTMTRDTSTSTVYMELSSLRSEDTAVYY | PSGVPDRFSGSIDSSTNSASLTISRLKTE |
| CARESIAAAGPYFDYWGQGTLVTVSS (SEQ ID | DEADYYCQSYDSGVVVVFGGGTKLTVL |
| NO: 662) | (SEQ ID NO: 663) |
| EVQLVESGGGLVQPGGSLRLSCAASGFTFNNF | NFMLTQPHSVSESPGKTVTISCTRSTGS |
| YMNWVRQAPGKGLEWISTISRGGNSVYYADS | IAGNYVQWYQQRPGSSPTTVILEDKQR |
| VKGRFTVSRDNAKNSLFLQMDSLRDEDTAVYY | PSGVPDRFSGSIDTSSNSATLTISGLKTE |
| CVRGGDFLYWGQGTLVTVSS (SEQ ID NO: 664) | DEADYYCQSYDSGNVIFGGGTKLTVL |
| | (SEQ ID NO: 665) |
| QVTLRESGGGLVKPGGSLRLSCSASGFTFRGF | NFMLTQPHSVSESPGKTVAISCSRSSG |
| YMAWIRQAPGKGLEWVAYISSSGSAVYHADSV | SVASNYVQWYQQRPGNVPTTIIYQDNQ |
| KGRFTVSRDNAKNLMYLEMNSLRADDTAVYYC | RPSGVPDRFSGSIDKSSNSASLTISGLK |
| ARDGGTGGFDYWGQGTLVTVSS (SEQ ID | TEDEADYYCQSYDGRRLIFGGGTKLTVL |
| NO: 666) | (SEQ ID NO: 667) |
| QVQLLQSAGGVVQPGRSLRLSCAASGFTFSTY | QSVLTQPPSVSAAPGQKVTISCSGSAS |
| AVHWVRQAPGKGLEWVAAISYDGAGKYYGDS | NIGNNYVSWYQQLPGTAPKLLIYDNNQ |
| VRGRFTISRDDSKNTVYLQMNSLRAEDTAVYY | RPSGIPDRFSGSKSGTSATLGITGLQTG |
| CAGEHYTLTDEEFDFWGQGTLVTVSS (SEQ ID | DEADYYCGTWDTSLRGVVFGGGTKVT |
| NO: 668) | VL (SEQ ID NO: 669) |
| HVQLVQSGGGSAQHGGSLRLSCAASGFTFNN | NFMLTQPHSVSESPGKTVTLSCTRSSG |
| SYMNWVRQAPGKRLEWISTISRGGNSVYYAD | SIASKYVQWYQQRPGSAPTIVIFDDDRR |
| SVKGRFTVSRDNTKNLTFLQMDSLRDEDTAVY | PSGVPDRFSGSIDRSSNSASLTIFGMKT |
| YCAKGGDYLYWGQGTLVTVSS (SEQ ID | EDEADYYCQSYDSSNWVFGGGTKLTVL |
| NO: 670) | (SEQ ID NO: 671) |
| AVQLVESAGGVVQLRRSLSLSSAASRFTFSSY | QSVLTQPPSVSAAPGQKATISCSGGSS |
| PMHWIRQAPVKGMEWVSAISGSGGSTYYADS | NLGNNYVSWYQQLPGTAPKLLIYDNNK |
| VKGRFTISRHNSKNTLYLQMNSLRAEDTGVYW | RPSGIPDRFSGSKSGTSASLAITGLQTG |
| GARDPHDFWGQGTLVTVSS (SEQ ID NO: 672) | DEADYYCGTWDSSLSAGVFGGGTKLTV |
| | L (SEQ ID NO: 673) |
| QVQLVQSGGGLVQPGRSLRLSCAASGFTFDD | QSVLTQPPSVSAAPGKKVTISCSGGSS |
| FAMHWVRQAPGKGLEWVSGITWNSAATIYAD | NIGKNYVSWYQQLPGTAPKLLIYDNNKR |
| SVKGRFTISRDNAKNSLYLQMNSLRPEDTAVY | PSGIPDRFSGSKSGTSATLGITGLQTGD |
| YCARDRGGPKDPFDIWGQGTMVTVSS (SEQ | EADYYCGTWDSSLSAVVFGGGTKLTVL |
| ID NO: 674) | (SEQ ID NO: 675) |
| QVQLQQSGAEVKKPGASLKVSCKASGYTFTRY | SYVLTQPPSVSVAPGQTAKITCGGDNIE |
| GFSWVRQAPGQGLEWMGWISAYNGNTNYAQ | NKYVHWYQQKPGQAPVVVVYDDSDRP |
| KFQGRVTMTTDTSTSTAYMEMRSLRSDDTAVY | SGIPDRFSGSNSGNTATLTITRVEAGDE |
| YCARDRGYCSGGSCPGIDAFDIWGQGTMVTV | ADYYCQVWDSSSDRVVFGGGTKLTVL |
| SS (SEQ ID NO: 676) | (SEQ ID NO: 677) |
| QVQLVQSGPEVKKPGASVNISCKTSGYTFRNY | NFMLTQPHSVSESPGKTVTISCTGSSG |
| YVHWVRQAPGQGFEYMGRVNPNSGNARYAQ | SIASNYVHWYQQRPGSAPTTVIYEDNQ |
| KFQGRVAMTSDTSTSTAYMELSSLRSEDTAVY | RPSGVPDRFSGSIDSSSNSASLTISGLK |
| YCAREPPATVAYYYGMDVWGQGTTVTVSS | TEDEADYYCRSYDSSNRNWVFGGGTK |
| (SEQ ID NO: 678) | LTVL (SEQ ID NO: 679) |
| EVQLVESGGGVVQPGRSLRLSCAASGFTFSSY | NFMLTQPHSVAESPGKTVTISCTRSSGS |
| GIHWVRQAPGKGLEWVSAISGSGGSTYYADS | IASNYVQWYQQRPGSAPTTVIYEDNQR |
| VKGRFTISRDNAKNSLYLQMNSLRDEDTAVYY | PSGVPDRFSGSIDSSSNSASLTISGLKT |
| CARERLIGMDVWGQGTTVTVSS (SEQ ID | EDEADYYCQSYDSGNPRVFGGGTKLTV |
| NO: 680) | L (SEQ ID NO: 681) |
| QVQLVQSGAEVKKPGASVKVSCKASGYTFTSY | QPVLTQPPSASGTPGQRVTISCSGSSS |
| GISWVRQAPGQGLEWMGWISAYNGNTNYAQK | NIGSKYVYWYQQLPGTAPKLLIYRNNQR |
| LQGRVTMTTDTPTSTAYMEVRSLRSDDTAVYY | PSGVPDRFSGSKSGTSASLVISGLRSED |
| CAREVNYGLDVWGQGTLVTVSS (SEQ ID | EADYHCAAWDDSLSGRVFGGGTKLTVL |
| NO: 682) | (SEQ ID NO: 683) |
| EVQLVESGGGLVQPGGSLRLSCAASGFTSNTY | NFMLTQPHSVSESPGKTVTISCTRNTG |
| AMSWVRQAPGKGLEWVSTISGSGSSTNYADS | GIAANYVQWYHQRPGSAPTTVIYEDKQ |
| VKGRFTISRDSSKNTLYLQMNSLRAEDTAVYY | RPSGVPGRFSGSIDGSSNSASLTISGLQ |
| CARIERYSYGIDYWGQGTLVTVSS (SEQ ID | AEDEGDYYCHSSDNNNQVFGGGTKLT |
| NO: 684) | VL (SEQ ID NO: 685) |
| QVQLVQSGGGVVQPARSLRLSSAASGFTFSSY | SYVLTQPPSVSVAPGQTARITCGGDNIG |
| GMHWVRQAPGKRLEWVAVISPDGSDKYYADS | LKSVHWYQQKPGQAPVLVVYDDVDRP |
| VKGRFSVSRGNSKNMLYLQMTGLTTEDTAVYY | SDIPERFSGSNSGNTATLTISRVEAGDE |
| CARNRKYTNFLDIWGQGTMVTVSS (SEQ ID | ADYYCQVWDDPSDQGSWVFGGGTKLT |
| NO: 686) | VL (SEQ ID NO: 687) |
| QVQLVQSGAEVKKPGASVKVSCKASGYTFTSY | EIVLTQSPGTLSLSPGERATLSCRASQS |
| YMHWVRQAPGQGLEWMGIINPSGGTTSYAQK | VSSSYLAWYQQKPGQAPRLLIYGASSR |
| FQGRVTMTRDTSTSTVYMELSSLRSEDTAVYY | ATGIPDRFSGSGSGTDFTLTISRLEPED |
| CARNWASFDYWGQGTLVTVSS (SEQ ID | FAVYYCQQYGSSPYTFGQGTKVDIK |
| NO: 688) | (SEQ ID NO: 689) |
| EVQLVQSGGGLVKPGRSLRLSCTASGFTFGDY | QSVLTQPPSVSAAPGQKVTISCSGSSS |
| AMSWFRQAPGKGLEWVGFIRSKAYGGTTEYA | NIGNNYVSWYQQLPGTAPKLLIYDNNKR |
| ASVKGRFTISRDDSKSIAYLQMNSLKTEDTAVY | PSGIPDRFSGSKSGTSATLGITGLQTGD |
| YCTRVSDIYGMDVWGQGTTVTVSS (SEQ ID | EADYYCATWDDSLKAGVFGGGTKLTVL |
| NO: 690) | (SEQ ID NO: 691) |
| QVNLRESGGGVVQPGGSLRLSCAASGFTFRR | NFMLTQPHSVSESPGRTVTISCTRSSG |
| YGMNWIRQAPGKGLEWVSSISSSSSYIYYADS | SIASNYVQWYQQRPGSSPTTVIYDDKK |
| VKGRFTISRDNAKNSLYLQMNSLRAEDTAMYY | RPSGVPDRFSGSIDSSTNSASLTISGLK |
| CVRDGRFINGLDVWGQGTLVTVSS (SEQ ID | TEDEADYYCQSYDSSNHWVFGGGTKV |
| NO: 692) | TVL (SEQ ID NO: 693) |
| QVQLVQSGGGLVQPGGSLRLSCAASGFTFSR | SYVLTQPPSVSASPGQTASITCSGHKLG |
| YSMSWVRQAPGKGLEWVSSISGGGGSTYYAD | DKYTSWYQQKPGQSPVLVIYQDNKRPS |
| SVKGRFTISRDNSDGSVYLKMNSLAAGDTAAY | GIPERFSGSNSGNTATLTISGTQAMDEA |
| YCVRGGWYRTPFEFWGRGTLVTVSS (SEQ ID | DYYCQAWDSSTNYVFGTGTKLTVL |
| NO: 694) | (SEQ ID NO: 695) |

71. The antibody or antigen-binding fragment thereof according to any one of items 59 to 70, wherein said antigen binding fragment is a Fab fragment, a F(ab′)₂ fragment, a Fd fragment, an Fv fragment, a single-chain Fv (scFv) molecule, or a single-domain antibody (dAb).
72. The antibody or antigen-binding fragment thereof according to any one of items 59 to 71, which is a multispecific antibody.
73. The antibody or antigen-binding fragment thereof according to item 72, wherein said multispecific antibody is a bispecific antibody.
74. The antibody or antigen-binding fragment thereof according to item 72 or 73, wherein the multispecific further comprises a second antibody or antigen-binding fragment thereof that specifically binds to a tumor antigen.
75. The antibody or antigen-binding fragment thereof according to item 74, wherein the tumor antigen is TrkB.
76. The antibody or antigen-binding fragment thereof according to item 75, wherein the second antibody or antigen-binding fragment thereof is the antibody or antigen-binding fragment thereof defined in any one of claims 1 to 14.
77. A conjugate comprising the antibody or antigen-binding fragment thereof defined in any one of items 59 to 76, and a therapeutic agent, such as an anti-inflammatory or immunosuppressive agent.
78. A pharmaceutical composition comprising the antibody or antigen-binding fragment thereof defined in any one of items 59 to 76, the conjugate of claim 77, and a pharmaceutically acceptable excipient.
79. A method for treating a CD3-associated autoimmune disease in a subject in need thereof, comprising administering to the subject an effective amount of the antibody or antigen-binding fragment thereof of any one of items 59 to 76, the conjugate of claim 77, or the pharmaceutical composition of item 78.
80. The method of item 79, wherein the CD3-associated autoimmune disease is multiple sclerosis (MS), rheumatoid arthritis (RA), systemic lupus erythematosus, Celiac disease, Sympathetic ophthalmia, Type 1 diabetes, or graft-versus-host disease (GvHD).
81. A method for treating cancer in a subject in need thereof, comprising administering to the subject an effective amount of the antibody or antigen-binding fragment thereof of any one of items 59 to 76, the conjugate of claim 77, or the pharmaceutical composition of item 78.
82. The method of item 81, wherein the antibody or antigen-binding fragment thereof is the multispecific antibody or antigen-binding fragment of any one of items 72 to 76.
83. The method of item 81 or 82, wherein the cancer is precursor T acute lymphoblastic leukemia/lymphoma, anaplastic large-cell lymphoma, lymphomatoid papulosis type A, Mycosis fungoides, pagetoid reticulosis, granulomatous slack skin, Sezary disease, adult T-cell leukemia/lymphoma, cutaneous large T-cell lymphoma, pleomorphic T-cell lymphoma, lymphomatoid papulosis type B, secondary cutaneous CD30+ large-cell lymphoma, hepatosplenic T-cell lymphoma, angioimmunoblastic T-cell lymphoma, enteropathy-associated T-cell lymphoma, peripheral T-cell lymphoma not otherwise specified, subcutaneous T-cell lymphoma, large granular lymphocytic leukemia, acute biphenotypic leukemia, adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, acute and chronic leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, or metastases thereof.
84. The method of any one of items 79 to 83, wherein the antibody or antigen binding-fragment is administered to the subject separately, sequentially or simultaneously with an additional therapeutic agent.
85. The method of item 84, wherein the additional therapeutic agent is one or more of non-steroidal anti-inflammatory drugs (NSAIDs), selective COX-2 inhibitors, glucocorticoids, conventional disease-modifying anti-rheumatic drugs (cDMARDs) and antitumor agents.
86. The method of item 85, wherein the antitumor agent is one or more of alkylating agents, platinum agents, taxanes, vinca agents, anti-estrogen drugs, aromatase inhibitors, ovarian suppression agents, VEGF/VEGFR inhibitors, EGF/EGFR inhibitors, PARP inhibitors, cytostatic alkaloids, cytotoxic antibiotics, antimetabolites, endocrine/hormonal agents, bisphosphonate therapy agents or a checkpoint inhibitor.
87. Use of the antibody or antigen-binding fragment thereof of any one of claims 59 to 76, the conjugate of item 77, or the pharmaceutical composition of item 78, for treating a CD3-associated autoimmune disease in a subject, or for the manufacture of a medicament for treating a CD3-associated autoimmune disease in a subject.
88. The use of item 87, wherein the CD3-associated autoimmune disease is multiple sclerosis (MS), rheumatoid arthritis (RA), systemic lupus erythematosus, Celiac disease, Sympathetic ophthalmia, Type 1 diabetes, or graft-versus-host disease (GvHD).
89. Use of the antibody or antigen-binding fragment thereof of any one of claims 59 to 76, the conjugate of item 77, or the pharmaceutical composition of item 78, for treating cancer in a subject, or for the manufacture of a medicament for treating cancer in a subject.
90. The use of item 89, wherein the antibody or antigen-binding fragment thereof is the multispecific antibody or antigen binding fragment of any one of items 72 to 76.
91. The use of item 89 or 90, wherein the cancer is precursor T acute lymphoblastic leukemia/lymphoma, anaplastic large-cell lymphoma, lymphomatoid papulosis type A, Mycosis fungoides, pagetoid reticulosis, granulomatous slack skin, Sezary disease, adult T-cell leukemia/lymphoma, cutaneous large T-cell lymphoma, pleomorphic T-cell lymphoma, lymphomatoid papulosis type B, secondary cutaneous CD30+ large-cell lymphoma, hepatosplenic T-cell lymphoma, angioimmunoblastic T-cell lymphoma, enteropathy-associated T-cell lymphoma, peripheral T-cell lymphoma not otherwise specified, subcutaneous T-cell lymphoma, large granular lymphocytic leukemia, acute biphenotypic leukemia, adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, acute and chronic leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, or metastases thereof.
92. The use of any one of items 87 to 91, wherein the antibody or antigen-binding fragment, conjugate or composition is for administration to the subject separately, sequentially or simultaneously with an additional therapeutic agent.
93. The use of item 92, wherein the additional therapeutic agent is one or more of non-steroidal anti-inflammatory drugs (NSAIDs), selective COX-2 inhibitors, glucocorticoids, and conventional disease-modifying anti-rheumatic drugs (cDMARDs).
94. The use of item 92, wherein the additional therapeutic agent is an antitumor agent.
95. The use of item 94, wherein the antitumor agent is one or more of alkylating agents, platinum agents, taxanes, vinca agents, anti-estrogen drugs, aromatase inhibitors, ovarian suppression agents, VEGF/VEGFR inhibitors, EGF/EGFR inhibitors, PARP inhibitors, cytostatic alkaloids, cytotoxic antibiotics, antimetabolites, endocrine/hormonal agents, bisphosphonate therapy agents or a checkpoint inhibitor.
96. The antibody or antigen-binding fragment thereof of any one of items 59 to 76, the conjugate of item 77, or the pharmaceutical composition of item 78, for use in treating a CD3-associated autoimmune disease in a subject.
97. The antibody or antigen-binding fragment thereof, conjugate or composition for use of item 96, wherein the CD3-associated autoimmune disease is multiple sclerosis (MS), rheumatoid arthritis (RA), systemic lupus erythematosus, Celiac disease, Sympathetic ophthalmia, Type 1 diabetes, or graft-versus-host disease (GvHD).
98. The antibody or antigen-binding fragment thereof or composition of any one of items 59 to 76, the conjugate of item 77, or the pharmaceutical composition of item 78, for use in treating cancer in a subject.
99. The antibody or antigen-binding fragment thereof, conjugate or composition for use of claim 98, wherein the antibody or antigen-binding fragment thereof is the multispecific antibody or antigen-binding fragment of any one of items 72 to 76.
100. The antibody or antigen-binding fragment thereof, conjugate or composition for use of item 98 or 99, wherein the cancer is precursor T acute lymphoblastic leukemia/lymphoma, anaplastic large-cell lymphoma, lymphomatoid papulosis type A, Mycosis fungoides, pagetoid reticulosis, granulomatous slack skin, Sezary disease, adult T-cell leukemia/lymphoma, cutaneous large T-cell lymphoma, pleomorphic T-cell lymphoma, lymphomatoid papulosis type B, secondary cutaneous CD30+ large-cell lymphoma, hepatosplenic T-cell lymphoma, angioimmunoblastic T-cell lymphoma, enteropathy-associated T-cell lymphoma, peripheral T-cell lymphoma not otherwise specified, subcutaneous T-cell lymphoma, large granular lymphocytic leukemia, acute biphenotypic leukemia, adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, acute and chronic leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, or metastases thereof.
101. The antibody or antigen-binding fragment thereof, conjugate or composition for use of any one of items 96 to 100, wherein the antibody or antigen binding fragment is for administration to the subject separately, sequentially or simultaneously with an additional therapeutic agent.
102. The antibody or antigen-binding fragment thereof, conjugate or composition for use of item 101, wherein the additional therapeutic agent is one or more of non-steroidal anti-inflammatory drugs (NSAIDs), selective COX-2 inhibitors, glucocorticoids, and conventional disease-modifying anti-rheumatic drugs (cDMARDs).
103. The antibody or antigen-binding fragment thereof, conjugate or composition for use of item 101, wherein the additional therapeutic agent is an antitumor agent.
104. The antibody or antigen-binding fragment thereof, conjugate or composition for use of item 103, wherein the antitumor agent is one or more of alkylating agents, platinum agents, taxanes, vinca agents, anti-estrogen drugs, aromatase inhibitors, ovarian suppression agents, VEGF/VEGFR inhibitors, EGF/EGFR inhibitors, PARP inhibitors, cytostatic alkaloids, cytotoxic antibiotics, antimetabolites, endocrine/hormonal agents, bisphosphonate therapy agents or a checkpoint inhibitor.
105. A method of detecting CD3 or a CD3-expressing cell in a sample comprising contacting the sample with the antibody or antigen-binding fragment thereof of any one of items 59 to 76.

Other objects, advantages and features of the present disclosure will become more apparent upon reading of the following non-restrictive description of specific embodiments thereof, given by way of example only with reference to the accompanying drawings.

### BRIEF DESCRIPTION OF DRAWINGS

In the appended drawings:

FIG. 1 depicts an SDS-PAGE analysis of 30 anti-TrKB IgG clones (1 μg per slot) under reducing conditions. Proteins were visualized using Coomassie Blue. Following transient expression in HEK293 cells, the recombinant anti-TrkB monoclonal antibodies were protein A-affinity purified and subsequently re-buffered to PBS by dialysis.

FIGS. 2A-E show an ELISA-screening of purified anti-TrkB antibodies discovered from human phage libraries. Human TrkB-ECD-GST (inverted triangles), mouse TrkB-ECD-GST (squares) and biotinylated GST (circles) were immobilized overnight on ELISA plates, which were then blocked and subsequently incubated with a seven-step semi-log dilution series starting at 10 μg/mL of the purified indicated recombinant antibodies. Antibody binding was detected with anti-human IgG-HRP and TMB staining. The average absorbance (A) at A 450 nm (and range of technical duplicates) is depicted. Reference anti-TrkB, reference anti-GST and secondary antibody (anti-human lgG-HRP) only were used as controls. The probed clone is indicated in top of each panel.

FIGS. 3A-C depict a dose-dependent specificity screening using ELISA of recombinant DNA full-length anti-TrkB antibodies obtained from human phage libraries. Proteins at a concentration of 1 μg/mL, were coated on the SBS plates overnight, blocked and subsequently incubated with an eleven-step semi-log dilution series of the recombinant DNA antibodies starting at 10 μg/mL. Reactivity against human TrkA-His (squares), human TrkB-His (dots), human TrkC-His (triangles) and biotinylated-BSA (inverted triangles) was assessed. The probed clone is indicated in top of each panel. Detection was accomplished with anti-human-IgG-HRP and visualization using TMB. Target coating was validated with proper anti-target antibodies, streptavidin-HRP for biotinylated-BSA. Anti-human-IgG-HRP only was included as negative control. The optical density at A 450 nm for each mAb is depicted.

FIGS. 4A-D depict flow cytometric analyses of synthesized anti-TrkB antibodies affinity-purified from transfected HEK293F cells. The reactivity of these antibodies was assessed towards parental CHO-K1 (triangles), and human TrkB-expressing (dots) and mouse TrkB-expressing (squares) CHO-K1 cells. For this purpose, an eight-step semi-log dilution series of the clones starting at 30 μg/mL were analyzed. Antibody binding was detected using anti-human-IgG-F(ab)-PE. Average median fluorescence intensity (MFI) and range of technical duplicates is shown. The probed clone is indicated above each panel.

FIG. 5A summarizes the results of pairwise epitope binning analysis of candidate clones from human phage libraries (Hu) or chicken B-cell libraries (Ch). Bins were arbitrary designated to 1a, 1b, 2a, 2b, 3a, 3b, and from 4 to 7 in a pairwise competition analysis of all mAb clones. Inter-bin blocking competition with benchmark antibodies, which are known to bind to TrkB extracellular domain (ECD) domains D1, D3, D5, and JM, and with selected mAb clones from each bin revealed the domains to which the clones within each bin putatively bind.

FIG. 5B is a schematic molecular representation of transmembrane protein TrkB indicating its domains D1 to D5, and JM, which represents the extracellular juxta-membrane motif. The experimentally predicted locations of the epitope bins of discovered anti-TrkB antibodies based on relationships to reference antibodies and patterns of blocking, are indicated on the right. The schematic structure is adapted from Ref. Amatu et al., Ann Oncol. 2019; 30: viii5-viii15, doi: 10.1093/annonc/mdz383.

FIG. 6A shows that none of the tested discovered anti-TrkB antibodies trigger TrkA-dependent signaling in TrkA-sensitive reporter cells. The antibodies were added to TrkA-activation reporter cells in a seven-step semi-log 10 dilution series of TrkB antibodies starting from 31.6 μg/mL.

FIG. 6B shows that TrkA-sensitive reporter cells were not activated by BDNF (at 4 nM) or by NISTmab, but were activated by TrkA's natural ligand nerve growth factor (NGF).

FIG. 7 shows the results of a sandwich ELISA-screening, detecting tyrosine-phosphorylated human TrkB in cell lysates of TrkB-expressing CHO cells after 15 min of incubation at 37° C. with a 3-step dilution series of anti-TrkB mAbs and one bsAb (at concentrations of 0.1, 1.0, and 10 μg/mL). An immobilized monoclonal capture mAb specific for TrkB was used to capture both phosphorylated and non-phosphorylated TrkB, a Tyr-P specific antibody was used to detect phosphorylated TrkB and stained for analysis at λ 450 nm. Results are shown for human phage library-derived clones, 3H11, 6E08, 3C12, 1F01, 2E02, 6B04, chicken-derived anti-TrkB mAb F5, as well as for F5×1A09 (anti-TrkB×anti-CD3) bsAb. The following controls were included: 10 nM BDNF as positive control, and 10 μg/mL hIgG1 and a 3-step dilution series MQR2.101× NISTmAb (bsAb control) as negative controls. Data are presented as technical replicates (N=2). The signal of control BDNF and of isotype control are indicated by dotted horizontal lines in each panel which represent different ELISA plates.

FIGS. 8A-I represent internalization capability of the indicated anti-TrkB chicken clones as determined by the residual receptor presence at the surface method. At each time point, the median fluorescence intensity (MFI) intensity representing the amount each antibody binding to CHO-TrkB and CHO parental cells at 4° C. was divided by the signal at 37° C. to obtain the ratio. Average ratio±sd of technical triplicates is shown.

FIGS. 9A-9B are bar graphs depicting the internalization capability of the indicated human anti-TrkB clone. The MFI representing the amount of each antibody binding to CHO-TrkB cells was measured at 4° C. and 37° C. The ratio of 4° C. to 37° C. MFI signals was calculated for all time points: 2h (first bars from the left), 4h (second bars from the left), 6h (third bars from the left), 16h (third bars from the right), 20h (second bars from the right), and 24h (first bars from the right). Average MFI ratio±sd of technical triplicates is shown.

FIG. 10 is a bar graph showing the internalization capability of two human anti-TrkB clones (2E02 and 1F01) in A549 a lung carcinoma derived cell line. IC: isotype control, 2nd only: no TrkB antibody added. The MFI representing the amount of each antibody binding to CHO-TrkB cells was measured at 4° C. and 37° C. The ratio of 4° C. to 37° C. readouts were calculated. Average MFI ratio±sd of technical replicates is shown.

FIGS. 11A-C show an SDS-PAGE analysis of Fab clones produced from 30 selected anti-CD3 scFvs under reducing conditions. Each lane was loaded with 2 μg of purified Fab antibody fragment. Protein was visualized using Instant-Blue protein staining. The 30 Fab clones were synthesized from 30 out of 39 unique scFv sequence combinations following the sequence analysis of 180 selected monoclonal phages.

FIGS. 12A-D show examples of ELISA-binding analyses of representative anti-CD3 Fabs identified by the screening (3-A03, FIG. 12B; 1-A09, FIG. 12C; 1-C12, FIG. 12D). ELISA-screening of purified Fab antibody fragments for their reactivity towards human CD30/8 (dots) and BSA (squares). As positive control the CD3-binding fragment (Fab) of OKT-3/blinatumomab (Blina, FIG. 12A) was used. Concentrations of 1 μg/mL biotinylated His-Avi-tagged CD30/8 heterodimer or BSA were coated overnight on ELISA plates, blocked and subsequently incubated with a seven-step semi-log dilution series of purified Fabs starting at a concentration of 10 μg/mL. Antibody binding was detected using anti-human-Lambda-HRP or anti-human-Kappa-HRP and visualized using TMB. The average optical density (OD) readout at A 450 nm (and range of technical duplicates) is plotted.

FIGS. 13A-E show examples of flow-cytometric analyses of the binding reactivity of representative anti-CD3 Fabs identified by the screening (3-A03, FIG. 13B; 1-A09, FIG. 13C; 1-C12, FIG. 13D). Flow-cytometric analyses of purified anti-CD30/8 human Fab antibody fragments expressed in HEK293 cells. The reactivity of these binders towards parental CHO-K1 parental, CD3δ/ε-expressing CHO-K1 cells and CD3-containing Jurkat cells was probed in an 11-step semi-log dilution series of purified Fabs (starting at 150 μg/mL). Antibody binding was detected using anti-human-IgG-F(ab)-PE. Anti-CD3 full-length mAb (OKT3) and Fab (blina, FIG. 13A)) were used as positive controls, while other antibodies were included as negative controls (FIG. 13E). Average median fluorescence intensity (MFI) and range of technical duplicates is shown.

FIGS. 14A-D show the CD3 activation potential of representative anti-CD3 Fabs identified by the screening in a Jurkat cell activation assay. Fab clones were coated overnight at 4° C. on an ELISA plate in eleven wells through an 11-step semi-log dilution series starting at 150 μg/mL. Following washing with PBS, 40×10³ Jurkat NFAT reporter cells were seeded into each well and incubated for 24 h at 37° C. After the addition of 50 μL PathHunter® detection solution (DiscoverX), chemiluminescence was measured in relative fluorescence units (RFU). Examples of a very strong (OKT3, FIG. 14A), strong (clone 1-A09, FIG. 14C), weak (clone 3-A03, FIG. 14B) and non-activator (clone 1C-12, FIG. 14D) are shown.

FIGS. 15A-D show examples of bio-layer interferometry (BLI) biosensor (Octet) off-rate analyses of representative anti-CD3 Fabs identified by the screening (3-A03, FIG. 15B; 1-A09, FIG. 15C; 1-C12, FIG. 15D). The surface of streptavidin-coated sensor chips was prepared with biotinylated His-Avi-tagged CD30/heterodimer. Fabs at 20 μg/mL were flowed over the sensor chip for 300 s followed by Fab-free running buffer for 150 s to measure the dissociation of the binder in the first 20 s. Sensorgrams were corrected and zeroed for the signal for the biosensor's response when only buffer passed the CD3δ/ε-coated sensor. Experiments were conducted at 30° C. in a run buffer of PBS containing 0.05% (v/v) Tween™-20 and 0.1% (m/v) BSA. The CD3-binding fragment (Fab) of blinatumomab (Blina, FIG. 15A) was used as a positive control.

FIG. 16A depicts an ELISA-screening of protein A-purified bsAbs composed of isotype control MQR2.101 as scFv-knob-Fc chains with anti-CD3 antibody or isotype control NISTmAb variable part as Fab-hole sequence. Following the immobilization of either human TrkB-His (huTrkB-His; dots), biotinylated CD3 (squares) or BSA (Biotin-BSA; triangles), wells were blocked with 1% (w/v) BSA in PBS. The immobilized proteins were incubated with a seven-step semi-log 10 dilution series of the indicated knob-in-hole derived bsAbs starting at 10 μg/mL. The ELISA performance was verified using anti-TrkB (clone 3H11), anti-His tag and streptavidin-HRP as coating controls. Anti-human-IgG-HRP or anti-mouse-IgG-HRP were included as single binding reagents to function as negative controls. Antibody binding was detected with anti-human IgG-HRP and TMB staining. The average absorbance (A) at A 450 nm (and range of technical duplicates) is depicted.

FIGS. 16B-E depict an ELISA-screening of protein A-purified bsAbs composed of discovered anti-TrkB as scFv-knob-Fc chains (clone name indicated in front of hyphen) with the variable part of anti-CD3 antibody (clone name indicated following hyphen) or isotype control NISTmAb as Fab-hole sequence. Following the immobilization of either human TrkB-His (huTrkB-His; dots), biotinylated CD3 (Biotin-CD3; squares) or BSA (Biotin-BSA; triangles), wells were blocked with 1% (w/v) BSA in PBS. The immobilized proteins were incubated with a seven-step semi-log 10 dilution series of the indicated knob-in-hole derived bsAbs starting at 10 μg/mL. The ELISA performance was verified using anti-TrkB (clone 3H11), anti-His tag and streptavidin-HRP as coating controls. Anti-human-IgG-HRP or anti-mouse-IgG-HRP were included as single binding reagents to function as negative controls. Antibody binding was detected with anti-human lgG-HRP and TMB staining. The average absorbance (A) at A 450 nm (and range of technical duplicates) is depicted.

FIG. 17A depicts a flow cytometric screening of protein A-purified bsAbs composed of isotype control MQR2.101 as scFv-knob-Fc chain joint with an anti-CD3 antibody or isotype control NISTmAb variable part as Fab-hole sequence. Flow cytometry-based antibody screening against CHO-k1 expressing human TrkB (CHO-k1 huTrkB, squares), CHO-k1 expressing human CD3d/e (CHO-k1 huCD3d/e, triangles), and CHO-k1 parental (CHO-k1 parental, dots) cells. Harvested cells were washed, re-suspended and subsequently incubated with a 6-step semi-log 10 dilution series of indicated knob-in-hole derived bsAbs (starting at 10 μg/mL). Average MFI±sd of technical replicates is shown.

FIGS. 17B-E depict flow cytometry-based screenings of protein A-purified bsAbs composed of discovered anti-TrkB mAbs as scFv-knob-Fc chains (clone name indicated in front of the hyphen) with the variable part of anti-CD3 antibody (clone name indicated following the hyphen) or isotype control NISTmAb as Fab-hole sequence. Flow cytometry-based antibody screening against CHO-k1 human TrkB (CHO-k1 huTrkB, squares), CHO-k1 human CD3d/e (CHO-k1 huCD3d/e, triangles), and CHO-k1 parental (CHO-k1 parental, dots) cells. Cells were harvested, washed, re-suspended and subsequently incubated with a 6-step semi-log 10 dilution series of the indicated discovered anti-TrkB/CD3 bispecific antibodies (starting at 10 μg/mL). Average MFI±sd of technical replicates is shown.

FIG. 18A depicts a flow cytometry-based bridging assay with protein A-purified bsAbs composed of isotype control MQR2. 101 as scFv-knob-Fc chains with anti-CD3 antibody or isotype control NISTmAb variable part as Fab-hole sequence. FL4-stained CHO-k1_CD3 cells were incubated with an 8-step semi-log 10 dilution series of the indicated knob-in-hole-derived bsAbs, starting at 100 μg/mL. These treated cells were then incubated with FL1-stained CHO-k1_TrkB cells (huCD3+huTrkB, triangles). Antibody bridging was measured by the detection of FL1/FL4 positive doublets on an iQue flow cytometer. Combinations with appropriately stained CHO-k1 parental cells were used as negative control (parental+huTrkB, squares; parental+huCD3d/e, dots).

FIG. 18B depicts a flow cytometry-based bridging assay with protein A-purified bsAbs composed of anti TrkB as scFv-knob-Fc chains (clone name indicated in front of the underscore) with isotype control NISTmAb variable part as Fab-hole sequence. FL4-stained CHO-k1_CD3 cells were incubated with an 8-step semi-log 10 dilution series of the indicated knob-in-hole-derived bsAbs (starting at 100 μg/mL). These cells were then incubated with FL1-stained CHO-k1 expressing TrkB (huCD3+huTrkB, triangles). Antibody bridging was measured by the detection of FL1/FL4-positive doublets on an iQue flow cytometer. Combinations with appropriately stained CHO-k1 parental cells were used as negative control (parental+huTrkB, squares; parental+huCD3d/e dots).

FIGS. 18C-E depict flow cytometry-based bridging assays of protein A-purified bsAbs composed of discovered anti-TrkB as scFv-knob-Fc chains (clone name indicated in front of the underscore) with the variable part of anti-CD3 antibody (clone name indicated following the underscore). FL4-stained CHO-k1_CD3 cells were incubated with an 8-step semi-log 10 dilution series of the discovered anti-TrkB/CD3 knob-in-hole-derived bsAbs, starting at 100 μg/mL. These cells were then incubated with FL1-stained CHO-k1_TrkB cells (huCD3+huTrkB, triangles). Antibody bridging was measured by the detection of FL1/FL4 positive doublets on an iQue flow cytometer. Combinations with appropriately stained CHO-k1 parental cells were used as negative control (parental+huTrkB, squares; parental+huCD3d/e dots).

FIG. 19A depicts a Jurkat NFAT reporter cell-based screening assay with protein A-purified bsAbs composed of isotype control MQR2. 101 as the scFv-knob-Fc chain with anti-CD3 antibody (clone name indicated following the forward slash) or isotype control NISTmAb variable part as Fab-hole sequence, and protein A-purified bsAb composed of anti TrkB as scFv-knob-Fc chain (clone name indicated in front of the forward slash) with isotype control NISTmAb variable part as Fab-hole sequence. Jurkat NFAT and CHO-k1 human TrkB (Jurkat NFAT+CHO huTrkB, squares) cells were mixed at a 1:1 ratio and seeded in 96-wells assay plates. Jurkat NFAT cells seeded alone (Jurkat NFAT, dots) or mixed with CHO-k1 parental cells (Jurkat NFAT+CHO parental, triangles) were included as control. Analysis was performed with an 11-step semi-log 10 dilution series of the indicated knob-in-hole-derived bsAbs (starting at 100 μg/mL). Average luminescence signals±sd of technical duplicates are shown.

FIGS. 19B-C depict a Jurkat NFAT reporter cell-based screening assay with protein A-purified bsAbs composed of discovered anti-TrkB as scFv-knob-Fc chains (clone name indicated in front of forward slash) with the variable part of anti-CD3 antibody (clone name indicated following forward slash). Jurkat NFAT and CHO-k1 human TrkB (Jurkat NFAT+CHO huTrkB, squares) cells were mixed at a 1:1 ratio. The results for Jurkat NFAT cells seeded alone (Jurkat NFAT, dots) or Jurkat NFAT cells mixed with CHO-k1 parental cells (Jurkat NFAT+CHO parental, triangles) as controls are shown. In addition, the response of 11-step semi-log 10 dilution series of indicated anti-TrkB/CD3 bsAbs (starting at 100 μg/mL) is exhibited. Average luminescence signals±sd of technical duplicates are shown.

### DETAILED DISCLOSURE

The use of the terms “a” and “an” and “the” and similar referents in the context of describing the technology (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

All methods described herein can be performed in any suitable order unless otherwise indicated herein or otherwise clearly contradicted by context.

The use of any and all examples, or exemplary language (“e.g.”, “such as”) provided herein, is intended merely to better illustrate embodiments of the claimed technology and does not pose a limitation on the scope unless otherwise claimed.

No language in the specification should be construed as indicating any non-claimed element as essential to the practice of embodiments of the claimed technology.

Herein, the term “about” has its ordinary meaning. The term “about” is used to indicate that a value includes an inherent variation of error for the device or the method being employed to determine the value, or encompass values close to the recited values, for example within 10% of the recited values (or range of values).

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

Where features or aspects of the disclosure are described in terms of Markush groups or list of alternatives, those skilled in the art will recognize that the disclosure is also thereby described in terms of any individual member, or subgroup of members, of the Markush group or list of alternatives.

Unless specifically defined otherwise, all technical and scientific terms used herein shall be taken to have the same meaning as commonly understood by one of ordinary skill in the art (e.g., in stem cell biology, cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

### Anti-TrkB Antibodies and Antigen-Binding Fragments Thereof

In an embodiment, the present disclosure provides an antibody (e.g., an anti-TrkB antibody) comprising a combination of heavy chain complementarity determining regions (CDR-H1, CDR-H2 and CDR-H3) and light chain complementarity determining regions (CDR-L1, CDR-L2 and CDR-L3) from any of the anti-TrkB antibody clones disclosed herein (e.g., Tables 4 and 7).

In an embodiment, the present disclosure provides an antibody (e.g., an anti-TrkB antibody) comprising a combination of heavy chain complementarity determining regions (CDR-H1, CDR-H2 and CDR-H3) and light chain complementarity determining regions (CDR-L1, CDR-L2 and CDR-L3), wherein said CDR-H1, CDR-H2, CDR-H3, CDR-L1, CDR-L2 and CDR-L3 comprise or consist of amino acid sequences having at least 70% identity with the sequences depicted in Tables 1A and 1b:

TABLE 1A

| | | SEQ | | SEQ | | SEQ |
|---|---|---|---|---|---|---|
| Clone | | ID | | ID | | ID |
| ID | CDR-H1 | NO | CDR-H2 | NO | CDR-H3 | NO |
| 1E02 | GYRFTSYW | 1 | IYPDDSDT | 24 | ASGEVAFDV | 40 |
| 1E07 | GYSFTNNW | 2 | IDPDDSYT | 25 | ARRAQGGGGYDY | 41 |
| 1F01 | GDSVSSKSAA | 3 | TYYRSKW | 26 | ARGIGSLNFDI | 42 |
| 2C07 | GFTFSNYA | 4 | ISYDGSNE | 27 | ARGTNYGMDV | 43 |
| 2E02 | GDSVSSKSAA | 3 | TYYRSKW | 26 | ARGIGSLNFDI | 42 |
| 2E08 | GYRFSHYW | 5 | IYPDDSDT | 24 | ATGVISGTDY | 44 |
| 2H05 | GYSFTNNW | 2 | IDPDDSYT | 25 | ARRAQGGGGYDY | 41 |
| 3A05 | GYSFTTYW | 6 | IYPGDSDT | 29 | ARLGGGMDV | 45 |
| 3A06 | GFTFSNAW | 7 | IKSKTDGGTT | 30 | TTENEVWPSLDV | 46 |
| 3B04 | GYSFPTYW | 8 | IYPGDSDT | 29 | ARGGSGLDY | 47 |
| 3B06 | GYTFTNYA | 9 | INPNTGGT | 31 | ARGGGGLVF | 48 |
| 3B09 | GYSFTSYW | 10 | IYPGDSDT | 29 | ATPMEDGMDV | 49 |
| 3C02 | GYSFTSYW | 10 | IYPGDSDT | 29 | ARGGPPDY | 50 |
| 3C12 | GYSFTSYW | 10 | IYPGDSDT | 29 | TRSSFNDH | 51 |
| 3D03 | GYSFTSYW | 10 | IYPGDSDT | 29 | ARLGGSLPIDY | 52 |
| 3D06 | GYTFTNYW | 11 | IYPGDSDT | 29 | AIGYDYGAFDI | 53 |
| 3D11 | GYSFPTYW | 8 | IYPADSDT | 31 | ARGGSGLDY | 47 |
| 3E04 | GYSFTSYW | 10 | IYPGDSDT | 29 | ASRLLFGDDAFDI | 54 |
| 3F06 | GYSFSTNW | 12 | IYPGDSDT | 29 | ARFYDSSGNDY | 55 |
| 3F09 | GYSFTSYW | 10 | IYPGDSDT | 29 | VVRPAAAGTRIDY | 56 |
| 4D01 | RYSFTSYW | 13 | IYPGDSDT | 29 | ARLKDGAFDI | 57 |
| 4F03 | GYSFTSYW | 10 | IYPGDSDT | 29 | ASWGMDV | 58 |
| 4G12 | GYSFTSYW | 10 | IYPGDSDT | 29 | ARHDYGMDV | 59 |
| 5A07 | GYSFTSYW | 10 | IYPGDSDT | 29 | ARHDYGMDV | 59 |
| 5C01 | GYSFTSYW | 10 | IYPGDSDT | 29 | ARLGQGSGPFDY | 60 |
| 5C07 | GYSFTNYW | 14 | IYPGDSDT | 29 | ASHTKDGTWDI | 61 |
| 6B04 | GYSFTSYW | 10 | IYPGDSDT | 29 | ARLGGAMDV | 62 |
| 6B11 | GDSVSSNSAA | 15 | TYYRSKW | 26 | TRGTGDKRAFDY | 63 |
| 6E08 | GFTFSSYA | 16 | ISYDGSNK | 32 | ARAFRRYDAFDI | 64 |
| 6F04 | GYSFTSYW | 10 | IYPGDSDT | 29 | ARGSGGYDY | 65 |
| A12 | GFTFSSFP | 17 | IVRTSTP | 33 | AKSATICGNCPEDIDA | 66 |
| B5 | GFTFSSFP | 17 | IVRTSTP | 33 | AKSATICGNCPEDIDA | 66 |
| B1.2 | GFTFSNHQ | 18 | INAAGSWT | 34 | AKDVYGYCSTCGATIDT | 67 |
| B11 | GFTFSSFN | 19 | IKRNGGET | 35 | AKDAGDNCRWNYCTTNIDA | 68 |
| B4.2 | GFTFSSYT | 710 | ICNDGTTT | 711 | TRGVDICWNSGNNIDA | 712 |
| D5 | GFTFSSYG | 20 | ISSTGINA | 36 | SKCAYSGSCSTSIDA | 69 |
| E2 | GFDFSSNA | 21 | ITTDGSYT | 37 | AKDSSIHWNGYDIDA | 70 |
| E4 | GFTFSSHA | 22 | INYDGIT | 38 | AKTTCSGCWDAAALIDA | 71 |
| F1.3 | GFTFSSFP | 17 | IVRTSTP | 33 | AKSATICGNCPEDIDA | 66 |
| F5 | GFTFSSSG | 23 | ITSSAGNT | 39 | AKDSNSGSPDASEIDA | 72 |
| 3H11 | GDSVSSNSAV | 28 | TYYRSKW | 26 | ARGRNGMDV | 696 |

TABLE 1B

| | | SEQ | | SEQ | | SEQ |
|---|---|---|---|---|---|---|
| Clone | | ID | | ID | | ID |
| ID | CDR-L1 | NO | CDR-L2 | NO | CDR-L3 | NO |
| 1E02 | KLGDKF | 73 | QDT | — | QAWDSSTGV | 99 |
| 1E07 | SSNIGAGFN | 74 | DNI | — | QSYDSSLSGWFA | 100 |
| 1F01 | SSDIGGYDY | 75 | DVS | — | SSYRAGSTLNLL | 101 |
| 2C07 | SLRSYY | 76 | GKN | — | QSRDTTGNYLL | 102 |
| 2E02 | SGDVGGYNY | 77 | EVT | — | SSYGGPNNLVL | 103 |
| 2E08 | KLGDKY | 78 | QDN | — | QTWDSGTGV | 104 |
| 2H05 | SSNIGAGYG | 79 | DDI | — | QSYDTSRGGLYV | 105 |
| 3A05 | SSNIGSNT | 80 | GNN | — | AAWDDSLSGYV | 106 |
| 3A06 | KLGNKY | 81 | QDT | — | QAWDSSTAVV | 107 |
| 3B04 | KLGTKY | 82 | QDS | — | QAWDSSTVV | 108 |
| 3B06 | QSLVDRDGNTY | 83 | KVS | — | MQATHWPPT | 109 |
| 3B09 | NLGDKY | 84 | QDT | — | QAWDSSTGV | 99 |
| 3C02 | RLGDKY | 85 | QDS | — | QAWDSSTAV | 110 |
| 3C12 | KLGNKF | 86 | QDT | — | QAWNTNTAL | 111 |
| 3D03 | KLGDKY | 78 | QDS | — | QAWDNNIGV | 112 |
| 3D06 | NSNIGSNS | 87 | RNN | — | AAWDDSLSGYV | 106 |
| 3D11 | KLGDKY | 78 | QDT | — | QAWDSNTMV | 113 |
| 3E04 | KLGDEY | 88 | QDS | — | QAWDSNAGA | 114 |
| 3F06 | KLGDKY | 78 | QDN | — | QTWDSSTAI | 115 |
| 3F09 | KLGDKY | 78 | QDN | — | QAWDSSTYV | 116 |
| 4D01 | SSNIEGNY | 89 | RNN | — | AAWDDSLNGYV | 117 |
| 4F03 | SSNIGTHT | 90 | STF | — | AAWDDSLNGPV | 118 |
| 4G12 | KLGDKF | 73 | QDN | — | QAWVNNTVV | 119 |
| 5A07 | RSNVGGNT | 91 | TNN | — | AAWDDSLNGYV | 117 |
| 5C01 | SSNIGSHS | 92 | GNS | — | ASWDDSLNGYV | 120 |
| 5C07 | RLGDKY | 85 | QDD | — | QAWDSSTSLYV | 121 |
| 6B04 | KLGDKY | 78 | QDK | — | AAWDDSLNGYYV | 122 |
| 6B11 | SSNIGSYF | 93 | RND | — | AAWDDSLSGRI | 123 |
| 6E08 | QGLVYSDGNTY | 94 | KVS | — | MQGTHWPPA | 124 |
| 6F04 | KLGDKF | 73 | EDN | — | QTWDVSSYV | 125 |
| A12 | SSD | — | YSD | — | GSRDSSNVAI | 126 |
| B5 | SSD | — | YSD | — | GSRDSSNVAI | 126 |
| B1.2 | TYD | — | YND | — | GTYEDNNGAS | 127 |
| B11 | GRWYGTYY | 95 | SND | — | GSWDSTDTTA | 128 |
| B4.2 | GSYYGSYY | 96 | NND | — | GSKDSSGAGI | 129 |
| D5 | SSGYG | 97 | NNN | — | GAWESSATDI | 130 |
| E2 | INN | — | QND | — | GSEDSRTYLGI | 131 |
| E4 | SSGYA | 98 | SND | — | GSFDSSSYI | 132 |
| F1.3 | SSD | — | YSD | — | GSGDNSYVG | 133 |
| F5 | KSY | — | DNT | — | GNADHSYVGV | 134 |
| 3H11 | SSNIGHKY | 697 | RNN | — | ATWDDSLNGVV | 698 |

or an antigen-binding fragment thereof.

In an embodiment, the CDR-H1, CDR-H2, CDR-H3, CDR-L1, CDR-L2 and/or CDR-L3 of the anti-TrkB antibody or antigen-binding fragment thereof comprise or consist of amino acid sequences having at least 75% identity with the sequences depicted in Tables 1A and 1B. In an embodiment, the CDR-H1, CDR-H2, CDR-H3, CDR-L1, CDR-L2 and/or CDR-L3 of the anti-TrkB antibody or antigen-binding fragment thereof comprise or consist of amino acid sequences having at least 80% identity with the sequences depicted in Tables 1A and 1B. In an embodiment, the CDR-H1, CDR-H2, CDR-H3, CDR-L1, CDR-L2 and/or CDR-L3 of the anti-TrkB antibody or antigen-binding fragment thereof comprise or consist of amino acid sequences having at least 85% identity with the sequences depicted in Tables 1A and 1b. In an embodiment, the CDR-H1, CDR-H2, CDR-H3, CDR-L1, CDR-L2 and/or CDR-L3 of the anti-TrkB antibody or antigen-binding fragment thereof comprise or consist of amino acid sequences having at least 90% identity with the sequences depicted in Tables 1A and 1B. In an embodiment, the CDR-H1, CDR-H2, CDR-H3, CDR-L1, CDR-L2 and/or CDR-L3 of the anti-TrkB antibody or antigen-binding fragment thereof comprise or consist of amino acid sequences having at least 95% identity with the sequences depicted in Tables 1A and 1B. In an embodiment, the CDR-H1, CDR-H2, CDR-H3, CDR-L1, CDR-L2 and/or CDR-L3 of the anti-TrkB antibody or antigen-binding fragment thereof comprise or consist of the amino acid sequences depicted in Tables 1A and 1B.

In an embodiment, the CDR-H1, CDR-H2, CDR-H3, CDR-L1, CDR-L2 and/or CDR-L3 comprises one, two or three amino acid substitutions. In an embodiment, the CDR-H1, CDR-H2, CDR-H3, CDR-L1, CDR-L2 and/or CDR-L3 comprises two amino acid substitutions. In an embodiment, the CDR-H1, CDR-H2, CDR-H3, CDR-L1, CDR-L2 and/or CDR-L3 comprises one amino acid substitution.

In an embodiment, the anti-TrkB antibody comprises the CDR-H1, CDR-H2, CDR-H3, CDR-L1, CDR-L2 and CDR-L3 of clones 2E02 or 1F01 described herein.

In an embodiment, the anti-TrkB antibody comprises the CDR-H1, CDR-H2, CDR-H3, CDR-L1, CDR-L2 and CDR-L3 of clones F5, 3H11, 6E08, or 3C12 described herein.

In an embodiment, the anti-TrkB antibody or antigen-binding fragment thereof comprises a heavy chain framework region 1 (VH FR1) comprising or consisting of an amino acid sequence having at least 60% identity with one of the sequences set forth in SEQ ID NOs: 135-166 and 392.

In an embodiment, the VH FR1 of the anti-TrkB antibody or antigen-binding fragment thereof comprises or consists of an amino acid sequence having at least 70% identity with one of the sequences set forth in SEQ ID NOs: 135-166 and 392. In an embodiment, the VH FR1 of the anti-TrkB antibody or antigen-binding fragment thereof comprises or consists of an amino acid sequence having at least 75% identity with one of the sequences set forth in SEQ ID NOs: 135-166 and 392. In an embodiment, the VH FR1 of the anti-TrkB antibody or antigen-binding fragment thereof comprises or consists of an amino acid sequence having at least 80% identity with one of the sequences set forth in SEQ ID NOs: 135-166 and 392. In an embodiment, the VH FR1 of the anti-TrkB antibody or antigen-binding fragment thereof comprises or consists of an amino acid sequence having at least 85% identity with one of the sequences set forth in SEQ ID NOs: 135-166 and 392. In an embodiment, the VH FR1 of the anti-TrkB antibody or antigen-binding fragment thereof comprises or consists of an amino acid sequence having at least 90% identity with one of the sequences set forth in SEQ ID NOs: 135-166 and 392. In an embodiment, the VH FR1 of the anti-TrkB antibody or antigen-binding fragment thereof comprises or consists of amino acid sequence having at least 95% identity with one of the sequences set forth in SEQ ID NOs: 135-166 and 392. In an embodiment, the VH FR1 of the anti-TrkB antibody or antigen-binding fragment thereof comprises or consists of one of the sequences set forth in SEQ ID NOs: 135-166 and 392.

In an embodiment, the anti-TrkB antibody or antigen-binding fragment thereof comprises a heavy chain framework region 2 (VH FR2) comprising or consisting of an amino acid sequence having at least 60% identity with one of the sequences set forth in SEQ ID NOs: 167-186.

In an embodiment, the VH FR2 of the anti-TrkB antibody or antigen-binding fragment thereof comprises or consists of an amino acid sequence having at least 70% identity with one of the sequences set forth in SEQ ID NOs: 167-186. In an embodiment, the VH FR2 of the anti-TrkB antibody or antigen-binding fragment thereof comprises or consists of an amino acid sequence having at least 75% identity with one of the sequences set forth in SEQ ID NOs: 167-186. In an embodiment, the VH FR2 of the anti-TrkB antibody or antigen-binding fragment thereof comprises or consists of an amino acid sequence having at least 80% identity with one of the sequences set forth in SEQ ID NOs: 167-186. In an embodiment, the VH FR2 of the anti-TrkB antibody or antigen-binding fragment thereof comprises or consists of an amino acid sequence having at least 85% identity with one of the sequences set forth in SEQ ID NOs: 167-186. In an embodiment, the VH FR2 of the anti-TrkB antibody or antigen-binding fragment thereof comprises or consists of an amino acid sequence having at least 90% identity with one of the sequences set forth in SEQ ID NOs: 167-186. In an embodiment, the VH FR2 of the anti-TrkB antibody or antigen-binding fragment thereof comprises or consists of amino acid sequence having at least 95% identity with one of the sequences set forth in SEQ ID NOs: 167-186. In an embodiment, the VH FR2 of the anti-TrkB antibody or antigen-binding fragment thereof comprises or consists of one of the sequences set forth in SEQ ID NOs: 167-186.

In an embodiment, the anti-TrkB antibody or antigen-binding fragment thereof comprises a heavy chain framework region 3 (VH FR3) comprising or consisting of an amino acid sequence having at least 60% identity with one of the sequences set forth in SEQ ID NOs: 187-207 and 699.

In an embodiment, the VH FR3 of the anti-TrkB antibody or antigen-binding fragment thereof comprises or consists of an amino acid sequence having at least 70% identity with one of the sequences set forth in SEQ ID NOs: 187-207 and 699. In an embodiment, the VH FR3 of the anti-TrkB antibody or antigen-binding fragment thereof comprises or consists of an amino acid sequence having at least 75% identity with one of the sequences set forth in SEQ ID NOs: 187-207 and 699. In an embodiment, the VH FR3 of the anti-TrkB antibody or antigen-binding fragment thereof comprises or consists of an amino acid sequence having at least 80% identity with one of the sequences set forth in SEQ ID NOs: 187-207 and 699. In an embodiment, the VH FR3 of the anti-TrkB antibody or antigen-binding fragment thereof comprises or consists of an amino acid sequence having at least 85% identity with one of the sequences set forth in SEQ ID NOs: 187-207 and 699. In an embodiment, the VH FR3 of the anti-TrkB antibody or antigen-binding fragment thereof comprises or consists of an amino acid sequence having at least 90% identity with one of the sequences set forth in SEQ ID NOs: 187-207 and 699. In an embodiment, the VH FR3 of the anti-TrkB antibody or antigen-binding fragment thereof comprises or consists of amino acid sequence having at least 95% identity with one of the sequences set forth in SEQ ID NOs: 187-207 and 699. In an embodiment, the VH FR3 of the anti-TrkB antibody or antigen-binding fragment thereof comprises or consists of one of the sequences set forth in SEQ ID NOs: 187-207 and 699.

In an embodiment, the anti-TrkB antibody or antigen-binding fragment thereof comprises a heavy chain framework region 4 (VH FR4) comprising or consisting of an amino acid sequence having at least 60% identity with one of the sequences set forth in SEQ ID NOs: 208-211.

In an embodiment, the VH FR4 of the anti-TrkB antibody or antigen-binding fragment thereof comprises or consists of an amino acid sequence having at least 70% identity with one of the sequences set forth in SEQ ID NOs: 208-211. In an embodiment, the VH FR4 of the anti-TrkB antibody or antigen-binding fragment thereof comprises or consists of an amino acid sequence having at least 75% identity with one of the sequences set forth in SEQ ID NOs: 208-211. In an embodiment, the VH FR4 of the anti-TrkB antibody or antigen-binding fragment thereof comprises or consists of an amino acid sequence having at least 80% identity with one of the sequences set forth in SEQ ID NOs: 208-211. In an embodiment, the VH FR4 of the anti-TrkB antibody or antigen-binding fragment thereof comprises or consists of an amino acid sequence having at least 85% identity with one of the sequences set forth in SEQ ID NOs: 208-211. In an embodiment, the VH FR4 of the anti-TrkB antibody or antigen-binding fragment thereof comprises or consists of an amino acid sequence having at least 90% identity with one of the sequences set forth in SEQ ID NOs: 208-211. In an embodiment, the VH FR4 of the anti-TrkB antibody or antigen-binding fragment thereof comprises or consists of amino acid sequence having at least 95% identity with one of the sequences set forth in SEQ ID NOs: 208-211. In an embodiment, the VH FR4 of the anti-TrkB antibody or antigen-binding fragment thereof comprises or consists of one of the sequences set forth in SEQ ID NOs: 208-211.

In an embodiment, the anti-TrkB antibody or antigen-binding fragment thereof comprises a light chain framework region 1 (VL FR1) comprising or consisting of an amino acid sequence having at least 60% identity with one of the sequences set forth in SEQ ID NOs: 212-243.

In an embodiment, the VL FR1 of the anti-TrkB antibody or antigen-binding fragment thereof comprises or consists of an amino acid sequence having at least 70% identity with one of the sequences set forth in SEQ ID NOs: 212-243. In an embodiment, the VL FR1 of the anti-TrkB antibody or antigen-binding fragment thereof comprises or consists of an amino acid sequence having at least 75% identity with one of the sequences set forth in SEQ ID NOs: 212-243. In an embodiment, the VL FR1 of the anti-TrkB antibody or antigen-binding fragment thereof comprises or consists of an amino acid sequence having at least 80% identity with one of the sequences set forth in SEQ ID NOs: 212-243. In an embodiment, the VL FR1 of the anti-TrkB antibody or antigen-binding fragment thereof comprises or consists of an amino acid sequence having at least 85% identity with one of the sequences set forth in SEQ ID NOs: 212-243. In an embodiment, the VL FR1 of the anti-TrkB antibody or antigen-binding fragment thereof comprises or consists of an amino acid sequence having at least 90% identity with one of the sequences set forth in SEQ ID NOs: 212-243. In an embodiment, the VL FR1 of the anti-TrkB antibody or antigen-binding fragment thereof comprises or consists of amino acid sequence having at least 95% identity with one of the sequences set forth in SEQ ID NOs: 212-243. In an embodiment, the VL FR1 of the anti-TrkB antibody or antigen-binding fragment thereof comprises or consists of one of the sequences set forth in SEQ ID NOs: 212-243.

In an embodiment, the anti-TrkB antibody or antigen-binding fragment thereof comprises a light chain framework region 2 (VL FR2) comprising or consisting of an amino acid sequence having at least 60% identity with one of the sequences set forth in SEQ ID NOs: 244-270 and 700.

In an embodiment, the VL FR2 of the anti-TrkB antibody or antigen-binding fragment thereof comprises or consists of an amino acid sequence having at least 70% identity with one of the sequences set forth in SEQ ID NOs: 244-270 and 700. In an embodiment, the VL FR2 of the anti-TrkB antibody or antigen-binding fragment thereof comprises or consists of an amino acid sequence having at least 75% identity with one of the sequences set forth in SEQ ID NOs: 244-270 and 700. In an embodiment, the VL FR2 of the anti-TrkB antibody or antigen-binding fragment thereof comprises or consists of an amino acid sequence having at least 80% identity with one of the sequences set forth in SEQ ID NOs: 244-270 and 700. In an embodiment, the VL FR2 of the anti-TrkB antibody or antigen-binding fragment thereof comprises or consists of an amino acid sequence having at least 85% identity with one of the sequences set forth in SEQ ID NOs: 244-270 and 700. In an embodiment, the VL FR2 of the anti-TrkB antibody or antigen-binding fragment thereof comprises or consists of an amino acid sequence having at least 90% identity with one of the sequences set forth in SEQ ID NOs: 244-270 and 700. In an embodiment, the VL FR2 of the anti-TrkB antibody or antigen-binding fragment thereof comprises or consists of amino acid sequence having at least 95% identity with one of the sequences set forth in SEQ ID NOs: 244-270 and 700. In an embodiment, the VL FR2 of the anti-TrkB antibody or antigen-binding fragment thereof comprises or consists of one of the sequences set forth in SEQ ID NOs: 244-270 and 700.

In an embodiment, the anti-TrkB antibody or antigen-binding fragment thereof comprises a light chain framework region 3 (VL FR3) comprising or consisting of an amino acid sequence having at least 60% identity with one of the sequences set forth in SEQ ID NOs: 271-302.

In an embodiment, the VL FR3 of the anti-TrkB antibody or antigen-binding fragment thereof comprises or consists of an amino acid sequence having at least 70% identity with one of the sequences set forth in SEQ ID NOs: 271-302. In an embodiment, the VL FR3 of the anti-TrkB antibody or antigen-binding fragment thereof comprises or consists of an amino acid sequence having at least 75% identity with one of the sequences set forth in SEQ ID NOs: 271-302. In an embodiment, the VL FR3 of the anti-TrkB antibody or antigen-binding fragment thereof comprises or consists of an amino acid sequence having at least 80% identity with one of the sequences set forth in SEQ ID NOs: 271-302. In an embodiment, the VL FR3 of the anti-TrkB antibody or antigen-binding fragment thereof comprises or consists of an amino acid sequence having at least 85% identity with one of the sequences set forth in SEQ ID NOs: 271-302. In an embodiment, the VL FR3 of the anti-TrkB antibody or antigen-binding fragment thereof comprises or consists of an amino acid sequence having at least 90% identity with one of the sequences set forth in SEQ ID NOs: 271-302. In an embodiment, the VL FR3 of the anti-TrkB antibody or antigen-binding fragment thereof comprises or consists of amino acid sequence having at least 95% identity with one of the sequences set forth in SEQ ID NOs: 271-302. In an embodiment, the VL FR3 of the anti-TrkB antibody or antigen-binding fragment thereof comprises or consists of one of the sequences set forth in SEQ ID NOs: 271-302.

In an embodiment, the anti-TrkB antibody or antigen-binding fragment thereof comprises a light chain framework region 4 (VL FR4) comprising or consisting of an amino acid sequence having at least 60% identity with one of the sequences set forth in SEQ ID NOs: 303-313.

In an embodiment, the VL FR4 of the anti-TrkB antibody or antigen-binding fragment thereof comprises or consists of an amino acid sequence having at least 70% identity with one of the sequences set forth in SEQ ID NOs: 303-313. In an embodiment, the VL FR4 of the anti-TrkB antibody or antigen-binding fragment thereof comprises or consists of an amino acid sequence having at least 75% identity with one of the sequences set forth in SEQ ID NOs: 303-313. In an embodiment, the VL FR4 of the anti-TrkB antibody or antigen-binding fragment thereof comprises or consists of an amino acid sequence having at least 80% identity with one of the sequences set forth in SEQ ID NOs: 303-313. In an embodiment, the VL FR4 of the anti-TrkB antibody or antigen-binding fragment thereof comprises or consists of an amino acid sequence having at least 85% identity with one of the sequences set forth in SEQ ID NOs: 303-313. In an embodiment, the VL FR4 of the anti-TrkB antibody or antigen-binding fragment thereof comprises or consists of an amino acid sequence having at least 90% identity with one of the sequences set forth in SEQ ID NOs: 303-313. In an embodiment, the VL FR4 of the anti-TrkB antibody or antigen-binding fragment thereof comprises or consists of amino acid sequence having at least 95% identity with one of the sequences set forth in SEQ ID NOs: 303-313. In an embodiment, the VL FR4 of the anti-TrkB antibody or antigen-binding fragment thereof comprises or consists of one of the sequences set forth in SEQ ID NOs: 303-313.

Methods and techniques for identifying CDRs within heavy chain variable region (HCVR) and light chain variable region (LCVR) amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, the international ImMunoGeneTics information System® (IMGT) definition, the Contact definition and the AbM definition. In general terms, the Kabat definition is based on sequence variability, the Chothia definition is based on the location of the structural loop regions, the AbM definition is a compromise between the Kabat and Chothia approaches, the Contact definition is based on an analysis of which residues contact antigen in crystal structures, and the IMGT definition is based on CDR and Framework definitions as defined by IMGT. See, e.g., Kabat, “Sequences of Proteins of Immunological Interest,” National Institutes of Health, Bethesda, Md. (1991); Al-Lazikani et al., J. Mol. Biol. 273:927-948 (1997); Martin et al., Proc. Natl. Acad. Sci. USA 86:9268-9272 (1989); Whitelegg N & Rees A R, Protein Eng. 13 (2000): 819-824; Whitelegg N & Rees AR, Methods Mol Biol. 248 (2004) 51-91; MacCallum R M, Martin A C R & Thornton J M, J. Mol. Biol. 262 (1996) 732-745. Public databases are also available for identifying CDR sequences within an antibody (e.g., abYsis; Swindells et al., J Mol Biol. 2017 Feb. 3; 429 (3): 356-364).

The amino acids defining the CDRs and FRs disclosed herein are based on the IMGT definition. However, the skilled person would understand that the amino acids forming the CDRs and FRs regions in the sequences of the HCVR and LCVR of the antibodies described herein may vary depending on the numbering scheme used. Using the HCVR of antibody clone 1E02 disclosed herein as an example, the sequences of the CDRs and FRs regions according to commonly used antibody numbering schemes are depicted in Table 3.

TABLE 3
Predicted FR and CDR sequences in the HCVR of antibody clone 1E02 according to
commonly used antibody numbering schemes

| Region | Definition | Sequence Fragment (SEQ ID NO: ) | Residues | Length |
|---|---|---|---|---|
| HFR1 | Chothia | QVQLLQSAAEVKKPGESLKISCKGS----- (392) | 1-25 | 25 |
| | AbM | QVQLLQSAAEVKKPGESLKISCKGS----- (392) | 1-25 | 26 |
| | Kabat | QVQLLQSAAEVKKPGESLKISCKGSGYRFT (393) | 1-30 | 30 |
| | Contact | QVQLLQSAAEVKKPGESLKISCKGSGYRF- (394) | 1-29 | 28 |
| | IMGT | QVQLLQSAAEVKKPGESLKISCKGS----- (392) | 1-25 | 25 |
| CDR-H1 | Chothia | GYRFTSY--- (395) | 26-32 | 7 |
| | AbM | GYRFTSYWIG (396) | 26-35 | 10 |
| | Kabat | -----SYWIG (397) | 31-35 | 5 |
| | Contact | ----TSYWIG (398) | 30-35 | 6 |
| | IMGT | GYRFTSYW-- (1) | 26-33 | 8 |
| HFR2 | Chothia | WIGWVRQMPGKGLEWVGII (399) | 33-51 | 19 |
| | AbM | ---WVRQMPGKGLEWVG-- (400) | 36-49 | 14 |
| | Kabat | ---WVRQMPGKGLEWVG-- (400) | 36-49 | 14 |
| | Contact | --VWVRQMPGKGLE----- (401) | 36-46 | 11 |
| | IMGT | -IGWVRQMPGKGLEWVGI- (167) | 34-50 | 17 |
| CDR-H2 | Chothia | ---YPDDSD----------- (402) | 52-57 | 6 |
| | AbM | ---IIYPDDSDTR------- (403) | 50-59 | 10 |
| | Kabat | ---IIYPDDSDTRYSPSFQG (404) | 50-66 | 17 |
| | Contact | WVGIIYPDDSDTR------- (405) | 47-59 | 13 |
| | IMGT | ----IYPDDSDT-------- (24) | 51-58 | 8 |
| HFR3 | Chothia | TRYSPSFQGQVTISADTSISTTYLQWSSLKASDTAIYYCAS (406) | 58-98 | 41 |
| | AbM | --YSPSFQGQVTISADTSISTTYLQWSSLKASDTAIYYCAS (407) | 60-98 | 39 |
| | Kabat | ---------QVTISADTSISTTYLQWSSLKASDTAIYYCAS (408) | 67-98 | 32 |
| | Contact | --YSPSFQGQVTISADTSISTTYLQWSSLKASDTAIYYC-- (409) | 60-96 | 37 |
| | IMGT | -RYSPSFQGQVTISADTSISTTYLQWSSLKASDTAIYYC-- (187) | 59-96 | 38 |
| CDR-H3 | Chothia | --GEVAFDV (410) | 99-105 | 7 |
| | AbM | --GEVAFDV (410) | 99-105 | 7 |
| | Kabat | --GEVAFDV (410) | 99-105 | 7 |
| | Contact | ASGEVAFD- (411) | 97-104 | 8 |
| | IMGT | ASGEVAPDV (40) | 97-105 | 9 |
| HFR4 | Chothia | -WGQGTTVTVSS (208) | 106-116 | 11 |
| | AbM | -WGQGTTVTVSS (208) | 106-116 | 11 |
| | Kabat | -WGQGTTVTVSS (208) | 106-116 | 11 |
| | Contact | VWGQGTTVTVSS (412) | 105-116 | 12 |
| | IMGT | -WGQGTTVTVSS (208) | 106-116 | 11 |

In an embodiment, the anti-TrkB antibody or antigen-binding fragment thereof comprises an HCVR comprising or consisting of an amino acid sequence having at least 70% identity with one of the sequences set forth in SEQ ID NOs: 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 701 or 713.

In an embodiment, the HCVR of the anti-TrkB antibody or antigen-binding fragment thereof comprises or consists of an amino acid sequence having at least 75% identity with one of the sequences set forth in SEQ ID NOs: 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 701 or 713. In an embodiment, the HCVR of the anti-TrkB antibody or antigen-binding fragment thereof comprises or consists of an amino acid sequence having at least 80% identity with one of the sequences set forth in SEQ ID NOs: 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 701 or 713. In an embodiment, the HCVR of the anti-TrkB antibody or antigen-binding fragment thereof comprises or consists of an amino acid sequence having at least 85% identity with one of the sequences set forth in SEQ ID NOs: 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 701 or 713. In an embodiment, the HCVR of the anti-TrkB antibody or antigen-binding fragment thereof comprises or consists of an amino acid sequence having at least 90% identity with one of the sequences set forth in SEQ ID NOs: 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 701 or 713. In an embodiment, the HCVR of the anti-TrkB antibody or antigen-binding fragment thereof comprises or consists of amino acid sequence having at least 95% identity with one of the sequences set forth in SEQ ID NOs: 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 701 or 713. In an embodiment, the HCVR of the anti-TrkB antibody or antigen-binding fragment thereof comprises or consists of one of the sequences set forth in SEQ ID NOs: 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 701 or 713.

In an embodiment, the anti-TrkB antibody or antigen-binding fragment thereof comprises a LCVR comprising or consisting of an amino acid sequence having at least 70% identity with one of the sequences set forth in SEQ ID NOs: 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 702 or 714.

In an embodiment, the LCVR of the anti-TrkB antibody or antigen-binding fragment thereof comprises or consists of an amino acid sequence having at least 75% identity with one of the sequences set forth in SEQ ID NOs: 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 702 or 714. In an embodiment, the LCVR of the anti-TrkB antibody or antigen-binding fragment thereof comprises or consists of an amino acid sequence having at least 80% identity with one of the sequences set forth in SEQ ID NOs: 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 702 or 714. In an embodiment, the LCVR of the anti-TrkB antibody or antigen-binding fragment thereof comprises or consists of an amino acid sequence having at least 85% identity with one of the sequences set forth in SEQ ID NOs: 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 702 or 714. In an embodiment, the LCVR of the anti-TrkB antibody or antigen-binding fragment thereof comprises or consists of an amino acid sequence having at least 90% identity with one of the sequences set forth in SEQ ID NOs: 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 702 or 714. In an embodiment, the LCVR of the anti-TrkB antibody or antigen-binding fragment thereof comprises or consists of amino acid sequence having at least 95% identity with one of the sequences set forth in SEQ ID NOs: 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 702 or 714. In an embodiment, the LCVR of the anti-TrkB antibody or antigen-binding fragment thereof comprises or consists of one of the sequences set forth in SEQ ID NOs: 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 702 or 714.

In an embodiment, the anti-TrkB antibody or antigen-binding fragment thereof comprises a combination of one the HCVRs and one of the LCVRs defined above. In a further embodiment, the anti-TrkB antibody or antigen-binding fragment thereof comprises one of the following combinations of HCVR and LCVR: SEQ ID NOs: 314 and 315; 316 and 317; 318 and 319; 320 and 321; 322 and 323; 324 and 325; 326 and 327; 328 and 329; 330 and 331; 332 and 333; 334 and 335; 336 and 337; 338 and 339; 340 and 341; 342 and 343; 344 and 345; 346 and 347; 348 and 349; 350 and 351; 352 and 353; 354 and 355; 356 and 357; 358 and 359; 360 and 361; 362 and 363; 364 and 365; 366 and 367; 368 and 369; 370 and 371; 372 and 373; 374 and 375; 376 and 377; 378 and 379; 380 and 381; 382 and 383; 384 and 385; 386 and 387; 388 and 389; 390 and 391, 701 and 702, or 713 and 714.

In an embodiment, the anti-TrkB antibody comprises the HCVR and LCVR of clones 2E02 or 1F01 described herein.

In an embodiment, the anti-TrkB antibody comprises the HCVR and LCVR of clones F5, 3H11, 6E08, or 3C12 described herein.

Tropomyosin-related kinase B (TrkB, also named NTRK2) is the receptor for brain-derived neurotrophic factor (BDNF), neurotrophin-4 (NTF4) and neurotrophin-3 (NTF3), It is involved in the development and the maturation of the central and the peripheral nervous systems through regulation of neuron survival, proliferation, migration, differentiation, and synapse formation and plasticity. Human TrkB has the following amino acid sequence:

(SEQ ID NO: 413)
MSSWIRWHGPAMARLWGFCWLVVGFWRAAFACPTSCKCSASRIWCSDPSPGIVAFPRLEPNSVDPENITE
IFIANQKRLEIINEDDVEAYVGLRNLTIVDSGLKFVAHKAFLKNSNLQHINFTRNKLTSLSRKHFRHLDL
SELILVGNPFTCSCDIMWIKTLQEAKSSPDTQDLYCLNESSKNIPLANLQIPNCGLPSANLAAPNLTVEE
GKSITLSCSVAGDPVPNMYWDVGNLVSKHMNETSHTQGSLRITNISSDDSGKQISCVAENLVGEDQDSVN
LTVHFAPTITFLESPTSDHHWCIPFTVKGNPKPALQWFYNGAILNESKYICTKIHVINHTEYHGCLQLDN
PTHMNNGDYTLIAKNEYGKDEKQISAHFMGWPGIDDGANPNYPDVIYEDYGTAANDIGDTTNRSNEIPST
DVTDKTGREHLSVYAVVVIASVVGFCLLVMLFLLKLARHSKFGMKGPASVISNDDDSASPLHHISNGSNT
PSSSEGGPDAVIIGMTKIPVIENPQYFGITNSQLKPDTFVQHIKRHNIVLKRELGEGAFGKVFLAECYNL
CPEQDKILVAVKTLKDASDNARKDFHREAELLTNLQHEHIVKFYGVCVEGDPLIMVFEYMKHGDLNKFLR
AHGPDAVLMAEGNPPTELTQSQMLHIAQQIAAGMVYLASQHFVHRDLATRNCLVGENLLVKIGDFGMSRD
VYSTDYYRVGGHTMLPIRWMPPESIMYRKFTTESDVWSLGVVLWEIFTYGKQPWYQLSNNEVIECITQGR
VLQRPRTCPQEVYELMLGCWQREPHMRKNIKGIHTLLQNLAKASPVYLDILG

The extracellular domain (ECD) of Trk is defined by residues 32-430.

The term “specifically binds”, or the like, means that an antibody or antigen-binding fragment thereof forms a complex with an antigen (TrkB) that is relatively stable under physiologic conditions. Specific binding can be characterized by an equilibrium dissociation constant (K_{D}) of about 1×10⁻⁶ M or less, preferably 1×10⁻⁷ M or 1×10⁻⁸ M or less. Methods for determining whether two molecules specifically bind are well known in the art and include, for example, equilibrium dialysis, surface plasmon resonance, and the like. Moreover, multi-specific antibodies that bind to TrkB and one or more additional antigens or a bi-specific that binds to two different regions of TrkB are considered antibodies that “specifically bind”, as used herein. In an embodiment, the anti-TrkB antibody or antigen-binding fragment thereof has a higher affinity for TrkB than for other Trk family members, e.g., TrkA and/or TrkC. In an embodiment, the anti-TrkB antibody or antigen-binding fragment thereof does not bind to TrkA and/or TrkC.

### Anti-CD3 Antibodies and Antigen-Binding Fragments Thereof

The present disclosure provides an antibody (e.g., an anti-CD3 antibody) comprising a combination of heavy chain complementarity determining regions (CDR-H1, CDR-H2 and CDR-H3) and light chain complementarity determining regions (CDR-L1, CDR-L2 and CDR-L3) from any of the anti-CD3 antibody clones disclosed herein (e.g., Table 8). In an embodiment, the disclosure provides an antibody (e.g., an anti-CD3 antibody) comprising a combination of heavy chain complementarity determining regions (CDR-H1, CDR-H2 and CDR-H3) and light chain complementarity determining regions (CDR-L1, CDR-L2 and CDR-L3) from anti-CD3 antibody clones 3-A03, 1-A09, 4-A01, 4-G03, 8-A05, 3-G07, 2-C02, 5-C03, 6-C04, 3-A05, 3-C04, 3-A06, 3-B04, 7-G01, 1-G11, 2-E11 or 7-B02 disclosed herein (e.g., Table 8).

The present disclosure provides an antibody (e.g., an anti-CD3 antibody) comprising a combination of heavy chain complementarity determining regions (CDR-H1, CDR-H2 and CDR-H3) and light chain complementarity determining regions (CDR-L1, CDR-L2 and CDR-L3), wherein said CDR-H1, CDR-H2, CDR-H3, CDR-L1, CDR-L2 and CDR-L3 comprise or consist of amino acid sequences having at least 70% identity with the sequences depicted in Tables 2A and 2B:

TABLE 2A

| | SEQ ID | | SEQ ID | | SEQ ID |
|---|---|---|---|---|---|
| CDR-H1 | NO: | CDR-H2 | NO: | CDR-H3 | NO: |
| GFTFSSYA | 16 | ISGSGGST | 432 | ARDAHDY | 448 |
| GFTFSSYS | 414 | ISSSSSYI | 433 | ARVKDYDSSLDY | 449 |
| GDSVSSNSAA | 15 | TYYRSKW | 26 | ARGITGTGYGMDV | 450 |
| GFSFGSSA | 415 | ISHSGSTT | 434 | AKIGAYGYYFHY | 451 |
| GYSFGSSA | 703 | ISHSGSTT | 434 | AKIGAYGYYFHY | 451 |
| GFSFGSSA | 415 | ISHSGSTT | 434 | AKIGAYGYYFHY | 451 |
| GFSFGSSA | 415 | ISHSGSTT | 434 | AKIGAYGYYFHY | 451 |
| GFSFGSSA | 415 | ISHSGSTT | 434 | AKIGAYGYYFHY | 451 |
| GGTFSSYA | 416 | IIPIFGTA | 435 | ARDAVVVADSGSMDV | 452 |
| GGTFSSYA | 416 | IIPIFGTA | 435 | ARDAVVVADSGSMDV | 452 |
| GDSVSSNSAA | 15 | TYYRSKW | 26 | ATWGMDV | 453 |
| GDSVSSNSAA | 15 | TYYRSKW | 26 | ATWGMDV | 453 |
| GDSVSSNSAA | 15 | TYYRSKW | 26 | ATWGMDV | 453 |
| GYTFTGHY | 417 | INPRGGST | 436 | ARESIAAAGPYFDY | 454 |
| GFTFNNFY | 418 | ISRGGNSV | 437 | VRGGDFLY | 455 |
| GFTFRGFY | 419 | ISSSGSAV | 438 | ARDGGTGGFDY | 456 |
| GFTFSTYA | 420 | ISYDGAGK | 439 | AGEHYTLTDEEFDF | 457 |
| GFTFNNSY | 421 | ISRGGNSV | 437 | AKGGDYLY | 458 |
| RFTFSSYP | 422 | ISGSGGST | 432 | ARDPHDF | 459 |
| GFTFDDFA | 423 | ITWNSAAT | 440 | ARDRGGPKDPFDI | 460 |
| GYTFTRYG | 424 | ISAYNGNT | 441 | ARDRGYCSGGSCPGIDAFDI | 461 |
| GYTFRNYY | 425 | VNPNSGNA | 442 | AREPPATVAYYYGMDV | 462 |
| GFTFSSYG | 20 | ISGSGGST | 432 | ARERLIGMDV | 463 |
| GYTFTSYG | 426 | ISAYNGNT | 441 | AREVNYGLDV | 464 |
| GFTSNTYA | 427 | ISGSGSST | 443 | ARIERYSYGIDY | 465 |
| GFTFSSYG | 20 | ISPDGSDK | 444 | ARNRKYTNFLDI | 466 |
| GYTFTSYY | 428 | INPSGGTT | 445 | ARNWASFDY | 467 |
| GFTFGDYA | 429 | IRSKAYGGTT | 446 | TRVSDIYGMDV | 468 |
| GFTFRRYG | 430 | ISSSSSYI | 433 | VRDGRFINGLDV | 469 |
| GFTFSRYS | 431 | ISGGGGST | 447 | VRGGWYRTPFEF | 470 |

TABLE 2B

| | SEQ | | SEQ | | SEQ |
|---|---|---|---|---|---|
| CDR-L1 | ID NO: | CDR-L2 | ID NO: | CDR-L3 | ID NO: |
| SSNLGNNY | 471 | DNN | — | GTWDSSLSAGV | 490 |
| KLGDKY | 78 | QDS | — | QAWDSSTVV | 108 |
| SSNIGRNH | 472 | NND | — | AAWDDSLSSGV | 491 |
| GGNIGNNY | 473 | DDS | — | QSSDSGRVV | 492 |
| GGNIASNY | 474 | DDS | — | QSSDNANVV | 493 |
| GGSIASNY | 475 | DDN | — | QSSDNINHVI | 494 |
| SGSIASKY | 476 | DDK | — | QSFDSTNQAVV | 495 |
| SGSIASNY | 477 | EDN | — | QSSDDSNHLV | 496 |
| SSNIGSNT | 80 | SNN | — | AAWDDSLNGWV | 497 |
| SSNIGSNT | 80 | SNN | — | AAWDDSLNGWV | 497 |
| SSNIGSNY | 478 | RNN | — | ASWDDSLRGPV | 498 |
| SSNIGSNY | 478 | RNN | — | AAWDDSLNGPV | 118 |
| SSNIGSNY | 478 | RNN | — | AAWDDSLSGPV | 499 |
| SGRIGSNY | 479 | EDN | — | QSYDSGVVVV | 500 |
| TGSIAGNY | 480 | EDK | — | QSYDSGNVI | 501 |
| SGSVASNY | 481 | QDN | — | QSYDGRRLI | 502 |
| ASNIGNNY | 482 | DNN | — | GTWDTSLRGVV | 503 |
| SGSIASKY | 476 | DDD | — | QSYDSSNWV | 504 |
| SSNLGNNY | 471 | DNN | — | GTWDSSLSAGV | 490 |
| SSNIGKNY | 483 | DNN | — | GTWDSSLSAVV | 505 |
| NIENKY | 484 | DDS | — | QVWDSSSDRVV | 506 |
| SGSIASNY | 477 | EDN | — | RSYDSSNRNWV | 507 |
| SGSIASNY | 477 | EDN | — | QSYDSGNPRV | 508 |
| SSNIGSKY | 485 | RNN | — | AAWDDSLSGRV | 509 |
| TGGIAANY | 486 | EDK | — | HSSDNNNQV | 510 |
| NIGLKS | 487 | DDV | — | QVWDDPSDQGSWV | 511 |
| QSVSSSY | 488 | GAS | — | QQYGSSPYT | 512 |
| SSNIGNNY | 489 | DNN | — | ATWDDSLKAGV | 513 |
| SGSIASNY | 477 | DDK | — | QSYDSSNHWV | 514 |
| KLGDKY | 78 | QDN | — | QAWDSSTNYV | 515 |

or an antigen-binding fragment thereof.

In an embodiment, the CDR-H1, CDR-H2, CDR-H3, CDR-L1, CDR-L2 and/or CDR-L3 comprises one, two or three amino acid substitutions. In an embodiment, the CDR-H1, CDR-H2, CDR-H3, CDR-L1, CDR-L2 and/or CDR-L3 comprises two amino acid substitutions. In an embodiment, the CDR-H1, CDR-H2, CDR-H3, CDR-L1, CDR-L2 and/or CDR-L3 comprises one amino acid substitution.

In an embodiment, the CDR-H1, CDR-H2, CDR-H3, CDR-L1, CDR-L2 and/or CDR-L3 of the anti-CD3 antibody or antigen-binding fragment thereof comprise or consist of amino acid sequences having at least 75% identity with the sequences depicted in Tables 2A and 2B. In an embodiment, the CDR-H1, CDR-H2, CDR-H3, CDR-L1, CDR-L2 and/or CDR-L3 of the anti-CD3 antibody or antigen-binding fragment thereof comprise or consist of amino acid sequences having at least 80% identity with the sequences depicted in Tables 2A and 2B. In an embodiment, the CDR-H1, CDR-H2, CDR-H3, CDR-L1, CDR-L2 and/or CDR-L3 of the anti-CD3 antibody or antigen-binding fragment thereof comprise or consist of amino acid sequences having at least 85% identity with the sequences depicted in Tables 2A and 2B. In an embodiment, the CDR-H1, CDR-H2, CDR-H3, CDR-L1, CDR-L2 and/or CDR-L3 of the anti-CD3 antibody or antigen-binding fragment thereof comprise or consist of amino acid sequences having at least 90% identity with the sequences depicted in Tables 2A and 2B. In an embodiment, the CDR-H1, CDR-H2, CDR-H3, CDR-L1, CDR-L2 and/or CDR-L3 of the anti-CD3 antibody or antigen-binding fragment thereof comprise or consist of amino acid sequences having at least 95% identity with the sequences depicted in Tables 2A and 2B. In an embodiment, the CDR-H1, CDR-H2, CDR-H3, CDR-L1, CDR-L2 and/or CDR-L3 of the anti-CD3 antibody or antigen-binding fragment thereof comprise or consist of the amino acid sequences depicted in Tables 2A and 2B.

In an embodiment, the CDR-H1, CDR-H2, CDR-H3, CDR-L1, CDR-L2 and/or CDR-L3 comprises one, two or three amino acid substitutions. In an embodiment, the CDR-H1, CDR-H2, CDR-H3, CDR-L1, CDR-L2 and/or CDR-L3 comprises two amino acid substitutions. In an embodiment, the CDR-H1, CDR-H2, CDR-H3, CDR-L1, CDR-L2 and/or CDR-L3 comprises one amino acid substitution.

In an embodiment, the anti-CD3 antibody or antigen-binding fragment thereof comprises the CDR-H1, CDR-H2, CDR-H3, CDR-L1, CDR-L2 and CDR-L3 of clones 1A09, 8A05 or 3G07 described herein.

In an embodiment, the anti-CD3 antibody or antigen-binding fragment thereof comprises a heavy chain framework region 1 (VH FR1) comprising or consisting of an amino acid sequence having at least 60% identity with one of the sequences set forth in SEQ ID NOs: 142 and 516-535.

In an embodiment, the VH FR1 of the anti-CD3 antibody or antigen-binding fragment thereof comprises or consists of an amino acid sequence having at least 70% identity with one of the sequences set forth in SEQ ID NOs: 142 and 516-535. In an embodiment, the VH FR1 of the anti-CD3 antibody or antigen-binding fragment thereof comprises or consists of an amino acid sequence having at least 75% identity with one of the sequences set forth in SEQ ID NOs: 142 and 516-535. In an embodiment, the VH FR1 of the anti-CD3 antibody or antigen-binding fragment thereof comprises or consists of an amino acid sequence having at least 80% identity with one of the sequences set forth in SEQ ID NOs: 142 and 516-535. In an embodiment, the VH FR1 of the anti-CD3 antibody or antigen-binding fragment thereof comprises or consists of an amino acid sequence having at least 85% identity with one of the sequences set forth in SEQ ID NOs: 142 and 516-535. In an embodiment, the VH FR1 of the anti-CD3 antibody or antigen-binding fragment thereof comprises or consists of an amino acid sequence having at least 90% identity with one of the sequences set forth in SEQ ID NOs: 142 and 516-535. In an embodiment, the VH FR1 of the anti-CD3 antibody or antigen-binding fragment thereof comprises or consists of amino acid sequence having at least 95% identity with one of the sequences set forth in SEQ ID NOs: 142 and 516-535. In an embodiment, the VH FR1 of the anti-CD3 antibody or antigen-binding fragment thereof comprises or consists of one of the sequences set forth in SEQ ID NOs: 142 and 516-535.

In an embodiment, the anti-CD3 antibody or antigen-binding fragment thereof comprises a heavy chain framework region 2 (VH FR2) comprising or consisting of an amino acid sequence having at least 60% identity with one of the sequences set forth in SEQ ID NOs: 178 and 536-555.

In an embodiment, the VH FR2 of the anti-CD3 antibody or antigen-binding fragment thereof comprises or consists of an amino acid sequence having at least 70% identity with one of the sequences set forth in SEQ ID NOs: 178 and 536-555. In an embodiment, the VH FR2 of the anti-CD3 antibody or antigen-binding fragment thereof comprises or consists of an amino acid sequence having at least 75% identity with one of the sequences set forth in SEQ ID NOs: 178 and 536-555. In an embodiment, the VH FR2 of the anti-CD3 antibody or antigen-binding fragment thereof comprises or consists of an amino acid sequence having at least 80% identity with one of the sequences set forth in SEQ ID NOs: 178 and 536-555. In an embodiment, the VH FR2 of the anti-CD3 antibody or antigen-binding fragment thereof comprises or consists of an amino acid sequence having at least 85% identity with one of the sequences set forth in SEQ ID NOs: 178 and 536-555. In an embodiment, the VH FR2 of the anti-CD3 antibody or antigen-binding fragment thereof comprises or consists of an amino acid sequence having at least 90% identity with one of the sequences set forth in SEQ ID NOs: 178 and 536-555. In an embodiment, the VH FR2 of the anti-CD3 antibody or antigen-binding fragment thereof comprises or consists of amino acid sequence having at least 95% identity with one of the sequences set forth in SEQ ID NOs: 178 and 536-555. In an embodiment, the VH FR2 of the anti-CD3 antibody or antigen-binding fragment thereof comprises or consists of one of the sequences set forth in SEQ ID NOs: 178 and 536-555.

In an embodiment, the anti-CD3 antibody or antigen-binding fragment thereof comprises a heavy chain framework region 3 (VH FR3) comprising or consisting of an amino acid sequence having at least 60% identity with one of the sequences set forth in SEQ ID NOs: 556-577.

In an embodiment, the VH FR3 of the anti-CD3 antibody or antigen-binding fragment thereof comprises or consists of an amino acid sequence having at least 70% identity with one of the sequences set forth in SEQ ID NOs: 556-577. In an embodiment, the VH FR3 of the anti-CD3 antibody or antigen-binding fragment thereof comprises or consists of an amino acid sequence having at least 75% identity with one of the sequences set forth in SEQ ID NOs: 556-577. In an embodiment, the VH FR3 of the anti-CD3 antibody or antigen-binding fragment thereof comprises or consists of an amino acid sequence having at least 80% identity with one of the sequences set forth in SEQ ID NOs: 556-577. In an embodiment, the VH FR3 of the anti-CD3 antibody or antigen-binding fragment thereof comprises or consists of an amino acid sequence having at least 85% identity with one of the sequences set forth in SEQ ID NOs: 556-577. In an embodiment, the VH FR3 of the anti-CD3 antibody or antigen-binding fragment thereof comprises or consists of an amino acid sequence having at least 90% identity with one of the sequences set forth in SEQ ID NOs: 556-577. In an embodiment, the VH FR3 of the anti-CD3 antibody or antigen-binding fragment thereof comprises or consists of amino acid sequence having at least 95% identity with one of the sequences set forth in SEQ ID NOs: 556-577. In an embodiment, the VH FR3 of the anti-CD3 antibody or antigen-binding fragment thereof comprises or consists of one of the sequences set forth in SEQ ID NOs: 556-577.

In an embodiment, the anti-CD3 antibody or antigen-binding fragment thereof comprises a heavy chain framework region 4 (VH FR4) comprising or consisting of an amino acid sequence having at least 60% identity with one of the sequences set forth in SEQ ID NOs: 208-210 and 578.

In an embodiment, the VH FR4 of the anti-CD3 antibody or antigen-binding fragment thereof comprises or consists of an amino acid sequence having at least 70% identity with one of the sequences set forth in SEQ ID NOs: 208-210 and 578. In an embodiment, the VH FR4 of the anti-CD3 antibody or antigen-binding fragment thereof comprises or consists of an amino acid sequence having at least 75% identity with one of the sequences set forth in SEQ ID NOs: 208-210 and 578. In an embodiment, the VH FR4 of the anti-CD3 antibody or antigen-binding fragment thereof comprises or consists of an amino acid sequence having at least 80% identity with one of the sequences set forth in SEQ ID NOs: 208-210 and 578. In an embodiment, the VH FR4 of the anti-CD3 antibody or antigen-binding fragment thereof comprises or consists of an amino acid sequence having at least 85% identity with one of the sequences set forth in SEQ ID NOs: 208-210 and 578. In an embodiment, the VH FR4 of the anti-CD3 antibody or antigen-binding fragment thereof comprises or consists of an amino acid sequence having at least 90% identity with one of the sequences set forth in SEQ ID NOs: 208-210 and 578. In an embodiment, the VH FR4 of the anti-CD3 antibody or antigen-binding fragment thereof comprises or consists of amino acid sequence having at least 95% identity with one of the sequences set forth in SEQ ID NOs: 208-210 and 578. In an embodiment, the VH FR4 of the anti-CD3 antibody or antigen-binding fragment thereof comprises or consists of one of the sequences set forth in SEQ ID NOs: 208-210 and 578.

In an embodiment, the anti-CD3 antibody or antigen-binding fragment thereof comprises a light chain framework region 1 (VL FR1) comprising or consisting of an amino acid sequence having at least 60% identity with one of the sequences set forth in SEQ ID NOs: 219, 230, 235, and 579-598.

In an embodiment, the VL FR1 of the anti-CD3 antibody or antigen-binding fragment thereof comprises or consists of an amino acid sequence having at least 70% identity with one of the sequences set forth in SEQ ID NOs: 219, 230, 235, and 579-598. In an embodiment, the VL FR1 of the anti-CD3 antibody or antigen-binding fragment thereof comprises or consists of an amino acid sequence having at least 75% identity with one of the sequences set forth in SEQ ID NOs: 219, 230, 235, and 579-598. In an embodiment, the VL FR1 of the anti-CD3 antibody or antigen-binding fragment thereof comprises or consists of an amino acid sequence having at least 80% identity with one of the sequences set forth in SEQ ID NOs: 219, 230, 235, and 579-598. In an embodiment, the VL FR1 of the anti-CD3 antibody or antigen-binding fragment thereof comprises or consists of an amino acid sequence having at least 85% identity with one of the sequences set forth in SEQ ID NOs: 219, 230, 235, and 579-598. In an embodiment, the VL FR1 of the anti-CD3 antibody or antigen-binding fragment thereof comprises or consists of an amino acid sequence having at least 90% identity with one of the sequences set forth in SEQ ID NOs: 219, 230, 235, and 579-598. In an embodiment, the VL FR1 of the anti-CD3 antibody or antigen-binding fragment thereof comprises or consists of amino acid sequence having at least 95% identity with one of the sequences set forth in SEQ ID NOs: 219, 230, 235, and 579-598. In an embodiment, the VL FR1 of the anti-CD3 antibody or antigen-binding fragment thereof comprises or consists of one of the sequences set forth in SEQ ID NOs: 219, 230, 235, and 579-598.

In an embodiment, the anti-CD3 antibody or antigen-binding fragment thereof comprises a light chain framework region 2 (VL FR2) comprising or consisting of an amino acid sequence having at least 60% identity with one of the sequences set forth in SEQ ID NOs: 244, 257, 266, and 599-613.

In an embodiment, the VL FR2 of the anti-CD3 antibody or antigen-binding fragment thereof comprises or consists of an amino acid sequence having at least 70% identity with one of the sequences set forth in SEQ ID NOs: 244, 257, 266, and 599-613. In an embodiment, the VL FR2 of the anti-CD3 antibody or antigen-binding fragment thereof comprises or consists of an amino acid sequence having at least 75% identity with one of the sequences set forth in SEQ ID NOs: 244, 257, 266, and 599-613. In an embodiment, the VL FR2 of the anti-CD3 antibody or antigen-binding fragment thereof comprises or consists of an amino acid sequence having at least 80% identity with one of the sequences set forth in SEQ ID NOs: 244, 257, 266, and 599-613. In an embodiment, the VL FR2 of the anti-CD3 antibody or antigen-binding fragment thereof comprises or consists of an amino acid sequence having at least 85% identity with one of the sequences set forth in SEQ ID NOs: 244, 257, 266, and 599-613. In an embodiment, the VL FR2 of the anti-CD3 antibody or antigen-binding fragment thereof comprises or consists of an amino acid sequence having at least 90% identity with one of the sequences set forth in SEQ ID NOs: 244, 257, 266, and 599-613. In an embodiment, the VL FR2 of the anti-CD3 antibody or antigen-binding fragment thereof comprises or consists of amino acid sequence having at least 95% identity with one of the sequences set forth in SEQ ID NOs: 244, 257, 266, and 599-613. In an embodiment, the VL FR2 of the anti-CD3 antibody or antigen-binding fragment thereof comprises or consists of one of the sequences set forth in SEQ ID NOs: 244, 257, 266, and 599-613.

In an embodiment, the anti-CD3 antibody or antigen-binding fragment thereof comprises a light chain framework region 3 (VL FR3) comprising or consisting of an amino acid sequence having at least 60% identity with one of the sequences set forth in SEQ ID NOs: 271, 284, 288, and 614-634.

In an embodiment, the VL FR3 of the anti-CD3 antibody or antigen-binding fragment thereof comprises or consists of an amino acid sequence having at least 70% identity with one of the sequences set forth in SEQ ID NOs: 271, 284, 288, and 614-634. In an embodiment, the VL FR3 of the anti-CD3 antibody or antigen-binding fragment thereof comprises or consists of an amino acid sequence having at least 75% identity with one of the sequences set forth in SEQ ID NOs: 271, 284, 288, and 614-634. In an embodiment, the VL FR3 of the anti-CD3 antibody or antigen-binding fragment thereof comprises or consists of an amino acid sequence having at least 80% identity with one of the sequences set forth in SEQ ID NOs: 271, 284, 288, and 614-634. In an embodiment, the VL FR3 of the anti-CD3 antibody or antigen-binding fragment thereof comprises or consists of an amino acid sequence having at least 85% identity with one of the sequences set forth in SEQ ID NOs: 271, 284, 288, and 614-634. In an embodiment, the VL FR3 of the anti-CD3 antibody or antigen-binding fragment thereof comprises or consists of an amino acid sequence having at least 90% identity with one of the sequences set forth in SEQ ID NOs: 271, 284, 288, and 614-634. In an embodiment, the VL FR3 of the anti-CD3 antibody or antigen-binding fragment thereof comprises or consists of amino acid sequence having at least 95% identity with one of the sequences set forth in SEQ ID NOs: 271, 284, 288, and 614-634. In an embodiment, the VL FR3 of the anti-CD3 antibody or antigen-binding fragment thereof comprises or consists of one of the sequences set forth in SEQ ID NOs: 271, 284, 288, and 614-634.

In an embodiment, the anti-CD3 antibody or antigen-binding fragment thereof comprises a light chain framework region 4 (VL FR4) comprising or consisting of an amino acid sequence having at least 60% identity with one of the sequences set forth in SEQ ID NOs: 303, 305, 306 or 635.

In an embodiment, the VL FR4 of the anti-CD3 antibody or antigen-binding fragment thereof comprises or consists of an amino acid sequence having at least 70% identity with one of the sequences set forth in SEQ ID NOs: 303, 305, 306 or 635. In an embodiment, the VL FR4 of the anti-CD3 antibody or antigen-binding fragment thereof comprises or consists of an amino acid sequence having at least 75% identity with one of the sequences set forth in SEQ ID NOs: 303, 305, 306 or 635. In an embodiment, the VL FR4 of the anti-CD3 antibody or antigen-binding fragment thereof comprises or consists of an amino acid sequence having at least 80% identity with one of the sequences set forth in SEQ ID NOs: 303, 305, 306 or 635. In an embodiment, the VL FR4 of the anti-CD3 antibody or antigen-binding fragment thereof comprises or consists of an amino acid sequence having at least 85% identity with one of the sequences set forth in SEQ ID NOs: 303, 305, 306 or 635. In an embodiment, the VL FR4 of the anti-CD3 antibody or antigen-binding fragment thereof comprises or consists of an amino acid sequence having at least 90% identity with one of the sequences set forth in SEQ ID NOs: 303, 305, 306 or 635. In an embodiment, the VL FR4 of the anti-CD3 antibody or antigen-binding fragment thereof comprises or consists of amino acid sequence having at least 95% identity with one of the sequences set forth in SEQ ID NOs: 303, 305, 306 or 635. In an embodiment, the VL FR4 of the anti-CD3 antibody or antigen-binding fragment thereof comprises or consists of one of the sequences set forth in SEQ ID NOs: 303, 305, 306 or 635.

In an embodiment, the anti-CD3 antibody or antigen-binding fragment thereof comprises an HCVR comprising or consisting of an amino acid sequence having at least 70% identity with one of the sequences set forth in SEQ ID NOs: 636, 638, 640, 642, 644, 646, 648, 650, 652, 654, 656, 658, 660, 662, 664, 666, 668, 670, 672, 674, 676, 678, 680, 682, 684, 686, 688, 690, 692, or 694.

In an embodiment, the HCVR of the anti-CD3 antibody or antigen-binding fragment thereof comprises or consists of an amino acid sequence having at least 75% identity with one of the sequences set forth in SEQ ID NOs: 636, 638, 640, 642, 644, 646, 648, 650, 652, 654, 656, 658, 660, 662, 664, 666, 668, 670, 672, 674, 676, 678, 680, 682, 684, 686, 688, 690, 692, or 694. In an embodiment, the HCVR of the anti-CD3 antibody or antigen-binding fragment thereof comprises or consists of an amino acid sequence having at least 80% identity with one of the sequences set forth in SEQ ID NOs: 636, 638, 640, 642, 644, 646, 648, 650, 652, 654, 656, 658, 660, 662, 664, 666, 668, 670, 672, 674, 676, 678, 680, 682, 684, 686, 688, 690, 692, or 694. In an embodiment, the HCVR of the anti-CD3 antibody or antigen-binding fragment thereof comprises or consists of an amino acid sequence having at least 85% identity with one of the sequences set forth in SEQ ID NOs: 636, 638, 640, 642, 644, 646, 648, 650, 652, 654, 656, 658, 660, 662, 664, 666, 668, 670, 672, 674, 676, 678, 680, 682, 684, 686, 688, 690, 692, or 694. In an embodiment, the HCVR of the anti-CD3 antibody or antigen-binding fragment thereof comprises or consists of an amino acid sequence having at least 90% identity with one of the sequences set forth in SEQ ID NOs: 636, 638, 640, 642, 644, 646, 648, 650, 652, 654, 656, 658, 660, 662, 664, 666, 668, 670, 672, 674, 676, 678, 680, 682, 684, 686, 688, 690, 692, or 694. In an embodiment, the HCVR of the anti-CD3 antibody or antigen-binding fragment thereof comprises or consists of amino acid sequence having at least 95% identity with one of the sequences set forth in SEQ ID NOs: 636, 638, 640, 642, 644, 646, 648, 650, 652, 654, 656, 658, 660, 662, 664, 666, 668, 670, 672, 674, 676, 678, 680, 682, 684, 686, 688, 690, 692, or 694. In an embodiment, the HCVR of the anti-CD3 antibody or antigen-binding fragment thereof comprises or consists of one of the sequences set forth in SEQ ID NOs: 636, 638, 640, 642, 644, 646, 648, 650, 652, 654, 656, 658, 660, 662, 664, 666, 668, 670, 672, 674, 676, 678, 680, 682, 684, 686, 688, 690, 692, or 694.

In an embodiment, the anti-CD3 antibody or antigen-binding fragment thereof comprises an LCVR comprising or consisting of an amino acid sequence having at least 70% identity with one of the sequences set forth in SEQ ID NOs: 637, 639, 641, 643, 645, 647, 649, 651, 653, 655, 657, 659, 661, 663, 665, 667, 669, 671, 673, 675, 677, 679, 681, 683, 685, 687, 689, 691, 693, or 695.

In an embodiment, the LCVR of the anti-CD3 antibody or antigen-binding fragment thereof comprises or consists of an amino acid sequence having at least 75% identity with one of the sequences set forth in SEQ ID NOs: 637, 639, 641, 643, 645, 647, 649, 651, 653, 655, 657, 659, 661, 663, 665, 667, 669, 671, 673, 675, 677, 679, 681, 683, 685, 687, 689, 691, 693, or 695. In an embodiment, the LCVR of the anti-CD3 antibody or antigen-binding fragment thereof comprises or consists of an amino acid sequence having at least 80% identity with one of the sequences set forth in SEQ ID NOs: 637, 639, 641, 643, 645, 647, 649, 651, 653, 655, 657, 659, 661, 663, 665, 667, 669, 671, 673, 675, 677, 679, 681, 683, 685, 687, 689, 691, 693, or 695. In an embodiment, the LCVR of the anti-CD3 antibody or antigen-binding fragment thereof comprises or consists of an amino acid sequence having at least 85% identity with one of the sequences set forth in SEQ ID NOs: 637, 639, 641, 643, 645, 647, 649, 651, 653, 655, 657, 659, 661, 663, 665, 667, 669, 671, 673, 675, 677, 679, 681, 683, 685, 687, 689, 691, 693, or 695. In an embodiment, the LCVR of the anti-CD3 antibody or antigen-binding fragment thereof comprises or consists of an amino acid sequence having at least 90% identity with one of the sequences set forth in SEQ ID NOs: 637, 639, 641, 643, 645, 647, 649, 651, 653, 655, 657, 659, 661, 663, 665, 667, 669, 671, 673, 675, 677, 679, 681, 683, 685, 687, 689, 691, 693, or 695. In an embodiment, the LCVR of the anti-CD3 antibody or antigen-binding fragment thereof comprises or consists of amino acid sequence having at least 95% identity with one of the sequences set forth in SEQ ID NOs: 637, 639, 641, 643, 645, 647, 649, 651, 653, 655, 657, 659, 661, 663, 665, 667, 669, 671, 673, 675, 677, 679, 681, 683, 685, 687, 689, 691, 693, or 695. In an embodiment, the LCVR of the anti-CD3 antibody or antigen-binding fragment thereof comprises or consists of one of the sequences set forth in SEQ ID NOs: 637, 639, 641, 643, 645, 647, 649, 651, 653, 655, 657, 659, 661, 663, 665, 667, 669, 671, 673, 675, 677, 679, 681, 683, 685, 687, 689, 691, 693, or 695.

In an embodiment, the anti-CD3 antibody or antigen-binding fragment thereof comprises a combination of one the HCVRs and one of the LCVRs defined above. In a further embodiment, the antibody or antigen-binding fragment thereof comprises one of the following combinations of HCVR and LCVR: 636 and 637, 638 and 639, 640 and 641, 642 and 643, 644 and 645, 646 and 647, 648 and 649, 650 and 651, 652 and 653, 654 and 655, 656 and 657, 658 and 659, 660 and 661, 662 and 663, 664 and 665, 666 and 667, 668 and 669, 670 and 671, 672 and 673, 674 and 675, 676 and 677, 678 and 679, 680 and 681, 682 and 683, 684 and 685, 686 and 687, 688 and 689, 690 and 691, 692 and 693, or 694 and 695.

In an embodiment, the anti-CD3 antibody or antigen-binding fragment thereof comprises the HCVR and LCVR of clones 1A09, 8A05 or 3G07 described herein.

### Antigen-Binding Fragments of Antibodies

The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen (e.g., TrkB, CD3) to form a complex. The terms “antigen-binding fragment” of an antibody, or “antibody fragment”, as used herein, refers to one or more fragments of an antibody that retain the ability to specifically bind to TrkB or CD3. An antibody fragment may include a Fab fragment, a F(ab′)₂ fragment, an Fv fragment, a dAb fragment, a fragment containing a CDR, or an isolated CDR. In certain embodiments, the term “antigen-binding fragment” refers to a polypeptide fragment of a multi-specific antigen-binding molecule. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and (optionally) constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)₂ fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR, which is adjacent to or in frame with one or more framework sequences. In antigen-binding fragments having a VH domain associated with a VL domain, the VH and VL domains may be situated relative to one another in any suitable arrangement. For example, the variable region may be dimeric and contain VH-VH, VH-VL or VL-VL dimers. Alternatively, the antigen-binding fragment of an antibody may contain a monomeric VH or VL domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) VH-CH1; (ii) VH-CH2; (iii) VH-CH3; (iv) VH-CH1-CH2; (v) VH-CH1-CH2-CH3, (vi) VH-CH2-CH3; (vii) VH-CL; (viii) VL-CH1; (ix) VL-CH2; (x) VL-CH3; (xi) VL-CH1-CH2; (xii) VL-CH1-CH2-CH3; (xiii) VL-CH2-CH3, and (xiv) VL-CL. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present disclosure may comprise a homodimer or heterodimer (or other multimer) of any of the variable and constant domain configurations listed above in non-covalent association with one another and/or with one or more monomeric VH or VL domain (e.g., by disulfide bond(s)).

As with full antibody molecules, antigen-binding fragments may be mono-specific or multi-specific (e.g., bi-specific). Multi-specific antibodies or antigen-binding fragments thereof may be specific for different epitopes of one target polypeptide or may contain antigen-binding domains specific for more than one target polypeptide. See, e.g., Tutt et al., 1991, J. Immunol. 147:60-69; Kufer et al., 2004, Trends Biotechnol. 22:238-244.

Any of the multi-specific antigen-binding molecules of the disclosure, or variants thereof, may be constructed using standard molecular biological techniques (e.g., recombinant DNA and protein expression technology), as will be known to a person of ordinary skill in the art.

An exemplary bispecific antibody format that can be used in the context of the present disclosure involves the use of a first immunoglobulin (lg) CH3 domain and a second Ig CH3 domain, wherein the first and second Ig CH3 domains differ from one another by at least one amino acid, and wherein at least one amino acid difference reduces binding of the bispecific antibody to Protein A as compared to a bispecific antibody lacking the amino acid difference. In one embodiment, the first Ig CH3 domain binds Protein A and the second Ig CH3 domain contains a mutation that reduces or abolishes Protein A binding such as an H95R modification (by IMGT exon numbering; H435R by EU numbering). The second CH3 may further comprise a Y96F modification (by IMGT; Y436F by EU). Further modifications that may be found within the second CH3 include: D16E, L18M, N44S, K52N, V57M, and V821 (by IMGT; D356E, L358M, N384S, K392N, V397M, and V422I by EU) in the case of IgG1 antibodies; N44S, K52N, and V821 (by IMGT; N384S, K392N, and V4221 by EU) in the case of lgG2 antibodies; and Q15R, N44S, K52N, V57M, R69K, E79Q, and V821 (by IMGT; Q355R, N384S, K392N, V397M, R409K, E419Q, and V4221 by EU) in the case of lgG4 antibodies. Variations on the bispecific antibody format described above are contemplated within the scope of the present disclosure.

Other exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, lgG1/lgG2, dual acting Fab (DAF)-IgG, and Mab2 bispecific formats (see, e.g., Klein et al. 2012, mAbs 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al. (2012) Site-specific DNA-antibody conjugates for specific and sensitive immuno-PCR, PNAS 109 (10) 3731-3736; DOI: 10.1073/pnas. 1120682109, and U.S. Pat. Nos. 4,496,689; 4,301,144; 4,670,417; 4,791,192 or 4,179,337). In an embodiment, the bispecific antibody or antigen-binding fragment thereof is of the knobs-into-holes format.

The bispecific antibody of the present disclosure may include any one of the combinations of CDRs and/or VL/VH regions defined herein.

A particular type of multispecific antibodies, also included herein, are bispecific antibodies designed to simultaneously bind to an immune cell receptor, such as a CD3, and to TrkB, for retargeting of T-cells to kill target (e.g., tumor) cells expressing TrkB. Hence, in preferred aspects, an antibody or antigen-binding fragment provided herein is a multispecific antibody or antigen-binding fragment, particularly a bispecific antibody or antigen-binding fragment, wherein one of the binding specificities is for TrkB and the other is for an immune cell receptor, such as a CD3.

Examples of bispecific antibody or antigen-binding fragment formats that may be useful for this purpose include, but are not limited to, the so-called “BiTE” (bispecific T-cell engager) molecules wherein two scFv molecules are fused by a flexible linker (see, e.g., WO 2004/106381, WO 2005/061547, WO 2007/042261, and WO 2008/119567, Nagorsen and Bauerle, Exp Cell Res 317, 1255-1260 (2011)); diabodies (Holliger et al., Prot Eng 9, 299-305 (1996)) and derivatives thereof, such as tandem diabodies (“TandAb”; Kipriyanov et al., J Mol Biol 293, 41-56 (1999)); “DART” (dual affinity retargeting) molecules which are based on the diabody format but feature a C-terminal disulfide bridge for additional stabilization (Johnson et al., J Mol Biol 399, 436-449 (2010)), and so-called triomabs, which are whole hybrid mouse/rat IgG molecules (reviewed in Seimetz et al., Cancer Treat Rev 36, 458-467 (2010)). Particular T-cell bispecific antibody formats included herein are described in WO 2013/026833, WO 2013/026839, WO 2016/020309, and Bacac et al., Oncoimmunology 5 (8) (2016) el203498.

In an aspect, the disclosure provides an antibody that binds to TrkB, comprising a first antigen binding domain that binds to TrkB as described herein, and comprising a second and optionally a third antigen binding domain that binds to a second (and optionally third) antigen.

In an embodiment according to the disclosure, the (multispecific) antibody is capable of simultaneous binding to the first antigen (i.e., CD3), and the second antigen (e.g., a target cell antigen). In one aspect, the (multispecific) antibody is capable of crosslinking a T-cell and a target cell by simultaneous binding to CD3 and a target cell antigen. In an embodiment according to the disclosure, such simultaneous binding results in lysis of the target cell, particularly a target cell antigen-expressing tumor cell. In one embodiment, such simultaneous binding results in activation of the T-cell. In other aspects, such simultaneous binding results in a cellular response of a T lymphocyte, particularly a cytotoxic T lymphocyte, such as proliferation, differentiation, cytokine secretion, cytotoxic effector molecule release, cytotoxic activity, and/or expression of activation markers. In one aspect, the (multispecific) antibody is capable of re-directing cytotoxic activity of a T-cell to a target cell. In a preferred aspect, said re-direction is independent of MHC-mediated peptide antigen presentation by the target cell and and/or specificity of the T-cell.

In some embodiments, the T-cell is a CD4⁺ or a CD8⁺ T-cell. In a further embodiment, the T-cell is a CD8⁺ T-cell, e.g., a cytotoxic T-cell. In an embodiment, the second and/or third antigen is a tumor antigen, i.e., a molecule (a protein, saccharide, lipid, etc.) expressed by a tumor cell.

In another aspect, the disclosure provides an antibody that binds to CD3, comprising a first antigen binding domain that binds to CD3 as described herein, and comprising a second and optionally a third antigen binding domain that binds to a second (and optionally third) antigen. In an embodiment, the second and/or third antigen is a tumor antigen, i.e., a molecule (a protein, saccharide, lipid, etc.) expressed by a tumor cell.

Examples of tumor antigens include MAGE 1, 3, and 4 or other MAGE antigens, PRAME, BAGE, LAGE (also known as NY-Eos-1) SAGE, and HAGE or GAGE. Such antigens are expressed in a wide range of tumor types such as melanoma, lung carcinoma, sarcoma, and bladder carcinoma. Other tumor-specific antigens include, but are not restricted to tumor-specific gangliosides such as GM2, GM3, or conjugates thereof to carrier proteins; prostate antigens such as prostate specific antigen (PSA), PAP, STEAP, PSCA, PCA3, PSMA, or Prostase, carcinoembryonic antigen (CEA), KSA (also known as EpCAM), gp100, Plu-1, HASH-1, HasH-2, Cripto, Criptin, mucin and mucin-derived peptides such as Muc1, Muc5, Muc16 and Muc17, HER2, EGFR, CD20, TROP2, BCMA, GPCR5D, CD19, CLDN18.2, CLL-1, CD37, CD33, CD123, GPNMB, GPC3, MSLN, TSPAN8, FOLR1, FOLR2, PLAP, FLT3, 5T4. The skilled person would understand that the second (and optionally third) antigen-binding domain of the multispecific antibody is selected based on the tumor antigen(s) expression by the tumor that is being treated. For example, a multispecific antibody comprising an antigen-binding domain specifically binding to CD19 may be designed for targeting CD19-expressing tumors. The tumor antigen may also be a peptide presented by MHC molecules at the surface of tumor cells.

In an embodiment, the multispecific antibody comprises a third antigen binding domain that binds to a third antigen. In an embodiment, the third antigen is another tumor antigen.

In another embodiment, the third antigen is a protein involved in the immune response, for example a protein involved in T-cell activation.

In an embodiment, the third antigen is a protein that stimulates the immune response, and the third antigen binding domain inhibits the activity of the third antigen. In an embodiment, the third antigen is a protein that inhibits the immune response, and the third antigen binding domain increases the activity of the third antigen. Antibodies according to these embodiments may be useful, e.g., for inhibiting the immune response, which may be beneficial, e.g., for the treatment of autoimmune diseases.

In an embodiment, the third antigen is a protein that stimulates the immune response, and the third antigen binding domain increases the activity of the third antigen. In an embodiment, the third antigen is a protein that inhibits the immune response, and the third antigen binding domain blocks the activity of the third antigen. Antibodies according to these embodiments may be useful, e.g., for stimulating or increasing the immune response, which may be beneficial, e.g., for the treatment of cancer and infectious diseases.

In an embodiment, the third antigen is a protein that stimulates the immune response such as TNF-α, IL-6/IL-6R, IL2/IL-2R, CD28, IL-15/IL-15R, CD137, B7-H4, TIM-3 IL-17/IL-17R, or IL-1/IL-1R.

In an embodiment, the third antigen is a protein that inhibits the immune response (e.g., checkpoint) such as TGF-β, PD-1, PD-L1, CTLA-4, or LAG-3.

In another embodiment, the second or third antigen is a protein involved in the immune response, for example a protein involved in T-cell activation, such as a CD3.

In an embodiment, the second or third antigen binding domain is a CD3 binding domain that binds to CD3, and in embodiments the CD3 binding domain is derived from the anti-CD3 antibody or an antigen-binding fragment thereof described herein.

In an embodiment, the CDR-H1, CDR-H2, CDR-H3, CDR-L1, CDR-L2 and/or CDR-L3 of the anti-CD3 antibody or antigen-binding fragment thereof or CD3 binding domain comprises or consists of the following amino acid sequences:
1) CDR-H1: SEQ ID NO:414; CDR-H2: SEQ ID NO:433; CDR-H3: SEQ ID NO: 449; CDR-L1: SEQ ID NO:78; CDR-L2: QDS; and CDR-L3: SEQ ID NO: 108;
2) CDR-H1: SEQ ID NO:415; CDR-H2: SEQ ID NO:434; CDR-H3: SEQ ID NO:451; CDR-L1: SEQ ID NO:475; CDR-L2: DDN; and CDR-L3: SEQ ID NO:494; or
3) CDR-H1: SEQ ID NO:415; CDR-H2: SEQ ID NO:434; CDR-H3: SEQ ID NO:451; CDR-L1: SEQ ID NO:476; CDR-L2: DDK; and CDR-L3: SEQ ID NO:495.

In an embodiment, the HCVR of the anti-CD3 antibody or antigen-binding fragment thereof or CD3 binding domain comprises or consists of one of the following sequences:
1) SEQ ID NO:638; 2) SEQ ID NO:646; or 3) SEQ ID NO:648.

In an embodiment, the LCVR of the anti-CD3 antibody or antigen-binding fragment thereof or CD3 binding domain comprises or consists of one of the following sequences:
1) SEQ ID NO:639; 2) SEQ ID NO:647; or 3) SEQ ID NO:649.

In a further embodiment, the anti-CD3 antibody or antigen-binding fragment thereof or CD3 binding domain comprises one of the following combinations of HCVR and LCVR:
1) HCVR: SEQ ID NO:638 and LCVR: SEQ ID NO:639;
2) HCVR: SEQ ID NO:646 and LCVR: SEQ ID NO:647; or
3) HCVR: SEQ ID NO:648 and LCVR: SEQ ID NO:649.

In an embodiment, the multispecific (e.g., bispecific) antibody comprises a TrkB-binding domain comprising the CDRs or HCVR/LCVR of clones F5, 3H11, 6E08 or 3C12 described herein, and a CD3-binding domain comprising the CDRs or HCVR/LCVR of clones 1-A09, 8-A05 or 3-G07 described herein.

The antibody or antigen-binding fragment thereof described herein may further comprise one or more modifications that confer additional biological properties to the antibody or antigen-binding fragment thereof such as increased protease resistance, reduced plasma protein binding, increased in vivo half-life, increased intracellular penetration, increased storage stability, increased expression, reduced aggregation, etc. Such modifications include, for example, covalent attachment of molecules/moiety to the antibody or antigen-binding fragment thereof such as fatty acids (e.g., C6-C18), attachment of proteins such as albumin (see, e.g., U.S. Pat. No. 7,268,113); sugars/polysaccharides (glycosylation), biotinylation or PEGylation (see, e.g., U.S. Pat. Nos. 7,256,258 and 6,528,485). The antibody or antigen-binding fragment thereof may also be mutated to remove an N- or O-glycosylation site, e.g., by mutating one or more asparagine residues, or one or more Ser or Thr residues, respectively, in the sequence of the heavy and/or light chain(s) of the antibody or antigen-binding fragment thereof.

### Conjugates

The above description of modification of the antibody or antigen-binding fragment thereof does not limit the scope of the approaches nor the possible modifications that can be engineered. Thus, in another aspect, the present disclosure provides a conjugate comprising the antibody or antigen-binding fragment thereof described herein and one or more additional molecules or agents (hereinafter secondary molecules or agents). The antibody or antigen-binding fragment thereof may be conjugated to any type of synthetic or natural secondary molecules or agents, such as peptides. proteins, saccharides/polysaccharides, lipids, drugs, naturally-occurring or synthetic polymers/co-polymers, etc. to modify one or more properties of the antibody or antigen-binding fragment thereof.

In an embodiment, the conjugate comprises a covalent link or bond between the antibody or antigen-binding fragment thereof and the molecule conjugated thereto. The molecule may be conjugated directly to the antibody or antigen-binding fragment thereof, or indirectly via a linker. The linker may be a polypeptide linker comprising one or more amino acids or another type of chemical linker (e.g., a carbohydrate linker, a lipid linker, a fatty acid linker, a polyether linker, PEG, etc.).

In another embodiment, the molecule may be conjugated/attached to the side chain of one the amino acids of the antibody or antigen-binding fragment thereof. Methods for conjugating moieties to side chains of amino acids are well known in the art. For example, chemical groups that react with primary amines (—NH₂) present in the side-chain of lysine residues such as isothiocyanates, isocyanates, acyl azides, NHS esters, sulfonyl chlorides, aldehydes, glyoxals, epoxides, oxiranes, carbonates, aryl halides, imidoesters, carbodiimides, anhydrides, and fluorophenyl esters may be used to conjugate the molecule to the antigenic peptide. Most of these groups conjugate to amines by either acylation or alkylation. Cysteine residues present in the antibody or antigen-binding fragment thereof may also be used to attach the molecule.

In an embodiment, the anti-CD3 or anti-TrkB antibody or antigen-binding fragment thereof or multispecific (e.g., bispecific) antibody described herein is labelled or conjugated with one or more moieties. The antibody or antigen-binding fragment thereof may be labeled with one or more labels such as a biotin label, a fluorescent label, an enzyme label, a coenzyme label, a chemiluminescent label, or a radioactive isotope label. In an embodiment, the antibody or antigen-binding fragment thereof is labelled with a detectable label, for example a fluorescent moiety (fluorophore). Useful detectable labels include fluorescent compounds (e.g., fluorescein isothiocyanate, Texas red, rhodamine, fluorescein, Alexa Fluor® dyes, and the like), radiolabels, enzymes (e.g., horseradish peroxidase, alkaline phosphatase and others commonly used in a protein detection assays), streptavidin/biotin, and colorimetric labels such as colloidal gold, colored glass or plastic beads (e.g., polystyrene, polypropylene, latex, etc.). Chemiluminescent compounds may also be used. Such labelled antibodies or antigen-binding fragments thereof may be useful, for example, for the detection of TrkB and/or TrkB-expressing cells in vivo or in vitro, e.g., by flow cytometry, immunohistochemistry, etc. The antibody or antigen-binding fragment thereof can also be conjugated to detectable or affinity tags that facilitate detection and/or purification of the antibody or antigen-binding fragment thereof. Such tags are well known in the art. Examples of detectable or affinity tags include polyhistidine tags (His-tags), polyarginine tags, polyaspartate tags, polycysteine tags, polyphenylalanine tags, glutathione S-transferase (GST) tags, maltose-binding protein (MBP) tags, calmodulin-binding peptide (CBP) tags, streptavidin/biotin-based tags, HaloTag®, Profinity eXact® tags, epitope tags (such as FLAG, hemagglutinin (HA), HSV, S/S1, c-myc, KT3, T7, V5, E2, and Glu-Glu epitope tags), reporter tags such as β-galactosidase (β-gal), alkaline phosphatase (AP), chloramphenicol acetyl transferase (CAT), and horseradish peroxidase (HRP) tags (see, e.g., Kimple et al., Curr Protoc Protein Sci. 2013; 73: Unit-9.9).

In some embodiments, the anti-CD3 or anti-TrkB antibody or antigen-binding fragment thereof or multispecific (e.g., bispecific) antibody described herein can be conjugated to a targeting moiety when employed in the therapeutic methods described herein. The targeting moieties can be a protein or a peptide which directs localization to a certain part of the body, e.g., to the central nervous system, brain or compartments therein, or to a tumor. In some embodiments, the anti-CD3 or anti-TrkB antibody or antigen-binding fragment thereof or multispecific (e.g., bispecific) antibody described herein can be attached or fused to a brain targeting moiety. The brain targeting moieties can be attached covalently (e.g., direct, translational fusion, or by chemical linkage either directly or through a spacer/linker molecule, which can be optionally cleavable) or non-covalently attached (e.g., through reversible interactions such as avidin, biotin, protein A, IgG, etc.). A brain targeting moiety conjugated to the anti-CD3 or anti-TrkB antibody or antigen-binding fragment thereof or multispecific (e.g., bispecific) antibody described herein can enhance brain delivery of the anti-CD3 or anti-TrkB antibody or antigen-binding fragment thereof or multispecific (e.g., bispecific) antibody. A number of agents can be employed as the brain targeting moiety. These include polypeptides or antibody fragments which can deliver a fused protein or therapeutic agent through the blood brain barrier (BBB). Examples of such agents include single domain antibody FC5 (Aabulrob et al. J. Neurochem. 95, 1201-1214, 2005); mAB 83-14, a monoclonal antibody to the human insulin receptor (Pardridge et al. Pharmacol. Res. 12, 807-816, 1995); the B2, B6 and B8 peptides which bind to the human transferrin receptor (hTfR) (Xia et al. J. Virol. 74, 11359-11366, 2000); the OX26 monoclonal antibody to the transferrin receptor (Pardridge et al. J. Pharmacol. Exp. Ther. 259, 66-70, 1991); and several polypeptides described in U.S. Pat. No. 6,306,365.

In an embodiment, the anti-TrkB antibody or antigen-binding fragment thereof or multispecific (e.g., bispecific) antibody described herein is conjugated to a drug, such as an antitumor agent (i.e., is an antibody-drug conjugate, ADC). The antitumor agent may be any compound that has the ability to inhibit the growth and/or kill tumor cells and includes, for example, small molecules, peptides, proteins, oligonucleotides (e.g., siRNA, shRNA), radionuclide agents (e.g., ¹⁷⁷Lu, ¹⁸F, ⁶⁸Ga, ⁹⁰Y, ^{99m}Tc, ¹¹¹In, ²¹³Bi, ²²¹At, ²²⁵Ac, ²²⁷Th), as well as drug delivery systems including nanoparticles (e.g., lipid nanoparticles), liposomes, nanotubes, etc., loaded with a therapeutic antitumor agent.

In an embodiment, the antitumor agent is a chemotherapeutic agent. The term “chemotherapeutic agent” refers to agents that kill tumor cells and/or inhibit their proliferation/growth. Examples in chemotherapeutic agents include alkylating agents (e.g., Cyclophosphamide, Ifosfamide, Mechlorethamine, Chlorambucil, Melphalan, Dacarbazine, Nitrosoureas, Temozolomide, Carmustine, Lomustine, Streptozocin, Busulfan, Procarbazine), anthracyclines (e.g., Daunorubicin, Doxorubicin, Epirubicin, Idarubicin, Mitoxantrone, Valrubicin), Monomethyl auristatin E (MMAE), monomethyl auristatin F (MMAF), cytoskeletal disruptors (e.g., taxanes such as Paclitaxel, Docetaxel, Abraxane, Taxotere, cabazitaxel), histone deacetylase inhibitors (e.g., Vorinostat, Romidepsin), topoisomerase I inhibitors (e.g., camptothecin analogs such as Irinotecan, Topotecan, SN-38, Silatecan, Exatecan, Lurtotecan, Deruxtecan), topoisomerase II inhibitors (e.g., Etoposide, Teniposide, Tafluposide), kinase inhibitors (e.g., Bortezomib, Erlotinib, Gefitinib, Imatinib, Vemurafenib, Vismodegib, Dasatinib, Nilotinib, Osimertinib, Crizotinib, Dabrafenib, Vemurafenib, Trametinib, Ibrutinib), nucleotide analogs and precursor analogs (e.g., Azacitidine, Azathioprine, Capecitabine, Cytarabine, Doxifluridine, Fluorouracil (5-FU), Gemcitabine, Hydroxyurea, Mercaptopurine, Methotrexate, Tioguanine (Thioguanine)), peptide antibiotics (e.g., Bleomycin, Actinomycin), platinum-based agents (e.g., Carboplatin, Cisplatin, Oxaliplatin), retinoids (Tretinoin, Alitretinoin, Bexarotene), SERCA inhibitors (e.g., Thapsigargin), mitotic inhibitors such as vinca alkaloids and derivative (e.g., Vinblastine. Vincristine, Vindesine, Vinorelbine), tubulin inhibitors such as Maytansinoids, maytansinoid derivatives such as Mertansine (DM1) or Ravtansine (DM4), Auristatins, Calicheamicins, Tubulysins, Amatoxin or Amanitin, as well as natural phytochemicals having anti-tumor properties such as curcumin, Alkaloids (e.g., Chlorogenic acid, Theobromine, Theophylline), Anthocyanins (e.g., Cyanidin, Malvidin, Carotenoids (Beta-Carotene, Lutein, Lycopene), Coumestans, Flavan-3-Ols, Flavonoids (e.g., Epicatechin, Hesperidin, Isorhamnetin, Kaempferol, Myricetin, Naringin, Nobiletin, Proanthocyanidins, Quercetin, Rutin, Tangeretin), Hydroxycinnamic Acids (e.g., Chicoric acid, Coumarin, Ferulic acid, Scopoletin), Isoflavones (e.g., Daidzein, Genistein), Lignans (e.g., Silymarin), Monoterpenes (e.g., Geraniol, Limonene), Organosulfides (e.g., Allicin, Glutathione, Indole-3-Carbinol, Isothiocyanates, Sulforaphane), Damnacanthal, Digoxin, Phytic acid, Phenolic Acids (e.g., Capsaicin, Ellagic Acid, Gallic acid, Rosmarinic acid, Tannic Acid), Phytosterols (e.g., Beta-Sitosterol), Saponins. Stylbenes (e.g., Pterostilbene, Resveratrol), Triterpenoids (e.g., Ursolic acid). Xanthophylls (e.g., Astaxanthin, Beta-Cryptoxanthin), and Monophenols (e.g., Hydroxytyrosol).

The antitumor agent or drug delivery system may be conjugated directly or indirectly (i.e., through a linker) to the antibody or antigen-binding fragment thereof. In an embodiment, the antitumor agent is conjugated through a linker to antibody or antigen-binding fragment thereof, optionally a cleavable linker.

The term “linker” as used herein means a chemical structure connecting the antibody or antigen-binding fragment thereof disclosed herein to at least one antitumor agent or drug delivery system. The linker can be connected to the antibody or antigen-binding fragment thereof at different functional groups on the antibody or antigen-binding fragment thereof. For example, the linker can be connected to the antibody or antigen-binding fragment thereof at the primary amines (amines (-NH₂): this group exists at the N-terminus of each polypeptide chain (called the alpha-amine) and in the side chain of lysine (Lys, K) residues (called the epsilon-amine). For example, the linker can be connected to the antibody or antigen-binding fragment thereof at the carboxyls (—COOH): this group exists at the C-terminus of each polypeptide chain and in the side chains of aspartic acid (Asp, D) and glutamic acid (Glu, E). For example, the linker can be connected to the antibody or antigen-binding fragment thereof at the Sulfhydryls (—SH): This group exists in the side chain of cysteine (Cys, C). Often, as part of a protein's secondary or tertiary structure, cysteines are joined together between their side chains via disulfide bonds (—S—S—). These must be reduced to sulfhydryls to make them available for crosslinking by most types of reactive groups. For example, the linker can be connected to the antibody or antigen-binding fragment thereof at the Carbonyls (-CHO): Ketone or aldehyde groups can be created in glycoproteins by oxidizing the polysaccharide post-translational modifications (glycosylation) with sodium meta-periodate.

### Nucleic Acids and Cells

A further aspect of the present disclosure provides nucleic acids encoding the anti-CD3 or anti-TrkB antibody or antigen-binding fragment thereof or multispecific (e.g., bispecific) antibody described herein, e.g., encoding the light and heavy chains of the antibody or antigen-binding fragment. The isolated nucleic acid may be a synthetic DNA, an mRNA (e.g., a non-naturally occurring mRNA), or a cDNA, for example. The nucleic acid may be inserted within a plasmid, vector, or transcription or expression cassette. The nucleic acids encoding the antibody or antigen-binding fragment described herein may be made and the expressed antibodies or antigen-binding fragments described may be tested using conventional techniques well known in the art. In some embodiments, the nucleic acid encoding the antibody or antigen-binding fragment described herein can be maintained in the vector in a host cell. In some embodiments, the nucleic acid is an expression vector. In some embodiments, the nucleic acid sequence encoding the antibody can be maintained in the vector in a host cell. In another embodiment, the nucleic acid(s) (DNA, mRNA) encoding the antibody or antigen-binding fragment described herein of the disclosure is comprised within a vesicle such as liposomes or lipid nanoparticles or any other suitable vehicle. In an embodiment, the nucleic acid(s) is/are mRNA and is/are encapsulated into nanoparticulate delivery vehicles (see, e.g., Van Hoecke and Roose (2019) How mRNA therapeutics are entering the monoclonal antibody field, J. Transl. Med. 17, 54; Sanz and Álvarez-Vallina (2021) Engineered mRNA and the Rise of Next-Generation Antibodies, Antibodies 10(4): 37.).

In another aspect, the present disclosure provides a cell, for example a recombinant DNA host cell, expressing the anti-CD3 or anti-TrkB antibody or antigen-binding fragment thereof or multispecific (e.g., bispecific) antibody described herein. Methods of preparing antibodies or antigen-binding fragments comprise expressing the encoding nucleic acid(s) in a host cell under conditions to produce the antibodies or antigen-binding fragments, and recovering the antibodies or antigen-binding fragments. The process of recovering the antibodies or antigen-binding fragments may comprise isolation and/or purification of the antibodies or antigen-binding fragments. The method of production may comprise formulating the antibodies or antigen-binding fragments into a composition including at least one additional component, such as a pharmaceutically acceptable excipient. In another aspect, provided herein is a cell expressing one or more antibodies of the disclosure.

The term “recombinant host cell” (or simply “host cell”), as used herein, is intended to refer to a cell into which exogenous DNA has been introduced. It should be understood that such terms are intended to refer not only to the particular subject cell, but, to the progeny of such a cell. Because certain modifications may occur in succeeding generations due to either mutation or environmental influences, such progeny may not, in fact, be identical to the parent cell, but are still included within the scope of the term “host cell” as used herein. Preferably host cells include prokaryotic and eukaryotic cells selected from any of the Kingdoms of life. To produce the antibody or antigen-binding fragment thereof recombinantly, the nucleic acid or nucleic acids encoding the light and heavy chains of the antibody or antigen-binding fragment thereof are introduced in a cell which is able to produce the recombinant antibody. Examples thereof include CHO-K1 (ATCC CCL-61), DUKXB11 (ATCC CCL-9096), Pro-5 (ATCC CCL-1781), CHO-S (Life Technologies®, Cat #11619), FreeStyle™ 293-F cells (ThermoFisher Scientific, Cat #R79007), rat myeloma cell YB2/3HL.P2.G11.16Ag.20 (also called YB2/0), mouse myeloma cell NSO, mouse myeloma cell SP2/0-Ag14 (ATCC No. CRL1581), mouse P3-X63-Ag8653 cell (ATCC No. CRL1580), CHO cell in which a dihydrofolate reductase gene is defective, lectin resistance-acquired Lec13, CHO cell in which a1,6-fucosyltransaferse gene is defective, rat YB2/3HL.P2.G11.16Ag.20 cell (ATCC No. CRL1662), CHO-3E7 cells (expressing a truncated but functional form of EBNA1, U.S. Pat. No. 8,637,315) or the like. After introduction of the expression vector, transformants which stably express a recombinant antibody are selected by culturing them in a medium for animal cell culture containing an agent such as G418 sulfate or the like. Examples of the medium for animal cell culture include RPMI1640 medium (manufactured by Invitrogen®), GIT medium (manufactured by Nihon Pharmaceutical®), EX-CELL301® medium (manufactured by JRH®), IMDM medium (manufactured by Invitrogen®), Hybridoma-SFM medium (manufactured by Invitrogen®), media obtained by adding various additives such as FBS to these media, or the like. The recombinant antibody can be produced and accumulated in a culture supernatant by culturing the obtained transformants in a medium. The expression level and antigen-binding activity of the recombinant antibody in the culture supernatant can be measured by ELISA or the like. Also, in the transformant, the expression level of the recombinant antibody can be increased by using DHFR amplification system or the like. The recombinant antibody can be purified from the culture supernatant of the transformant by using a protein A column. In addition, the recombinant antibody can be purified by combining the protein purification methods such as gel filtration, affinity chromatography (Protein A immobilized columns) ion-exchange chromatography, ultrafiltration or the like. The molecular weight and integrity of the H chain or the L chain of the purified recombinant antibody or the antibody molecule as a whole is determined by polyacrylamide gel electrophoresis, Western blotting, mass spectrometry or the like.

Suitable vectors comprising nucleic acid(s) encoding the antibody or antigen-binding fragment described herein can be chosen or constructed, containing appropriate regulatory sequences, including promoter sequences, terminator sequences, polyadenylation sequences, enhancer sequences, marker genes and other sequences as appropriate. Vectors may be plasmids, phage, phagemids, adenoviral, AAV, lentiviral, for example. Techniques and protocols for manipulation of nucleic acid, for example in preparation of nucleic acid constructs, mutagenesis, sequencing, introduction of DNA into cells, and gene expression, are well known in the art.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome.

Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the disclosure is intended to include such other forms of expression vectors, such as viral vectors (e.g., replication defective retroviruses, adenoviruses and adeno-associated viruses), which serve equivalent functions.

Introducing such nucleic acids into a host cell can be accomplished using techniques well known in the art. For eukaryotic cells, suitable techniques may include calcium phosphate transfection, DEAE-Dextran, electroporation, liposome-mediated transfection, and transduction using retroviruses or other viruses, for example. For bacterial cells, suitable techniques may include calcium chloride transformation, electroporation, and transfection using bacteriophage. The introduction may be followed by causing or allowing expression from the nucleic acid, e.g., by culturing host cells under conditions for expression of the gene. In one embodiment, the nucleic acid of the disclosure is integrated into the genome, e.g., chromosome, of the host cell. Integration may be promoted by inclusion of sequences which promote recombination with the genome, in accordance with standard techniques.

### Pharmaceutical/Therapeutic Compositions

Also provided are pharmaceutical or therapeutic compositions comprising the anti-CD3 or anti-TrkB antibodies or antigen-binding fragments thereof or multispecific (e.g., bispecific) antibodies described herein, or conjugates, or nucleic acids encoding such antibodies or antigen-binding fragments thereof, as described herein. Pharmaceutical compositions are generally administered with suitable carriers, excipients, and other agents that are incorporated into formulations to provide e.g. improved transfer, delivery, tolerance, and include formulations described in Remington's Pharmaceutical Sciences, Mack Publishing Company, Easton, Pa, which formulations include, for example, powders, pastes, ointments, jellies, waxes, oils, lipids, lipid (cationic or anionic) containing vesicles, DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The carrier/excipient can be suitable for administration of the antibody or an antigen-binding fragment thereof, or the nucleic acid(s) encoding the antibody or antigen-binding fragment thereof, or conjugates, by any conventional administration route, for example, for oral, intravenous, parenteral, subcutaneous, intramuscular, intracranial, intraorbital, ophthalmic, intraventricular, intracapsular, intraspinal, intrathecal, epidural, intracisternal, intraperitoneal, intranasal or pulmonary (e.g., aerosol) administration. In an embodiment, the carrier/excipient is adapted for administration of the antibody or an antigen-binding fragment thereof by the intravenous or subcutaneous route. In an embodiment, the carriers/excipients are adapted for administration of the antibody or an antigen-binding fragment thereof, or the nucleic acid(s) encoding the antibody or antigen-binding fragment thereof, by the intravenous route. In another embodiment, the carriers/excipients are adapted for administration of the antibody or an antigen-binding fragment thereof, or the nucleic acid(s) encoding the antibody or antigen-binding fragment thereof, by the subcutaneous route.

An “excipient” as used herein has its normal meaning in the art and is any ingredient that is not a bioactive ingredient (drug) itself. Excipients include for example binders, lubricants, diluents, fillers, thickening agents, disintegrants, plasticizers, coatings, barrier layer formulations, lubricants, stabilizing agent, release-delaying agents and other components. “Pharmaceutically acceptable excipient” as used herein refers to any excipient that does not interfere with effectiveness of the biological activity of the active ingredients (the antibody or an antigen-binding fragment thereof, or the nucleic acid(s) encoding the antibody or antigen-binding fragment thereof) and that is not toxic to the subject, i.e., is a type of excipient and/or is for use in an amount which is not toxic to the subject. Excipients are well known in the art, and the present system is not limited in these respects. In certain embodiments, one or more formulations of the dosage form include excipients, including for example and without limitation, one or more binders (binding agents), thickening agents, surfactants, diluents, release-delaying agents, colorants, flavoring agents, fillers, disintegrants/dissolution promoting agents, lubricants, plasticizers, silica flow conditioners, glidants, anti-caking agents, anti-tacking agents, stabilizing agents, anti-static agents, swelling agents and any combinations thereof. As those of skill would recognize, a single excipient can fulfill more than two functions at once, e.g., can act as both a binding agent and a thickening agent. As those of skill will also recognize, these terms are not necessarily mutually exclusive. Examples of commonly used excipients include water, saline, phosphate buffered saline, dextrose, glycerol, ethanol, and the like, as well as combinations thereof. In many cases, it will be preferable to include isotonic agents, for example, saccharides, polyalcohols, such as mannitol, sorbitol, or sodium chloride in the composition. Additional examples of pharmaceutically acceptable substances are wetting agents or auxiliary substances, such as emulsifying agents, preservatives, or buffers, which increase the shelf life or effectiveness.

In an embodiment, the antibody or antigen-binding fragment thereof defined herein, or the nucleic acid(s) encoding the antibody or antigen-binding fragment thereof, is/are encapsulated in a vesicle or vesicle-like particle, such as a lipid vesicle (e.g., liposome). The term “lipid vesicle” (or “lipid-based vesicle”) as used herein encompasses macromolecular structures which as the main constituent include lipid or lipid derivatives. Suitable examples hereof are liposomes and micelles including detergent micelles/lipid emulsion, liposomes prepared from palmitoyloleoylphosphatidylcholine, hydrogenated soy phosphatidylcholine, and solid lipid nanoparticles prepared from steric acid or tripalmitin. The term liposome is used herein in accordance with its usual meaning, referring to microscopic lipid vesicles composed of a bilayer of phospholipids or any similar amphipathic lipids encapsulating an internal aqueous medium. The liposomes may be unilamellar vesicles such as small unilamellar vesicles (SUVs), which typically have a diameter of less than 0.2 μm (e.g., between 0.02 and 0.2 μm), and large unilamellar vesicles (LUVs), and multilamellar vesicles (MLV), which typically have a diameter greater than 0.45 μm (in some cases greater than 1 μm). No particular limitation is imposed on the liposomal membrane structure in the present disclosure. The term liposomal membrane refers to the bilayer of phospholipids separating the internal aqueous medium from the external aqueous medium.

The dose of antibody or antigen-binding fragment (or conjugate) may vary depending upon, e.g., the age and the size of a subject to be administered, target disease, conditions, and route of administration. Antibodies or antigen-binding fragments as described herein (e.g., administered prophylactically or therapeutically) may be administered at a single dose of about 0.1 to about 60 mg/kg body weight, more preferably about 5 to about 60, about 10 to about 50, or about 20 to about 50 mg/kg body weight. Depending on the severity of the condition, the frequency and the duration of the treatment can be adjusted. In certain embodiments, the antibody or antigen-binding fragment can be administered as an initial dose of at least about 0.1 mg to about 800 mg, about 1 to about 500 mg, about 5 to about 300 mg, or about 10 to about 200 mg, to about 100 mg, or to about 50 mg. In certain embodiments, the initial dose may be followed by administration of a second or a plurality of subsequent doses of the antibody or antigen-binding fragment thereof in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

Various delivery systems are known and can be used to administer the pharmaceutical compositions including but not limited to encapsulation in liposomes, microparticles, microcapsules, recombinant cells capable of expressing the mutant viruses, receptor mediated endocytosis (see, e.g., Wu et al. (1987) J. Biol. Chem. 262:4429-4432). Methods of introduction include, but are not limited to, intradermal, transdermal (e.g., using a microinjection device), intramuscular, intraperitoneal, intravenous, subcutaneous, intranasal, intrapulmonary, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. The pharmaceutical composition can be also delivered in a vesicle, in particular a liposome (see, for example, Langer (1990) Science 249:1527-1533).

The use of nanoparticles to deliver the antibodies or antigen-binding fragments (or conjugate) of the present disclosure is also contemplated herein. Antibody-conjugated nanoparticles may be used both for therapeutic and diagnostic applications. Antibody-conjugated nanoparticles and methods of preparation and use are described in detail by Arruebo et al. (2009) Antibody-conjugated nanoparticles for biomedical applications, J. Nanomat., 439389 (doi: 10.1155/2009/439389), incorporated herein by reference. Nanoparticles may be developed and conjugated to antibodies contained in pharmaceutical compositions to target virally infected cells. Nanoparticles for drug delivery have also been described in, for example, U.S. Pat. No. 8,257,740, or U.S. Pat. No. 8,246,995.

In certain situations, the pharmaceutical composition can be delivered in a controlled release system. In one embodiment, a pump may be used. In another embodiment, polymeric materials can be used. In yet another embodiment, a controlled release system can be placed in proximity of the composition's target, thus requiring only a fraction of the systemic dose.

Injectable preparations comprising one or more antibodies or antigen-binding fragments thereof may include dosage forms for intravenous, subcutaneous, intracutaneous, intranasal (e.g., nasal spray or drop), intracranial, intraperitoneal and intramuscular injections, and drip infusions. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant (e.g., polysorbate 80, HCO-50 (polyoxyethylene (50 mol) adduct of hydrogenated castor oil), etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

The pharmaceutical composition can be delivered subcutaneously or intravenously with a standard needle and syringe. In addition, with respect to subcutaneous delivery, a pen delivery device (reusable or disposable) can be used to deliver the pharmaceutical composition.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the antibody contained is generally about 5 to about 500 mg per dosage form in a unit dose; especially in the form of injection, it is preferred that the antibody is contained in about 5 to about 100 mg and in about 10 to about 250 mg for the other dosage forms.

According to certain embodiments, a single dose of an anti-CD3 or anti-TrkB antibody(ies) or antigen-binding fragment(s) thereof, or multispecific (e.g., bispecific) antibody(ies), or nucleic acid(s) encoding such antibody(ies) or antigen-binding fragment(s), as herein described (or a single dose of a pharmaceutical combination as herein described) may be administered to a subject in need thereof. According to certain embodiments of the present disclosure, multiple doses of the antibody(ies), antigen-binding fragment(s) or nucleic acid(s) (or multiple doses of a pharmaceutical as herein described) may be administered to a subject over a defined time course. The methods comprise sequentially administering to a subject multiple doses of the antibody(ies), antigen-binding fragment(s) or nucleic acid(s) (or multiple doses of a pharmaceutical combination as herein described). By, “sequentially administering” it is meant that each dose of antibody(ies), antigen-binding fragment(s) or nucleic acid(s) (or each dose of a pharmaceutical combination) is administered to the subject at a different point in time, e.g., on different days separated by a predetermined interval (e.g., hours, days, weeks or months). The present disclosure includes methods which comprise sequentially administering to the patient a single initial dose of antibody(ies), antigen-binding fragment(s) or nucleic acid(s), followed by one or more secondary doses of the antibody(ies), antigen-binding fragment(s) or nucleic acid(s), and optionally followed by one or more tertiary doses of the antibody(ies), antigen-binding fragment(s) or nucleic acid(s).

The terms “initial dose,” “secondary doses,” and “tertiary doses,” refer to the temporal sequence of administration of the antibody. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of antibody, but generally may differ from one another in terms of frequency of administration. In certain embodiments, however, the amount of antibody contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In certain exemplary embodiments of the present disclosure, each secondary and/or tertiary dose is administered 1 to 48 hours after the immediately preceding dose. The phrase “the immediately preceding dose,” as used herein, means, in a sequence of multiple administrations, the dose of antibody which is administered to a patient prior to the administration of the very next dose in the sequence with no intervening doses.

The methods according to this aspect of the disclosure may comprise administering to a patient any number of secondary and/or tertiary doses of antibody(ies), antigen-binding fragment(s) or nucleic acid(s). For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In certain embodiments of the disclosure, the frequency at which the secondary and/or tertiary doses are administered to a patient can vary over the course of the treatment regimen. The frequency of administration may also be adjusted during the course of treatment by a physician depending on the needs of the individual patient following clinical examination.

### Therapeutic Uses

The present disclosure also provides a method for enhancing a biological activity mediated by TrkB, the method comprising contacting TrkB with a biologically effective amount of an anti-TrkB antibody or antigen-binding fragment thereof, nucleic acid or pharmaceutical composition described herein. The present disclosure also provides the use of an anti-TrkB antibody or antigen-binding fragment thereof, nucleic acid or pharmaceutical composition described herein, for enhancing a biological activity mediated by TrkB. The present disclosure also provides the use of an anti-TrkB antibody or antigen-binding fragment thereof, nucleic acid or pharmaceutical composition described herein for the manufacture of a medicament for enhancing a biological activity mediated by TrkB. The present disclosure also provides the anti-TrkB antibody or antigen-binding fragment thereof, nucleic acid or pharmaceutical composition described herein, for use in enhancing a biological activity mediated by TrkB.

In certain embodiments, the biological activity is neuronal protection or neuronal survival and neuronal protection or neuronal survival is enhanced upon contact of TrkB with an anti-TrkB antibody or antigen-binding fragment thereof.

In certain embodiments, the biological activity is neuroprotection and survival of retinal ganglion cells (RGCs).

In various aspect, the anti-TrkB antibody or antigen-binding fragment thereof, nucleic acid or pharmaceutical composition, described herein, may be used to improve nerve function and for treating any disease or disorder characterized in part by cellular degeneration, including nerve cell damage associated with nervous system injury and/or chronic neurodegenerative diseases.

The present disclosure also provides a method for treating a disease or disorder associated with or related to TrkB activity or expression comprising administering a therapeutically effective amount of an anti-TrkB antibody or antigen-binding fragment thereof, nucleic acid or pharmaceutical composition, described herein to a subject in need thereof. The present disclosure also provides the use of an anti-TrkB antibody or antigen-binding fragment thereof, nucleic acid or pharmaceutical composition, described herein, for treating a disease or disorder associated with or related to TrkB activity or expression. The present disclosure also provides the use of an anti-TrkB antibody or antigen-binding fragment thereof, nucleic acid or pharmaceutical composition, described herein, for the manufacture of a medicament for treating a disease or disorder associated with or related to TrkB activity or expression. The present disclosure also provides the anti-TrkB antibody or antigen-binding fragment thereof, nucleic acid or pharmaceutical composition, described herein, for use in treating a disease or disorder associated with or related to TrkB activity or expression.

The disorder treated is any disease or condition which is improved, ameliorated, inhibited or prevented by targeting TrkB and/or by activating TrkB-mediated cell signaling. The antibodies of the invention are useful, inter alia, for the treatment, prevention and/or amelioration of any disease or disorder related to, associated with or mediated by TrkB expression or activity. The anti-TrkB antibody or antigen-binding fragment thereof, nucleic acid or pharmaceutical composition, described herein, may be used to improve nerve function and may be used to treat or prevent any disease or condition that is characterized in part by cellular degradation, in particular by nerve cell injury or nerve cell degeneration, e.g., an acute nervous system injury or a chronic neurodegenerative disease.

The present disclosure also provides a method for preventing or reducing injury or death of retinal neurons, e.g., for preventing or reducing photoreceptor injury and degeneration, comprising administering a therapeutically effective amount of an anti-TrkB antibody or antigen-binding fragment thereof described herein, or pharmaceutical composition comprising same, described herein to a subject in need thereof. The present disclosure provides the use of an anti-TrkB antibody or antigen-binding fragment thereof, nucleic acid or pharmaceutical composition, described herein, for preventing or reducing injury or death of retinal neurons, e.g., for preventing or reducing photoreceptor injury and degeneration. The present disclosure provides the use of an anti-TrkB antibody or antigen-binding fragment thereof, nucleic acid or pharmaceutical composition, described herein, for the manufacture of a medicament for preventing or reducing injury or death of retinal neurons, e.g., for preventing or reducing photoreceptor injury and degeneration. The present disclosure provides an anti-TrkB antibody or antigen-binding fragment thereof, nucleic acid or pharmaceutical composition, described herein, for use in preventing or reducing injury or death of retinal neurons, e.g., for preventing or reducing photoreceptor injury and degeneration.

In another aspect, the present disclosure also provides a method of preventing or treating a pathological disease associated with degeneration of the retina comprising administering a therapeutically effective amount of an anti-TrkB antibody or antigen-binding fragment thereof described herein, or pharmaceutical composition comprising same, described herein to a subject in need thereof. In another aspect, the present disclosure also provides the use of an anti-TrkB antibody or antigen-binding fragment thereof described herein, or pharmaceutical composition comprising same, for preventing or treating a pathological disease associated with degeneration of the retina. In another aspect, the present disclosure also provides the use of an anti-TrkB antibody or antigen-binding fragment thereof described herein, or pharmaceutical composition comprising same, for the manufacture of a medicament for preventing or treating a pathological disease associated with degeneration of the retina. In another aspect, the present disclosure also provides an anti-TrkB antibody or antigen-binding fragment thereof described herein, or pharmaceutical composition comprising same, for use in preventing or treating a pathological disease associated with degeneration of the retina.

In an embodiment, the anti-TrkB antibody or antigen-binding fragment thereof described herein, or pharmaceutical composition comprising same is administered or for use prior to, during and/or after an eye trauma or injury. In an embodiment, the eye trauma or injury is caused by ocular surgery. In an embodiment, the eye trauma or injury is caused by exposure to light.

In an embodiment, the disease or disorder is an optic neuropathy. Optic neuropathies include, for example conditions impacting retinal ganglion cells (RGC) and/or the optic nerve. Particular optic neuropathies include: glaucoma (for example open angle glaucoma, wide angle glaucoma, angle closure glaucoma (acute and chronic), normal tension glaucoma), anterior ischaemic optic neuropathy (AION) (for example, non-arteritic ischeamic optic neuropathy (NAION)), posterior ischemic optic neuropathy, radiation optic neuropathy, compressive optic neuropathy (for example, papilledema), infiltrative optic neuropathy, traumatic optic neuropathy, mitochondrial optic neuropathy, toxic optic neuropathies, hereditary optic neuropathies (for example, autosomal dominant optic atrophy (ADOA; optic atrophy type Kjer), Leber hereditary optic neuropathy. Rosenberg Chutorian syndrome, Wolfram syndrome, optic nerve hypoplasia), optic neuritis (for example, neuromyelitis optica, papillitis), photoreceptor degeneration, and retinitis pigmentosa.

In an embodiment, the disease or disorder is a retinopathy such as surgery-induced retinopathy, toxic retinopathy, retinal detachment, photic retinopathy, viral retinopathy (e.g., CMV or HIV retinopathy); retinopathy due to trauma or lesion of the eye, peripheral vitreoretinopathy or inherited retinal degeneration.

In an embodiment, the disease or disorder is an ischemic retinopathy. The term “ischemic retinopathy” refers to a disease or condition associated with reduced blood flow to the retina, leading to retinal cells' death. In various embodiments, the ischemic retinopathy is glaucoma, diabetic retinopathy, retinopathy of prematurity, age-related macular degeneration (dry or wet form), ischemic optic neuropathy (anterior ischemic optic neuropathy, AION, non-arteritic anterior ischemic optic neuropathy, NAION) and retinal artery or vein occlusions such as central retinal venous obstruction (CRVO) or branch retinal vein occlusion (BRVO).

In other embodiments, the disease or disorder is optic neuritis, retinitis pigmentosa, Leber Congenital Amaurosis, Leber's hereditary optic neuropathy, Usher Syndrome or Stargardt disease.

In another aspect, the anti-TrkB antibody or antigen-binding fragment thereof, or pharmaceutical composition comprising same, described herein, may be used to inhibit or slow the progression of cognitive function decline in a subject, such as a subject suffering from a neurodegenerative disease.

In other embodiments, the disease or disorder is a neurodegenerative disease, such as Parkinson's Disease (PD), mild cognitive impairment (MCI), Alzheimer's disease (AD), Huntington's disease (HD), motor neuron disease, Tourette's syndrome, dementia (including fronto-temporal dementia). Examples of motor neuron diseases include, e.g., amyotrophic Lateral Sclerosis (ALS), idiopathic motor neuropathy, hereditary spastic paraplegia (HSP), primary lateral sclerosis (PLS), progressive muscular atrophy (PMA), progressive bulbar palsy (PBP), pseudobulbar palsy, Bell's palsy, and spinal muscular atrophy (SMA).

In other embodiments, the TrkB-related disease or disorder is a mental disorder, such as a mood disorder (e.g., depression, major depressive disorder, bipolar disorder, seasonal affective disorder (SAD), etc.). In an embodiment, the mental disorder is depression or major depressive disorder.

In an embodiment, the anti-TrkB antibody or antigen-binding fragment thereof, or pharmaceutical composition comprising same, described herein, can be used in combination with other known medications or regiments for treating optic neuropathies or retinal degenerative disorders such as glaucoma. For example, the anti-TrkB antibody or antigen-binding fragment thereof, or pharmaceutical composition comprising same, can be used together with any of the existing intraocular pressure (IOP) lowering drugs. These known drugs include, e.g., prostaglandin analogues such as Xalatan (latanoprost) and Lumigan (bimatroprost), Beta blockers such as timolol, betaxolol and metipranolol. In addition to therapeutic IOP lowering drugs, there are many approved drainage devices which can be used in combination with the anti-TrkB antibody or antigen-binding fragment thereof, or pharmaceutical composition comprising same, described herein. The anti-TrkB antibody or antigen-binding fragment thereof, or pharmaceutical composition comprising same, can be used together with antiangiogenic drugs such as anti-VEGF agents (e.g., aflibercept, bevacizumab, or ranibizumab).

In another aspect, the present disclosure also provides a method of reducing body weight or body weight gain comprising administering a therapeutically effective amount of an anti-TrkB antibody or antigen-binding fragment thereof described herein, or pharmaceutical composition comprising same, described herein to a subject in need thereof. In another aspect, the present disclosure also provides the use of an anti-TrkB antibody or antigen-binding fragment thereof described herein, or pharmaceutical composition comprising same, for reducing body weight or body weight gain. In another aspect, the present disclosure also provides the use of an anti-TrkB antibody or antigen-binding fragment thereof described herein, or pharmaceutical composition comprising same, for the manufacture of a medicament for reducing body weight or body weight gain. In another aspect, the present disclosure also provides an anti-TrkB antibody or antigen-binding fragment thereof described herein, or pharmaceutical composition comprising same, for use in reducing body weight or body weight gain. The term “reducing body weight gain” as used herein means that over a period of time, the weight gained in a subject treated with the anti-TrkB antibody or antigen-binding fragment thereof described herein, or pharmaceutical composition comprising same, is less than the weight that the subject would have gained without the treatment with the anti-TrkB antibody or antigen-binding fragment thereof described herein.

In another aspect, the present disclosure also provides a method of treating obesity and/or a related metabolic disease (e.g., type 2 diabetes) comprising administering a therapeutically effective amount of an anti-TrkB antibody or antigen-binding fragment thereof described herein, or pharmaceutical composition comprising same, described herein to a subject in need thereof. In another aspect, the present disclosure also provides the use of an anti-TrkB antibody or antigen-binding fragment thereof described herein, or pharmaceutical composition comprising same, for treating obesity and/or a related metabolic disease (e.g., type 2 diabetes). In another aspect, the present disclosure also provides the use of an anti-TrkB antibody or antigen-binding fragment thereof described herein, or pharmaceutical composition comprising same, for the manufacture of a medicament for treating obesity and/or a related metabolic disease (e.g., type 2 diabetes).

In another aspect, the present disclosure also provides an anti-TrkB antibody or antigen-binding fragment thereof described herein, or pharmaceutical composition comprising same, for use in treating obesity and/or a related metabolic disease (e.g., type 2 diabetes). TrkB has been shown to be up-regulated and to have oncogenic effects by increasing cancer cell growth, proliferation, survival, migration and epithelial to mesenchymal transition in various cancers, including breast, lung, colorectal, pancreas, prostate, liver, neural (e.g., neuroblastoma), myelomas and lymphoid tumors (see, e.g., Meldolesi J. Neurotrophin Trk receptors: New targets for cancer therapy. Rev Physiol Biochem Pharmacol. 2018; 174:67-79). Pan-Trk inhibitors Entrectinib and Larotrectinib, which are not specific for TrkB, are being tested for the treatment of various cancer types including breast cancer, cholangiocarcinoma, colorectal cancer, head and neck neoplasms, lymphoma (e.g., non-Hodgkin lymphoma), melanoma, neuroendocrine tumors, sarcoma, non-small cell lung cancer, ovarian cancer, pancreatic cancer, papillary thyroid cancer, primary brain tumors, renal cell carcinoma, sarcomas, salivary gland cancer, and adult solid tumor (see, e.g., Meng L, Liu B, Ji R, Jiang X, Yan X, Xin Y. Targeting the BDNF/TrkB pathway for the treatment of tumors. Oncol Lett. 2019; 17 (2): 2031-2039).

The present disclosure also provides a method for suppressing a biological activity mediated by TrkB, the method comprising contacting TrkB with a biologically effective amount of an anti-TrkB antibody or antigen-binding fragment thereof, nucleic acid or pharmaceutical composition described herein. The present disclosure also provides the use of an anti-TrkB antibody or antigen-binding fragment thereof, nucleic acid or pharmaceutical composition described herein, for suppressing a biological activity mediated by TrkB. The present disclosure also provides the use of an anti-TrkB antibody or antigen-binding fragment thereof, nucleic acid or pharmaceutical composition described herein for the manufacture of a medicament for suppressing a biological activity mediated by TrkB. The present disclosure also provides the anti-TrkB antibody or antigen-binding fragment thereof, nucleic acid or pharmaceutical composition described herein, for use in suppressing a biological activity mediated by TrkB.

Accordingly, the anti-TrkB antibody, antigen-binding fragment thereof or conjugates described herein, or pharmaceutical composition comprising same, may be used for the treatment of TrkB-expressing cancers, e.g., by targeting antitumor agents to TrkB-expressing tumor cells, by blocking BDNF binding to TrkB and/or by inducing the immune-mediated killing of TrkB-expressing tumor cells by antibody-dependent cell-mediated cytotoxicity (ADCC), complement-dependent cytotoxicity (CDC), antibody-dependent cellular phagocytosis (ADCP) and/or cytotoxic T-cell-mediated killing. In an embodiment, the cancer is breast cancer, cholangiocarcinoma, gastrointestinal cancer, colorectal cancer, head and neck neoplasms, lymphoma (e.g., non-Hodgkin lymphoma), melanoma, neuroendocrine tumors, sarcoma, non-small cell lung cancer, ovarian cancer, pancreatic cancer, papillary thyroid cancer, primary brain tumor, renal cell carcinoma, sarcoma, salivary gland cancer, or adult solid tumor.

In an embodiment, the anti-TrkB antibody or antigen-binding fragment thereof is a multispecific (e.g., bispecific) antibody. In an embodiment, the multispecific (e.g., bispecific) antibody further comprises a domain binding to a tumor antigen. In an embodiment, the multispecific (e.g., bispecific) antibody further comprises a domain binding to CD3 as described herein.

The anti-CD3 antibodies, antigen-binding fragments thereof (including combinations thereof) and nucleic acids encoding same (or pharmaceutical compositions) as herein described may be useful for inhibiting or stimulating the activity of CD3-expressing cells such as T-cells.

Anti-CD3 antibodies or antigen-binding fragments thereof that inhibit or antagonize CD3 activation may be used for inhibiting the activity of CD3-expressing cells such as T-cells, which may be beneficial in conditions or diseases associated with dysregulated T-cell activity. Accordingly, in another aspect, the present disclosure provides a method for inhibiting the activity of CD3-expressing cells such as T-cells in a subject in need thereof comprising administering to the subject an effective amount of the antibodies, antigen-binding fragments thereof (including combinations thereof) and nucleic acids encoding same (or pharmaceutical compositions) as herein described. The present disclosure also provides the use of the antibodies, antigen-binding fragments thereof (including combinations thereof) and nucleic acids encoding same (or pharmaceutical compositions) as herein described for inhibiting the activity of CD3-expressing cells such as T-cells in a subject, or for the manufacture of a medicament for inhibiting the activity of CD3-expressing cells such as T-cells in a subject. The present disclosure also provides the antibodies, antigen-binding fragments thereof (including combinations thereof) and nucleic acids encoding same (or pharmaceutical compositions) as herein described for use in inhibiting the activity of CD3-expressing cells such as T-cells in a subject. Examples of diseases or conditions in which inhibition of the activity of CD3-expressing cells such as T-cells may be useful include autoimmune diseases or conditions.

In an aspect, the present disclosure provides a method for treating and/or preventing an autoimmune disease or condition in a subject in need thereof comprising administering to the subject an effective amount of the antibodies, antigen-binding fragments thereof (including combinations thereof) and nucleic acids encoding same (or pharmaceutical compositions) as herein described. The present disclosure also provides the use of the antibodies, antigen-binding fragments thereof (including combinations thereof) and nucleic acids encoding same (or pharmaceutical compositions) as herein described for treating and/or preventing an autoimmune disease or condition in a subject, or for the manufacture of a medicament for treating and/or preventing an autoimmune disease or condition in a subject. The present disclosure also provides the antibodies, antigen-binding fragments thereof (including combinations thereof) and nucleic acids encoding same (or pharmaceutical compositions) as herein described for use in treating and/or preventing an autoimmune disease or condition in a subject.

The term “autoimmune disease or condition” as used refers to a disease or condition associated with an abnormal or dysregulated immune response (and more specifically an immune response involving T-cells) against normal tissues or organs. Examples of autoimmune diseases or conditions includes multiple sclerosis, human systemic lupus, rheumatoid arthritis, inflammatory bowel disease (Crohn's disease, ulcerative colitis), psoriasis, Type 1 diabetes, acute disseminated encephalomyelitis, Addison's disease, alopecia universalis, ankylosing spondylitis, antiphospholipid antibody syndrome, aplastic anemia, autoimmune hemolytic anemia, autoimmune hepatitis, autoimmune inner ear disease, autoimmune lymphoproliferative syndrome, autoimmune oophoritis, Balo disease, Behcet's disease, bullous pemphigoid, cardiomyopathy, Chagas' disease, chronic fatigue immune dysfunction syndrome, chronic inflammatory demyelinating polyneuropathy, cicatrical pemphigoid, dermatitis herpetiformis, cold agglutinin disease, CREST syndrome, Degos disease, discoid lupus, dysautonomia, endometriosis, essential mixed cryoglobulinemia, fibromyalgia-fibromyositis, Goodpasture's syndrome, Grave's disease, Guillain-Barre syndrome, Hashimoto's thyroiditis, Hidradenitis suppurativa, idiopathic and/or acute thrombocytopenia purpura, idiopathic pulmonary fibrosis, IgA neuropathy, interstitial cystitis, juvenile arthritis, Kawasaki's disease, lichen planus, Lyme disease, Meniere disease, mixed connective tissue disease, myasthenia gravis, neuromyotonia, opsoclonus myoclonus syndrome, optic neuritis, Ord's thyroiditis, pemphigus vulgaris, pernicious anemia, polychondritis, polymyositis, dermatomyositis, primary biliary cirrhosis, polyarteritis nodosa, polyglandular syndromes, polymyalgia rheumatica, primary agammaglobulinemia, Raynaud phenomenon, Reiter's syndrome, rheumatic fever, sarcoidosis, scleroderma, Sjogren's syndrome, stiff person syndrome, Takayasu's arteritis, temporal arteritis, uveitis, vasculitis, vitiligo, vulvodynia, and Wegener's granulomatosis. In an embodiment, the autoimmune disease is psoriasis, inflammatory bowel disease (Crohn's disease or ulcerative colitis), ankylosing spondylitis, lupus, arthritis (e.g., rheumatoid arthritis), or a neuroinflammatory disease (e.g., multiple sclerosis).

Anti-CD3 antibodies or antigen-binding fragments thereof that increase or stimulate CD3 activation may be used for stimulating the activity of CD3-expressing cells such as T-cells, which may be beneficial in conditions or diseases for which increased T-cell activity is desired. Accordingly, in another aspect, the present disclosure provides a method for increasing or stimulating the activity of CD3-expressing cells such as T-cells in a subject in need thereof comprising administering to the subject an effective amount of the antibodies, antigen-binding fragments thereof (including combinations thereof) and nucleic acids encoding same (or pharmaceutical compositions) as herein described. The present disclosure also provides the use of the antibodies, antigen-binding fragments thereof (including combinations thereof) and nucleic acids encoding same (or pharmaceutical compositions) as herein described for increasing or stimulating the activity of CD3-expressing cells such as T-cells in a subject, or for the manufacture of a medicament for increasing or stimulating the activity of CD3-expressing cells such as T-cells in a subject. The present disclosure also provides the antibodies, antigen-binding fragments thereof (including combinations thereof) and nucleic acids encoding same (or pharmaceutical compositions) as herein described for use in increasing or stimulating the activity of CD3-expressing cells such as T-cells in a subject. Examples of diseases or conditions in which enhancement or stimulation of the activity of CD3-expressing cells such as T-cells may be useful include infectious diseases and cancer.

The anti-CD3 antibodies, antigen-binding fragments thereof (including combinations thereof) and nucleic acids encoding same (or pharmaceutical compositions) as herein described may be administered or used in combination with one or more additional active agents or therapies for the target disease.

In an embodiment, the anti-CD3 antibodies, antigen-binding fragments thereof (including combinations thereof) and nucleic acids encoding same (or pharmaceutical compositions) as herein described are administered or used in combination with one or more additional active agents or therapies to treat an autoimmune or inflammatory diseases including anti-inflammatory drugs and immunosuppressant drugs such as steroids (e.g., corticosteroids such as prednisone, methylprednisolone and dexamethasone), colchicine, hydroxychloroquine, sulfasalazine, dapsone, methotrexate, mycophenolate mofetil, azathioprine, IL-1 inhibitors (e.g., anti-IL-1 biologics such as Anakinra, Canakinumab and Rilonacep), TNF inhibitors (e.g., anti-TNF biologics such as Infliximab, Adalimumab, Golimumab, Etanercept and Certolizumab), IL-4/13 inhibitors (e.g., anti-IL-4/13 biologics such as Dupilumab), IL-5 inhibitors (e.g., anti-IL-5 biologics such as Mepolizumab, Reslizumab, and Benralizumab), IL-6 inhibitors (e.g., anti-IL-6 biologics such as Tocilizumab and Sarilumab), IL-17 inhibitors (e.g., anti-IL-17 biologics such as Secukinumab, Ixekizumab, and Brodalumab), IL-12/23 inhibitors (e.g., anti-IL-12/23 biologics such as Guselkumab and Ustekinumab), BLyS inhibitors (e.g., anti-BLys biologics such as Belimumab), cyclosporine, T-cell co-stimulation inhibitors (e.g., anti-CD28 biologics such as Abatacept), α4β7 integrin inhibitors (e.g., anti-α4β7 biologics such as Vedolizumab), and/or JAK inhibitors (e.g., Tofacitinib, Upadacitinib, and Baricitinib).

In other embodiments, the anti-CD3 and/or anti-TrkB antibodies, antigen-binding fragments thereof (including combinations thereof) and nucleic acids encoding same (or pharmaceutical compositions) as herein described are administered or used in combination with one or more additional active agents or therapies to treat cancer such as chemotherapy (e.g., vinca alkaloids, agents that disrupt microtubule formation (such as colchicines and its derivatives), anti-angiogenic agents, therapeutic antibodies, EGFR targeting agents, tyrosine kinase targeting agent (such as tyrosine kinase inhibitors), transitional metal complexes, proteasome inhibitors, antimetabolites (such as nucleoside analogs), alkylating agents, platinum-based agents, anthracycline antibiotics, topoisomerase inhibitors, macrolides, retinoids (such as all-trans retinoic acids or a derivatives thereof), geldanamycin or a derivative thereof (such as 17-AAG), surgery, immune checkpoint inhibitors or immunotherapeutic agents (e.g., PD-1/PD-L1 inhibitors such as anti-PD-1/PD-L1 antibodies, CTLA-4 inhibitors such as anti-CTLA-4 antibodies, B7-1/B7-2 inhibitors such as anti-B7-1/B7-2 antibodies, TIM-3 inhibitors such as anti-TIM-3 antibodies, BTLA inhibitors such as anti-BTLA antibodies, CD47 inhibitors such as anti-CD47 antibodies, GITR inhibitors such as anti-GITR antibodies), antibodies against tumor antigens (e.g., anti-CD19, anti-CD22 antibodies), cell-based therapies (e.g., CAR T-cells, CAR NK cells), and/or cytokines such as IL-2, IL-7, IL-21, and/or IL-15. In an embodiment, the antibodies, antigen-binding fragments thereof (including combinations thereof) and nucleic acids encoding same (or pharmaceutical compositions) as herein described are administered/used in combination with an immune checkpoint inhibitor.

### Diagnostic Uses of the Antibodies or Antigen-Binding Fragments Thereof

The anti-TrkB antibodies or antigen-binding fragments thereof described herein may be used to detect and/or measure TrkB or TrkB-expressing cells, in a sample, e.g., for diagnostic or prognostic purposes. Some embodiments contemplate the use of one or more of the antibodies or antigen-binding fragments thereof in assays to detect a disease or disorder associated with or related to TrkB expression. Exemplary assays may comprise, e.g., contacting a sample, obtained from a patient, with an anti-TrkB antibody or antigen-binding fragment thereof described herein, wherein the antibody or antigen-binding fragment thereof is labeled with a detectable label or reporter molecule or used as a capture ligand to selectively isolate TrkB or TrkB-expressing cells from patient samples. Alternatively, an unlabeled anti-TrkB antibody or antigen-binding fragment thereof can be used in diagnostic applications in combination with a secondary antibody which is itself detectably labeled. The detectable label or reporter molecule can be a radioisotope, such as ³H, ¹⁴C, ³²P, ³⁵S, or ¹²⁵I; a fluorescent or chemiluminescent moiety such as fluorescein isothiocyanate, or rhodamine; or an enzyme such as alkaline phosphatase, beta-galactosidase, horseradish peroxidase, or luciferase. Specific exemplary assays that can be used to detect or measure TrkB or TrkB-expressing cells in a sample include enzyme-linked immunosorbent assay (ELISA), radioimmunoassay (RIA), (bead-based) flow cytometry and fluorescence-activated cell sorting (FACS).

Samples that can be used in assays according to the present disclosure include any tissue or fluid sample obtainable from a patient (e.g., blood, plasma, saliva, nasal secretion), which contains detectable quantities of either TrkB or TrkB-expressing cells under normal or pathological conditions. Generally, levels of TrkB or TrkB-expressing cells in a particular sample obtained from a healthy patient (e.g., a patient not afflicted with a disease associated with or related to TrkB or TrkB-expressing cells) will be measured to initially establish a baseline, or standard, level of TrkB or TrkB-expressing cells. This baseline level of TrkB or TrkB-expressing cells can then be compared against the levels of TrkB or TrkB-expressing cells measured in samples obtained from individuals suspected of having a TrkB-associated or -related condition, or symptoms associated with such condition.

### EXAMPLES

The present disclosure is illustrated in further details by the following non-limiting examples.

### Example 1: Materials and Methods

### Recombinant Proteins for Identification and Characterization of Anti-TrkB Antibodies

Protein targets used as panning, screening and analytical reagents for ELISA, flow cytometry and Octet binding assays, and reference proteins, such as TrkA-His and TrkC-His, or benchmark binders, including mouse anti-His, rat and mouse anti-TrkB, mouse anti-human-TrkB, anti-mouse and anti-human IgG-HRP, anti-GST-HRP, anti-rat igG2b-HRP, mouse anti-TrkA, mouse anti-TrkC, mouse and rat isotype controls, were purchased from various vendors as indicated. Reactivity, stability following a freeze/thaw cycle and purity of the materials was verified by SDS-PAGE and ELISA where appropriate, for all reference proteins.

A total of six synthetic gene constructs of fusion products of lgG-derived Fc part, glutathione S-transferase (GST) and poly-His (His) with the extracellular domain (ECD) of TrkB of either a mouse or human origin were cloned into a eukaryotic expression vector. The endotoxin-free sequences were verified, transiently expressed in HEK293 cells and proteins harvested with high yields, purified using appropriate affinity material, and integrity, purity, stability following freeze/thaw cycles and (cross-) reactivity were checked by SDS-PAGE, and size-exclusion chromatography (SEC) in the case of human and mouse TrkB-ECD-GST, and ELISA. A part of each of the human and mouse fusion proteins, except TrkB-ECD-His, was biotinylated adequately to allow target enrichment-based antibody discovery and screening procedures. The degree of biotinylation was determined following cycles of pull-down using magnetic streptavidin-beads and SDS-PAGE of the supernatants. Maintained reactivity in ELISA analyses suggested that biotinylation did not affect the conformation of the TrkB-ECD.

### Recombinant Proteins for Identification and Characterization of Anti-CD3 Antibodies

Protein targets used as panning, screening and analytical reagents for ELISA and Octet binding assays, and reference or benchmark binders were purchased from various vendors as indicated. The reactivity of mouse anti-CD3ε OKT3 antibody (16-0037-85, Fisher Emergo) towards biotinylated recombinant DNA-produced human CD30/8 heterodimer expressed from HEK293 cells (CD3E & CD3D-377H, Creative BioMart) was verified in direct and indirect ELISA assays. In addition, the molecular integrity and purity of the biotinylated reference was confirmed by SDS-PAGE under (non-) reducing conditions and Western blotting analyses. The degree of biotinylation was determined following cycles of pull-down using magnetic streptavidin-beads, and SDS-PAGE and Western blotting analyses of the supernatants. Anti-human kappa and anti-human lambda light chain antibodies were purchased from SouthernBiotech (Birmingham, AL, USA). Benchmark antibody F2B [Trinklein et al., 2019] was generated from a published sequence, expressed via the rPEx® platform (IPA, Utrecht, The Netherlands) and produced in-house in HEK293 cells.

### Generation of TrkB and CD3-Expressing Cells

The full length extracellular domain (ECD) sequences as provided by Uniprot for human CD30 (Uniprot ID: P04234), CD3E (Uniprot ID: P07766) and CD3y (Uniprot ID: P09693), human TrkB (Uniprot ID: Q16620) and mouse TrkB (Uniprot ID P15209), were encoded in synthetic gene constructs and obtained from GeneArt.

CD3δ (Uniprot ID: P04234):
(SEQ ID NO: 704)
MEHSTFLSGLVLATLLSQVSPFKIPIEELEDRVFVNCNTSITWVEGTVG
TLLSDITRLDLGKRILDPRGIYRCNGTDIYKDKESTVQVHYRMCQSCVE
LDPATVAGIIVTDVIATLLLALGVFCFAGHETGRLSGAADTQALLRNDQ
VYQPLRDRDDAQYSHLGGNWARNK
Residues 1-21: Signal sequence
Residues 22-171: Mature polypeptide
CD3ϵ (Uniprot ID: P07766):
(SEQ ID NO: 705)
MQSGTHWRVLGLCLLSVGVWGQDGNEEMGGITQTPYKVSISGTTVILTC
PQYPGSEILWQHNDKNIGGDEDDKNIGSDEDHLSLKEFSELEQSGYYVC
YPRGSKPEDANFYLYLRARVCENCMEMDVMSVATIVIVDICITGGLLLL
VYYWSKNRKAKAKPVTRGAGAGGRQRGQNKERPPPVPNPDYEPIRKGQR
DLYSGLNQRRI
Residues 1-22: Signal sequence
Residues 23-207: Mature polypeptide
CD3γ (Uniprot ID: P09693):
(SEQ ID NO: 706)
MEQGKGLAVLILAIILLQGTLAQSIKGNHLVKVYDYQEDGSVLLTCDAE
AKNITWFKDGKMIGFLTEDKKKWNLGSNAKDPRGMYQCKGSQNKSKPLQ
VYYRMCQNCIELNAATISGFLFAEIVSIFVLAVGVYFIAGQDGVRQSRA
SDKQTLLPNDQLYQPLKDREDDQYSHLQGNQLRRN
-Residues 1-22: Signal sequence
-Residues 23-182: Mature polypeptide

The synthetic gene constructs were cloned into a eukaryotic expression vector (IPA, Oss, NL) and sequence verified. Following transfection with CD3& and appropriate selection, CHO-K1 cells were subsequently transfection with the CD30 or with the CD3y plasmid. CHO-k1 cells were also transfected with plasmids encoding for hTrkB and mTrkB separately. The CHO-K1 cells were transfected using Amaxa Nucleofector Kit system per the manufacture (Lonza). Stable clones were generated and selected based on expression levels using flow cytometry, and target protein stability and expression level, one clone per heterodimer, CD3δ/ε- or CD3γ/ε-expressing CHO-K1 or TrkB variant was selected for manufacturing a working stock. Cells were cryopreserved until use.

### Reactivity of Anti-CD3 Antibodies

The binding of discovered and purified anti-CD3 Fab candidate molecules and benchmark antibodies was probed using CD30/8-expressing CHO-K1 and Jurkat cells (93-1141C19, DiscoverX) on an Octet HTX system (Sartorius, Göttingen, Germany) employing biolayer interferometry (BLI) detection.

### Off-Rate Kinetic Measurements of Full-Length Anti-TrkB Antibodies

Off-rate kinetic scout assays were performed using an Octet system (Sartorius) employing biolayer interferometry (BLI) detection. Sensor surface was prepared for measurement by presenting the tested human anti-TrkB mAb clone to an anti-human IgG Fc-coated (AHC) biosensor pre-wetted in PBS containing 0.1% BSA and 0.05% Tween-20, at 1 μg/mL for 600 s. Surplus of anti-TrkB antibodies was washed away for 60 s using the same PBS buffer before allowing the binding of 300 nM human TrkB-His for 600 s followed by the enriched PBS buffer for 600 s to allow the monitoring of the dissociation of the antigen. Sensorgrams were zeroed by correcting the signal for the biosensor's response when only buffer passed the sensor so that the dissociation process over the first 30 s could be modelled to determine the dissociation constant for each anti-TrkB antibody assuming an equimolecular binding.

### Off-Rate Kinetic Measurements of Full-Length Anti-CD3 Antibodies

Off-rate scout assays were performed using an Octet HTX system (Sartorius, Göttingen, Germany) employing biolayer interferometry (BLI) detection. Pre-wetted streptavidin-coated (SAX) biosensors were loaded with 2.0 ug/mL biotinylated His-Avi-tagged CD30/8 heterodimer for 300 s. Surface was washed for 150 s before association and dissociation properties of the discovered Fabs were investigated. For this purpose, 20 μg/mL Fab was run over the sensor chip for 300 s followed by Fab-free running buffer for 150 s to measure the dissociation of the binder in the first 20 s. Sensorgrams were zeroed by correcting the signal for the biosensor's response when only buffer passed the sensor. The resulting data were fitted using the Kinetics module of Fortebio's Data Analysis HT software (version 12.0.2.59) assuming a simple 1:1 Langmuir binding model to deduce the K_{D} value from the ratio of the kinetic rate constants (K_{D}=k_{d}/kₐ), where kₐ and kₐ are the dissociation and association rate constants, respectively. Experiments were conducted at 30° C. in a run buffer of PBS containing 0.05% (v/v) Tween™-20 and 0.1% (m/v) BSA. The CD3-binding fragment of blinatumomab and full mAb OKT3 were used as positive controls.

### Discovery of Anti-TrkB Chicken-Human Chimeric Antibodies

Mouse and human TrkB-ECD fused with either Fc or GST proteins were used as immunogens to activate B-cells in chickens. For this purpose, chickens were housed in an animal facility and cared for in accordance with the guidelines of the Dutch Regulatory Authorities (NVWA), and with the Dutch Law on animal experimentation (Wet op de Dierproeven), which is based upon European Directive 2010/63/EU.

Barnevelder chickens were immunized with intramuscular (i.m.) injection with hTrkb-ECD-GST and boosted four times with either hTrkB-ECD-GST again or a mixture or hTrkB-ECD-GST and mTrkB-ECD-GST using an appropriate adjuvant. At day 0 At day 58 and 160, blood samples were collected and anti-TrkB titers were determined. After the final boost, blood was collected, and chickens were euthanized by pentobarbital injection. Subsequently, spleens and bone marrow were harvested, and flushed manually to recover a B-cell containing eluate which was passed through a 30-μm filter to obtain a tissue-free cell suspension. The isolated intact white blood cells were counted prior to further processing.

Subsequently the cells were seeded. with an anti-chicken IgY-AF647 antibody (Jackson ImmunoResearch, cat. #303-605-008, 5 μg/mL) and hTrkB-ECD-Fc-biotin-coated streptavidin beads. After imaging using the CellCelector™ platform and identified antigen-binding nanowells, i.e., anti-chicken-IgY-AF647-positive wells, were picked, RNA was isolated and the DNA fragments representing VH and VL sequences were generated and sequenced. The sequences were cloned into DNA plasmid vectors which were each used to transfect another series of HEK293 cells. Supernatants of these cultures were used to discover the chicken-derived TrkB-specific antibodies.

### Combinatorial Pairwise Epitope Binning of Anti-TrkB Antibodies

Reference antibodies TrkB-agoAb1104, TrkB-agoAb303, TrkB-agoAb2908 and TrkB-agoAb7431 targeting epitope bins D1, D3, D5 and JM, respectively, are presented in PCT patent publication No. WO2018/166495 filed by the Tsinghua University (Beijing, China). Benchmark antibody C2 (blocking BDNF) is described in U.S. patent publication No. US20100196390A1 filed by Rinat Neuroscience Corporation. These benchmark antibodies were generated from their published sequences, cloned in a mammalian expression vector (IPA, The Netherlands) and expressed as an intact, human IgG1 via the rPEx® platform (IPA, The Netherlands). The purified proteins were checked for integrity, purity and binding reactivity in ELISA and flow cytometric analysis assays.

Combinatorial pairwise mAb competition or “epitope binning” assays were performed as follows. In a “classical sandwich” assay format, AR2G sensors (Sartorius, cat #18-5092) were loaded with 20 μg/mL of mAb candidates in pH 5 sodium acetate in the presence of NHS/EDC. HuTrkB-His was subsequently immobilized at 20 μg/mL. Select mAbs were presented at 30 μg/mL as the mAb sandwich partner. Alternatively, for a tandem approach, anti-GST biosensors (Sartorius, cat. #18-5096) were loaded with GST-hu-TrkB at 10 g/mL. Antibodies were presented as either the primary or secondary analyte at 30 μg/mL. All mAbs were tested as both primary and secondary analyte. The assay buffer consisted of 4 mM PBS at pH 7.4 enriched with 0.1% (w/v) BSA and 0.05% (v/v) Tween™ 20. Between cycles, sensors surfaces were regenerated with 1:100 (v/v) dilution of 85% phosphoric acid. Sensors were used four to five times.

Alternatively, anti-penta-HIS biosensors (Sartorius Cat. #18-5120) were loaded with huTrkB-His at 20 g/mL. Subsequently, antibodies were presented as primary analyte at 30 μg/mL, followed by an analyte sample containing both the respective primary antibody and a secondary candidate antibody at 30 μg/mL. All mAbs were tested as both primary and secondary analyte. The assay buffer consisted of 10 mM HEPES at pH 7.4 fortified with 150 mM ammonium sulfate, 0.05% (v/v) Tween™-20, and 1 mg/mL BSA.

For the analysis of competition with benchmark antibodies, surface-immobilization of in-house produced His-tagged TrkB on an anti-penta-HIS biosensor (HIS1K, Fortebio) was accomplished using standard coupling conditions. Immobilized TrkB was allowed to capture Analyte 1 for 300 s, followed by injection of Analyte 1 plus Analyte 2 for 150 s. If Analyte 1 or Analyte 2 was an antibody, its concentration was 30 μg/mL. In the case Analyte 1 and/or 2 was/were BDNF, the concentration was 50 nM.

All biosensor data were analyzed using the Fortebio Data Analysis HT software.

### ELISA Assessment of Anti-CD3 Antibodies

ELISA-based reactivity screening of amplified polyclonal phage outputs at a seven-step semi-log dilution series of primary phage output starting at 1:20 was conducted to identify candidate anti-CD3 antibodies. Targets diluted to final concentrations 1 μg/mL in carbonate binding buffer were added (100 ng/well) to Bio-One High Bind ELISA plates (Greiner) and incubated overnight at 4° C. Plates were blocked with 1% BSA in PBS for 60 min. If an additional capture step was performed, it was conducted in PBS for 1 h at room temperature (RT). Coated plates were finally washed with PBS-Tween™ (PBS-T) before serial dilutions of phage or recombinant reference antibodies were added in duplicate in PBS supplemented with 5% (v/v) skim milk or 1% (m/v) BSA and incubated at RT for 60 min. After washing with PBS-T, target-bound scFvs from periplasmic fractions were detected by incubation with mouse anti-VSV followed by anti-mouse IgG-HRP.

Alternatively, antibody binding to target polypeptides was detected using anti-human-Lambda-HRP or anti-human-Kappa-HRP and visualized using TMB. Anti-CD3 (OKT3) and/or CD3-binding fragment (Fab) of blinatumomab (Blina) were used as coating controls, and streptavidin-HRP was used to check biotinylation.

After final washing, wells were typically stained with 50 μL TMB substrate for 5 min, and the reaction was stopped by adding 100 μL 2M H₂SO₄. Absorbance was read at A 450 nm on an Envision™ multimode plate reader. Results were processed in GraphPad Prism.

### Jurkat Cell Activation Assay of CD3 Targeting Fabs

In order to assess the CD3-activation potential of the discovered anti-CD30/8 Fabs, luminescence-producing Jurkat cells were used. Potential activators were coated overnight at 4° C. on an ELISA plate in eleven wells through an 11-step semi-log dilution series. The dilution started at 150 μg/mL in case of Fabs, or 31.6 ug/mL in the case of the murine anti-CD3 OKT3 antibody. Following washing of the plate with PBS, 40×10³ nuclear factor of activated T-Cells (NFAT) Jurkat reporter cells (93-1141C19, DiscoverX) were seeded into each well and incubated for 24 h at 37° C. After the addition of 50 μL PathHunter® detection solution, chemiluminescence was measured in relative fluorescence units (RFU) on an Envision plate reader without setting a wavelength readout per instruction of the manufacturer of the PathHunter® Detection Kit (93-0001, DiscoverX).

### Construct Design and Bispecific Antibody (BsAb) Production

Anti-TrkB Fv candidate sequences were designed as a scFv-Fc (KiH) gene format employing a VH-linker-VL scFv strategy containing a SSGGGGSGGGGSGGSAL linker sequence (SEQ ID NO:707) between the VH and VL domain. Here, KiH refers to the knob-in-hole strategy as described in PCT patent publication No. WO 1998/050431A2. The human IgG1-Fc contained second generation knob mutations (T366W, S354C) [WO 1998/050431A2]. Anti-CD3 Fv candidate sequences were designed as Fab-Fc (KiH) genes format consisting of a human Ig light chain gene and human IgG1 heavy chain gene. The Fc of the heavy chain contained the second generation hole mutations (T366S, L368A, Y407V, Y349C) as described in WO1998050431A2.

The various gene constructs were individually cloned into an expression vector. For the production of bsAb, anti-TrkB plasmid (1 construct) and anti-CD3 plasmids (2 constructs, namely VL and VH) were transiently transfected into HEK293 at a 1:1:1 molar ratio. After 6-8 days, production supernatants were purified using protein A. The obtained IgG product was characterized by SDS-PAGE and SEC-HPLC for bsAb purity and integrity. Concentration of proteins was determined using absorbance spectroscopy at λ 280 nm.

ELISA-Based bsAb Screening

Human TrkB-His was immobilized directly on ELISA plates, whereas biotinylated CD3 and BSA were captured on neutravidin-coated ELISA plates overnight (all at 1 μg/mL). Subsequently, wells were blocked (1% (w/v) BSA in PBS) followed by incubation with a 7-step semi-log 10 dilution series of KiH TrkB×CD3 bsAbs, starting at 10 μg/mL. Antibody binding was detected using anti-human-IgG-HRP. Anti-His and streptavidin-HRP were included as coating controls for TrkB, CD3 and bio-BSA, respectively. Anti-human-IgG-HRP or anti-mouse-IgG-HRP only were included as negative controls.

Flow Cytometry-Based bsAb Screening

Antibody screening against CHO-k1 human TrkB, CHO-k1 human CD3d/e, and CHO-k1 parental was conducted by flow cytometry. Cells were harvested, washed, re-suspended in 2% FBS in PBS and seeded at 5×10⁴ cells/well. Subsequently, cells were incubated with a 6-step semi-¹⁰ log dilution series of anti-TrkB×CD3 bispecific antibodies (starting at 10 μg/mL). Antibody binding was detected with anti-human IgG-PE. Cells were analyzed on an iQue flow cytometer.

### Flow Cytometry-Based Bridging Assay

CHO-k1 target cells were harvested, washed, stained with 1:2,000 diluted Multicyt FL4 proliferation dye (Cat. #90360, Essen BioScience) or stained with 1:2,000 diluted Multicyt FL1 encoder dye (Cat. #90354, Essen BioScience), which are both membrane-permeable dyes. The cells were incubated for 10 min, washed again, and re-suspended in 2% FBS in PBS. FL4-stained CHO-k1_CD3 cells were seeded at 5×10⁴ cells/well to a 384-wells V-bottom microplate (Cat. #781281, Greiner) and subsequently incubated with an 8-step semi-log 10 dilution series of anti-TrkB×CD3 bispecific antibodies, starting at 100 μg/mL, for 1 h at 4° C. Next, FL1-stained CHO-k1_TrkB cells 5×10⁴ cells/well were seeded on top, and plates were incubated for another hour at 4° C. Antibody bridging was measured by the detection of FL1/FL4 positive doublets on an iQue flow cytometer. CHO-k1 parental cells stained with the same dyes were taken along as negative control.

### Jurkat NFAT Cell Activation Assay

A number of 20×10³ Jurkat NFAT cells in 25 UL and 20×10³ huTrkB-expressing CHO cells in 25 μL were combined, mixed and seeded in 96-wells assay plates. Jurkat NFAT cells without huTrkB-CHO were seeded as the negative control. An 11-step semi-log 10 dilution series of anti-TrkB×CD3 bsAbs (starting at 100 μg/mL, volume 50 μL) was subsequently added to cells and incubated overnight at 37° C. Next, freshly prepared detection solution was added, and plates were incubated for 1 h protected from light at RT. The amount of luminescence was determined on an Envision plate reader.

### Cell-Based TrkA Activation Assay

A PathHunter® eXpress TrkA Functional Assay (Cat #93-0462E3CP16) was purchased from DiscoverX (Eurofins) and used to probe TrkA activation by the anti-TrkB antibody candidates. The assay was conducted according to the instructions of the supplier. In brief, reporter cells were plated in a 96-well cell culture plate and resuscitated at 37° C. for 48 h. Cells were treated with either exclusively medium, negative controls BDNF (TrkB ligand; Cat. #450-02, PeproTech) or NISTmab (non-TrkA and non-TrkB binding antibody), nerve growth factor (Cat. #A42578, ThermoFisher) as positive control, or selected anti-TrkB antibody leads for 3 h at RT. Following stimulation and/or inhibition, detection reagent was added to the wells containing cells, and luminescence was read.

### Cell-Based TrkB Activation Assay

A PathHunter® express TrkB Functional Assay (Cat. #93-0463E3CP16, DiscoverX (Eurofins)) was applied to screen the activation of TrkB by various anti-TrkB antibodies. Ligand BDNF was taken along as positive control. In brief, supplied, modified TrkB-sensitive cells were transferred to a 96-wells cell culture plate, and vitalized at 37° C. for 48 h. Prior to a 3 h stimulation at ambient temperature, anti-TrkB antibodies, recombinant TrkB-ligand BDNF or negative control antibody NISTmab were added. Luminescence as a measure of the level of activation was determined after addition of detection reagent.

### Cell-Based Phosphorylation ELISA Assay

A number of 4× 105 of in-house made huTrkB-expressing CHO-K1 or CHO-K1 parental cells, were seeded in a volume of 400 μL/well medium in 24-wells SBS plates. Following overnight incubation, medium was removed and cells were acclimatized in serum-free medium for 2 h. After addition of 100 L medium containing 0, 0.1, 1.0, or 10 μg/mL anti-TrkB Abs, 10 μg/mL negative control antibody (isotype control hIgG1), or 10 nM BDNF as positive control, cells were incubated for another 15 min. Immediately after aspiration of the liquid phase, cells were lysed in 200 μL lysis buffer. ELISA was performed according to the manufacture's protocol (Cat. #DYC688-2, Bio-techne R&D Systems). Briefly, plates were coated overnight with a mouse anti-human TrkB antibody prior to the addition of 100 L/well lysate. Following 2 h incubation, plates were washed, incubated with mouse-anti-pY HRP mAb for another 2 h, and then visualized with TMB. The amount of generated chromophore was determined at A 450 nm.

### Internalization Assays

Chicken anti-TrkB clones in CHO-k1 cells. CHO-k1 huTrkB cells and CHO-k1 parental cells were seeded in 96-well plates. Each of the 9 anti-TrkB chicken clones was added to the cells at the following concentrations: 10, 3.3, 1 and 0.4 ug/ml. Plates were incubated at 4° C. and 37° C. for 1 h and 4 h. After incubation, plates were put on ice and cells were washed with ice-cold 2% FBS/PBS three times. The cells were subsequently incubated with secondary antibody Donkey F(ab′)₂ Anti-Human IgG-PE, (1:300) in culture medium for 1h on ice then washed with ice cold PBS. Cells were fixed and resuspended in PBS/2% PFA for further analysis by flow cytometry to determine the change in mean surface receptor presence.

Human anti-TrkB clone in CHO-k1 cells. CHO parental and CHO-TrkB cells were seeded in 96-well plates. Cells were incubated with anti-TrkB clones (10 ug/ml) at 4° C. and 37° C. for the following time points: 2 h, 4 h, 6 h, 16, 20h, and 24h. Three wells were used per condition. After incubation time, plates were washed with ice cold 2% FBS/PBS three times. The cells were subsequently incubated with secondary antibody Donkey F(ab′)₂ Anti-Human IgG-PE, (1:300) in culture medium for 1h on ice then washed with ice cold PBS. Cells were fixed and resuspended in PBS/2% PFA for further analysis by flow cytometry to determine changes in mean surface receptor presence.

Human anti-TrkB clone in lung carcinoma derived A549 cells. A549 cells which endogenously express TrkB were seeded at 5×10⁴ cells/well. Anti-TrkB antibodies were prepared with a 5-step dilution series to 31.6 ug/ml and added to the wells. Plates were incubated at 4° C. and 37° C. for 4 hr and 22 hr. Cells were washed with FACS buffer. Subsequently detection antibody was added and incubated for 1 h at 4° C. Cells were washed with FACS buffer and 7-AAD/PBS (1:500) was added and incubated for 10 minutes.

### Example 2: Results-Identification and Characterization of TrkB-Specific Antibodies

To acquire mAbs recognizing epitopes on human and on murine variants of the extracellular domain (ECD) of TrkB, two discovery approaches were applied: i) interrogation scFv fragments present phage display libraries for development into full-length TrkB-specific human antibodies, and ii) bioengineering of antibodies derived from TrkB-immunized chicken. The rationale for the latter approach is that chicken-derived antibodies bind commonly to unique epitopes compared to those from other species.

### A) Identification of TrkB-Specific Antibodies by Phage Display

From the phage display screening experiments, 31 sequence-unique scFv clones were selected for the development of full-length IgG sequences transiently expressed in HEK293 cells. Before transfection, sequences were verified. The sequences of the heavy and light chains of the 31 clones are depicted in Table 4.

TABLE 4
Amino acid sequences of the heavy and light chain variable regions of the 31
sequence-unique anti-TrkB scFv clones selected for the development of full-length
IgG sequences

| Clone | | LCVR |
|---|---|---|
| ID | HCVR | |
| 1E02 | QVQLLQSAAEVKKPGESLKISCKGSGYR | QSVLTQPPSLSVSPRQTASITCSGDKLGD |
| | FTSYWIGWVRQMPGKGLEWVGIIYPDD | KFVSWYQQKPGQSPVLVIYQDTKRPSGI |
| | SDTRYSPSFQGQVTISADTSISTTYLQW | PERFSGSNSGNTATLTISGTQAMDEADY |
| | SSLKASDTAIYYCASGEVAFDVWGQGTT | YCQAWDSSTGVFGGGTKLTVL (SEQ ID |
| | VTVSS (SEQ ID NO: 314) | NO: 315) |
| 1E07 | QVQLVQSGAEVKKPGESLRISCKGSGY | QSVLTQPPSVSGAPGQRVTISCTGSSSNI |
| | SFTNNWINWVRQMPGKGLEWMGRIDP | GAGFNVHWYQQVAGLAPKLLIHDNINRP |
| | DDSYTNYSPSFQGHVIFSADKSSNTVYL | SGVPDRFSGSKSGASASLTITGLQAEDE |
| | QWSSLEASDTAMYYCARRAQGGGGYD | ADYYCQSYDSSLSGWFAFGTGTKVTVL |
| | YWGQGTLVTVSS (SEQ ID NO: 316) | (SEQ ID NO: 317) |
| 1F01 | QVQLQQSGPGLVKPAQTLSLTCDISGDS | QSVVTQPASVSGSPGQSITISCTGTSSDI |
| | VSSKSAAWHWIRQSPSRGLEWLGRTYY | GGYDYVSWYQQHPGKAPKLMIFDVSNR |
| | RSKWYSDYGESVKSRVVINADTSNNQIS | PSGVSNRFSGSKSGNTASLTISGLQADD |
| | LQLSSVTPEDTAAYYCARGIGSLNFDIW | EADYYCSSYRAGSTLNLLFGGGTKLTVL |
| | GQGTMVTVSS (SEQ ID NO: 318) | (SEQ ID NO: 319) |
| 2C07 | QVQLQQSGGGVVQPGRSLRLSCAASGF | LSELTQDPAVSVALGQTVRITCQGDSLRS |
| | TFSNYAMHWVRQAPGKGLEWVAVISYD | YYASWYQQKPGQAPVLVIYGKNNRPSGI |
| | GSNEYYADSVKGRFTISRDNSRNTLDLQ | PDRFSGSDSGKTASLTITGAQAEDEADY |
| | MNSLRAEDTAVYYCARGTNYGMDVWG | YCQSRDTTGNYLLFGGGTKLTVL (SEQ ID |
| | QGTMVTVSS (SEQ ID NO: 320) | NO: 321) |
| 2E02 | QVQLQQSGPGLVKPSQTLSVTCVISGDS | QSVLTQPPSASGSPGQSVTISCTGTSGD |
| | VSSKSAAWHWIRQSPSRGLEWLGRTYY | VGGYNYVSWYQQHPGKAPKLILYEVTKR |
| | RSKWYSDYGESVKSRVVINADTSNNQIS | PSGVPDRFSGSKSGNTASLTVSGLQAED |
| | LQLSSVTPEDTAAYYCARGIGSLNFDIW | EADYYCSSYGGPNNLVLFGGGTKVTVL |
| | GQGTMVTVSS (SEQ ID NO: 322) | (SEQ ID NO: 323) |
| 2E08 | QVQLVQSGAEVKKPGESLKISCTGSGYR | SYVLTQPPSVSVSPGQTARITCSGDKLG |
| | FSHYWIGWVRQMPGKGLEWMGMIYPD | DKYVSWYQLKPGQSPVVVIYQDNKRPSG |
| | DSDTRYSPSFQGQVTISADKSINTAYLQ | IPERFSASNSENTGTLTISGTQAMDEADY |
| | WTSLRASDSAIYYCATGVISGTDYWGQ | YCQTWDSGTGVFGTGTKLTVL (SEQ ID |
| | GTLVTVSS (SEQ ID NO: 324) | NO: 325) |
| 2H05 | EVQLVESGAEVKKPGESLRISCKGSGYS | QSVLTQPSSVSGAPGQRVTISCTGSSSNI |
| | FTNNWINWVRQMPGKGLEWMGRIDPD | GAGYGVHWYQQLPGTAPKLLIYDDINRP |
| | DSYTNYSPSFQGHVIFSADKSSNTVYLQ | SGVPDRFSGSKSGTSASLAITGLRAEDEA |
| | WSSLEASDTAMYYCARRAQGGGGYDY | DYYCQSYDTSRGGLYVFGSGTKLTVL |
| | WGQGTLVTVSS (SEQ ID NO: 326) | (SEQ ID NO: 327) |
| 3A05 | QVQLVESGAEVKKPGESLKISCKGSGYS | QAGLTQPPSASGTPGQRVTISCSGSSSNI |
| | FTTYWIAWVRQMPGKGLEWMGIIYPGD | GSNTVSWYRQIPGTAPKFLLYGNNQRPS |
| | SDTRYSPSFQGQVTISADKSISTAYLQW | GVPARFSGSKSGTSASLAVDGLQSDDEA |
| | SSLKASDTAMYYCARLGGGMDVWGQG | DYYCAAWDDSLSGYVFGTGTKLTVL |
| | TMVTVSS (SEQ ID NO: 328) | (SEQ ID NO: 329) |
| 3A06 | EVQLVQSGGGLVKPGGSLRLSCAASGF | QSVLTQPPSVSVSPGQTASITCSGDKLG |
| | TFSNAWMSWVRQAPGKGLEWVGRIKS | NKYASWYRQKPGQSPVLVIYQDTKRPSG |
| | KTDGGTTDYAAPVKGRFTISRDDSKNTL | IPERFSGSNSGNTATLTISGTQAMDEGDY |
| | YLQMNSLKTEDTAVYYCTTENEVWPSL | YCQAWDSSTAVVFGGGTKLTVL (SEQ ID |
| | DVWGQGTTVTVSS (SEQ ID NO: 330) | NO: 331) |
| 3B04 | QVQLVQSGAEVKKPGEPLKISCKGSGYS | SYELMQPPSVSVSPGQTASITCSGDKLG |
| | FPTYWIAWWVRQMPGKGLEWMGIIYPGD | TKYVSWYQQKPGQSPVLVIYQDSKRPSG |
| | SDTRYSPSFQGQVTISADKSISTAYLQW | IPERFSGSNSGNTATLTISGTQAMDEADY |
| | GSLRASDTAMYYCARGGSGLDYWGQG | YCQAWDSSTVVFGGGTKLTVL (SEQ ID |
| | TLVTVSS (SEQ ID NO: 332) | NO: 333) |
| 3B06 | EVQLVQSGAEVKEPGASVKVSCKASGY | DIVMTQTPLSLPVTLGQPASISCRSSQSL |
| | TFTNYAMHWVRQAPGQRLEWMGWINP | VDRDGNTYLNWFRQRPGQSPRRLIYKVS |
| | NTGGTNYAQKFQGRVTMTRDTSISTAY | NRDSGVPGTFSGSGSGTDFTLKISRVEA |
| | MELSSLRSDDTAVYYCARGGGGLVFWG | DDVGVYFCMQATHWPPTFGQGTKVEIK |
| | QGTLVTVSS (SEQ ID NO: 334) | (SEQ ID NO: 335) |
| 3B09 | EVQLVQSGAEVKKPGESLKISCKGSGYS | QAGLTQPPSVSVSPGQTVTITCSADNLG |
| | FTSYWIGWVRQMPGKGLEWMGIIYPGD | DKYTSWYQQRPGQSPVLVIYQDTKRPSG |
| | SDTRYSPSFQGQVTISADKSISTAYLQW | IPARFSGSNSGNTATLTISGTQAMDEADY |
| | SSLKASDTAMYYCATPMEDGMDVWGQ | YCQAWDSSTGVFGTGTKVTVL (SEQ ID |
| | GTTVTVSS (SEQ ID NO: 336) | NO: 337) |
| 3C02 | QVQLVQSGAEVKKPGESLKISCKGSGYS | LPVLTQPPSVSVSPGQTASITCSGDRLGD |
| | FTSYWIGWVRQMPGKGLEWMGIIYPGD | KYASWYQQKPGQSPVLVIYQDSKRPSGI |
| | SDTRYSPSFQGQVTISADKSISTAYLQW | PERFSGSNSGNTATLTISGTQAMDEADY |
| | SSLKASDTAMYYCARGGPPDYWGQGTL | YCQAWDSSTAVFGGGTKLTVL (SEQ ID |
| | VTVSS (SEQ ID NO: 338) | NO: 339) |
| 3C12 | QVQLVQSGAEVKKPGESLKISCKGSGYS | QPVLTQPPSVSVSPGQTASITCSGDKLG |
| | FTSYWIGWRQMPGKGLEWMGIIYPGD | NKFVSWFQQKPGQSPVLVIYQDTKRPSG |
| | SDTRYSPSFQGQVTISADKSISTAYLQW | IPERFSGSNSGSTATLTITGTQAMDDADY |
| | SSLKASDTAMYYCTRSSFNDHWGQGTL | YCQAWNTNTALFGGGTKLTVL (SEQ ID |
| | VTVSS (SEQ ID NO: 340) | NO: 341) |
| 3D03 | EVQLVQSGAEVKKPGESLKISCKGSGYS | QSVLTQPPSVSVSPGQTASITCSGDKLG |
| | FTSYWIGWVRQMPGKGLEWMGIIYPGD | DKYVSWYQQKPGQSPVLVIYQDSKRPS |
| | SDTRYSPSFQGQVTISADKSISTAYLQW | GIPERFSGSNSGSTATLTISGTQALDEAD |
| | SSLKASDTAMYYCARLGGSLPIDYWGQ | YYCQAWDNNIGVFGGGTKLTVL (SEQ ID |
| | GTLVTVSS (SEQ ID NO: 342) | NO: 343) |
| 3D06 | EVQLVQSGAEVKKPGESLKISCQGSGYT | QPVLTQPPSASGTPGQRVSISCSGSNSNI |
| | FTNYWIAWVRQMPGKGLEWMGIIYPGD | GSNSVYWYQQLPGTAPKLLIYRNNQRPS |
| | SDTRYSPSFQGQVTISADKSISTAYLQW | GVPDRFSGSKSGTSASLAISGLRSEDEA |
| | SSLKASDTAMYYCAIGYDYGAFDIWGQG | DYYCAAWDDSLSGYVFGTGTQLTVL |
| | TMVTVSS (SEQ ID NO: 344) | (SEQ ID NO: 345) |
| 3D11 | QVQLVESGAEVKKPGEPLKISCKGSGYS | QAGLTQPPSVSVSPGQTAIIPCSGEKLGD |
| | FPTYWIAWVRQMPGKGLEWMGIIYPAD | KYVSWYQQKPGESPLSIIYQDTERPSGIP |
| | SDTRYSPSFQGQVTISADKSISTAYLQW | ERFSGSNSGNTATLTIRGTQAMDEADYY |
| | GSLRASDTAMYYCARGGSGLDYWGQG | CQAWDSNTMVFGGGTKLTVL (SEQ ID |
| | TLVTVSS (SEQ ID NO: 346) | NO: 347) |
| 3E04 | EVQLVQSGAEVKKPGESLKISCKGSGYS | SYVLTQPPSVSVSPGQTATITCSGDKLGD |
| | FTSYWIGWVRQMPGKGLEWMGIIYPGD | EYVSWYQQKPGQSPVLVIYQDSKRPSGI |
| | SDTRYSPSFQGQVTISADKSISTAYLQW | PERFSGSNSGNTATLTISGAQAVDEADY |
| | SSLKASDTAMYYCASRLLFGDDAFDIWG | YCQAWDSNAGAFGTGTKLTVL (SEQ ID |
| | QGTMVTVSS (SEQ ID NO: 348) | NO: 349) |
| 3F06 | QMQLVQSGAEVKKPGESLKISCKGSGY | QSVLTQPPSVSVSPGQTASITCSGDKLG |
| | SFSTNWIGWVRQMPGKGLEWMGIIYPG | DKYASWYQQKPGQSPVLVIFQDNKRPS |
| | DSDTRYSPSFQGQVTISADKSISTAYLQ | GIPERFSGSNSGNTATLTISGTQAMDEAD |
| | WSSLKASDTAMYYCARFYDSSGNDYW | YYCQTWDSSTAIFGGGTKLTVL (SEQ ID |
| | GQGTLVTVSS (SEQ ID NO: 350) | NO: 351) |
| 3F09 | EVQLVQSGAEVKKPGESLKISCKGSGYS | QSVLTQPPSVSVSPGQTASITCSGDKLG |
| | FTSYWIGWVRQMPGKGLEWMGIIYPGD | DKYASWYQQKPGQSPVLIIYQDNKRPSGI |
| | SDTRYSPSFQGQVTISADKSISTAYLQW | PERFSGSNSGNTATLTISGTQAMDEADY |
| | SSLKASDTAMYYCVVRPAAAGTRIDYW | YCQAWDSSTYVFGTGTKLTVL (SEQ ID |
| | GQGTLVTVSS (SEQ ID NO: 352) | NO: 353) |
| 3H11 | QVQLQQSGPGLVKPSQTLSLTCAISGDS | LPVLTQPPSASGTPGQRVTISCSGSSSNI |
| | VSSNSAVWNWIRQSPSRGLEWLGRTYY | GHKYVYWYQQLPGAAPKLLIYRNNQRPS |
| | RSKWYTDFAVSVKSRITINPDTSKNQFSL | GVPDRFSASKSGTSASLAISGLRSEDEAD |
| | QLNSVTPEDTAVYYCARGRNGMDVWG | YYCATWDDSLNGVVFGGGTKVTVL (SEQ |
| | QGTMVTVST (SEQ ID NO: 701) | ID NO: 702) |
| 4D01 | EVQLVQSGAEVKKPGESLKISCKGSRYS | QAVLTQPPSASGTPGQRVTISCSGSSSNI |
| | FTSYWIGWVRQMPGKGLEWMGIIYPGD | EGNYVHWYQQLPGAAPKVLIYRNNQRPS |
| | SDTRYSPSFQGQVTISADKSISTAYLQW | GVPDRFSGSKSGTSASLAISGLRSEDET |
| | SSLKASDTAMYYCARLKDGAFDIWGQG | DYFCAAWDDSLNGYVFGAGTKVTVL |
| | TMVTVSS (SEQ ID NO: 354) | (SEQ ID NO: 355) |
| 4F03 | EVQLVQSGAEVKKPGESLKISCKGSGYS | QPVLTQPPSASGTPGQRVTISCSGSSSNI |
| | FTSYWIGWVRQMPGKGLEWMGIIYPGD | GTHTVNWYQQFPGTAPKLLMYSTFQRP |
| | SDTRYSPSFQGQVTISADKSISTAYLQW | SGVPDRFSGSKSGTSASLAISGLQSEDE |
| | SSLKASDTAMYYCASWGMDVWGQGTL | ADYYCAAWDDSLNGPVFGGGTQLTVL |
| | VTVSS (SEQ ID NO: 356) | (SEQ ID NO: 357) |
| 4G12 | EVQLVQSGAEVKKPGESLKISCKGSGYS | QSVLTQPPSVSVSPGQTASITCSGDKLG |
| | FTSYWIGWVRQMPGKGLEWMGIIYPGD | DKFASWYQQKPGQSPVLVIYQDNKRPSR |
| | SDTRYSPSFQGQVTISADKSISTAYLQW | VPERFSGSNSGNTATLTISETQAMDEAD |
| | SSLKASDTAMYYCARHDYGMDVWGQG | YYCQAWVNNTVVFGGGTKVTVL (SEQ ID |
| | TTVTVSS (SEQ ID NO: 358) | NO: 359) |
| 5A07 | QVQLVQSGAEVKKPGESLKISCKGSGYS | LPVLTQPPSASGTPGQRVTISCSGSRSN |
| | FTSYWIGWVRQMPGKGLEWMGIIYPGD | VGGNTVSWYQQLPGSAPKLLIYTNNQRP |
| | SDTRYSPSFQGQVTISADKSISTAYLQW | SGVPDRFSGSKSGTSASLAISGLQSEDE |
| | SSLKASDTAMYYCARHDYGMDVWGQG | ADYYCAAWDDSLNGYVFGTGTQLTVL |
| | TMVTVSS (SEQ ID NO: 360) | (SEQ ID NO: 361) |
| 5C01 | QMQLVQSGAEVKKPGESLKISCKGSGY | QSVVTQPPSVSGTPGQRVSISCSGSSSN |
| | SFTSYWIGWVRQMPGKGLEWLGIIYPG | IGSHSVTWFQQLPGTAPKLLIYGNSQRPS |
| | DSDTRYSPSFQGQVTISADKSISTAYLQ | GVPDRFSGSKSGTSASLAISGLQSEDEA |
| | WSSLKASDTAMYYCARLGQGSGPFDY | DYYCASWDDSLNGYVFGTGTKLTVL |
| | WGQGTLVTVSS (SEQ ID NO: 362) | (SEQ ID NO: 363) |
| 5C07 | QVQLVQSGAEVKKPGESLKISCKGFGYS | QAGLTQPPSVSVSPGQTASITCSGERLG |
| | FTNYWIGWVRQMPGKGLEWMGIIYPGD | DKYVSWYQQKPGQSPVLVIYQDDKRPS |
| | SDTRYSPSFQGQVTISADKSITTAYLQW | GVPERFSGSNSGNTATLNISGTQPMDEA |
| | SSLKASDTAMYYCASHTKDGTWDIWGQ | DYYCQAWDSSTSLYVFGTGTKLTVL |
| | GTMVTVSS (SEQ ID NO: 364) | (SEQ ID NO: 365) |
| 6B04 | QVQLVQSGAEVKKPGESLKISCKGSGYS | SYELMQPPSLSVSPGQTASITCSGDKLG |
| | FTSYWIGWVRQMPGKGLEWMGIIYPGD | DKYVSWYQQRPGQSPVLVMYQDKKRPS |
| | SDTRYSPSFQGQVTISADKSISTAYLQW | GIPKRFSGSNSGNTATLTISGTQAMDEAD |
| | SSLKASDTAIYYCARLGGAMDVWGQGT | YYCAAWDDSLNGYYVFGTGTQLTVL |
| | LVTVSS (SEQ ID NO: 366) | (SEQ ID NO: 367) |
| 6B11 | QVQLQQSGAGLVKASQTLALTCAISGDS | QSVLTQPPSASGTPGQRVTISCSGSSSNI |
| | VSSNSAAWNWIRQSPSRGLEWLGRTYY | GSYFVSWYQQLPGTAPKLLIYRNDKRPS |
| | RSKWYNDYAVSVKSRITINPDTSTNQFS | GVPERFSGSKSGTSASLAISGLQSEDEA |
| | LQLNSVTPEDTAVYYCTRGTGDKRAFDY | DYYCAAWDDSLSGRIFGGGTKLTVL |
| | WGQGTLVTVSS (SEQ ID NO: 368) | (SEQ ID NO: 369) |
| 6E08 | QVQLVQSGGGVVQPGRSLRLSCAASGF | DIQMTQTPLSLSVTLGQPASISCRSSQGL |
| | TFSSYAMHWVRQAPGKGLEWVAVISYD | VYSDGNTYLNWFQQRPGQSPRRLIYKVS |
| | GSNKYYADSVKGRFTISRDNSKNTLYLQ | NRDSGVPDRFSGSGSGTDFTLKISRVEA |
| | MNSLRAEDTAVYYCARAFRRYDAFDIW | EDVGVYYCMQGTHWPPAFGQGTKVEIK |
| | GQGTLVTVSS (SEQ ID NO: 370) | (SEQ ID NO: 371) |
| 6F04 | EVQLVQSGAEVKKPGESLKISCKGSGYS | QSVLTQPPSVSVSPGQTASITCSGNKLG |
| | FTSYWIGWVRQMPGKGLEWMGIIYPGD | DKFVSWYHQRPGQSPVLVIYEDNKRPSG |
| | SDTRYSPSFQGQVTISADKSISTAYLQW | IPERFSGSNSGNTATLTISGTQAMDEGDY |
| | SSLKASDTAMYYCARGSGGYDYWGQG | YCQTWDVSSYVFGTGTKVTVL (SEQ ID |
| | TLVTVSS (SEQ ID NO: 372) | NO: 373) |

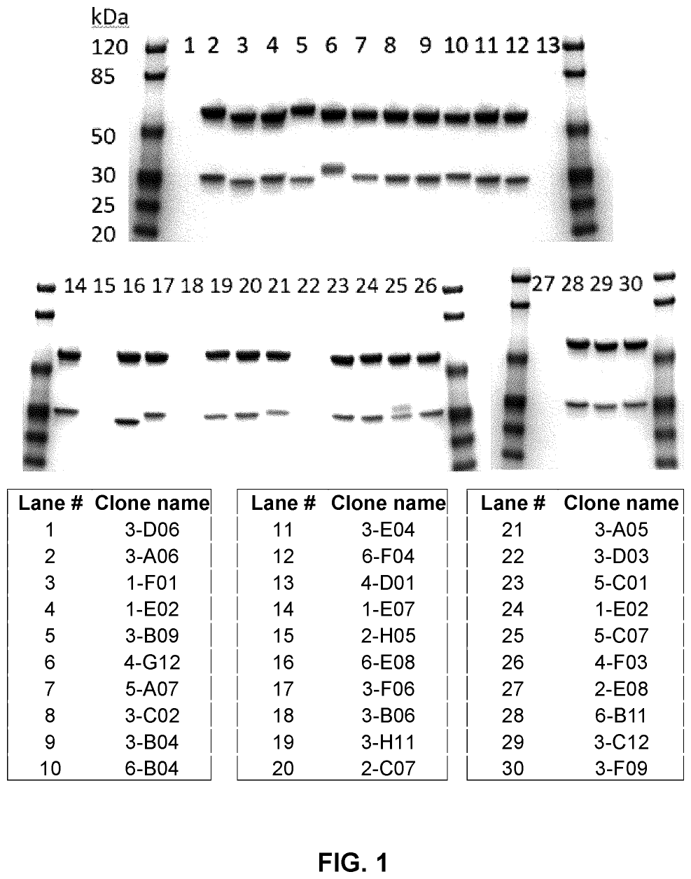

Cell-produced anti-TrkB IgGs were isolated through protein A-affinity chromatography, and their integrity and purity were verified by SDS-PAGE (FIG. 1). Visualized bands reflect heavy and light chains with variations for their apparent molecular masses probably as a result of varying lengths in the VH and VL chains, and co- and post-translational modifications of the heavy (HC) and light (LC) chains.

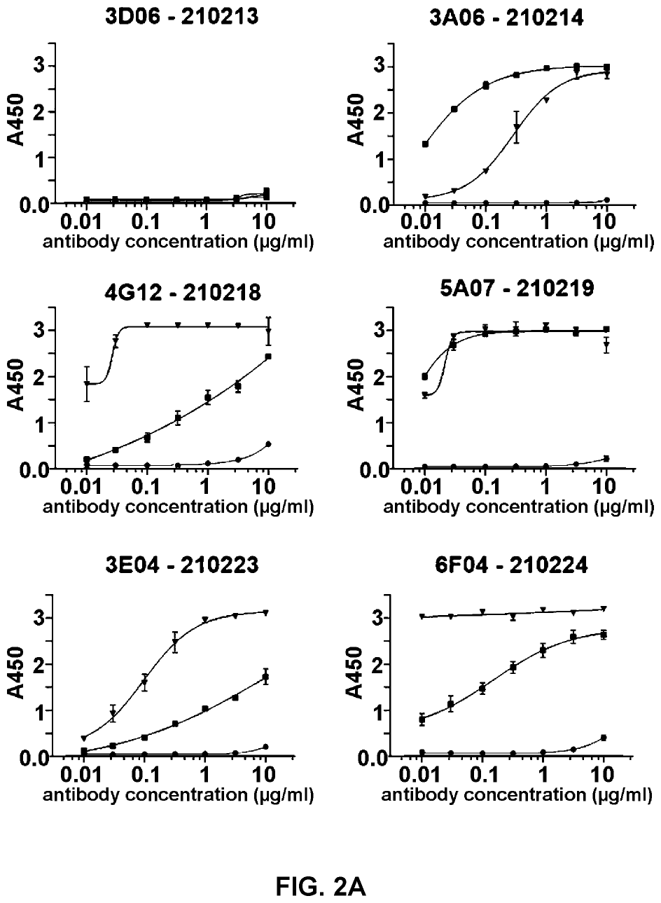

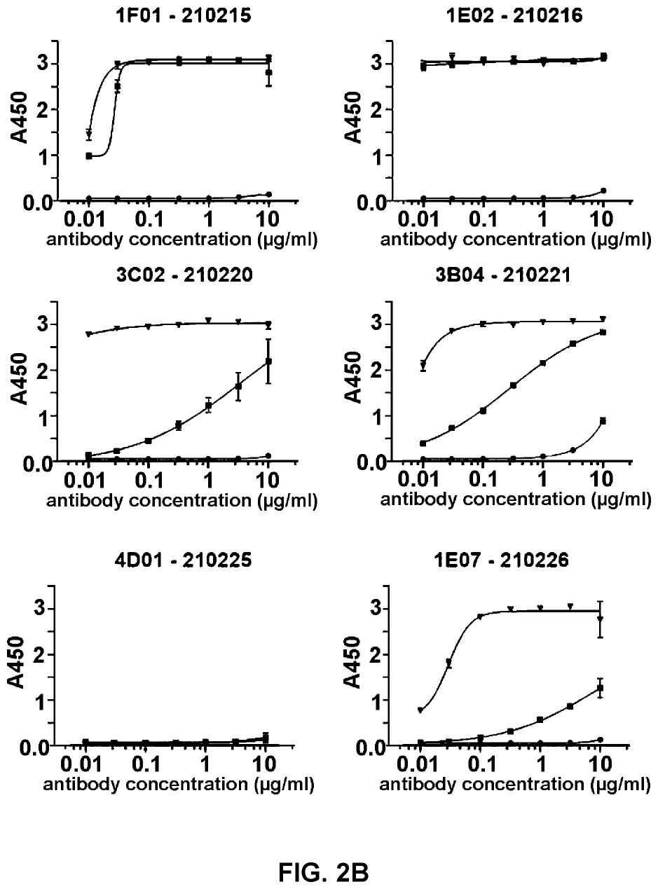

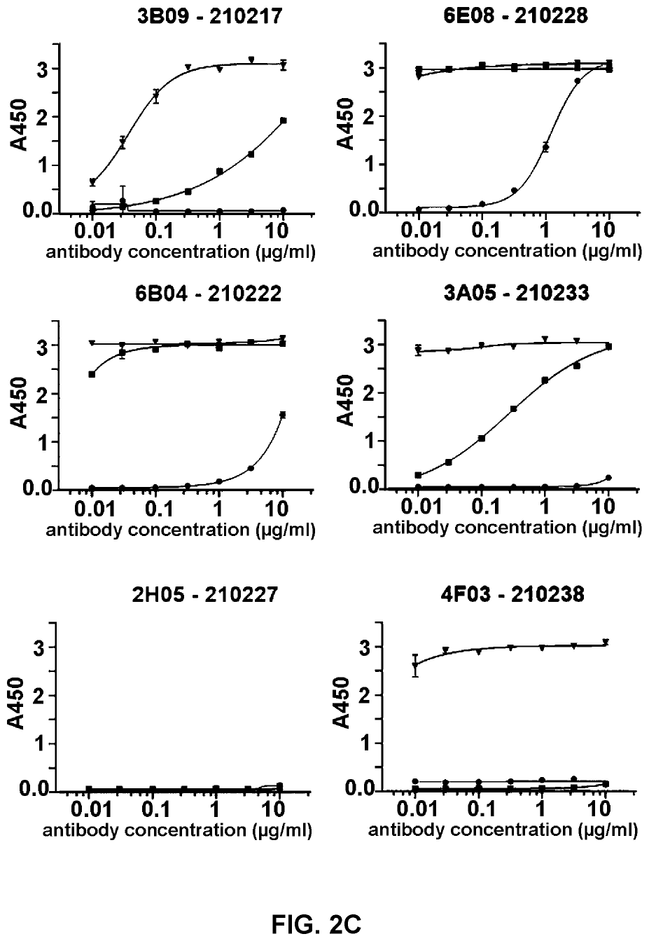

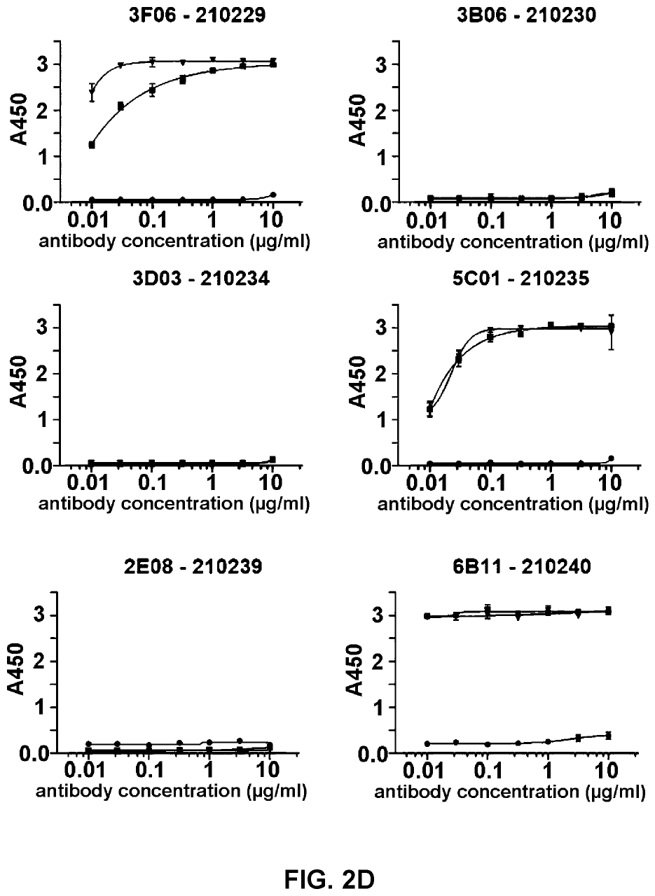

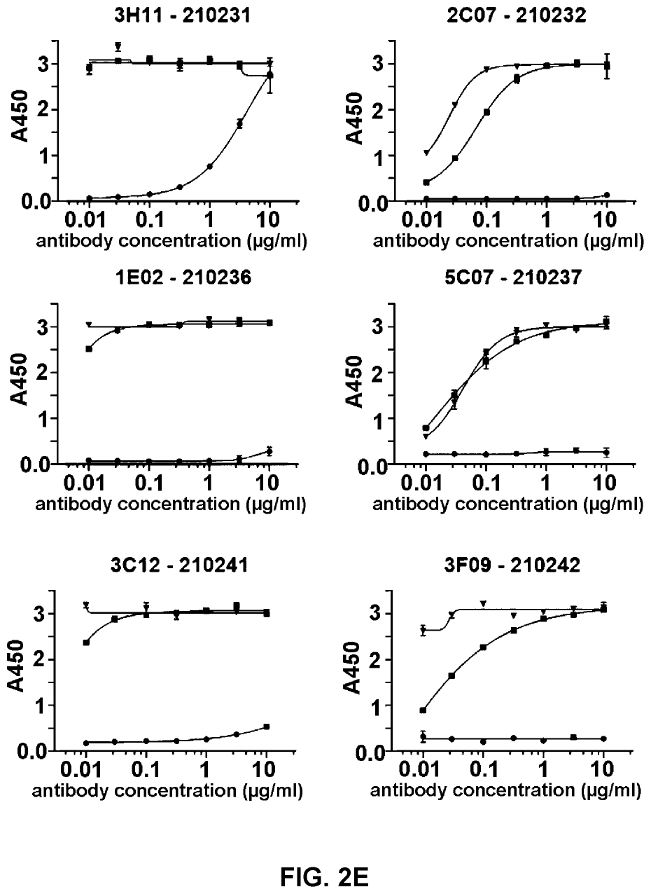

ELISA analysis of each mAb against coated mTrkB and hTrkB confirmed failing antibodies 3-D06, 4-D01, 2-H05, 3-B06, 3-D03 and 2-E08. Of the 24 remaining clones, 23 synthesized antibodies were co-reactive to murine and human variants of TrkB in particular at higher concentrations (FIGS. 2A-E). One clone, 4-F03, binds exclusively to human TrkB-GST, whereas this exclusivity towards the mouse variant is not observed. Several clones were reactive to immobilized reference GST, in particular at higher concentrations.

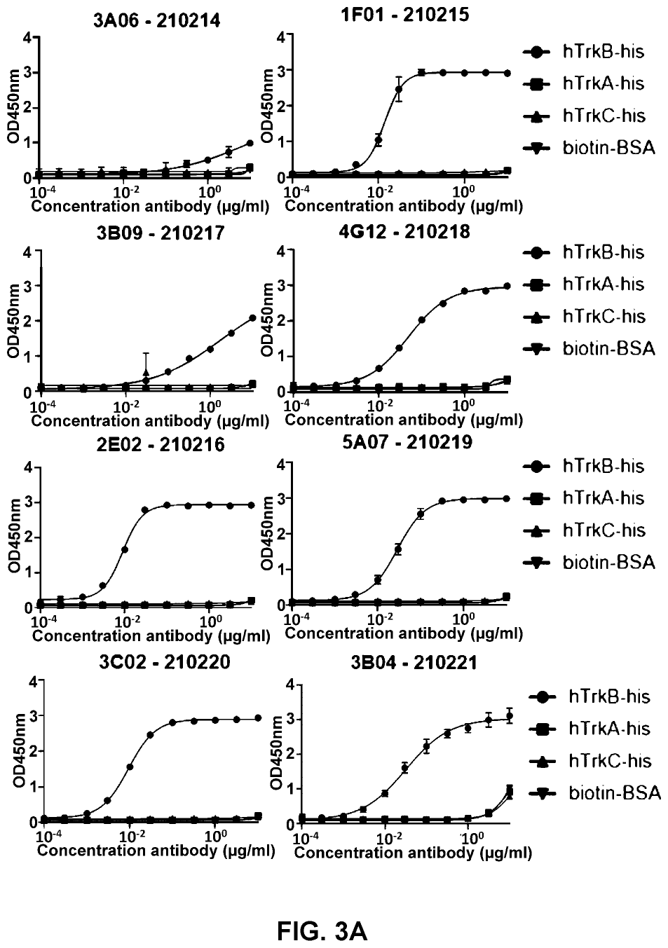

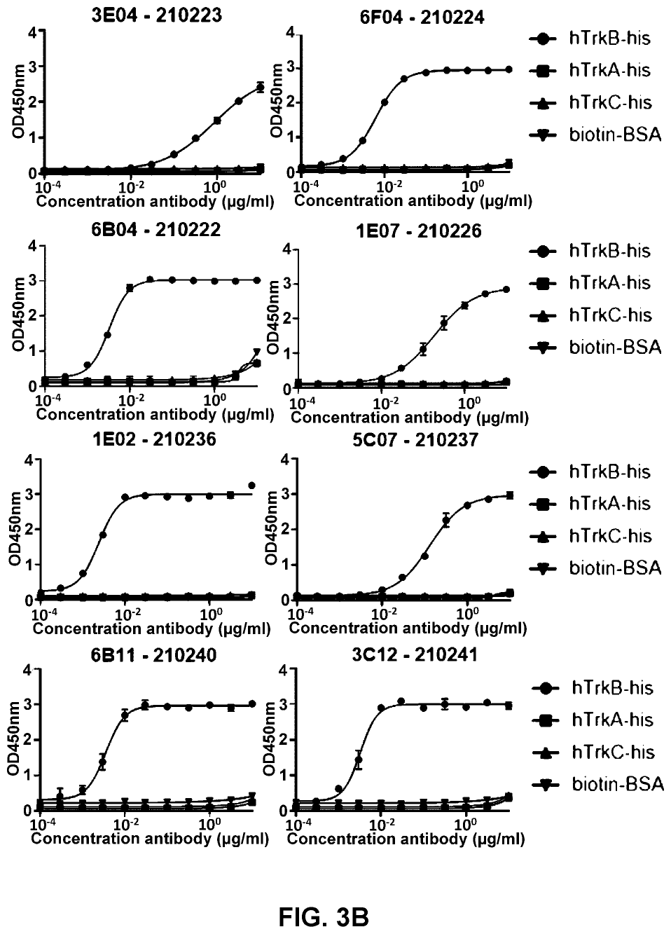

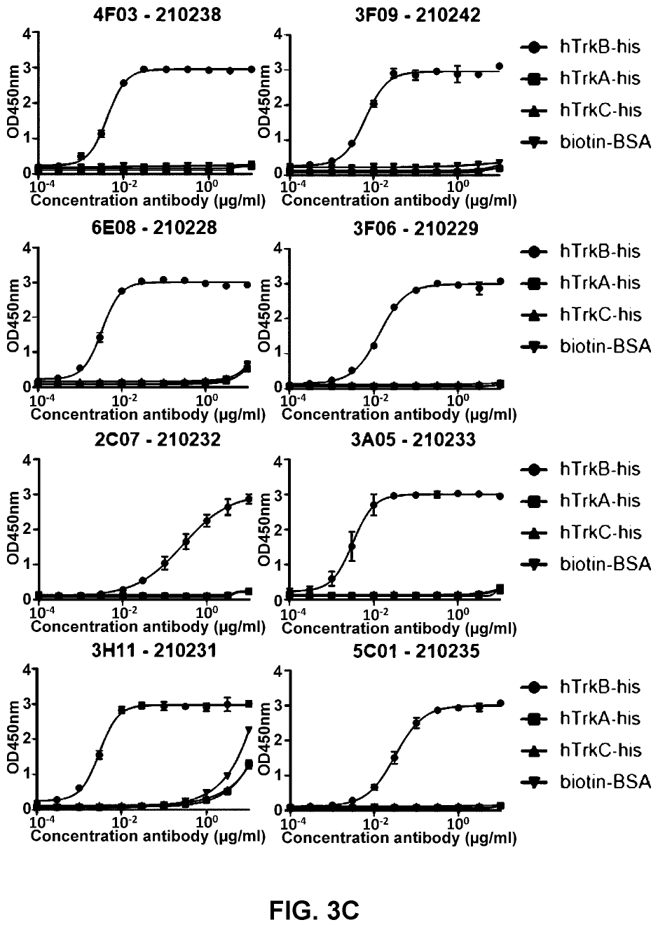

The dose-dependent specificity of the 24 clones of the corresponding full-length antibodies for TrkB was inspected using closely related proteins from the Trk receptor family (FIGS. 3A-C). Cross-reactivity between the Trk receptor proteins is absent, but a- and non-specific binding, including to BSA, may occur at the highest tested concentration (>1 μg/mL) for at least four clones, i.e., 6-B04, 3-B04, 6-E08 and 3-H11. In all tested cases, TrkB binding was more significant than the non-specific binding.

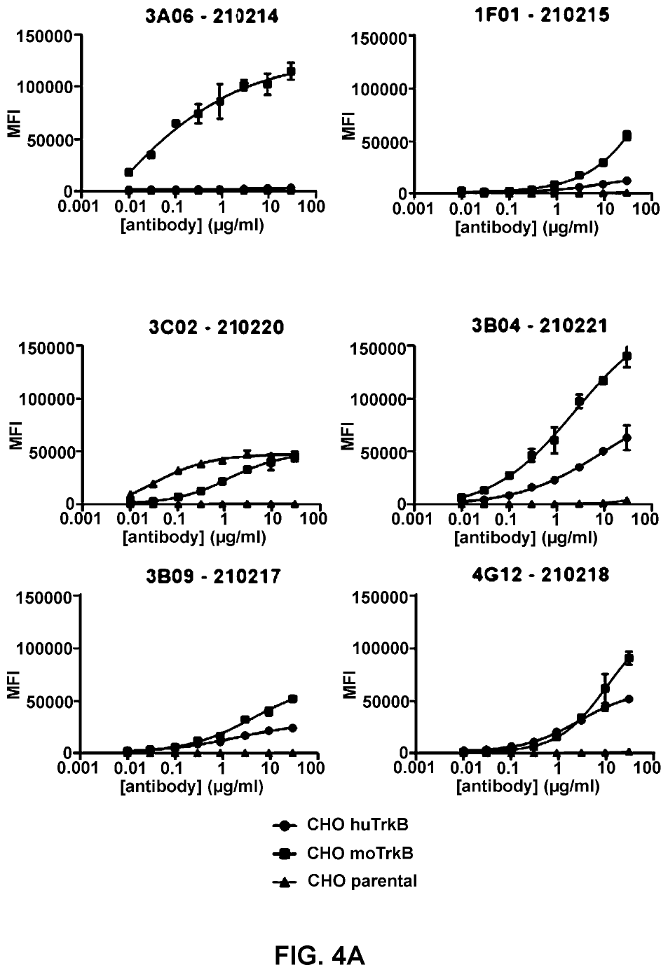

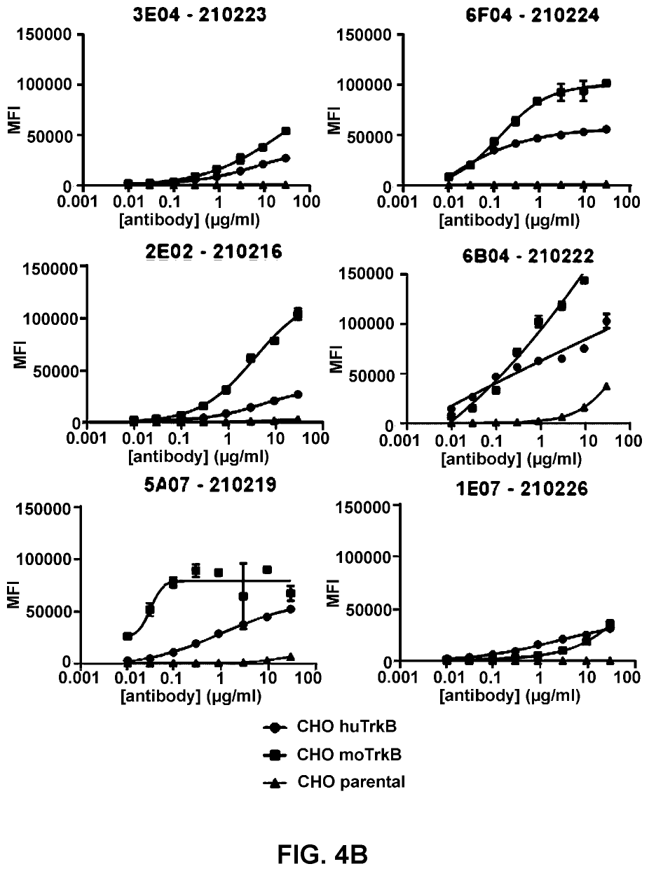

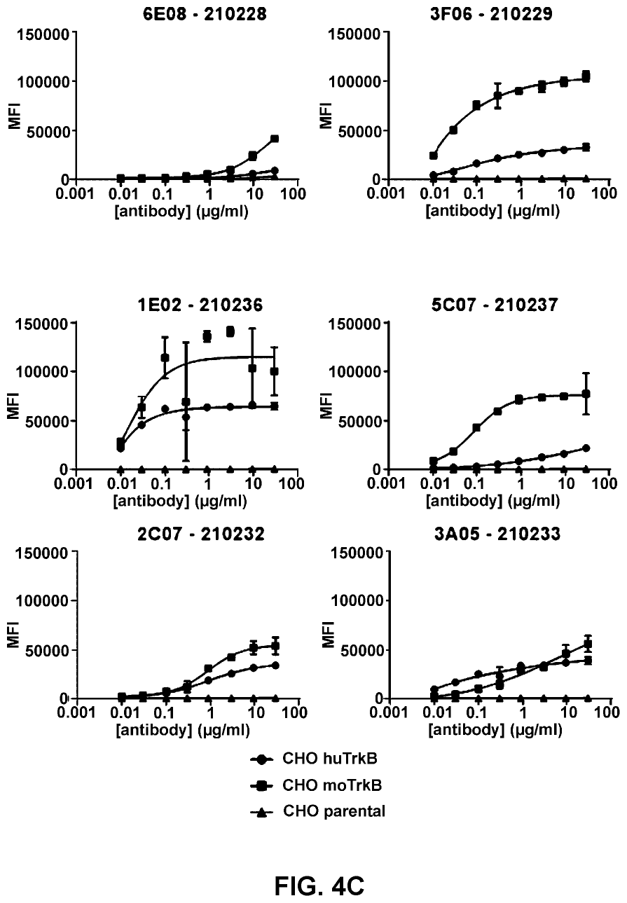

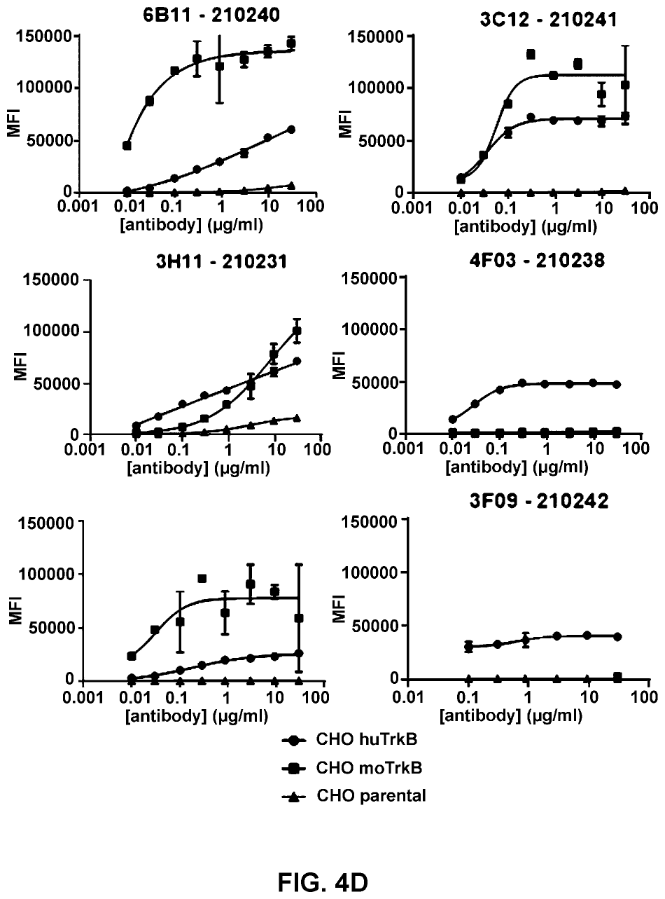

Finally, and in a similar way, the 24 antibody-containing clone samples were screened by flow cytometry for their reactivity against CHO cell-associated TrkB (FIGS. 4A-D). In case of reactivity, it was always discriminative from the parental cells not expressing the antigen. Specific binding activity was apparently not dose-dependent for one clone 3-F09. Weak (e.g., 6-E08) to strong (e.g., 3-C12) binding was observed against TrkB from both species, except clone 3-A06 which was only binding to the mouse variant, while clones 4-F03 (also observed in the ELISA assay) and 3-F09 (both species in ELISA) docked exclusively to the human TrkB type attached to CHO-K1 cells. In the case of the mouse form, clone 3-A06 was exclusive, unlike its ELISA behavior when it showed also significant binding to human TrkB. The biochemical characteristics of a portion of the 24 selected human anti-Trk mAbs are summarized in Table 5. Off-rate kinetics were determined using Octet biosensors coated with anti-human IgG Fc (AHC) to capture the tested mAb before exposing it to the extracellular domain of human TrkB-His antigen. Results of fitting the first 30 s of the dissociation are listed in Table 5.

TABLE 5
Summary of measured biological and biochemical properties of a selection
of the discovered phage display-derived human anti-TrkB antibodies.
TrkB activation, activation of a TrkB signaling pathway in modified
functional U2OS cells in a TrkB PathHunter ® eXpress assay.

| | Cell- | Cell- | | | | |
|---|---|---|---|---|---|---|
| | associated | associated | hTrkB | mTrkB | | |
| mAbs | hTrkB | mTrkB | reactivity | reactivity | K_{dis} | TrkB activation |
| clone | reactivity | reactivity | (ELISA) | (ELISA) | (1/s) | (EC₅₀ in μg/mL) |
| 3A05 | ++ | + | +++++ | ++++ | 4.35E−03 | 6.0E−03 |
| 6B04 | +++++ | un | +++++ | +++++ | 6.78E−03 | 3.9E−02 |
| 3C12 | +++++ | +++++ | +++++ | +++++ | 5.86E−04 | 1.8E−02 |
| 4G12 | ++ | ++ | +++++ | +++ | 5.45E−03 | 7.0E−02 |
| 5C01 | ++ | +++++ | +++++ | +++++ | 4.91E−03 | 8.0E−02 |
| 1E02 | +++++ | +++++ | +++++ | +++++ | 3.18E−03 | 4.7E−02 |
| 4F03 | +++++ | − | +++++ | + | 6.39E−03 | 4.1E−02 |
| 5A07 | ++ | +++++ | +++++ | +++++ | 5.44E−03 | 1.3E−01 |
| 3F09 | ++++ | nd | +++++ | +++++ | 6.31E−03 | 5.3E−02 |
| 3B09 | ++ | ++ | +++++ | ++ | 4.72E−03 | 9.4E−02 |
| 6F04 | ++++ | ++++ | +++++ | ++++ | 4.73E−03 | 3.5E−02 |
| 3A06 | − | +++ | +++ | +++++ | 4.53E−03 | 1.5E−01 |
| 5C07 | + | +++++ | ++++ | ++++ | 4.57E−03 | 4.7E−01 |
| 3E04 | + | + | ++++ | +++ | 5.34E−03 | 1.1E−01 |
| 3B04 | ++ | ++ | +++++ | +++ | 8.02E−03 | 1.0E−01 |
| 3C02 | +++++ | +++ | +++++ | +++ | 4.94E−03 | 1.5E−01 |
| 3F06 | +++ | +++++ | +++++ | +++++ | 4.79E−03 | 1.4E−01 |
| 1E07 | + | + | ++++ | +++ | 6.13E−03 | 1.3E−01 |
| 3H11 | un | + | +++++^{#} | +++++^{#} | 5.70E−03 | 2.0E−02 |
| 6E08 | − | + | +++++^{#} | +++++^{#} | 6.40E−03 | 4.2E+00 |
| 6B11 | + | +++++ | +++++ | +++++ | 6.87E−03 | 1.1E−01 |
| 2E02 | + | +++ | +++++ | +++++ | 1.13E−02 | 1.2E−01 |
| 1F01 | − | + | +++++ | +++++ | 1.10E−02 | 5.1E−01 |
| 2C07 | +++ | +++ | +++++ | ++++ | 3.99E−03 | 3.6E−02 |

Reactivity towards cell-associated human or murine TrkB is performed by flow cytometry and demonstrated in FIGS. 4A-B. The binding to plate-immobilized antigens by ELISA is further elaborated in FIGS. 3A-D. Hu, human origin; nd, not determined; un, unresolved.
^{#}at the highest mAb concentrations, high co-reactivity towards GST observed as well, but abolishes fast when antibodies were diluted.
+++++ = strong;
++++ = medium/strong;
+++ = medium;
++ = low/medium;
+ = low/weak

B) Generation of TrkB-Specific Antibodies Derived from TrkB-Immunized Chicken

For the generation of antibodies derived from TrkB-immunized chicken, chickens were immunized with multiple i.m. injections of hTrkb-GST, mTrkB-Fc, and mixtures of hTrkB-GST and mTrkB-GST, and of hTrkB-Fc and mTrkB-Fc with intermittent periods as detailed in Example 1. Following the animal experiments, B-cell containing fractions were isolated from bone marrow and splenocytes. These fractions were applied to a Cellcelector™ to select and isolate cells each producing potentially different anti-TrkB antibodies. The RNA encoding VL and VH was isolated from each single cell, and reverse-transcribed into DNA copies. The copies were then cloned into DNA plasmid vectors used to transiently transfect HEK293 cells. Supernatants of the HEK293 cultures were used to discover unique TrkB-specific antibodies. Sequence analysis of all combinations of VL/VH sequences, and subsequent ELISA and flow-cytometric screening revealed the discovery of 9 potential TrkB-binders.

The 10 chimeric chicken-human antibodies were further investigated for their cross-reactivity with BSA and with closely related TrkB proteins. ELISA analyses showed that all 9 clones show low to strong specific binding to TrkB.

Finally, the association and dissociation binding properties of the discovered chimeric human-chicken anti-TrkB mAbs were assessed using an Octet biosensor on which the antibodies were immobilized and exposed to 300 nM human TrkB-His to accomplish the off-rate kinetic measurements. The dissociation process was monitored and the first 30 s were modelled to determine the dissociation constants. These constants and other biochemical characteristics of selected chimeric anti-TrkB mAbs are summarized in Table 6.

TABLE 6
Summary of measured biological and biochemical properties of a selection
of the discovered chicken B-cell-derived anti-TrkB antibodies. TrkB
activation: activation of a TrkB signaling pathway in modified functional
U2OS cells in a TrkB PathHunter ® eXpress assay.

| | Cell- | Cell- | | | | |
|---|---|---|---|---|---|---|
| | associated | associated | hTrkB | mTrkB | | TrkB |
| mAbs | hTrkB | mTrkB | reactivity | reactivity | K_{dis} | activation |
| clone | reactivity | reactivity | (ELISA) | (ELISA) | (1/s) | (EC₅₀ in μg/mL) |
| F1.3 | + | ++ | ++++ | ++ | 1.56E−02 | 3.8E−01 |
| B11 | ++++ | ++++ | ++++ | +++ | 4.34E−03 | 8.6E−07 |
| A12 | ++++ | ++++ | +++++ | +++ | 1.76E−03 | 4.5E−03 |
| B5 | ++++ | ++++ | +++++ | +++ | 1.14E−03 | 8.9E−03 |
| D5 | ++ | ++ | ++++ | +++ | 2.19E−04 | 1.8E−01 |
| B1.2 | ++++ | ++++ | +++++ | +++ | 4.83E−04 | 7.5E−03 |
| B4.2 | ++++ | ++++ | +++++ | +++ | 1.36E−03 | 8.2E−09 |
| E2 | ++++ | ++++ | +++ | + | 5.75E−04 | 1.7E−01 |
| F5 | ++++ | ++++ | ++++ | +++ | 1.63E−03 | 4.6E−02 |
| E4 | +++ | ++ | ++++ | ++ | ND | ND |

Reactivity towards cell-associated human or murine TrkB is performed by flow cytometry. The binding to plate-immobilized antigens was assessed by ELISA. Ch, chicken origin; nd, not determined.
+++++ = strong;
++++ = medium/strong;
+++ = medium;
++ = low/medium;
+ = low/weak

C) Epitope binning of anti-TrkB antibodies

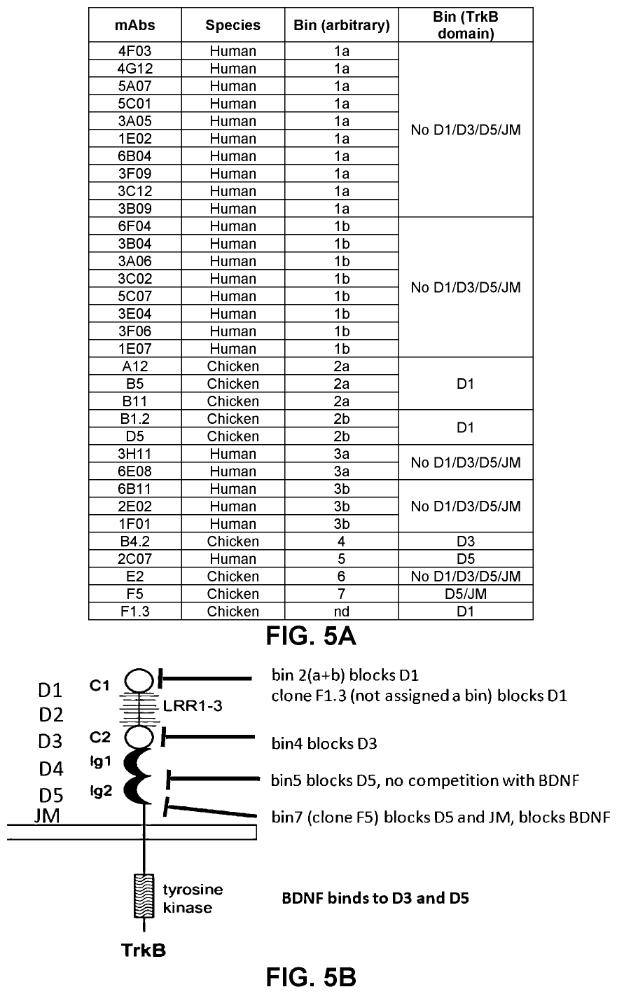

Part of the triage workflow involved the label-free biosensor screenings of pairwise competition analysis of all mAb clones. All mAb clones were used both as primary and as secondary analyte. This combinatorial pairwise mAb competition or “epitope binning” revealed several distinct epitope clusters of mAbs sharing similar blocking profiles, viz. bins 1 to 7. Epitope bins 1, 2 and 3 clones showed nuanced interference with each other, and so were assigned to sub-bins 1a or 1b, 2a or 2b, and 3a or 3b (FIG. 5A). For clone F1.3, no distinct bin could be distinguished due to self-blocking in the tandem assay and poor binding in the sandwich assay (indicated with ‘nd’).

Subsequently, a panel of reference mAbs with described epitopes, or BDNF known to bind to TrkB domains D3 and D5, were tested for competition with selected mAbs from each bin for binding to TrkB immobilized to the sensor surface. The inter-bin blocking relationships using the ‘structural’ benchmark mAbs revealed that the selected clones from bin 2, and by inference all clones in this bin, and clone F1.3 presumably bind to TrkB domain D1, while the selected clones from bin 4, and by inference all clones in bin 4, block domain D3. Remarkably, clone 2C07, which falls in bin 5, blocked D5, but did not compete with BDNF. Clone F5, which falls in bin 7, competes for binding to TrkB domains D5 and JM, and competes with BDNF (FIGS. 5A and 5B).

### D) Functional Activity of Anti-TrkB Antibodies in a Cell-Based TrkA Activity Assay

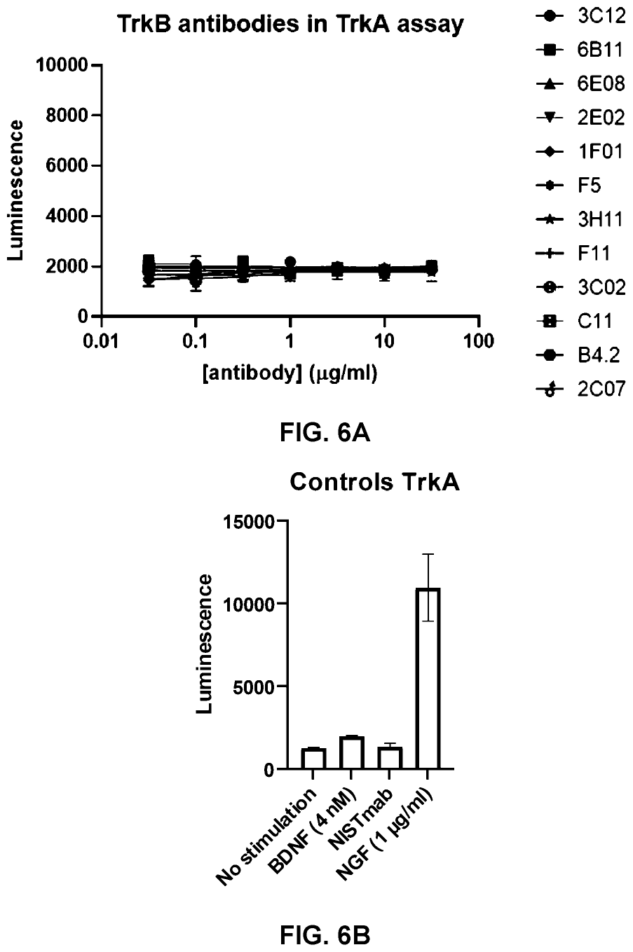

The TrkA receptor is a homologues member in the Trk-family of neurotrophin receptors, and TrkA and TrkB have considerable structural similarities. Binding and activation of TrkA by anti-TrkB antibodies should be avoided to prevent adverse effects from the therapeutic use of anti-TrkB antibodies. None of the tested anti-TrkB antibodies was able to trigger TrkA-dependent signaling in dedicated, modified U2OS cells in a PathHunter® eXpress assay (FIG. 6A), whereas TrkA's natural ligand, NGF, triggered a significant signal (FIG. 6B).

### E) Functional Activity of Anti-TrkB Antibodies in a Cell-Based TrkB Activity Assay

In a functional cell-based assay probing activation of exclusively TrkB (PathHunter® express), the ability of the anti-TrkB antibody candidates to stimulate a TrkB-signaling pathway was tested. In this assay, control antibody NISTmab failed to induce a significant signal. In contrast, all tested anti-TrkB antibodies triggered the activation of a specific acceptor-enzyme in the cell system showing different activation profiles. All anti-TrkB antibodies discovered from the exploited human phage display libraries revealed a concentration-dependent activation with a hook effect for some of the candidates, such as clones 3H11, 4G12, and 5A07. However, for chicken-derived antibodies, a concentration-dependent activation is less pronounced and less evident for several clones, such as A12, B1.1, or B5. The EC50-values as a way to express the potency of the anti-TrkB antibodies to activate the TrkB-signaling pathway in this cell system, are summarized in Tables 5 and 6.

### F) Induction of Phosphorylation of Cell-Attached TrkB by Anti-TrkB Antibodies

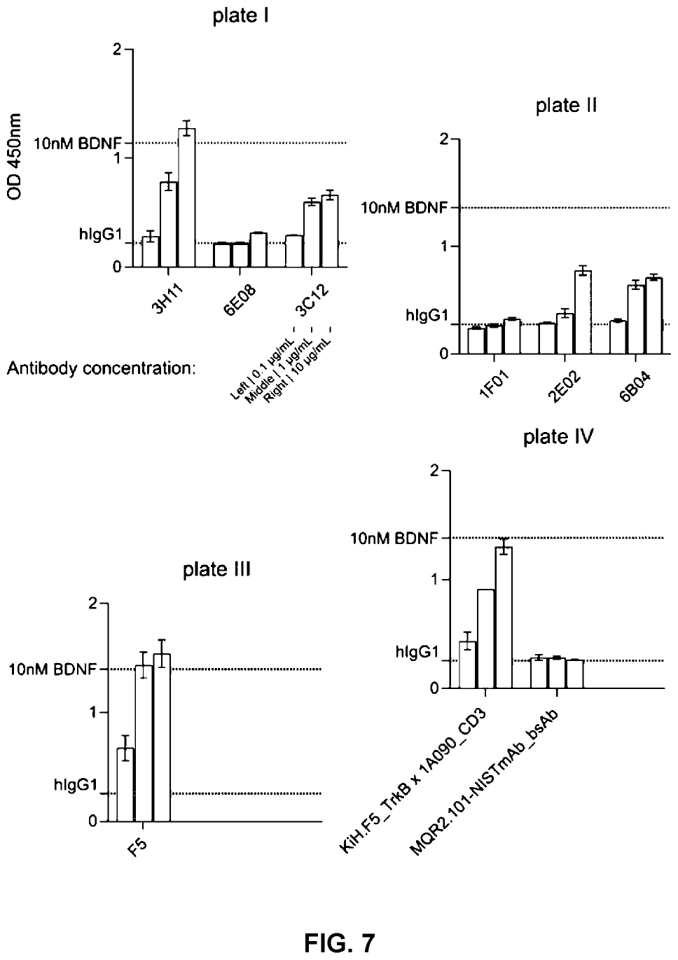

Activation of functional TrkB was investigated in an alternative way by determining the capacity of anti-TrkB antibodies to induce auto-phosphorylation of cellular TrkB (FIG. 7). At a concentration of 10 μg/mL, all tested anti-TrkB antibodies (clones 3H11, 6E08, 3C12, 1F01, 2E02, 6B04, and F5) were able to significantly trigger phosphorylation of the TrkB target. Potent phosphorylation of the protein was observed for clones 3H11 and F5 in excess of positive control, TrkB-ligand BDNF, at 10 nM. At the lowest test concentration, 0.1 μg/mL, a substantial signal for phosphorylation was only found for chicken-derived clone F5.

### G) Internalization Assay

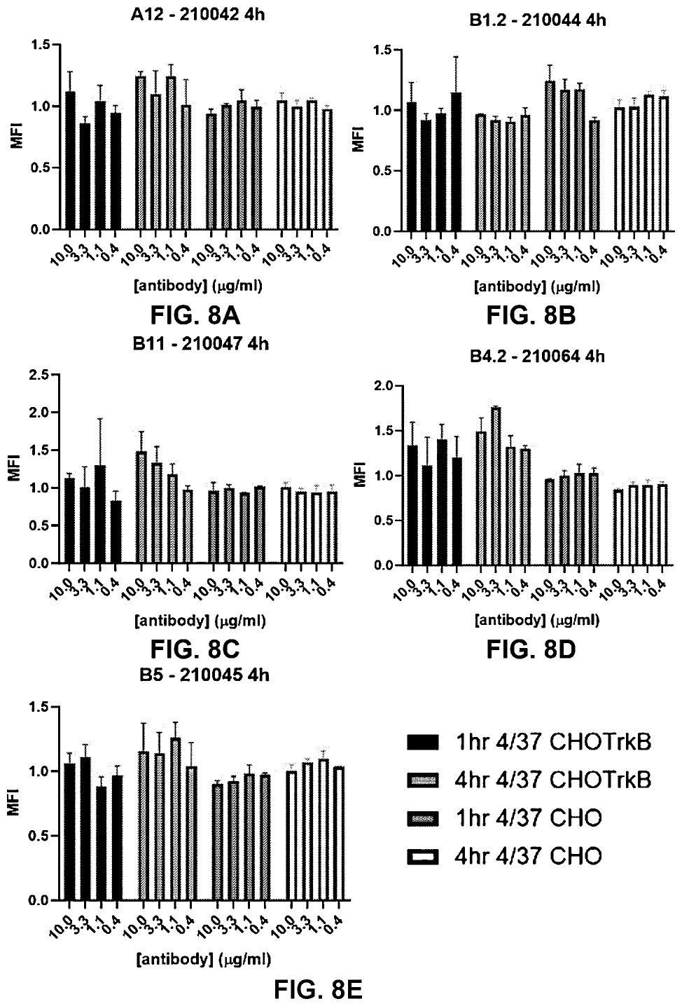

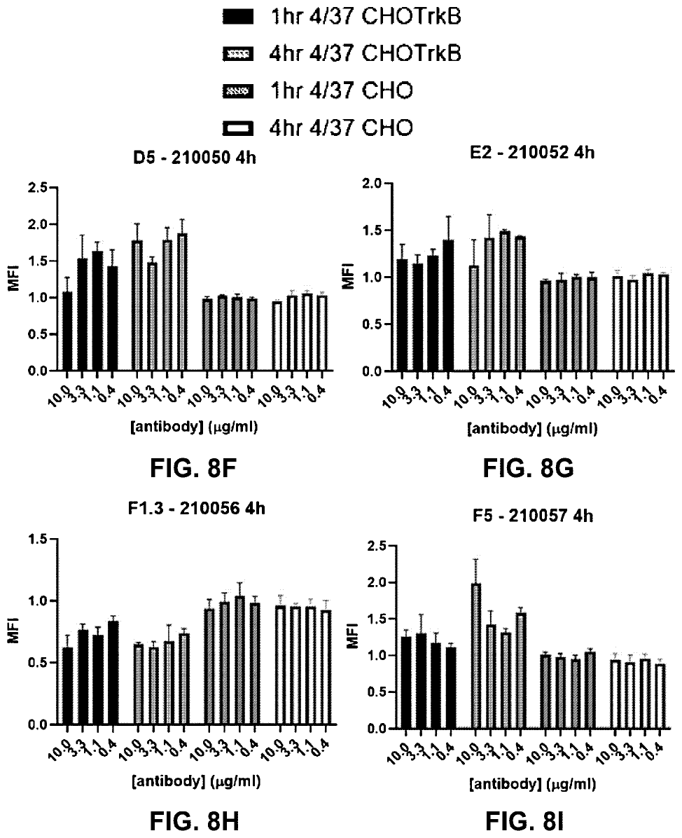

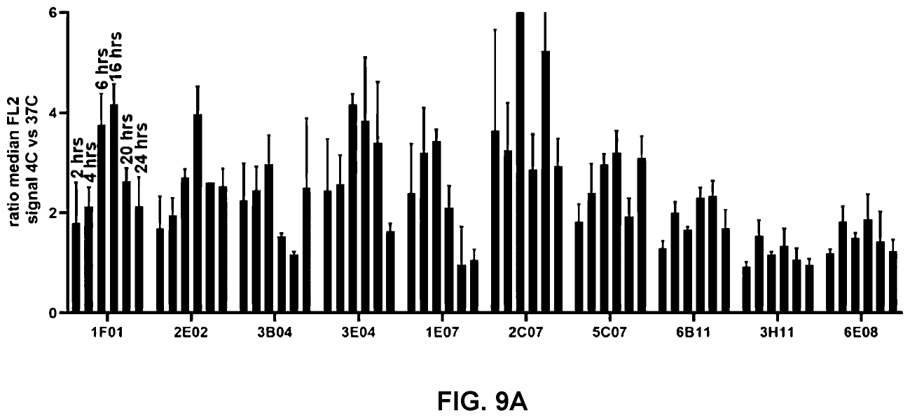

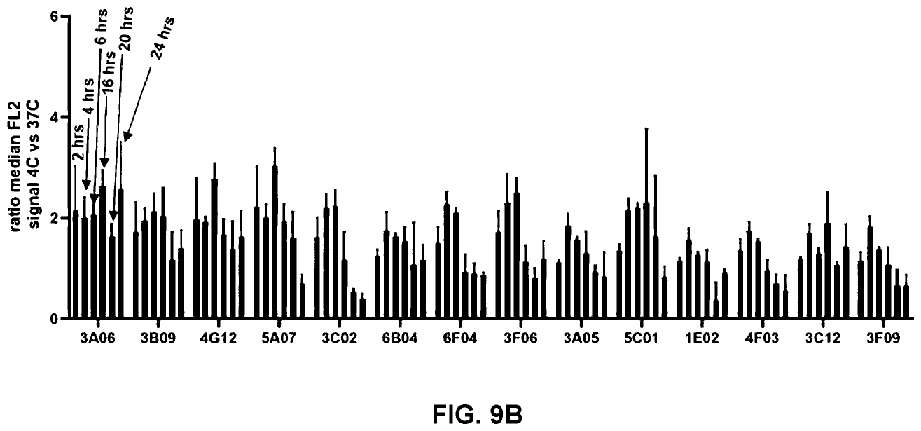

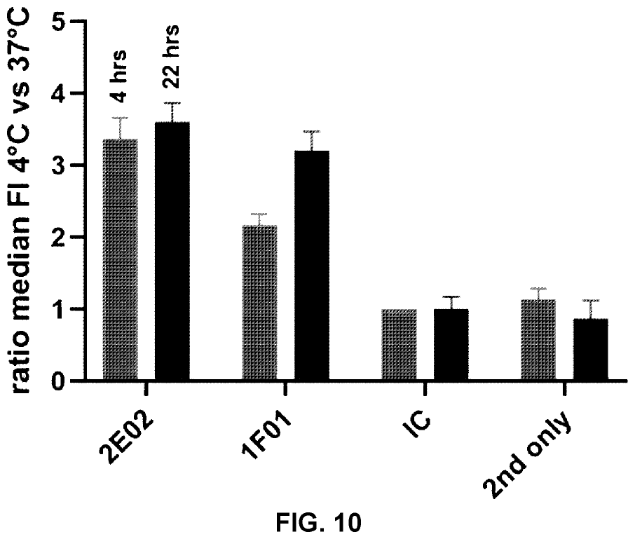

The ability of the anti-TrkB antibodies to internalize was determined by the residual receptor presence at the surface method using CHO-k1 huTrkB cells and CHO-k1 parental cells. The results are reported in FIGS. 8A-81 (chicken clones) and FIGS. 9A-B (human antibodies). The ability to internalize of anti-TrkB human antibody clones 2E02 and 1F01 in a lung carcinoma derived cell line (A549) was also tested; the results are reported in FIG. 10.

The sequences of the heavy and light chain variable regions of the 10 anti-TrkB chicken-derived clones with reactivity to TrkB are depicted in Table 7.

TABLE 7
Amino acid sequences of the heavy and light chain variable regions of the 10
anti-TrkB chicken-derived clones.

| Clone | | |
|---|---|---|
| ID | HCVR | LCVR |
| A12 | AVTLDESGGGLQTPGGALSLVCKASGFT | QAALTQPASVSANPGETVKITCSGG |
| | FSSFPMVWVRQAPGKGLEFVAGIVRTST | SSDYGWFQQKSPGSAPVTVIYYSDK |
| | PKYGAAVTGRATISRDNGQSTVRLQLNN | RPSDIPSRFSGSASGSMGALIITGVQ |
| | LRAEDTGTYFCAKSATICGNCPEDIDAW | AEDEAVYFCGSRDSSNVAIFGAGTT |
| | GHGTEVIVSS (SEQ ID NO: 374) | LTVL (SEQ ID NO: 375) |
| B5 | AVTLDESGGGLQTPGGALSLVCKASGFT | QAALTQPASVSANPGETVKITCSGG |
| | FSSFPMVWVRQAPGKGLEFVAGIVRTST | SSDYGWFQQKSPGSAPVTVIYYSDK |
| | PKYGAAVTGRATISRDNGQSTVRLQLNN | RPSDIPSRFSGSTSGSMGALIITGVQ |
| | LRAEDTGTYFCAKSATICGNCPEDIDAW | AEDEAVYFCGSRDSSNVAIFGAGTT |
| | GHGTEVIVSS (SEQ ID NO: 376) | LTVL (SEQ ID NO: 377) |
| B1.2 | AVTLDESGGGLQTPGGGLSLVCKASGF | QAALTQPSSVSANPGETVKITCSGP |
| | TFSNHQMAWVRQAPGKGLEYVGLINAA | TYDYGWFQQKSPGSAPVTVIYYNDN |
| | GSWTYYGTAVKGRATISRDNGQSTVRL | RPSDIPSRFSGSKSGSTATLTITGVQ |
| | QLNNLRAEDTATYYCAKDVYGYCSTCG | AEDEAVYYCGTYEDNNGASFGAGT |
| | ATIDTWGHGTEVIVSS (SEQ ID NO: 378) | TLTVL (SEQ ID NO: 379) |
| B11 | AVTLDESGGGLQTPGGALSLVCKASGFT | QAALTQPASVSANPGETVKITCSGG |
| | FSSFNMFWVRQAPGKGLEFVAHIKRNG | GRWYGTYYYGWYQQKSPGSAPVT |
| | GETDYGAAVKGRATISRDDGQSTVRLQL | VIYSNDKRPSDIPSRFSGSASGSTAT |
| | NNLRAEDTATYFCAKDAGDNCRWNYCT | LTITGVQADDEAVYYCGSWDSTDTT |
| | TNIDAWGHGTEVIVSS (SEQ ID NO: 380) | AFGAGTTLTVL (SEQ ID NO: 381) |
| B4.2 | AVTLDESGGGLQTPGGGLSLVCKASGF | QAALTQPASVSANPGETVKITCSGG |
| | TFSSYTMQWVRQAPGKGLEWVAGICND | GSYYGSYYYGWYQQKSPGSAPVTV |
| | GTTTNYGSAVKGRATISRDNGQSTLRLQ | IYNNDKRPSDIPSRFSGSLSGSTNTL |
| | LNNLRAEDTGIYFCTRGVDICWNSGNNI | TITGVQVEDESVYFCGSKDSSGAGI |
| | DAWGHGTEVIVSS (SEQ ID NO: 713) | YGAGTTLTVL (SEQ ID NO: 714) |
| D5 | AVTLDESGGGLQTPGGPLSLVCEGSGF | QAALTQPASVSANLGGTVEITCSGG |
| | TFSSYGMAWVRQAPGKRLEWVASISST | SSGYGYGWYQQKSPGSAPVTVIYN |
| | GINAYYGAAVKGRATISRDNGQSTVRLQ | NNNRPSDIPSRFSGSLSDSTHTLTIT |
| | LNNLRAEDTGIYYCSKCAYSGSCSTSIDA | GVRAEDEAVYFCGAWESSATDIFGA |
| | WGHGTEVIVSS (SEQ ID NO: 382) | GTTLTVL (SEQ ID NO: 383) |
| E2 | AVTLDESGGGLQTPGGALSLVCKASGF | QAALTQPSSVSANPGETVKITCSGSI |
| | DFSSNAMGWVRQAPGKGLEFIASITTDG | NNYGWYQQKSPGSAPVTVIYQNDK |
| | SYTTYGAAVKGRATISRDDGQSTLRLQL | RPSDIPSRFSGSKSGSTGTLTITGVQ |
| | NNLRAEDTATYYCAKDSSIHWNGYDIDA | AEDEAVYFCGSEDSRTYLGIFGAGT |
| | WGHGTEVIVSS (SEQ ID NO: 384) | TLTVL (SEQ ID NO: 385) |
| E4 | AVTLDESGGGLQTPGGGLSLVCKASGF | QAALTQPASVSSNPGETVKITCSGG |
| | TFSSHAMFWVRQAPGKGLEYVAYINYD | SSGYAYGWYQQKSPGSAPVTLIYSN |
| | GITFYGAAVKGRATISRDNGQSTVRLQL | DKRPSDIPSRFSGSKSGSTGTLTITG |
| | NNLRAEDTATYFCAKTTCSGCWDAAALI | VQAEDEAVYYCGSFDSSSYIFGAGT |
| | DAWGHGTEVIVSS (SEQ ID NO: 386) | TLTVL (SEQ ID NO: 387) |
| F1.3 | AVTLDESGGGLQTPGGALSLVCKASGFT | QAALTQPASVSANPGETVKITCSGG |
| | FSSFPMVWVRQAPGKGLEFVAGIVRTST | SSDYGWFQQKSPGSAPVTVIYYSDK |
| | PKYGAAVTGRATISRDNGQSTVRLQLNN | RPSDIPSRFSGSTSGSTNTLTITGVQ |
| | LRAEDTGTYFCAKSATICGNCPEDIDAW | VEDEAVYFCGSGDNSYVGFGAGTT |
| | GHGTEVIVSS (SEQ ID NO: 388) | LTVL (SEQ ID NO: 389) |
| F5 | AVTLDESGGGLQTPGGALSLVCKASGFT | QAALTQPASVSANPGETVKITCSGY |
| | FSSSGMAWVRQAPGKGLEFVAGITSSA | KSYYGWYQQKSPGSAPVTVIYDNTN |
| | GNTYYAPAVKGRATISRDNGQSTVRLQL | RPSNIPSRFSGSTSGSTATLTITGVQ |
| | NNLRAADTGTYFCAKDSNSGSPDASEID | ADDEAVYYCGNADHSYVGVFGAGT |
| | AWGHGTEVIVSS (SEQ ID NO: 390) | TLTVL (SEQ ID NO: 391) |

### Example 3: Results-Identification and Characterization of CD3—Specific Antibodies

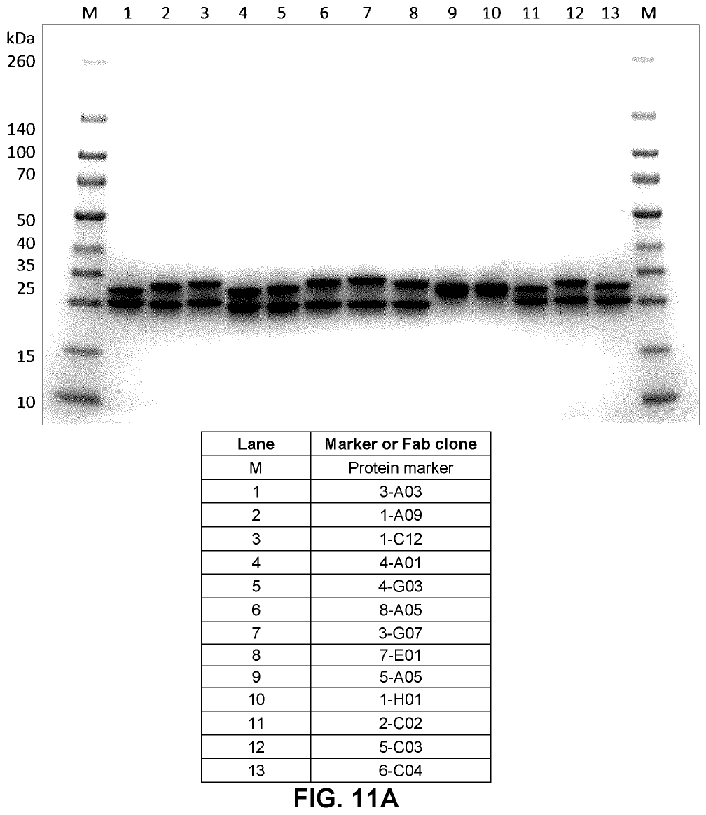

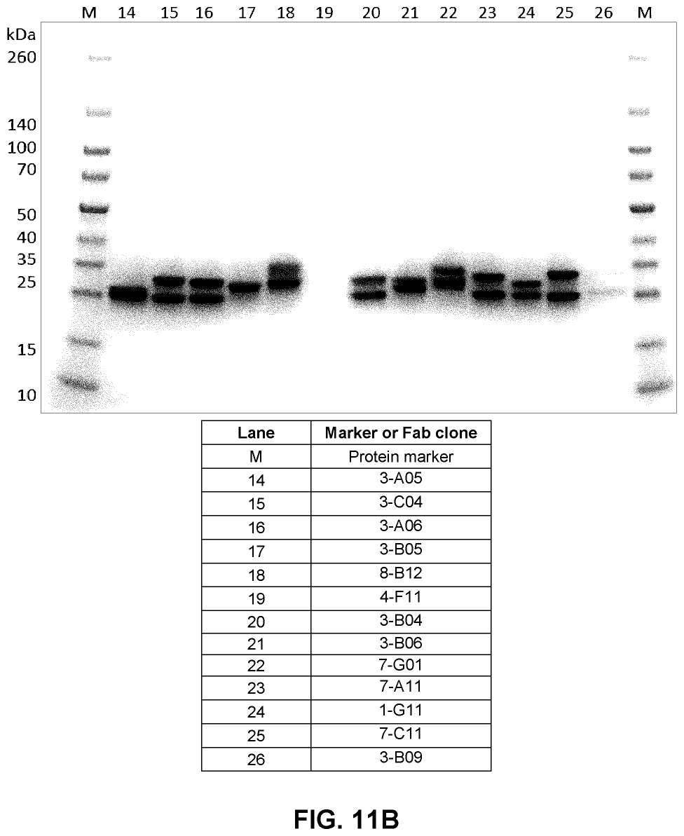

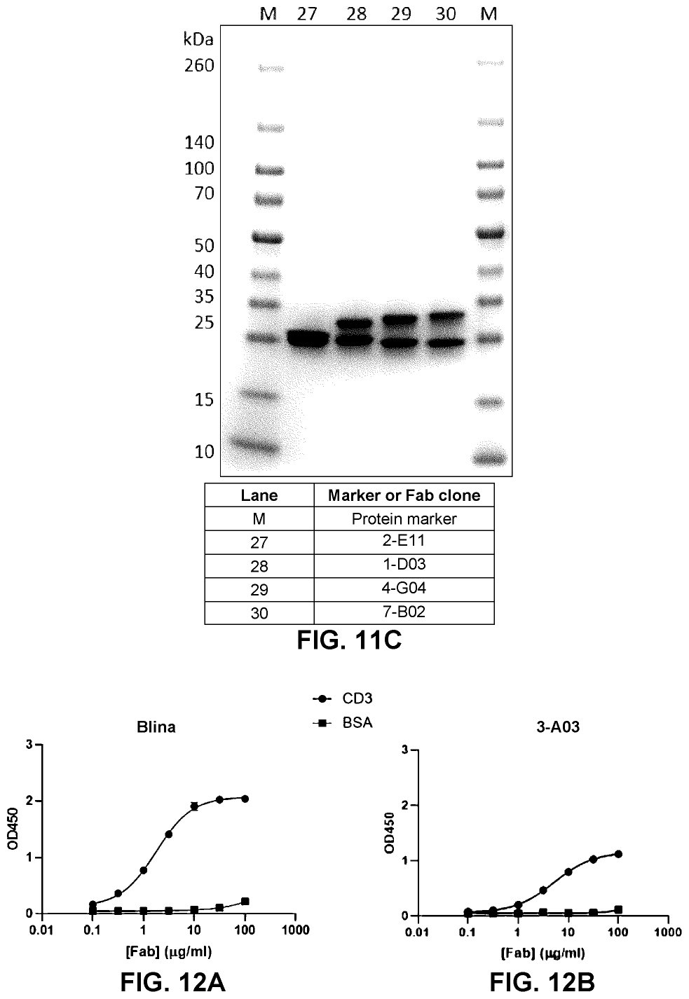

30 sequence-unique scFv clones (Table 8) were selected for their recombinant expression as Fab proteins in HEK293 cells. Cell-produced Fabs were isolated through CH1-affinity chromatography, and their integrity and purity were verified by SDS-PAGE (FIGS. 11A-C) and size-exclusion chromatography analyses.

TABLE 8
Amino acid sequences of the HCVR and LCVR of anti-CD3 scFv clones.

| Clone | | |
|---|---|---|
| ID | HCVR | LCVR |
| 3-A03 | EVQLVESGGGVVQPGRSLRLSCAASGF | QSVLTQPPSVSAAPGQKATISCSGGSSN |
| | TFSSYAMHWVRQAPGKGLEWVSAISGS | LGNNYVSWYQQLPGTAPKLLIYDNNKRP |
| | GGSTYYADSVKGRFTISRDNSKNTLYLQ | SGIPDRFSGSKSGTSAALAITGLQTGDEA |
| | MNSLRAEDTAVYYCARDAHDYWGQGTL | DYYCGTWDSSLSAGVFGGGTKLTVL |
| | VTVSS (SEQ ID NO: 636) | (SEQ ID NO: 637) |
| 1-A09 | EVQLVQSGGGLVKPGGSLRLSCAASGF | SYELMQPPSVSVSPGQTARITCSGFKLG |
| | TFSSYSMNWVRQAPGKGLEWVSSISSS | DKYVSWYQQKPGQSPVLVIYQDSKRPS |
| | SSYIYYADSVKGRFTISRDNSKNTLYLQM | GIPERFSGSNSGNTATLTISGTQAMDEAD |
| | NSLRAEDTAVYYCARVKDYDSSLDYWG | YYCQAWDSSTVVFGGGTKLTVL (SEQ ID |
| | QGTLVTVSS (SEQ ID NO: 638) | NO: 639) |
| 1-C12 | QVQLQQSGPGLVKPSQTLSLTCAISGDS | QSVLTQPPSASGTPGQRVTISCSGSSSNI |
| | VSSNSAAWNWIRQSPSRGLEWLGRTYY | GRNHVYWYQQLPGTAPKLLIYNNDQRPS |
| | RSKWYNDYAVSVKSRITINPDTSKNQFS | GVPDRFSGSKSGTSGSLTISGLQAEDEA |
| | LQLNSVTPEDTAVYYCARGITGTGYGMD | DYYCAAWDDSLSSGVFGGGTKVTVL |
| | VWGQGTLVTVSS (SEQ ID NO: 640) | (SEQ ID NO: 641) |
| 4-A01 | QVQLVQSEGGLVQPGGSLRLSCAASGF | NFMLTQPHSVSESPGKTVTISCTGSGGNI |
| | SFGSSAMSWVRQAPGRGLEWVSGISHS | GNNYVQWYQQRPDSPPSTVIYDDSQRP |
| | GSTTYYADSVKGRFTISRDNSKNTLYLQ | SGVPDRFSGSIDRSSNSASLTISGLRTED |
| | MNSLRVEDTAVYYCAKIGAYGYYFHYW | EADYYCQSSDSGRVVFGGGTKLTVL |
| | GQGTLVTVSS (SEQ ID NO: 642) | (SEQ ID NO: 643) |
| 4-G03 | QVQLLQSAGGLVQPGGSLRLSCAASGY | NFMLTQPHSVSESPGKTVTISCTGNGGNI |
| | SFGSSAMSWVRQAPGRGLEWVSGISHS | ASNYVQWYQHRPDSAPIAVIYDDSQRPS |
| | GSTTYYADSVKGRFTISRDNSKNTLYLQ | GIPDRFSGSIDRSSNSASLTISGLRPEDE |
| | MNSLRAEDTAVYYCAKIGAYGYYFHYW | ADYYCQSSDNANVVFGGGTKLTVL (SEQ |
| | GQGTLVTVSS (SEQ ID NO: 644) | ID NO: 645) |
| 8-A05 | QVQLQQSGGGLVQSGGSLRLSCAASGF | NFMLTQPHSVSESPGKTVTISCTRSGGSI |
| | SFGSSAMSWVRQAPGRGLEWVSGISHS | ASNYVQWYQQRPGSSPTTVIYDDNQRP |
| | GSTTYYADSVKGRFTISRDNSKNTLYLQ | SGVPDRFSGSIDSSSNSASLTISGLKTED |
| | MNSLRVEDTAVYYCAKIGAYGYYFHYW | EADYYCQSSDNINHVIFGGGTKLTVL |
| | GQGTLVTVSS (SEQ ID NO: 646) | (SEQ ID NO: 647) |
| 3-G07 | QVQLQQSGGGLVQPGGSLRLSCAASGF | NFMLTQPHSLSESPGKTVTISCTRSSGSI |
| | SFGSSAMSWVRQAPGRGLEWVSGISHS | ASKYVQWYQQRPGSAPTTVIYDDKQRP |
| | GSTTYYADSVKGRFTISRDNSKNTLYLQ | SGVPDRFSGSFDSSSNSASLTISGLKTED |
| | MNSLRVEDTAVYYCAKIGAYGYYFHYW | EADYYCQSFDSTNQAVVFGGGTKLTVL |
| | GQGTLVTVSS (SEQ ID NO: 648) | (SEQ ID NO: 649) |
| 7-E01 | QVQLQQSGGGLVQPGGSLRLSCAASGF | NFMLTQPHSVSESPGRTVTISCTRSSGSI |
| | SFGSSAMSWVRQAPGRGLEWVSGISHS | ASNYVQWYQQRPGSAPTTVIYEDNQRLS |
| | GSTTYYADSVKGRFTISRDNSKNTLYLQ | GVPDRFSGSIDRSSNSASLTISGLKTEDE |
| | MNSLRVEDTAVYYCAKIGAYGYYFHYW | ADYYCQSSDDSNHLVFGGGTKLTVL |
| | GQGTLVTVSS (SEQ ID NO: 650) | (SEQ ID NO: 651) |
| 5-A05 | QVQLVQSGAEGKKPGSSVKVSCKASGG | QPVLTQSPSASGTPGQRVTISCSGSSSNI |
| | TFSSYAISWVRQAPGQGLEWMGGIIPIF | GSNTVNWYQQLPGTAPKLLIYSNNQRPS |
| | GTANYAQKFQGRVTITADESTSTAYMEL | GVPDRFSGSKSGTSASLAISGLQSEDEA |
| | RSLRSDDTAVYYCARDAVVVADSGSMD | DYYCAAWDDSLNGWVFGGGTKVTVL |
| | VWGQGTTVTVSS (SEQ ID NO: 652) | (SEQ ID NO: 653) |
| 1-H01 | QVQLVQSGAEVKKPGSSVKVSCKASGG | QAGLTQPPSASGTPGQRVTISCSGSSSN |
| | TFSSYAISWVRQAPGQGLEWMGGIIPIF | IGSNTVNWYQQLPGTAPKLLIYSNNQRP |
| | GTANYAQKFQGRVTITADESTSTAYMEL | SGVPDRFSGSKSGTSASLAISGLQSEDE |
| | SSLRSEDTAVYYCARDAVVVADSGSMD | ADYYCAAWDDSLNGWVFGGGTKLTVL |
| | VWGQGTTVTVSS (SEQ ID NO: 654) | (SEQ ID NO: 655) |
| 2-C02 | QVQLQQSGPGLVKPSQTLSLTCAISGDS | QSVLTQPPSASGTPGQRVTISCSGSSSNI |
| | VSSNSAAWNWIRQSPSRGLEWLGRTYY | GSNYVYWYQQLPGTAPKLLIYRNNQRPS |
| | RSKWYNDYAVSVKSRITINPDTSKNQFS | GVPDRFSASKSGTSASLAISGLRSEDEA |
| | LHLNSVTPEDTAVYYCATWGMDVWGQ | DYYCASWDDSLRGPVFGGGTKLTVL |
| | GTTVTVSS (SEQ ID NO: 656) | (SEQ ID NO: 657) |
| 5-C03 | QVQLQQSGPGLVKPSQTLSLTCAISGDS | QPVLTQPPSASGTPGQRVTISCSGSSSNI |
| | VSSNSAAWNWIRQSPSRGLEWLGRTYY | GSNYVYWYQQLPGTAPKLLIYRNNQRPS |
| | RSKWYNDYAVSVKSRITINPDTSKNQFS | GVPDRFSGSKSGTSASLAISGLQSEDEA |
| | LQLNSVTPEDTAVYYCATWGMDVWGQ | DYYCAAWDDSLNGPVFGGGTKLTVL |
| | GTTVTVSS (SEQ ID NO: 658) | (SEQ ID NO: 659) |
| 6-C04 | QVQLQQSGPGLVKPSQTLSLTCAISGDS | QPVLTQPPSASGTPEQRVTISCSGSSSNI |
| | VSSNSAAWNWIRQSPSRGLEWLGRTYY | GSNYVYWYQQLPGTAPKLLIYRNNQRPS |
| | RSKWYNDYAVSVKSRITINPDTSKNQFS | GVPDRFSGSKSGTSASLAISGLRSEDEA |
| | LHLNSVTPEDTAVYYCATWGMDVWGQ | DYYCAAWDDSLSGPVFGGGTKLTVL |
| | GTMVTVSS (SEQ ID NO: 660) | (SEQ ID NO: 661) |
| 3-A05 | QVQLLQSAAEVKKPGASVKVPCKASGY | QSALTQPHSVSESPGKTVTISCTRTSGRI |
| | TFTGHYMHWVRQAPGQGLEWMGIINPR | GSNYVQWYQQRPGSAPTVVIYEDNQRP |
| | GGSTSYAQKFQGRVTMTRDTSTSTVYM | SGVPDRFSGSIDSSTNSASLTISRLKTED |
| | ELSSLRSEDTAVYYCARESIAAAGPYFD | EADYYCQSYDSGVVVVFGGGTKLTVL |
| | YWGQGTLVTVSS (SEQ ID NO: 662) | (SEQ ID NO: 663) |
| 3-C04 | EVQLVESGGGLVQPGGSLRLSCAASGF | NFMLTQPHSVSESPGKTVTISCTRSTGSI |
| | TFNNFYMNWVRQAPGKGLEWISTISRG | AGNYVQWYQQRPGSSPTTVILEDKQRP |
| | GNSVYYADSVKGRFTVSRDNAKNSLFL | SGVPDRFSGSIDTSSNSATLTISGLKTED |
| | QMDSLRDEDTAVYYCVRGGDFLYWGQ | EADYYCQSYDSGNVIFGGGTKLTVL |
| | GTLVTVSS (SEQ ID NO: 664) | (SEQ ID NO: 665) |
| 3-A06 | QVTLRESGGGLVKPGGSLRLSCSASGF | NFMLTQPHSVSESPGKTVAISCSRSSGS |
| | TFRGFYMAWIRQAPGKGLEWVAYISSS | VASNYVQWYQQRPGNVPTTIIYQDNQRP |
| | GSAVYHADSVKGRFTVSRDNAKNLMYL | SGVPDRFSGSIDKSSNSASLTISGLKTED |
| | EMNSLRADDTAVYYCARDGGTGGFDY | EADYYCQSYDGRRLIFGGGTKLTVL |
| | WGQGTLVTVSS (SEQ ID NO: 666) | (SEQ ID NO: 667) |
| 3-B05 | QVQLLQSAGGVVQPGRSLRLSCAASGF | QSVLTQPPSVSAAPGQKVTISCSGSASNI |
| | TFSTYAVHWVRQAPGKGLEWVAAISYD | GNNYVSWYQQLPGTAPKLLIYDNNQRPS |
| | GAGKYYGDSVRGRFTISRDDSKNTVYLQ | GIPDRFSGSKSGTSATLGITGLQTGDEAD |
| | MNSLRAEDTAVYYCAGEHYTLTDEEFDF | YYCGTWDTSLRGVVFGGGTKVTVL (SEQ |
| | WGQGTLVTVSS (SEQ ID NO: 668) | ID NO: 669) |
| 8-B12 | HVQLVQSGGGSAQHGGSLRLSCAASGF | NFMLTQPHSVSESPGKTVTLSCTRSSGSI |
| | TFNNSYMNWVRQAPGKRLEWISTISRG | ASKYVQWYQQRPGSAPTIVIFDDDRRPS |
| | GNSVYYADSVKGRFTVSRDNTKNLTFLQ | GVPDRFSGSIDRSSNSASLTIFGMKTEDE |
| | MDSLRDEDTAVYYCAKGGDYLYWGQG | ADYYCQSYDSSNWVFGGGTKLTVL |
| | TLVTVSS (SEQ ID NO: 670) | (SEQ ID NO: 671) |
| 4-F11 | AVQLVESAGGVVQLRRSLSLSSAASRFT | QSVLTQPPSVSAAPGQKATISCSGGSSN |
| | FSSYPMHWIRQAPVKGMEWVSAISGSG | LGNNYVSWYQQLPGTAPKLLIYDNNKRP |
| | GSTYYADSVKGRFTISRHNSKNTLYLQM | SGIPDRFSGSKSGTSASLAITGLQTGDEA |
| | NSLRAEDTGVYWGARDPHDFWGQGTL | DYYCGTWDSSLSAGVFGGGTKLTVL |
| | VTVSS (SEQ ID NO: 672) | (SEQ ID NO: 673) |
| 3-B04 | QVQLVQSGGGLVQPGRSLRLSCAASGF | QSVLTQPPSVSAAPGKKVTISCSGGSSNI |
| | TFDDFAMHWVRQAPGKGLEWVSGITW | GKNYVSWYQQLPGTAPKLLIYDNNKRPS |
| | NSAATIYADSVKGRFTISRDNAKNSLYLQ | GIPDRFSGSKSGTSATLGITGLQTGDEAD |
| | MNSLRPEDTAVYYCARDRGGPKDPFDI | YYCGTWDSSLSAVVFGGGTKLTVL (SEQ |
| | WGQGTMVTVSS (SEQ ID NO: 674) | ID NO: 675) |
| 3-B06 | QVQLQQSGAEVKKPGASLKVSCKASGY | SYVLTQPPSVSVAPGQTAKITCGGDNIEN |
| | TFTRYGFSWVRQAPGQGLEWMGWISA | KYVHWYQQKPGQAPVVVVYDDSDRPSG |
| | YNGNTNYAQKFQGRVTMTTDTSTSTAY | IPDRFSGSNSGNTATLTITRVEAGDEADY |
| | MEMRSLRSDDTAVYYCARDRGYCSGG | YCQVWDSSSDRVVFGGGTKLTVL (SEQ |
| | SCPGIDAFDIWGQGTMVTVSS (SEQ ID | ID NO: 677) |
| | NO: 676) | |
| 7-G01 | QVQLVQSGPEVKKPGASVNISCKTSGYT | NFMLTQPHSVSESPGKTVTISCTGSSGSI |
| | FRNYYVHWVRQAPGQGFEYMGRVNPN | ASNYVHWYQQRPGSAPTTVIYEDNQRP |
| | SGNARYAQKFQGRVAMTSDTSTSTAYM | SGVPDRFSGSIDSSSNSASLTISGLKTED |
| | ELSSLRSEDTAVYYCAREPPATVAYYYG | EADYYCRSYDSSNRNWVFGGGTKLTVL |
| | MDVWGQGTTVTVSS (SEQ ID NO: 678) | (SEQ ID NO: 679) |
| 7-A11 | EVQLVESGGGVVQPGRSLRLSCAASGF | NFMLTQPHSVAESPGKTVTISCTRSSGSI |
| | TFSSYGIHWVRQAPGKGLEWVSAISGS | ASNYVQWYQQRPGSAPTTVIYEDNQRP |
| | GGSTYYADSVKGRFTISRDNAKNSLYLQ | SGVPDRFSGSIDSSSNSASLTISGLKTED |
| | MNSLRDEDTAVYYCARERLIGMDVWGQ | EADYYCQSYDSGNPRVFGGGTKLTVL |
| | GTTVTVSS (SEQ ID NO: 680) | (SEQ ID NO: 681) |
| 1-G11 | QVQLVQSGAEVKKPGASVKVSCKASGY | QPVLTQPPSASGTPGQRVTISCSGSSSNI |
| | TFTSYGISWVRQAPGQGLEWMGWISAY | GSKYVYWYQQLPGTAPKLLIYRNNQRPS |
| | NGNTNYAQKLQGRVTMTTDTPTSTAYM | GVPDRFSGSKSGTSASLVISGLRSEDEA |
| | EVRSLRSDDTAVYYCAREVNYGLDVWG | DYHCAAWDDSLSGRVFGGGTKLTVL |
| | QGTLVTVSS (SEQ ID NO: 682) | (SEQ ID NO: 683) |
| 7-C11 | EVQLVESGGGLVQPGGSLRLSCAASGF | NFMLTQPHSVSESPGKTVTISCTRNTGGI |
| | TSNTYAMSWVRQAPGKGLEWVSTISGS | AANYVQWYHQRPGSAPTTVIYEDKQRPS |
| | GSSTNYADSVKGRFTISRDSSKNTLYLQ | GVPGRFSGSIDGSSNSASLTISGLQAEDE |
| | MNSLRAEDTAVYYCARIERYSYGIDYWG | GDYYCHSSDNNNQVFGGGTKLTVL |
| | QGTLVTVSS (SEQ ID NO: 684) | (SEQ ID NO: 685) |
| 3-B09 | QVQLVQSGGGVVQPARSLRLSSAASGF | SYVLTQPPSVSVAPGQTARITCGGDNIGL |
| | TFSSYGMHWVRQAPGKRLEWVAVISPD | KSVHWYQQKPGQAPVLVVYDDVDRPSD |
| | GSDKYYADSVKGRFSVSRGNSKNMLYL | IPERFSGSNSGNTATLTISRVEAGDEADY |
| | QMTGLTTEDTAVYYCARNRKYTNFLDIW | YCQVWDDPSDQGSWVFGGGTKLTVL |
| | GQGTMVTVSS (SEQ ID NO: 686) | (SEQ ID NO: 687) |
| 2-E11 | QVQLVQSGAEVKKPGASVKVSCKASGY | EIVLTQSPGTLSLSPGERATLSCRASQSV |
| | TFTSYYMHWVRQAPGQGLEWMGIINPS | SSSYLAWYQQKPGQAPRLLIYGASSRAT |
| | GGTTSYAQKFQGRVTMTRDTSTSTVYM | GIPDRFSGSGSGTDFTLTISRLEPEDFAV |
| | ELSSLRSEDTAVYYCARNWASFDYWGQ | YYCQQYGSSPYTFGQGTKVDIK (SEQ ID |
| | GTLVTVSS (SEQ ID NO: 688) | NO: 689) |
| 1-D03 | EVQLVQSGGGLVKPGRSLRLSCTASGF | QSVLTQPPSVSAAPGQKVTISCSGSSSNI |
| | TFGDYAMSWFRQAPGKGLEWVGFIRSK | GNNYVSWYQQLPGTAPKLLIYDNNKRPS |
| | AYGGTTEYAASVKGRFTISRDDSKSIAYL | GIPDRFSGSKSGTSATLGITGLQTGDEAD |
| | QMNSLKTEDTAVYYCTRVSDIYGMDVW | YYCATWDDSLKAGVFGGGTKLTVL (SEQ |
| | GQGTTVTVSS (SEQ ID NO: 690) | ID NO: 691) |
| 4-G04 | QVNLRESGGGVVQPGGSLRLSCAASGF | NFMLTQPHSVSESPGRTVTISCTRSSGSI |
| | TFRRYGMNWIRQAPGKGLEWVSSISSS | ASNYVQWYQQRPGSSPTTVIYDDKKRPS |
| | SSYIYYADSVKGRFTISRDNAKNSLYLQM | GVPDRFSGSIDSSTNSASLTISGLKTEDE |
| | NSLRAEDTAMYYCVRDGRFINGLDVWG | ADYYCQSYDSSNHWVFGGGTKVTVL |
| | QGTLVTVSS (SEQ ID NO: 692) | (SEQ ID NO: 693) |
| 7-B02 | QVQLVQSGGGLVQPGGSLRLSCAASGF | SYVLTQPPSVSASPGQTASITCSGHKLG |
| | TFSRYSMSWVRQAPGKGLEWVSSISGG | DKYTSWYQQKPGQSPVLVIYQDNKRPS |
| | GGSTYYADSVKGRFTISRDNSDGSVYLK | GIPERFSGSNSGNTATLTISGTQAMDEAD |
| | MNSLAAGDTAAYYCVRGGWYRTPFEF | YYCQAWDSSTNYVFGTGTKLTVL (SEQ |
| | WGRGTLVTVSS (SEQ ID NO: 694) | ID NO: 695) |

The 28 clones produced with sufficient amounts for further analyses were characterized in more detail in several binding reactivity and activation assays.

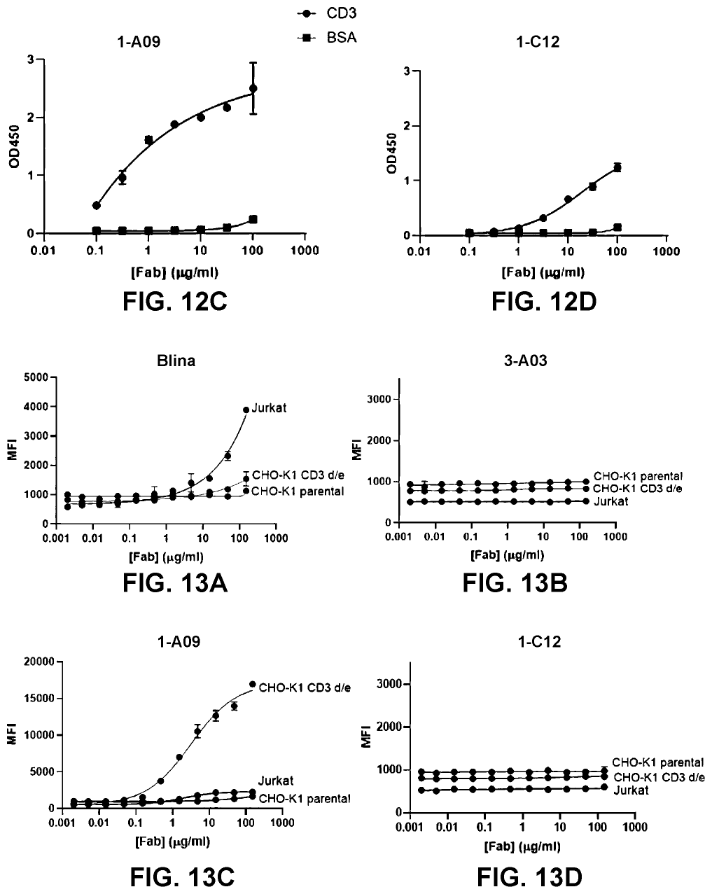

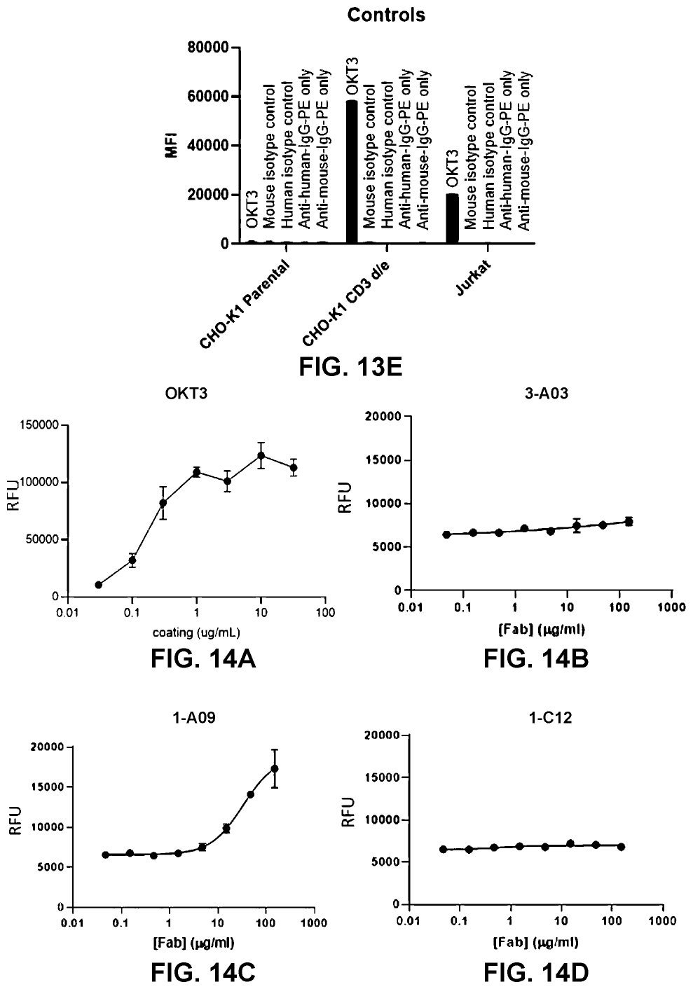

A dose-dependent CD30/8 reactivity for all 28 tested Fabs was determined by ELISA (FIGS. 12A-D, Table 9). However, although the CD3δ/ε-discriminatory reactivity was preserved, off-target reactivity to BSA was observed for 15 of the Fabs at higher concentrations. In contrast to ELISA, five Fab clones (1-A09, 8-A05, 3-G07, 3-A06 and 1-G11) showed dose-dependent reactivity towards CHO-K1 CD30/8-cells in flow cytometric analyses (FIGS. 13A-E, Table 9). The CD3-mediated activation potential of the 28 Fabs was investigated using nuclear factor of activated T-Cells (NFAT) Jurkat reporter cells (FIGS. 14A-D, Table 9). While reference full-length antibody OKT3 induced a very strong activation of the Jurkat cells, only clone 1-A09 out of all 28 tested synthesized Fabs gave a significant CD3 activation.

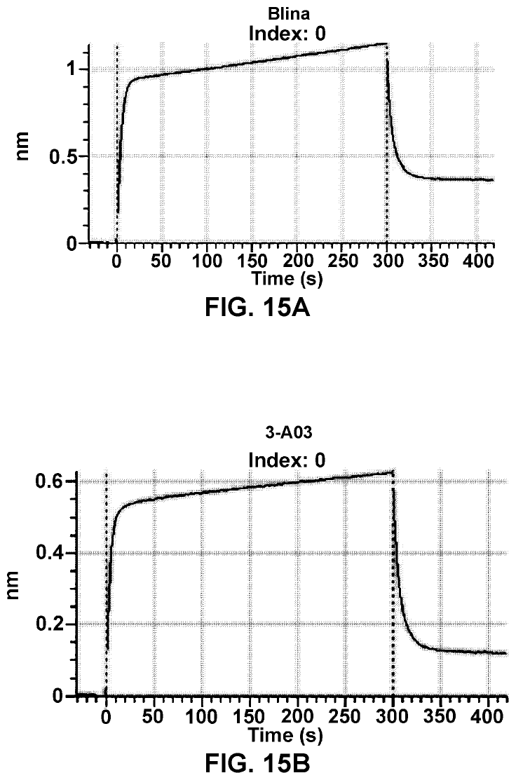

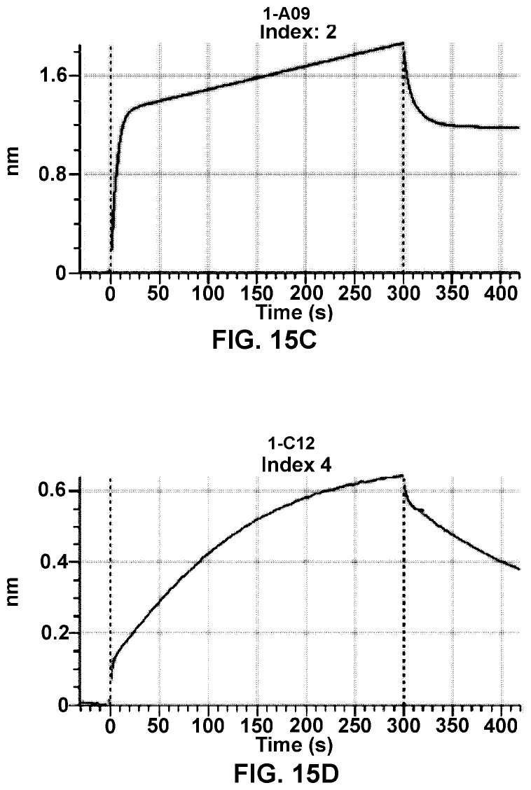

Finally, the association and dissociation binding properties of the discovered anti-CD3 Fabs were assessed using an Octet biosensor (FIGS. 15A-D, Table 9). The sensorgrams revealed a diverse binding profile for the Fabs reacting with sensor chip-immobilized biotinylated CD30/8, while not reacting with the sensor chip failing the antigen. Six candidates showed a slower dissociation rate relative to the Blina reference anti-CD3 Fab.

The properties of the 28 investigated monoclonal CD30/8-binding Fab entities and of the Blina CD3-binding reference are summarized in Table 9.

TABLE 9
Overview of the biochemical and biological characteristics
of 28 anti-CD3δ/ε Fab clones identified.

| | EC₅₀ | | CHO | | | |
|---|---|---|---|---|---|---|
| Fab clone | CD3δ/ε | Jurkat | CD3δ/ε | CHO par | Jurkat | |
| ID | (μg/mL) | reactivity | reactivity | reactivity | activation | Off-rate |
| 3-A03 | 5.2 | − | − + | − | + − | 1.65E−01 |
| 1-A09 | 2e−8* | + | ++ | − + | ++ | 1.38E−01 |
| 1-C12 | 19.2 | − | − | − | − | 2.07E−01 |
| 4-A01 | 19.5 | − | − +** | − + | − + | 6.14E−01 |
| 4-G03 | 16.8 | − | − +** | − + | − + | 6.12E−01 |
| 8-A05 | 0.5 | + − | + | − | − | 3.25E−01 |
| 3-G07 | 4.4 | + − | + | + − | − + | 5.02E−01 |
| 7-E01 | 7.9 | − | + −** | − + | − | 5.68E−01 |
| 5-A05 | 4.3 | − | − +** | − + | − | 1.34E−01 |
| 1-H01 | 6.8 | − | − +** | − + | − | 1.65E−01 |
| 2-C02 | 23.9 | + | + | + | − | 2.56E−01 |
| 5-C03 | 23.9 | + − | + −** | + − | − | 4.15E−01 |
| 6-C04 | 47.7 | + | +** | + | − + | 3.78E−01 |
| 3-A05 | 11.4 | − | − + | − | − | 3.25E−01 |
| 3-C04 | 5.9 | − | − + | − | − + | 4.60E−01 |
| 3-A06 | 3.6 | + − | + | − | − | 2.11E−01 |
| 3-B05 | 7.2 | − | − +** | − + | − | 4.11E−01 |
| 8-B12 | 38.2 | − | − | − | − | 5.14E−01 |
| 3-B04 | 10.9 | + − | +** | + | − | 4.77E−01 |
| 3-B06 | 16.9 | − | − | − | − | 3.47E−01 |
| 7-G01 | 6.3 | − + | − + | − | − + | 2.38E−01 |
| 7-A11 | 34.9 | − | − | − | − | 8.80E−01 |
| 1-G11 | 7.9 | n.d. | + | − | − | 5.81E−01 |
| 7-C11 | 52.3 | − | − | − | − | 7.99E−01 |
| 2-E11 | 53.6 | − | − | − | − + | 1.21E−01 |
| 1-D03 | 174.2 | − | − | − | − | 4.36E−01 |
| 4-G04 | 73.8 | − | − | − | − | 8.84E−01 |
| 7-B02 | 12.1 | + | +** | + | − | 1.68E−01 |
| Blina-Fab | 1.8 | ++ | + − | − | n.d. | 1.80E−01 |

*EC₅₀ value not accurately determined.
**no specific reactivity towards CHO-K1-associated CD3δ/ε observed.
Binding reactivities are indicated as: no reactivity or specific binding (−), weak (−+), significant (+) and strong (++). Off-rate data were obtained by fitting the course of association and dissociation of the anti-CD3δ/ε Fab using a CD3δ/ε-coated sensor in an Octet BLI biosensor. Blina-Fab (last row) was used as reference anti-CD3δ/ε Fab in all experiments, except the Jurkat cell activation assay.
n.d., not determined.

### Example 4: Results-Design of Bispecific Antibodies Directed Against TrkB and CD3

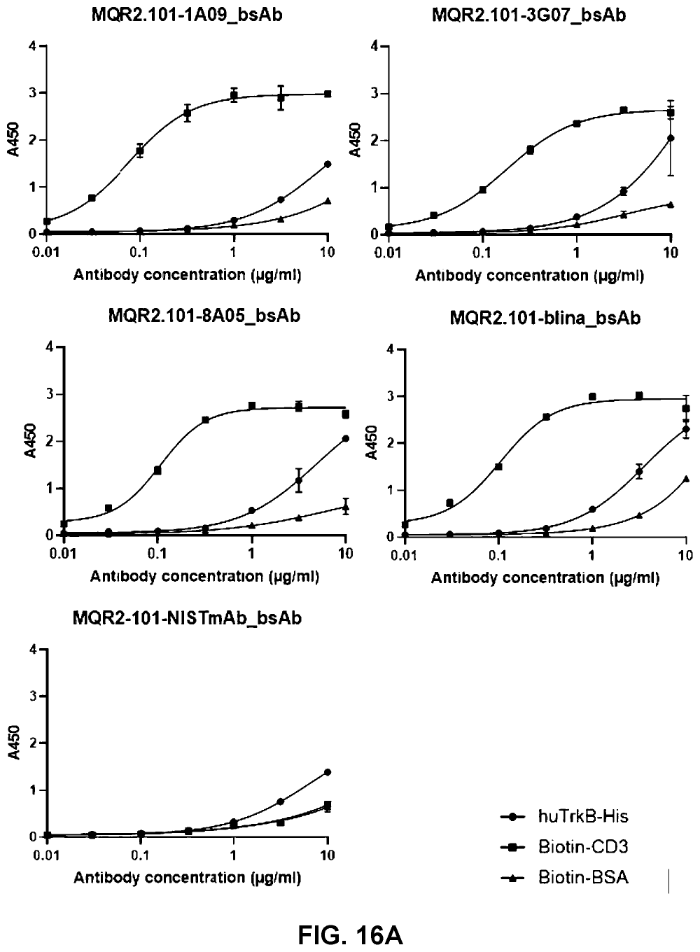

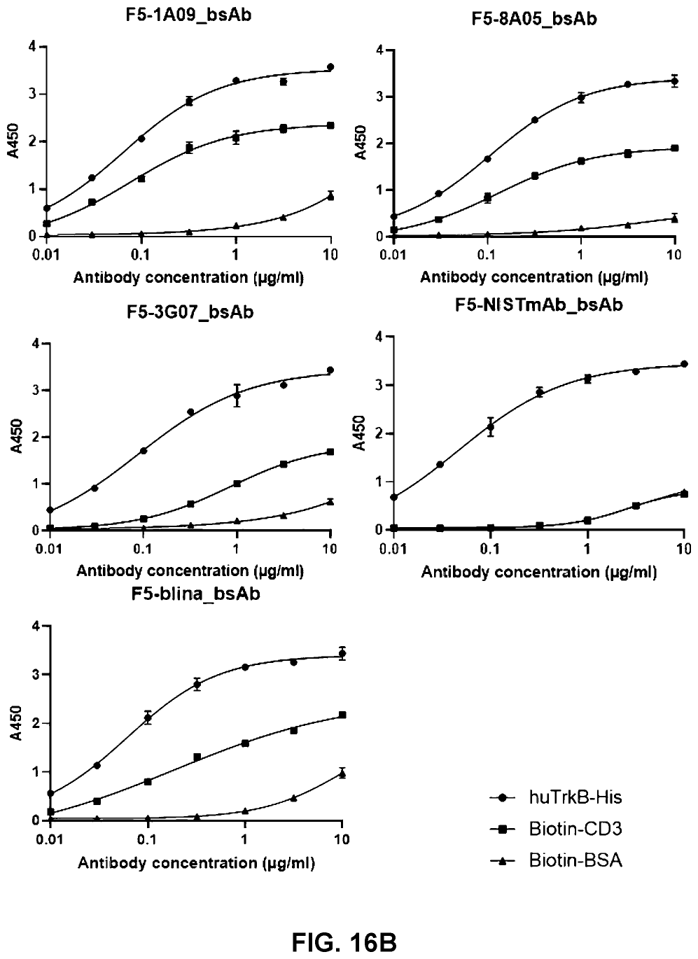

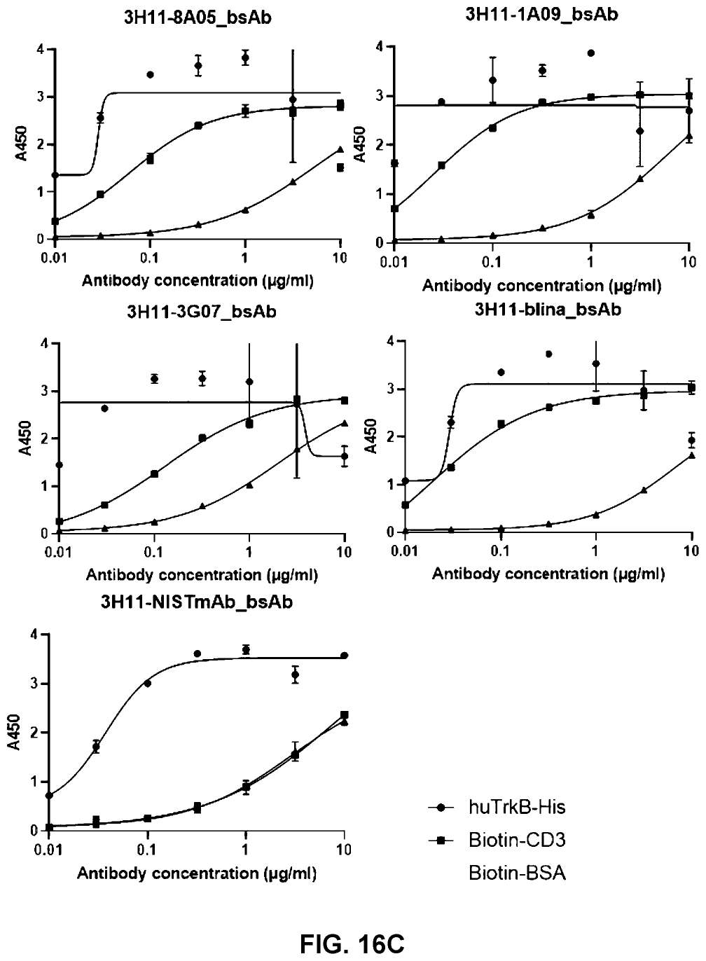

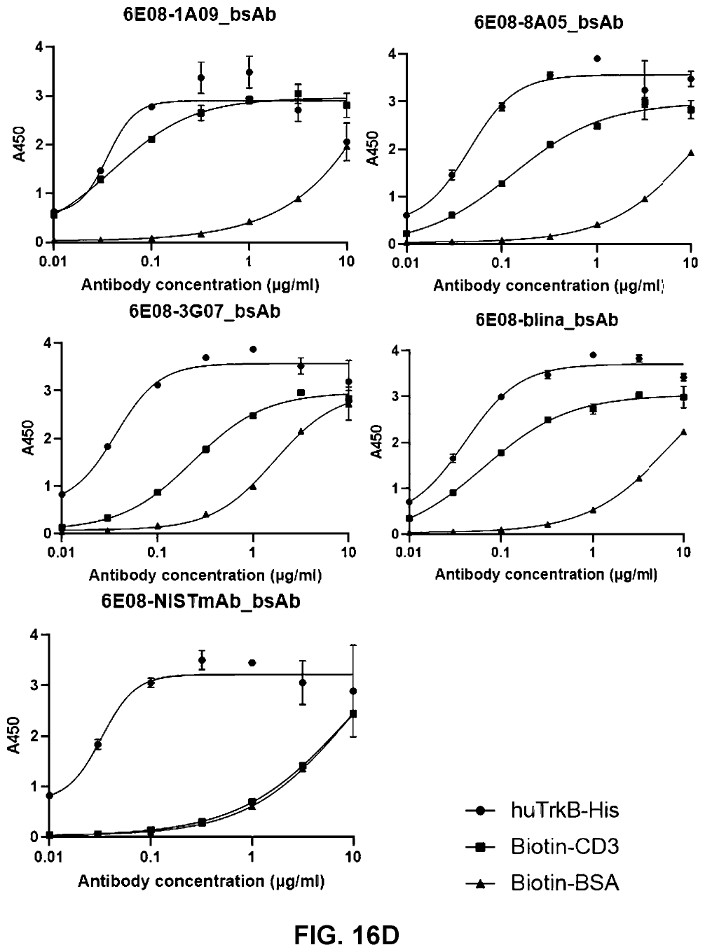

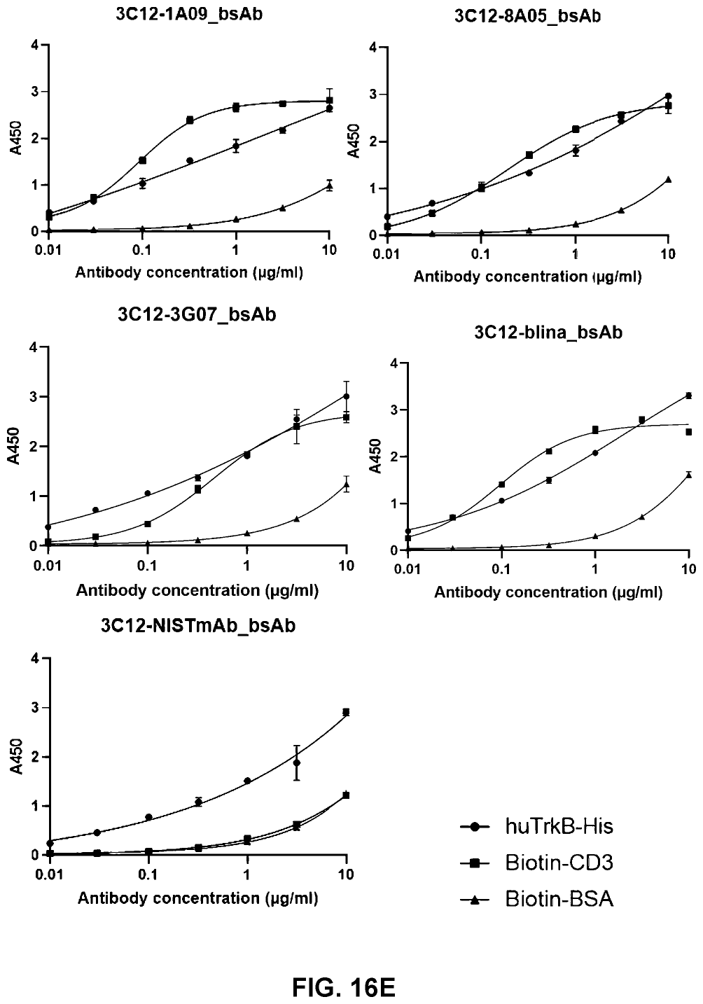

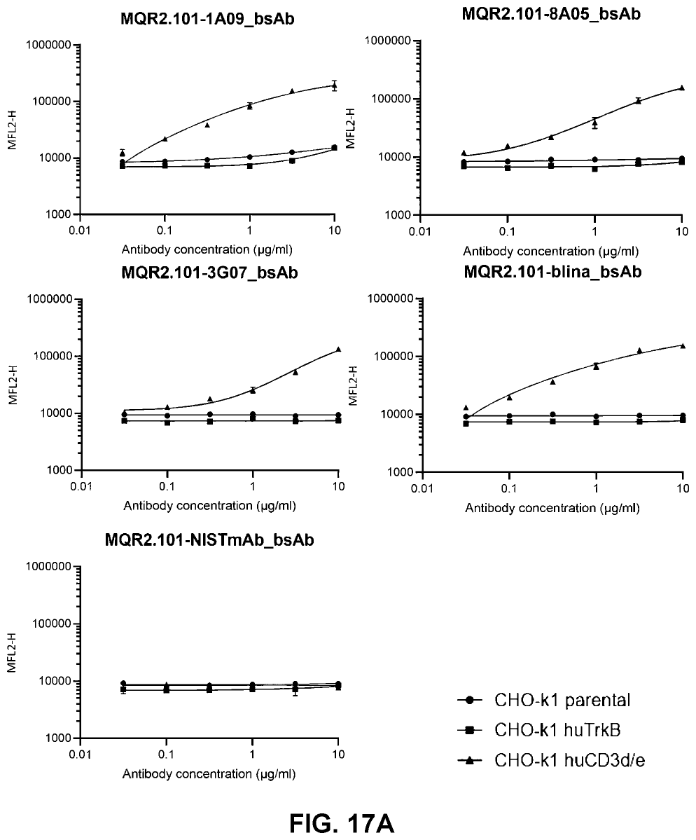

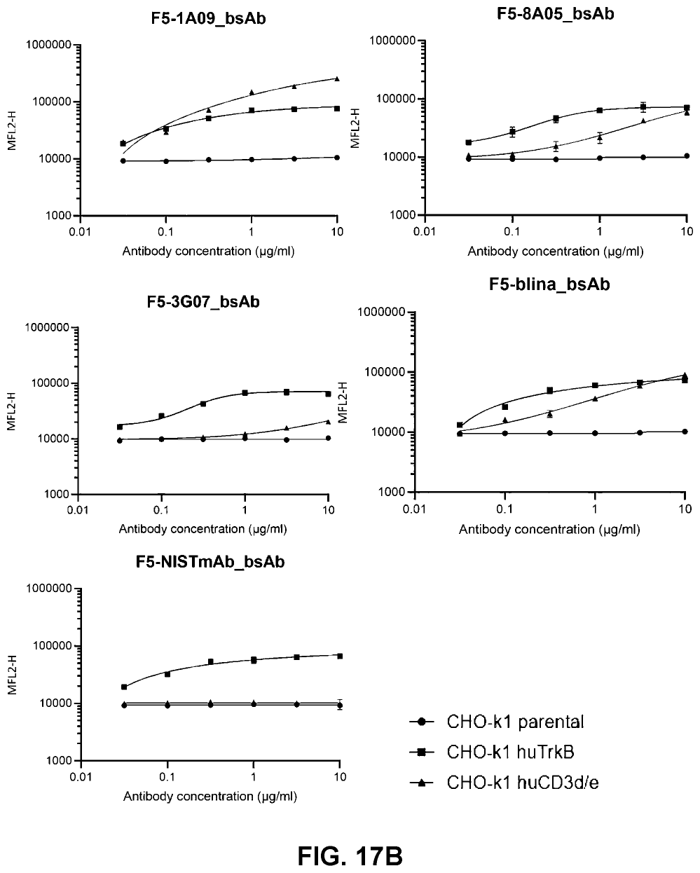

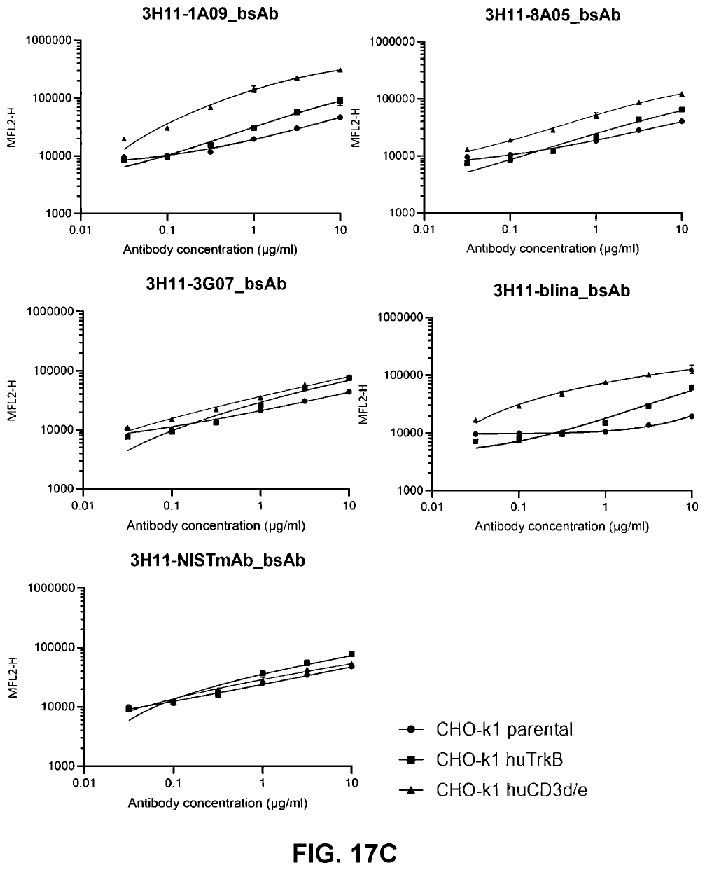

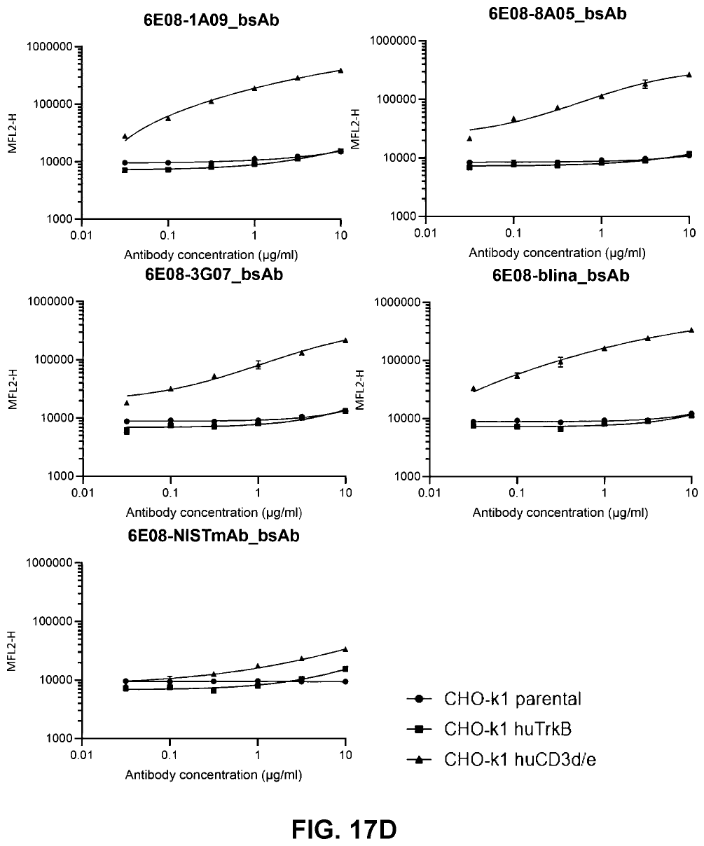

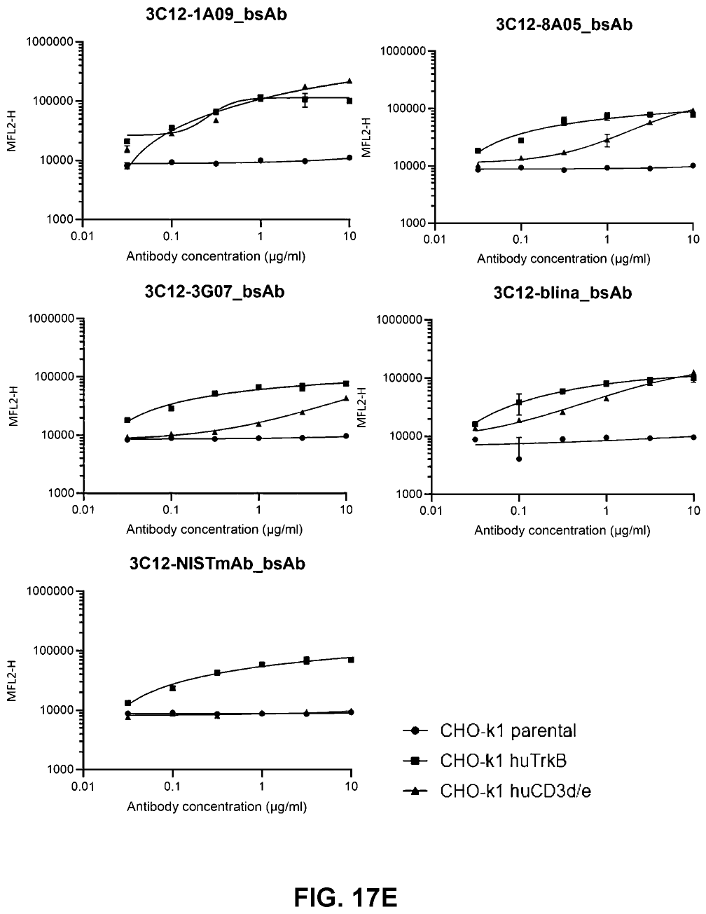

Using the knob-in-hole (KiH) approach, the binding sequences of TrkB were combined with CD3 and controls as summarized in Table 10. Those of the discovered anti-TrkB mAbs and of isotype control MQR2.101 were encoded as scFv-knob-Fc chains with a SSGGGGSGGGGSGGSAL linker sequence (SEQ ID NO:707) between the VH and VL domains. Here, the human IgG1-Fc part contained second generation knob mutations, namely T366W and S354C, as described in WO 1998/050431A2. At the other hand, the sequences of CD3 and of isotype control NISTmAb were encoded as Fab-Fc containing second generation hole-mutations, namely T366S, L368A, Y407V, and Y349C [WO 1998/050431A2]. All constructs were cloned into an appropriate vector and expressed in HEK293 cells. Integrity and purity of the harvested protein A-purified proteins were assessed by SDS-PAGE and size-exclusion chromatography. The binding capacity of the assembled xenobiotic bsAbs was assessed by ELISA using plates coated with either human TrkB-His or biotinylated CD3, or biotinylated BSA as negative control (FIGS. 16A-E). All bsAbs were shown to react specifically with their antigens. However, a specific binding with BSA is observed at the higher antibody concentrations as well. Additionally, the binding capacity of the same antibodies was assessed with a flow-based assay using CHO-k1 cells expressing either huTrkB, huCD3d/e or no recombinant protein (parental) (FIGS. 17A-E). The bsAbs containing the anti-TrkB clones F5 and 3C12 were shown to react specifically with their antigens. The bsAbs containing the anti-TrkB 3H11 clone also bound the antigens although the degree of specific binding is unclear as the background binding to the parental CHO-k1 cells was significant as well (FIG. 17C). The bsAbs were not polished which may have resulted in a high background due to the presence of parental mAbs and/or mAb aggregates. The bsAbs containing the anti-TrkB 6E08 clone did not bind to the TrkB antigen (FIG. 17D).

### Example 5: Results-Characterization of Bispecific Antibodies Against TrkB and CD3

### A) Pairing of Cells by Engineered Bispecific Antibodies Reactive Towards TrkB and CD3

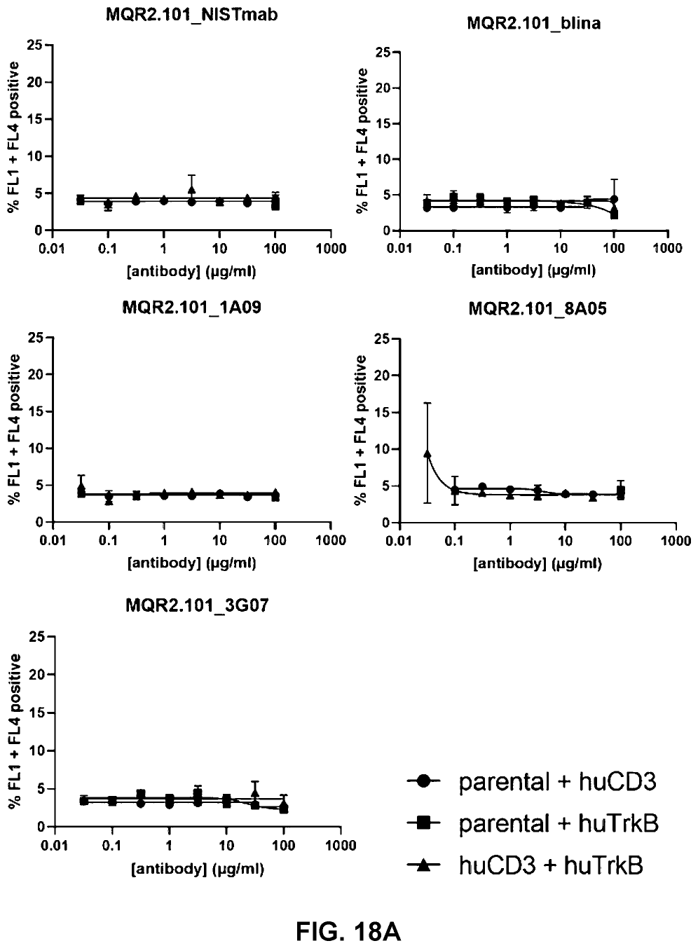

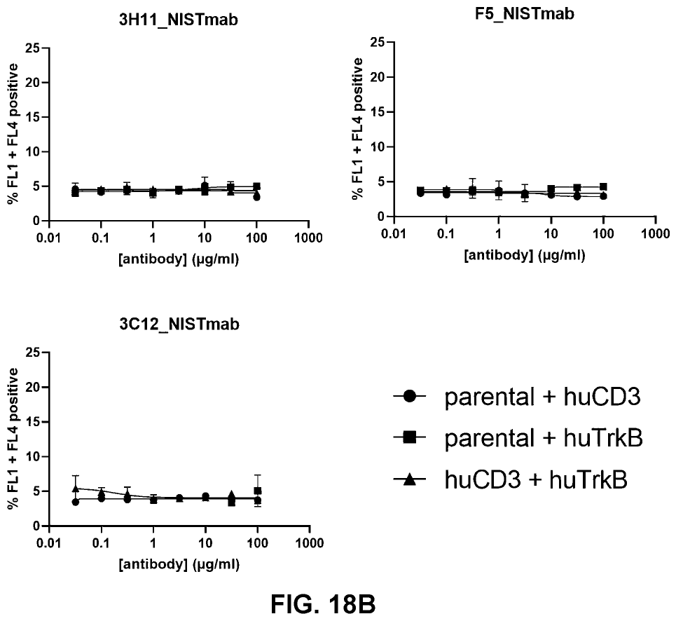

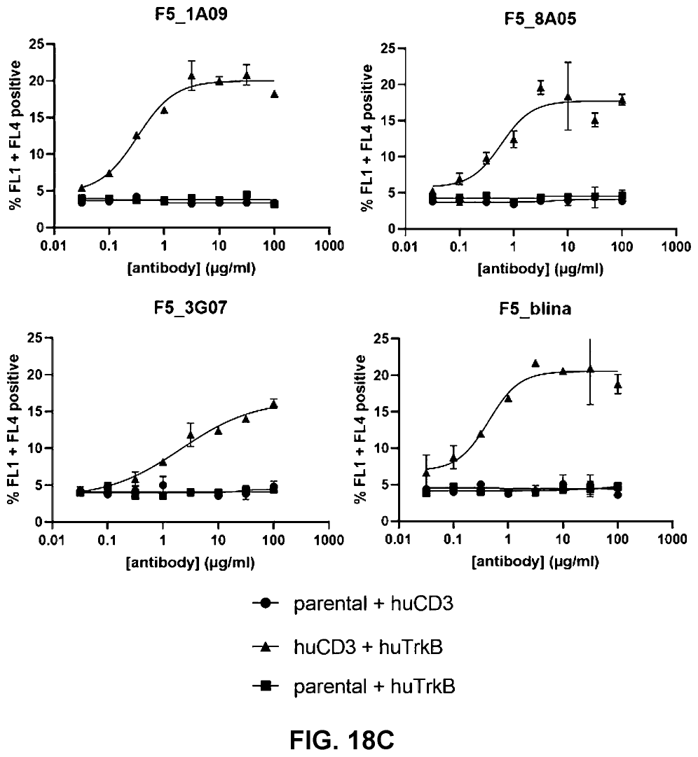

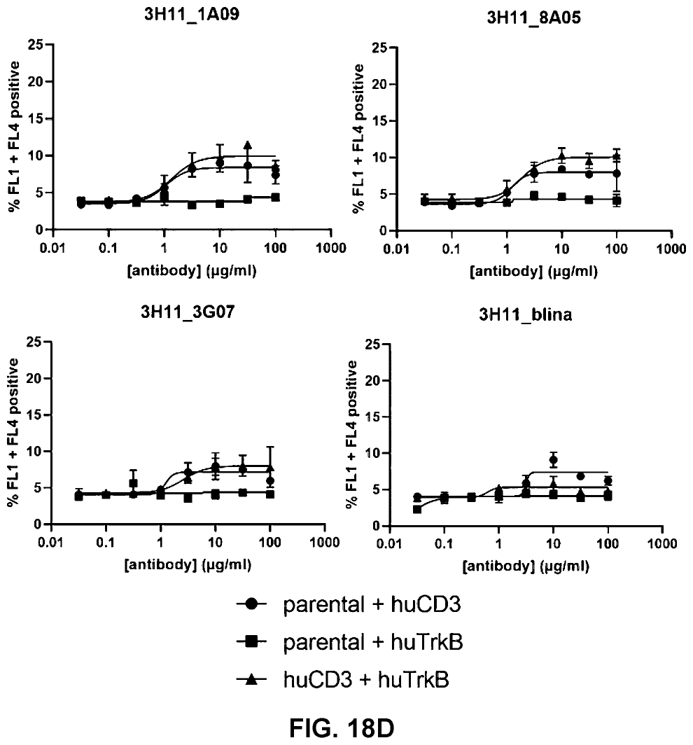

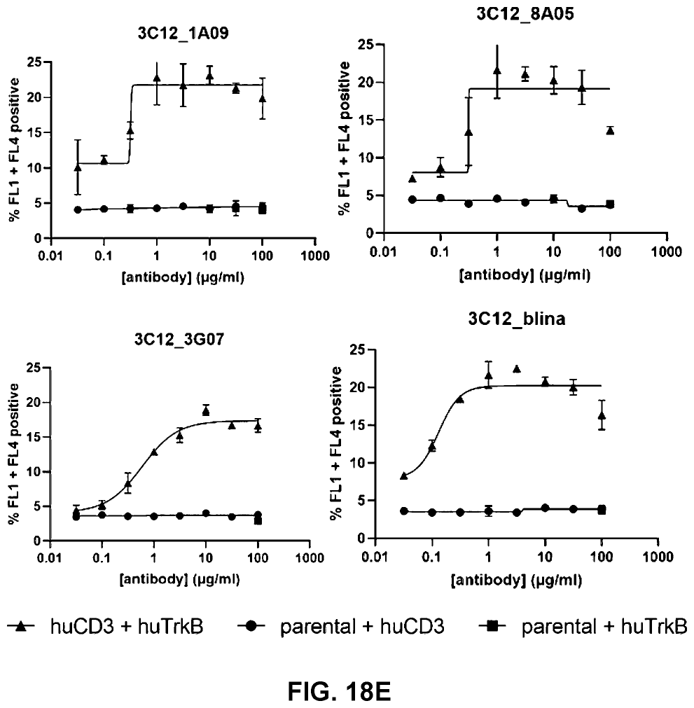

Using a cell-based bridging assay, the ability of the bsAbs to simultaneously bind both targets on different cells was investigated (FIGS. 18A-E). The double fluorescent signals in excess of the background observed for appropriate controls, indicated simultaneous binding of the respective antigens by both targeting arms of the bsAbs. The bsAbs containing the anti-TrkB clones F5 and 3C12 were able to simultaneously bind both huTrkB and huCD3d/e expressed by two different CHO-k1 cells (FIGS. 18C and 18E). Binding of the bsAbs containing the anti-TrkB clone 3H11 to these cells was a specific as evidenced by similar signals when cells expressing huCD3d/e were combined with parental CHO-k1 (FIG. 18D).

### B) Activation of Jurkat NFAT Reporter Cells by Developed TrkB×CD3 Bispecific Antibodies

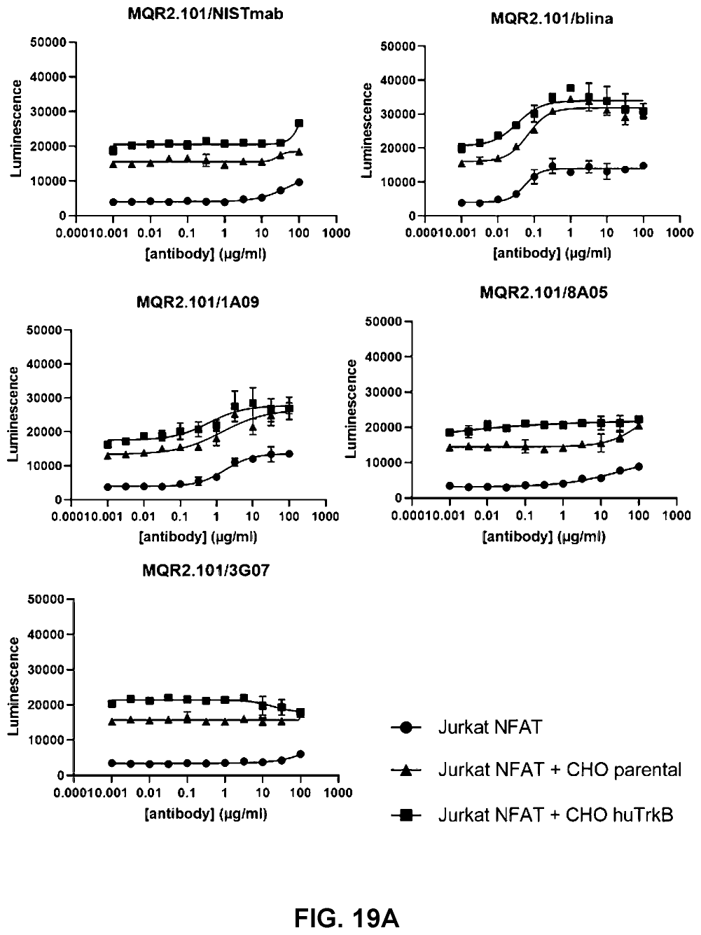

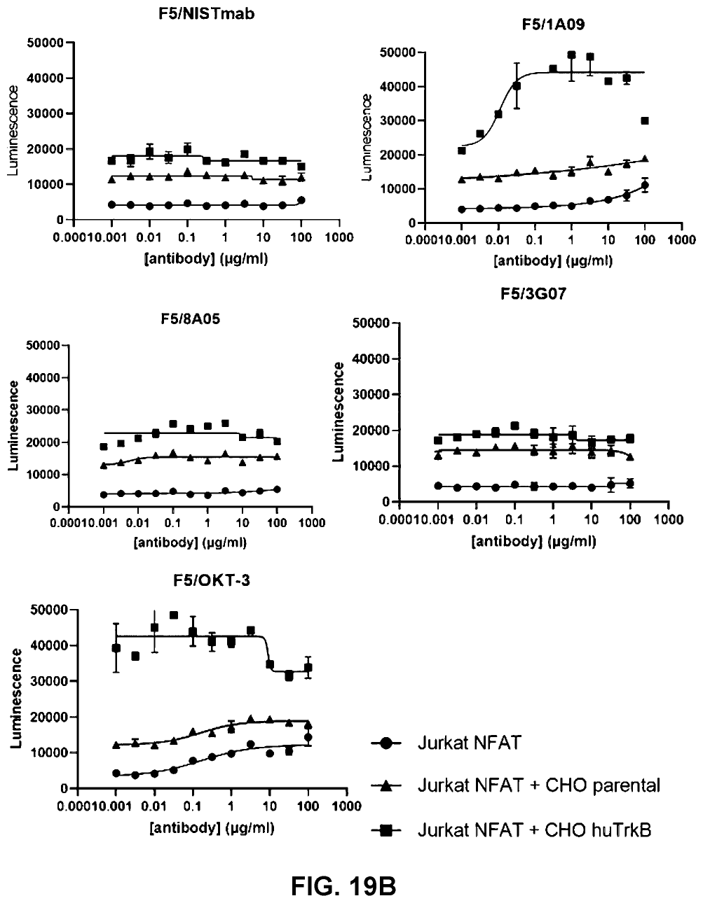

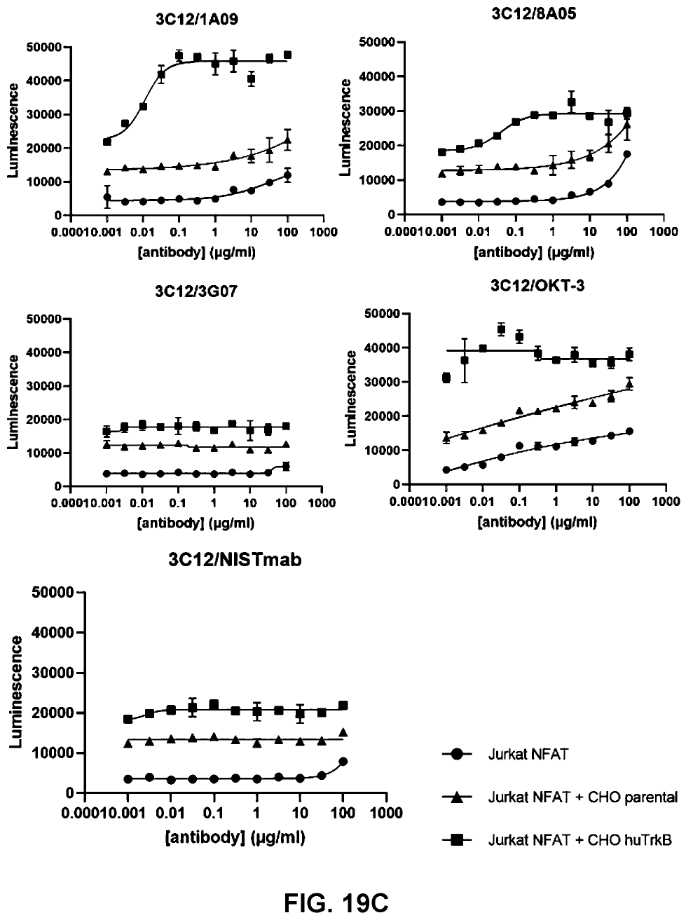

Using Jurkat NFAT reporter cells, the ability of the bsAbs to activate the CD3 receptor expressed on immortalized T cells was determined (FIGS. 19A-C). The bsAbs containing the anti-TrkB clones 3C12 and F5 in combination with the anti-CD3d/e clones 1A09 and OKT-3 were able to activate the CD3 receptor. For the anti-CD3d/e clone 8A05, activation was dependent on the anti-TrkB clone; bsAb comprising the 3C12 and 8A05 TrkB targeting arms did activate the Jurkat cells in a concentration-dependent way, whereas the F5 and 8A05 combination did not. None of the bsAbs containing the anti CD3d/e clone 3G07 was able to induce activation.

### C) Phosphorylation of Membrane-Anchored Human TrkB in CHO-k1 Cells

The auto-phosphorylation of cellular TrkB engendered by the candidate F5×1A09 bsAb was shown in human TrkB-expressing CHO-k1 cells (FIG. 7, plate IV). A significant phosphorylation of TrkB was observed at the lowest concentration of the F5×1A09 bsAb, 0.1 μg/mL, relative to the signal of the non-specific, negative controls antibody hIgG1 and MQR2. 101×NISTmAb bsAb. At the highest tested concentration, 10 μg/mL, the F5×1A09 bsAb was able to trigger phosphorylation of the TrkB target almost as potently as the positive control BDNF at 10 nM. Furthermore, several anti-TrkB mAbs, including 3C12 which was also used for the generation of a bsAb, were able to induce the phosphorylation of TrkB to various extent.

TABLE 10
List of bsAbs prepared and tested in the studies described herein.

| Clone name of bsAb | TrkB Clone ID | CD3 Clone ID |
|---|---|---|
| KiH.F5_TrkB × NISTmAb | F5 | Isotype Ctrl |
| KiH.F5_TrkB × 1A09_CD3 | F5 | 1A09 |
| KiH.F5_TrkB × 8A05_CD3 | F5 | 8A05 |
| KiH.F5_TrkB × 3G07_CD3 | F5 | 3G07 |
| KiH.F5_TrkB × blina_CD3 | F5 | blina |
| KiH.3H11_TrkB × NISTmAb | 3H11 | Isotype Ctrl |
| KiH.3H11_TrkB × 1A09_CD3 | 3H11 | 1A09 |
| KiH.3H11_TrkB × 8A05_CD3 | 3H11 | 8A05 |
| KiH.3H11_TrkB × 3G07_CD3 | 3H11 | 3G07 |
| KiH.3H11_TrkB × blina_CD3 | 3H11 | blina |
| KiH.6E08_TrkB × NISTmAb | 6E08 | Isotype Ctrl |
| KiH.6E08_TrkB × 1A09_CD3 | 6E08 | 1A09 |
| KiH.6E08_TrkB × 8A05_CD3 | 6E08 | 8A05 |
| KiH.6E08_TrkB × 3G07_CD3 | 6E08 | 3G07 |
| KiH.6E08_TrkB × blina_CD3 | 6E08 | blina |
| KiH.3C12_TrkB × NISTmAb | 3C12 | Isotype Ctrl |
| KiH.3C12_TrkB × 1A09_CD3 | 3C12 | 1A09 |
| KiH.3C12_TrkB × 8A05_CD3 | 3C12 | 8A05 |
| KiH.3C12_TrkB × 3G07_CD3 | 3C12 | 3G07 |
| KiH.3C12_TrkB × blina_CD3 | 3C12 | blina |
| KiH.MQR2.101 × NISTmAb | Isotype Ctrl | Isotype Ctrl |
| KiH.MQR2.101 × 1A09_CD3 | Isotype Ctrl | 1A09 |
| KiH.MQR2.101 × 8A05_CD3 | Isotype Ctrl | 8A05 |
| KiH.MQR2.101 × 3G07_CD3 | Isotype Ctrl | 3G07 |
| KiH.MQR2.101 × blina_CD3 | Isotype Ctrl | blina* |

*blina = blinatumomab

The sequences of the heavy and light chain variable regions of the CD3 antibody Blinatumomab (Blina) is depicted in Table 11.

TABLE 11

| | HCVR | LCVR |
|---|---|---|
| Blina | DIKLQQSGAELARPGASVKMSCKTSG | GGVDDIQLTQSPAIMSASPGEKVTMTCR |
| | YTFTRYTMHWVKQRPGQGLEWIGYI | ASSSVSYMNWYQQKSGTSPKRWIYDTS |
| | NPSRGYTNYNQKFKDKATLTTDKSSS | KVASGVPYRFSGSGSGTSYSLTISSMEA |
| | TAYMQLSSLTSEDSAVYYCARYYDDH | EDAATYYCQQWSSNPLTFGAGTKLELK |
| | YCLDYWGQGTTLTVSSVE (SEQ ID | (SEQ ID NO: 709) |
| | NO: 708) | |

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

1. An antibody that binds to tropomyosin receptor kinase B (TrkB) comprising a combination of heavy chain complementarity determining regions (CDR-H1, CDR-H2 and CDR-H3) and light chain complementarity determining regions (CDR-L1, CDR-L2 and CDR-L3), wherein said CDR-H1, CDR-H2, CDR-H3, CDR-L1, CDR-L2 and CDR-L3 comprise or consist of amino acid sequences having at least 70% identity with the sequences depicted in Table 1:
TABLE 1

| CDR-H1 | CDR-H2 | CDR-H3 | CDR-L1 | | CDR-L3 |
|---|---|---|---|---|---|
| (SEQ ID | (SEQ ID | (SEQ ID | (SEQ ID | | (SEQ ID |
| NO: ) | NO: ) | NO: ) | NO: ) | CDR-L2 | NO: ) |
| 23 | 39 | 72 | KSY | DNT | 134 |
| 1 | 24 | 40 | 73 | QDT | 99 |
| 2 | 25 | 41 | 74 | DNI | 100 |
| 3 | 26 | 42 | 75 | DVS | 101 |
| 4 | 27 | 43 | 76 | GKN | 102 |
| 3 | 26 | 42 | 77 | EVT | 103 |
| 5 | 24 | 44 | 78 | QDN | 104 |
| 2 | 25 | 41 | 79 | DDI | 105 |
| 6 | 29 | 45 | 80 | GNN | 106 |
| 7 | 30 | 46 | 81 | QDT | 107 |
| 8 | 29 | 47 | 82 | QDS | 108 |
| 9 | 31 | 48 | 83 | KVS | 109 |
| 10 | 29 | 49 | 84 | QDT | 99 |
| 10 | 29 | 50 | 85 | QDS | 110 |
| 10 | 29 | 51 | 86 | QDT | 111 |
| 10 | 29 | 52 | 78 | QDS | 112 |
| 11 | 29 | 53 | 87 | RNN | 106 |
| 8 | 31 | 47 | 78 | QDT | 113 |
| 10 | 29 | 54 | 88 | QDS | 114 |
| 12 | 29 | 55 | 78 | QDN | 115 |
| 10 | 29 | 56 | 78 | QDN | 116 |
| 13 | 29 | 57 | 89 | RNN | 117 |
| 10 | 29 | 58 | 90 | STF | 118 |
| 10 | 29 | 59 | 73 | QDN | 119 |
| 10 | 29 | 59 | 91 | TNN | 117 |
| 10 | 29 | 60 | 92 | GNS | 120 |
| 14 | 29 | 61 | 85 | QDD | 121 |
| 10 | 29 | 62 | 78 | QDK | 122 |
| 15 | 26 | 63 | 93 | RND | 123 |
| 16 | 32 | 64 | 94 | KVS | 124 |
| 10 | 29 | 65 | 73 | EDN | 125 |
| 17 | 33 | 66 | SSD | YSD | 126 |
| 17 | 33 | 66 | SSD | YSD | 126 |
| 17 | 33 | 66 | SSD | YSD | 133 |
| 18 | 34 | 67 | TYD | YND | 127 |
| 19 | 35 | 68 | 95 | SND | 128 |
| 710 | 711 | 712 | 96 | NND | 129 |
| 20 | 36 | 69 | 97 | NNN | 130 |
| 21 | 37 | 70 | INN | QND | 131 |
| 22 | 38 | 71 | 98 | SND | 132 |
| 28 | 26 | 696 | 697 | RNN | 698 |

or an antigen-binding fragment thereof.

2-3. (canceled)

4. The antibody or antigen-binding fragment thereof of claim 1, wherein the CDR-H1, CDR-H2, CDR-H3, CDR-L1, CDR-L2 and CDR-L3 comprise or consist of the sequences depicted in Table 1.

5-12. (canceled)

13. The antibody or antigen binding fragment thereof according claim 1, wherein said antibody comprises one of the following combinations of heavy chain variable region (HCVR) and light chain variable region (LCVR): SEQ ID NOs: 390 and 391; SEQ ID NOs: 314 and 315; SEQ ID NOs: 316 and 317; SEQ ID NOs: 318 and 319; SEQ ID NOs: 320 and 321; SEQ ID NOs: 322 and 323; SEQ ID NOs: 324 and 325; SEQ ID NOs: 326 and 327; SEQ ID NOs: 328 and 329; SEQ ID NOs: 330 and 331; SEQ ID NOs: 332 and 333; SEQ ID NOs: 334 and 335; SEQ ID NOs: 336 and 337; SEQ ID NOs: 338 and 339; SEQ ID NOs: 340 and 341; SEQ ID NOs: 342 and 343; SEQ ID NOs: 344 and 345; SEQ ID NOs: 346 and 347; SEQ ID NOs: 348 and 349; SEQ ID NOs: 350 and 351; SEQ ID NOs: 352 and 353; SEQ ID NOs: 354 and 355; SEQ ID NOs: 356 and 357; SEQ ID NOs: 358 and 359; SEQ ID NOs: 360 and 361; SEQ ID NOs: 362 and 363; SEQ ID NOs: 364 and 365; SEQ ID NOs: 366 and 367; SEQ ID NOs: 368 and 369; SEQ ID NOs: 370 and 371; SEQ ID NOs: 372 and 373; SEQ ID NOs: 374 and 375; SEQ ID NOs: 376 and 377; SEQ ID NOs: 378 and 379; SEQ ID NOs: 380 and 381; SEQ ID NOs: 382 and 383; SEQ ID NOs: 384 and 385; SEQ ID NOs: 386 and 387; SEQ ID NOs: 388 and 389; SEQ ID NOs: 701 and 702, or SEQ ID NOs: 713 and 714.

14-15. (canceled)

16. The antibody or antigen binding fragment thereof according to claim 1, wherein said antibody is a bispecific antibody.

17. (canceled)

18. The antibody or antigen binding fragment thereof of claim 16, wherein the bispecific antibody further comprises a second antibody or antigen binding fragment thereof or binding domain that specifically binds to CD3.

19. (canceled)

20. The antibody or antigen binding fragment thereof of claim 18, wherein the second antibody or antigen binding fragment thereof or binding domain comprises a combination of CDR-H1, CDR-H2, CDR-H3, CDR-L1, CDR-L2 and CDR-L3 comprising or consisting of the following amino acid sequences:
1) CDR-H1: SEQ ID NO:414; CDR-H2: SEQ ID NO:433; CDR-H3: SEQ ID NO:449; CDR-L1: SEQ ID NO:78; CDR-L2: QDS; and CDR-L3: SEQ ID NO:108;
2) CDR-H1: SEQ ID NO:415; CDR-H2: SEQ ID NO:434; CDR-H3: SEQ ID NO:451; CDR-L1: SEQ ID NO:475; CDR-L2: DDN; and CDR-L3: SEQ ID NO:494; or
3) CDR-H1: SEQ ID NO:415; CDR-H2: SEQ ID NO:434; CDR-H3: SEQ ID NO:451; CDR-L1: SEQ ID NO:476; CDR-L2: DDK; and CDR-L3: SEQ ID NO:495.

21. (canceled)

22. A conjugate comprising the antibody or antigen-binding fragment thereof of claim 1, and a therapeutic agent.

23. A pharmaceutical composition comprising:
(i) the antibody or antigen-binding fragment thereof of claim 1 and a pharmaceutically acceptable excipient, or
(ii) a conjugate comprising the antibody or antigen-binding fragment thereof and a therapeutic agent, and a pharmaceutically acceptable excipient.

24. A method for treating a TrkB-related disease or disorder in a subject in need thereof, comprising administering to the subject an effective amount of the antibody or antigen-binding fragment thereof of claim 1.

25-58. (canceled)

59. An antibody that binds to cluster of differentiation 3 (CD3) comprising a combination of heavy chain complementarity determining regions (CDR-H1, CDR-H2 and CDR-H3) and light chain complementarity determining regions (CDR-L1, CDR-L2 and CDR-L3), wherein said CDR-H1, CDR-H2, CDR-H3, CDR-L1, CDR-L2 and CDR-L3 comprise or consist of amino acid sequences having at least 70% identity with the sequences depicted in Table 2:
TABLE 2

| CDR-H1 | CDR-H2 | CDR-H3 | CDR-L1 | | CDR-L3 |
|---|---|---|---|---|---|
| (SEQ ID | (SEQ ID | (SEQ ID | (SEQ ID | | (SEQ ID |
| NO: ) | NO: ) | NO: ) | NO: ) | CDR-L2 | NO: ) |
| 16 | 432 | 448 | 471 | DNN | 490 |
| 414 | 433 | 449 | 78 | QDS | 108 |
| 15 | 26 | 450 | 472 | NND | 491 |
| 415 | 434 | 451 | 473 | DDS | 492 |
| 703 | 434 | 451 | 474 | DDS | 493 |
| 415 | 434 | 451 | 475 | DDN | 494 |
| 415 | 434 | 451 | 476 | DDK | 495 |
| 415 | 434 | 451 | 477 | EDN | 496 |
| 416 | 435 | 452 | 80 | SNN | 497 |
| 416 | 435 | 452 | 80 | SNN | 497 |
| 15 | 26 | 453 | 478 | RNN | 498 |
| 15 | 26 | 453 | 478 | RNN | 118 |
| 15 | 26 | 453 | 478 | RNN | 499 |
| 417 | 436 | 454 | 479 | EDN | 500 |
| 418 | 437 | 455 | 480 | EDK | 501 |
| 419 | 438 | 456 | 481 | QDN | 502 |
| 420 | 439 | 457 | 482 | DNN | 503 |
| 421 | 437 | 458 | 476 | DDD | 504 |
| 422 | 432 | 459 | 471 | DNN | 490 |
| 423 | 440 | 460 | 483 | DNN | 505 |
| 424 | 441 | 461 | 484 | DDS | 506 |
| 425 | 442 | 462 | 477 | EDN | 507 |
| 20 | 432 | 463 | 477 | EDN | 508 |
| 426 | 441 | 464 | 485 | RNN | 509 |
| 427 | 443 | 465 | 486 | EDK | 510 |
| 20 | 444 | 466 | 487 | DDV | 511 |
| 428 | 445 | 467 | 488 | GAS | 512 |
| 429 | 446 | 468 | 489 | DNN | 513 |
| 430 | 433 | 469 | 477 | DDK | 514 |
| 431 | 447 | 470 | 78 | QDN | 515 |

or an antigen-binding fragment thereof.

60. (canceled)

61. The antibody or antigen binding fragment thereof according to claim 59, wherein the CDR-H1, CDR-H2, CDR-H3, CDR-L1, CDR-L2 and CDR-L3 comprise or consist of the amino acid sequences depicted in Table 2.

62-69. (canceled)

70. The antibody or antigen binding fragment thereof according to claim 59, wherein said antibody comprises one of the following combinations of heavy chain variable region (HCVR) and light chain variable region (LCVR): SEQ ID NOs: 636 and 637, SEQ ID NOs: 638 and 639, SEQ ID NOs: 640 and 641, SEQ ID NOs: 642 and 643, SEQ ID NOs: 644 and 645, SEQ ID NOs: 646 and 647, SEQ ID NOs: 648 and 649, SEQ ID NOs: 650 and 651, SEQ ID NOs: 652 and 653, SEQ ID NOs: 654 and 655, SEQ ID NOs: 656 and 657, SEQ ID NOs: 658 and 659, SEQ ID NOs: 660 and 661, SEQ ID NOs: 662 and 663, SEQ ID NOs: 664 and 665, SEQ ID NOs: 666 and 667, SEQ ID NOs: 668 and 669, SEQ ID NOs: 670 and 671, SEQ ID NOs: 672 and 673, SEQ ID NOs: 674 and 675, SEQ ID NOs: 676 and 677, SEQ ID NOs: 678 and 679, SEQ ID NOs: 680 and 681, SEQ ID NOs: 682 and 683, SEQ ID NOs: 684 and 685, SEQ ID NOs: 686 and 687, SEQ ID NOs: 688 and 689, SEQ ID NOs: 690 and 691, SEQ ID NOs: 692 and 693, or SEQ ID NOs: 694 and 695.

71-72. (canceled)

73. The antibody or antigen-binding fragment thereof according to claim 59, wherein said antibody is a bispecific antibody.

74. The antibody or antigen-binding fragment thereof according to claim 73, wherein the bispecific antibody further comprises a second antibody or antigen-binding fragment thereof that specifically binds to TrkB.

75. (canceled)

76. The antibody or antigen-binding fragment thereof according to claim 74, wherein the second antibody or antigen-binding fragment thereof is an antibody comprising a combination of heavy chain complementarity determining regions (CDR-H1, CDR-H2 and CDR-H3) and light chain complementarity determining regions (CDR-L1, CDR-L2 and CDR-L3), wherein said CDR-H1, CDR-H2, CDR-H3, CDR-L1, CDR-L2 and CDR-L3 comprise or consist of amino acid sequences having at least 70% identity with the sequences depicted in Table 1:
TABLE 1

| CDR-H1 | CDR-H2 | CDR-H3 | CDR-L1 | | CDR-L3 |
|---|---|---|---|---|---|
| SEQ ID | SEQ ID | SEQ ID | (SEQ ID | | (SEQ ID |
| NO: ) | NO: ) | NO: ) | NO: ) | CDR-L2 | NO: ) |
| 23 | 39 | 72 | KSY | DNT | 134 |
| 1 | 24 | 40 | 73 | QDT | 99 |
| 2 | 25 | 41 | 74 | DNI | 100 |
| 3 | 26 | 42 | 75 | DVS | 101 |
| 4 | 27 | 43 | 76 | GKN | 102 |
| 3 | 26 | 42 | 77 | EVT | 103 |
| 5 | 24 | 44 | 78 | QDN | 104 |
| 2 | 25 | 41 | 79 | DDI | 105 |
| 6 | 29 | 45 | 80 | GNN | 106 |
| 7 | 30 | 46 | 81 | QDT | 107 |
| 8 | 29 | 47 | 82 | QDS | 108 |
| 9 | 31 | 48 | 83 | KVS | 109 |
| 10 | 29 | 49 | 84 | QDT | 99 |
| 10 | 29 | 50 | 85 | QDS | 110 |
| 10 | 29 | 51 | 86 | QDT | 111 |
| 10 | 29 | 52 | 78 | QDS | 112 |
| 11 | 29 | 53 | 87 | RNN | 106 |
| 8 | 31 | 47 | 78 | QDT | 113 |
| 10 | 29 | 54 | 88 | QDS | 114 |
| 12 | 29 | 55 | 78 | QDN | 115 |
| 10 | 29 | 56 | 78 | QDN | 116 |
| 13 | 29 | 57 | 89 | RNN | 117 |
| 10 | 29 | 58 | 90 | STF | 118 |
| 10 | 29 | 59 | 73 | QDN | 119 |
| 10 | 29 | 59 | 91 | TNN | 117 |
| 10 | 29 | 60 | 92 | GNS | 120 |
| 14 | 29 | 61 | 85 | QDD | 121 |
| 10 | 29 | 62 | 78 | QDK | 122 |
| 15 | 26 | 63 | 93 | RND | 123 |
| 16 | 32 | 64 | 94 | KVS | 124 |
| 10 | 29 | 65 | 73 | EDN | 125 |
| 17 | 33 | 66 | SSD | YSD | 126 |
| 17 | 33 | 66 | SSD | YSD | 126 |
| 17 | 33 | 66 | SSD | YSD | 133 |
| 18 | 34 | 67 | TYD | YND | 127 |
| 19 | 35 | 68 | 95 | SND | 128 |
| 710 | 711 | 712 | 96 | NND | 129 |
| 20 | 36 | 69 | 97 | NNN | 130 |
| 21 | 37 | 70 | INN | QND | 131 |
| 22 | 38 | 71 | 98 | SND | 132 |
| 28 | 26 | 696 | 697 | RNN | 698 |

or an antigen-binding fragment thereof.

77. A conjugate comprising the antibody or antigen-binding fragment thereof of claim 59, and a therapeutic agent.

78. A pharmaceutical composition comprising:
(i) the antibody or antigen-binding fragment thereof of claim 59 and a pharmaceutically acceptable excipient, or
(ii) a conjugate comprising the antibody or antigen-binding fragment thereof and a therapeutic agent, and a pharmaceutically acceptable excipient.

79. A method for treating cancer or a CD3-associated autoimmune disease in a subject in need thereof, comprising administering to the subject an effective amount of the antibody or antigen-binding fragment thereof of claim 59.

80-105. (canceled)

106. A nucleic acid encoding the antibody or antigen-binding fragment thereof of claim 1.

107. A nucleic acid encoding the antibody or antigen-binding fragment thereof of claim 59.


SEQUENCE LISTING (714 sequences)

SEQ ID NO: 1 - protein, 8 aa, Homo sapiens
GYRFTSYW

SEQ ID NO: 2 - protein, 8 aa, Homo sapiens
GYSFTNNW

SEQ ID NO: 3 - protein, 10 aa, Homo sapiens
GDSVSSKSAA

SEQ ID NO: 4 - protein, 8 aa, Homo sapiens
GFTFSNYA

SEQ ID NO: 5 - protein, 8 aa, Homo sapiens
GYRFSHYW

SEQ ID NO: 6 - protein, 8 aa, Homo sapiens
GYSFTTYW

SEQ ID NO: 7 - protein, 8 aa, Homo sapiens
GFTFSNAW

SEQ ID NO: 8 - protein, 8 aa, Homo sapiens
GYSFPTYW

SEQ ID NO: 9 - protein, 8 aa, Homo sapiens
GYTFTNYA

SEQ ID NO: 10 - protein, 8 aa, Homo sapiens
GYSFTSYW

SEQ ID NO: 11 - protein, 8 aa, Homo sapiens
GYTFTNYW

SEQ ID NO: 12 - protein, 8 aa, Homo sapiens
GYSFSTNW

SEQ ID NO: 13 - protein, 8 aa, Homo sapiens
RYSFTSYW

SEQ ID NO: 14 - protein, 8 aa, Homo sapiens
GYSFTNYW

SEQ ID NO: 15 - protein, 10 aa, Homo sapiens
GDSVSSNSAA

SEQ ID NO: 16 - protein, 8 aa, Homo sapiens
GFTFSSYA

SEQ ID NO: 17 - protein, 8 aa, Homo sapiens
GFTFSSFP

SEQ ID NO: 18 - protein, 8 aa, Homo sapiens
GFTFSNHQ

SEQ ID NO: 19 - protein, 8 aa, Homo sapiens
GFTFSSFN

SEQ ID NO: 20 - protein, 8 aa, Homo sapiens
GFTFSSYG

SEQ ID NO: 21 - protein, 8 aa, Homo sapiens
GFDFSSNA

SEQ ID NO: 22 - protein, 8 aa, Homo sapiens
GFTFSSHA

SEQ ID NO: 23 - protein, 8 aa, Homo sapiens
GFTFSSSG

SEQ ID NO: 24 - protein, 8 aa, Homo sapiens
IYPDDSDT

SEQ ID NO: 25 - protein, 8 aa, Homo sapiens
IDPDDSYT

SEQ ID NO: 26 - protein, 7 aa, Homo sapiens
TYYRSKW

SEQ ID NO: 27 - protein, 8 aa, Homo sapiens
ISYDGSNE

SEQ ID NO: 28 - protein, 10 aa, Homo sapiens
GDSVSSNSAV

SEQ ID NO: 29 - protein, 8 aa, Homo sapiens
IYPGDSDT

SEQ ID NO: 30 - protein, 10 aa, Homo sapiens
IKSKTDGGTT

SEQ ID NO: 31 - protein, 8 aa, Homo sapiens
INPNTGGT

SEQ ID NO: 32 - protein, 8 aa, Homo sapiens
ISYDGSNK

SEQ ID NO: 33 - protein, 7 aa, Homo sapiens
IVRTSTP

SEQ ID NO: 34 - protein, 8 aa, Homo sapiens
INAAGSWT

SEQ ID NO: 35 - protein, 8 aa, Homo sapiens
IKRNGGET

SEQ ID NO: 36 - protein, 8 aa, Homo sapiens
ISSTGINA

SEQ ID NO: 37 - protein, 8 aa, Homo sapiens
ITTDGSYT

SEQ ID NO: 38 - protein, 7 aa, Homo sapiens
INYDGIT

SEQ ID NO: 39 - protein, 8 aa, Homo sapiens
ITSSAGNT

SEQ ID NO: 40 - protein, 9 aa, Homo sapiens
ASGEVAFDV

SEQ ID NO: 41 - protein, 12 aa, Homo sapiens
ARRAQGGGGYDY

SEQ ID NO: 42 - protein, 11 aa, Homo sapiens
ARGIGSLNFDI

SEQ ID NO: 43 - protein, 10 aa, Homo sapiens
ARGTNYGMDV

SEQ ID NO: 44 - protein, 10 aa, Homo sapiens
ATGVISGTDY

SEQ ID NO: 45 - protein, 9 aa, Homo sapiens
ARLGGGMDV

SEQ ID NO: 46 - protein, 12 aa, Homo sapiens
TTENEVWPSLDV

SEQ ID NO: 47 - protein, 9 aa, Homo sapiens
ARGGSGLDY

SEQ ID NO: 48 - protein, 9 aa, Homo sapiens
ARGGGGLVF

SEQ ID NO: 49 - protein, 10 aa, Homo sapiens
ATPMEDGMDV

SEQ ID NO: 50 - protein, 8 aa, Homo sapiens
ARGGPPDY

SEQ ID NO: 51 - protein, 8 aa, Homo sapiens
TRSSFNDH

SEQ ID NO: 52 - protein, 11 aa, Homo sapiens
ARLGGSLPIDY

SEQ ID NO: 53 - protein, 11 aa, Homo sapiens
AIGYDYGAFDI

SEQ ID NO: 54 - protein, 13 aa, Homo sapiens
ASRLLFGDDAFDI

SEQ ID NO: 55 - protein, 11 aa, Homo sapiens
ARFYDSSGNDY

SEQ ID NO: 56 - protein, 13 aa, Homo sapiens
VVRPAAAGTRIDY

SEQ ID NO: 57 - protein, 10 aa, Homo sapiens
ARLKDGAFDI

SEQ ID NO: 58 - protein, 7 aa, Homo sapiens
ASWGMDV

SEQ ID NO: 59 - protein, 9 aa, Homo sapiens
ARHDYGMDV

SEQ ID NO: 60 - protein, 12 aa, Homo sapiens
ARLGQGSGPFDY

SEQ ID NO: 61 - protein, 11 aa, Homo sapiens
ASHTKDGTWDI

SEQ ID NO: 62 - protein, 9 aa, Homo sapiens
ARLGGAMDV

SEQ ID NO: 63 - protein, 12 aa, Homo sapiens
TRGTGDKRAFDY

SEQ ID NO: 64 - protein, 12 aa, Homo sapiens
ARAFRRYDAFDI

SEQ ID NO: 65 - protein, 9 aa, Homo sapiens
ARGSGGYDY

SEQ ID NO: 66 - protein, 16 aa, Homo sapiens
AKSATICGNCPEDIDA

SEQ ID NO: 67 - protein, 17 aa, Homo sapiens
AKDVYGYCSTCGATIDT

SEQ ID NO: 68 - protein, 19 aa, Homo sapiens
AKDAGDNCRWNYCTTNIDA

SEQ ID NO: 69 - protein, 15 aa, Homo sapiens
SKCAYSGSCSTSIDA

SEQ ID NO: 70 - protein, 15 aa, Homo sapiens
AKDSSIHWNGYDIDA

SEQ ID NO: 71 - protein, 17 aa, Homo sapiens
AKTTCSGCWDAAALIDA

SEQ ID NO: 72 - protein, 16 aa, Homo sapiens
AKDSNSGSPDASEIDA

SEQ ID NO: 73 - protein, 6 aa, Homo sapiens
KLGDKF

SEQ ID NO: 74 - protein, 9 aa, Homo sapiens
SSNIGAGFN

SEQ ID NO: 75 - protein, 9 aa, Homo sapiens
SSDIGGYDY

SEQ ID NO: 76 - protein, 6 aa, Homo sapiens
SLRSYY

SEQ ID NO: 77 - protein, 9 aa, Homo sapiens
SGDVGGYNY

SEQ ID NO: 78 - protein, 6 aa, Homo sapiens
KLGDKY

SEQ ID NO: 79 - protein, 9 aa, Homo sapiens
SSNIGAGYG

SEQ ID NO: 80 - protein, 8 aa, Homo sapiens
SSNIGSNT

SEQ ID NO: 81 - protein, 6 aa, Homo sapiens
KLGNKY

SEQ ID NO: 82 - protein, 6 aa, Homo sapiens
KLGTKY

SEQ ID NO: 83 - protein, 11 aa, Homo sapiens
QSLVDRDGNTY

SEQ ID NO: 84 - protein, 6 aa, Homo sapiens
NLGDKY

SEQ ID NO: 85 - protein, 6 aa, Homo sapiens
RLGDKY

SEQ ID NO: 86 - protein, 6 aa, Homo sapiens
KLGNKF

SEQ ID NO: 87 - protein, 8 aa, Homo sapiens
NSNIGSNS

SEQ ID NO: 88 - protein, 6 aa, Homo sapiens
KLGDEY

SEQ ID NO: 89 - protein, 8 aa, Homo sapiens
SSNIEGNY

SEQ ID NO: 90 - protein, 8 aa, Homo sapiens
SSNIGTHT

SEQ ID NO: 91 - protein, 8 aa, Homo sapiens
RSNVGGNT

SEQ ID NO: 92 - protein, 8 aa, Homo sapiens
SSNIGSHS

SEQ ID NO: 93 - protein, 8 aa, Homo sapiens
SSNIGSYF

SEQ ID NO: 94 - protein, 11 aa, Homo sapiens
QGLVYSDGNTY

SEQ ID NO: 95 - protein, 8 aa, Homo sapiens
GRWYGTYY

SEQ ID NO: 96 - protein, 8 aa, Homo sapiens
GSYYGSYY

SEQ ID NO: 97 - protein, 5 aa, Homo sapiens
SSGYG

SEQ ID NO: 98 - protein, 5 aa, Homo sapiens
SSGYA

SEQ ID NO: 99 - protein, 9 aa, Homo sapiens
QAWDSSTGV

SEQ ID NO: 100 - protein, 12 aa, Homo sapiens
QSYDSSLSGWFA

SEQ ID NO: 101 - protein, 12 aa, Homo sapiens
SSYRAGSTLNLL

SEQ ID NO: 102 - protein, 11 aa, Homo sapiens
QSRDTTGNYLL

SEQ ID NO: 103 - protein, 11 aa, Homo sapiens
SSYGGPNNLVL

SEQ ID NO: 104 - protein, 9 aa, Homo sapiens
QTWDSGTGV

SEQ ID NO: 105 - protein, 12 aa, Homo sapiens
QSYDTSRGGLYV

SEQ ID NO: 106 - protein, 11 aa, Homo sapiens
AAWDDSLSGYV

SEQ ID NO: 107 - protein, 10 aa, Homo sapiens
QAWDSSTAVV

SEQ ID NO: 108 - protein, 9 aa, Homo sapiens
QAWDSSTVV

SEQ ID NO: 109 - protein, 9 aa, Homo sapiens
MQATHWPPT

SEQ ID NO: 110 - protein, 9 aa, Homo sapiens
QAWDSSTAV

SEQ ID NO: 111 - protein, 9 aa, Homo sapiens
QAWNTNTAL

SEQ ID NO: 112 - protein, 9 aa, Homo sapiens
QAWDNNIGV

SEQ ID NO: 113 - protein, 9 aa, Homo sapiens
QAWDSNTMV

SEQ ID NO: 114 - protein, 9 aa, Homo sapiens
QAWDSNAGA

SEQ ID NO: 115 - protein, 9 aa, Homo sapiens
QTWDSSTAI

SEQ ID NO: 116 - protein, 9 aa, Homo sapiens
QAWDSSTYV

SEQ ID NO: 117 - protein, 11 aa, Homo sapiens
AAWDDSLNGYV

SEQ ID NO: 118 - protein, 11 aa, Homo sapiens
AAWDDSLNGPV

SEQ ID NO: 119 - protein, 9 aa, Homo sapiens
QAWVNNTVV

SEQ ID NO: 120 - protein, 11 aa, Homo sapiens
ASWDDSLNGYV

SEQ ID NO: 121 - protein, 11 aa, Homo sapiens
QAWDSSTSLYV

SEQ ID NO: 122 - protein, 12 aa, Homo sapiens
AAWDDSLNGYYV

SEQ ID NO: 123 - protein, 11 aa, Homo sapiens
AAWDDSLSGRI

SEQ ID NO: 124 - protein, 9 aa, Homo sapiens
MQGTHWPPA

SEQ ID NO: 125 - protein, 9 aa, Homo sapiens
QTWDVSSYV

SEQ ID NO: 126 - protein, 10 aa, Homo sapiens
GSRDSSNVAI

SEQ ID NO: 127 - protein, 10 aa, Homo sapiens
GTYEDNNGAS

SEQ ID NO: 128 - protein, 10 aa, Homo sapiens
GSWDSTDTTA

SEQ ID NO: 129 - protein, 10 aa, Homo sapiens
GSKDSSGAGI

SEQ ID NO: 130 - protein, 10 aa, Homo sapiens
GAWESSATDI

SEQ ID NO: 131 - protein, 11 aa, Homo sapiens
GSEDSRTYLGI

SEQ ID NO: 132 - protein, 9 aa, Homo sapiens
GSFDSSSYI

SEQ ID NO: 133 - protein, 9 aa, Homo sapiens
GSGDNSYVG

SEQ ID NO: 134 - protein, 10 aa, Homo sapiens
GNADHSYVGV

SEQ ID NO: 135 - protein, 25 aa, Homo sapiens
QVQLVQSGAEVKKPGESLRISCKGS

SEQ ID NO: 136 - protein, 25 aa, Homo sapiens
QVQLQQSGPGLVKPAQTLSLTCDIS

SEQ ID NO: 137 - protein, 25 aa, Homo sapiens
QVQLQQSGGGVVQPGRSLRLSCAAS

SEQ ID NO: 138 - protein, 25 aa, Homo sapiens
QVQLQQSGPGLVKPSQTLSVTCVIS

SEQ ID NO: 139 - protein, 25 aa, Homo sapiens
QVQLVQSGAEVKKPGESLKISCTGS

SEQ ID NO: 140 - protein, 25 aa, Homo sapiens
EVQLVESGAEVKKPGESLRISCKGS

SEQ ID NO: 141 - protein, 25 aa, Homo sapiens
QVQLVESGAEVKKPGESLKISCKGS

SEQ ID NO: 142 - protein, 25 aa, Homo sapiens
EVQLVQSGGGLVKPGGSLRLSCAAS

SEQ ID NO: 143 - protein, 25 aa, Homo sapiens
QVQLVQSGAEVKKPGEPLKISCKGS

SEQ ID NO: 144 - protein, 25 aa, Homo sapiens
EVQLVQSGAEVKEPGASVKVSCKAS

SEQ ID NO: 145 - protein, 25 aa, Homo sapiens
EVQLVQSGAEVKKPGESLKISCKGS

SEQ ID NO: 146 - protein, 25 aa, Homo sapiens
QVQLVQSGAEVKKPGESLKISCKGS

SEQ ID NO: 147 - protein, 25 aa, Homo sapiens
QVQLVQSGAEVKKPGESLKISCKGS

SEQ ID NO: 148 - protein, 25 aa, Homo sapiens
EVQLVQSGAEVKKPGESLKISCKGS

SEQ ID NO: 149 - protein, 25 aa, Homo sapiens
EVQLVQSGAEVKKPGESLKISCQGS

SEQ ID NO: 150 - protein, 25 aa, Homo sapiens
QVQLVESGAEVKKPGEPLKISCKGS

SEQ ID NO: 151 - protein, 25 aa, Homo sapiens
EVQLVQSGAEVKKPGESLKISCKGS

SEQ ID NO: 152 - protein, 25 aa, Homo sapiens
QMQLVQSGAEVKKPGESLKISCKGS

SEQ ID NO: 153 - protein, 25 aa, Homo sapiens
EVQLVQSGAEVKKPGESLKISCKGS

SEQ ID NO: 154 - protein, 25 aa, Homo sapiens
EVQLVQSGAEVKKPGESLKISCKGS

SEQ ID NO: 155 - protein, 25 aa, Homo sapiens
EVQLVQSGAEVKKPGESLKISCKGS

SEQ ID NO: 156 - protein, 25 aa, Homo sapiens
EVQLVQSGAEVKKPGESLKISCKGS

SEQ ID NO: 157 - protein, 25 aa, Homo sapiens
QVQLVQSGAEVKKPGESLKISCKGS

SEQ ID NO: 158 - protein, 25 aa, Homo sapiens
QMQLVQSGAEVKKPGESLKISCKGS

SEQ ID NO: 159 - protein, 25 aa, Homo sapiens
QVQLVQSGAEVKKPGESLKISCKGF

SEQ ID NO: 160 - protein, 25 aa, Homo sapiens
QVQLVQSGAEVKKPGESLKISCKGS

SEQ ID NO: 161 - protein, 25 aa, Homo sapiens
QVQLQQSGAGLVKASQTLALTCAIS

SEQ ID NO: 162 - protein, 25 aa, Homo sapiens
QVQLVQSGGGVVQPGRSLRLSCAAS

SEQ ID NO: 163 - protein, 25 aa, Homo sapiens
EVQLVQSGAEVKKPGESLKISCKGS

SEQ ID NO: 164 - protein, 25 aa, Homo sapiens
AVTLDESGGGLQTPGGALSLVCKAS

SEQ ID NO: 165 - protein, 25 aa, Homo sapiens
AVTLDESGGGLQTPGGGLSLVCKAS

SEQ ID NO: 166 - protein, 25 aa, Homo sapiens
AVTLDESGGGLQTPGGPLSLVCEGS

SEQ ID NO: 167 - protein, 17 aa, Homo sapiens
IGWVRQMPGKGLEWVGI

SEQ ID NO: 168 - protein, 17 aa, Homo sapiens
INWVRQMPGKGLEWMGR

SEQ ID NO: 169 - protein, 17 aa, Homo sapiens
WHWIRQSPSRGLEWLGR

SEQ ID NO: 170 - protein, 17 aa, Homo sapiens
MHWVRQAPGKGLEWVAV

SEQ ID NO: 171 - protein, 17 aa, Homo sapiens
IGWVRQMPGKGLEWMGM

SEQ ID NO: 172 - protein, 17 aa, Homo sapiens
INWVRQMPGKGLEWMGR

SEQ ID NO: 173 - protein, 17 aa, Homo sapiens
IAWVRQMPGKGLEWMGI

SEQ ID NO: 174 - protein, 17 aa, Homo sapiens
MSWVRQAPGKGLEWVGR

SEQ ID NO: 175 - protein, 17 aa, Homo sapiens
MHWVRQAPGQRLEWMGW

SEQ ID NO: 176 - protein, 17 aa, Homo sapiens
IGWVRQMPGKGLEWMGI

SEQ ID NO: 177 - protein, 17 aa, Homo sapiens
IGWVRQMPGKGLEWLGI

SEQ ID NO: 178 - protein, 17 aa, Homo sapiens
WNWIRQSPSRGLEWLGR

SEQ ID NO: 179 - protein, 17 aa, Homo sapiens
MVWVRQAPGKGLEFVAG

SEQ ID NO: 180 - protein, 17 aa, Homo sapiens
MGWVRQAPGKGLEFIAS

SEQ ID NO: 181 - protein, 17 aa, Homo sapiens
MAWVRQAPGKGLEYVGL

SEQ ID NO: 182 - protein, 17 aa, Homo sapiens
MFWVRQAPGKGLEFVAH

SEQ ID NO: 183 - protein, 17 aa, Homo sapiens
MQWVRQAPGKGLEWVAG

SEQ ID NO: 184 - protein, 17 aa, Homo sapiens
MAWVRQAPGKRLEWVAS

SEQ ID NO: 185 - protein, 17 aa, Homo sapiens
MAWVRQAPGKGLEFVAG

SEQ ID NO: 186 - protein, 17 aa, Homo sapiens
MFWVRQAPGKGLEYVAY

SEQ ID NO: 187 - protein, 38 aa, Homo sapiens
RYSPSFQGQVTISADTSISTTYLQWSSLKASDTAIYYC

SEQ ID NO: 188 - protein, 38 aa, Homo sapiens
NYSPSFQGHVIFSADKSSNTVYLQWSSLEASDTAMYYC

SEQ ID NO: 189 - protein, 40 aa, Homo sapiens
YSDYGESVKSRVVINADTSNNQISLQLSSVTPEDTAAYYC

SEQ ID NO: 190 - protein, 38 aa, Homo sapiens
YYADSVKGRFTISRDNSRNTLDLQMNSLRAEDTAVYYC

SEQ ID NO: 191 - protein, 38 aa, Homo sapiens
RYSPSFQGQVTISADKSINTAYLQWTSLRASDSAIYYC

SEQ ID NO: 192 - protein, 38 aa, Homo sapiens; identical to SEQ ID NO: 188

SEQ ID NO: 193 - protein, 38 aa, Homo sapiens
RYSPSFQGQVTISADKSISTAYLQWSSLKASDTAMYYC

SEQ ID NO: 194 - protein, 38 aa, Homo sapiens
DYAAPVKGRFTISRDDSKNTLYLQMNSLKTEDTAVYYC

SEQ ID NO: 195 - protein, 38 aa, Homo sapiens
RYSPSFQGQVTISADKSISTAYLQWGSLRASDTAMYYC

SEQ ID NO: 196 - protein, 38 aa, Homo sapiens
NYAQKFQGRVTMTRDTSISTAYMELSSLRSDDTAVYYC

SEQ ID NO: 197 - protein, 38 aa, Homo sapiens; identical to SEQ ID NO: 195

SEQ ID NO: 198 - protein, 38 aa, Homo sapiens
RYSPSFQGQVTISADKSITTAYLQWSSLKASDTAMYYC

SEQ ID NO: 199 - protein, 38 aa, Homo sapiens
RYSPSFQGQVTISADKSISTAYLQWSSLKASDTAIYYC

SEQ ID NO: 200 - protein, 38 aa, Homo sapiens
KYGAAVTGRATISRDNGQSTVRLQLNNLRAEDTGTYFC

SEQ ID NO: 201 - protein, 38 aa, Homo sapiens
YYGTAVKGRATISRDNGQSTVRLQLNNLRAEDTATYYC

SEQ ID NO: 202 - protein, 38 aa, Homo sapiens
DYGAAVKGRATISRDDGQSTVRLQLNNLRAEDTATYFC

SEQ ID NO: 203 - protein, 38 aa, Homo sapiens
NYGSAVKGRATISRDNGQSTLRLQLNNLRAEDTGIYFC

SEQ ID NO: 204 - protein, 38 aa, Homo sapiens
YYGAAVKGRATISRDNGQSTVRLQLNNLRAEDTGIYYC

SEQ ID NO: 205 - protein, 38 aa, Homo sapiens
TYGAAVKGRATISRDDGQSTLRLQLNNLRAEDTATYYC

SEQ ID NO: 206 - protein, 38 aa, Homo sapiens
YYAPAVKGRATISRDNGQSTVRLQLNNLRAADTGTYFC

SEQ ID NO: 207 - protein, 38 aa, Homo sapiens
FYGAAVKGRATISRDNGQSTVRLQLNNLRAEDTATYFC

SEQ ID NO: 208 - protein, 11 aa, Homo sapiens
WGQGTTVTVSS

SEQ ID NO: 209 - protein, 11 aa, Homo sapiens
WGQGTLVTVSS

SEQ ID NO: 210 - protein, 11 aa, Homo sapiens
WGQGTMVTVSS

SEQ ID NO: 211 - protein, 11 aa, Homo sapiens
WGHGTEVIVSS

SEQ ID NO: 212 - protein, 25 aa, Homo sapiens
QSVLTQPPSLSVSPRQTASITCSGD

SEQ ID NO: 213 - protein, 25 aa, Homo sapiens
QSVLTQPPSVSGAPGQRVTISCTGS

SEQ ID NO: 214 - protein, 25 aa, Homo sapiens
QSVVTQPASVSGSPGQSITISCTGT

SEQ ID NO: 215 - protein, 25 aa, Homo sapiens
LSELTQDPAVSVALGQTVRITCQGD

SEQ ID NO: 216 - protein, 25 aa, Homo sapiens
QSVLTQPPSASGSPGQSVTISCTGT

SEQ ID NO: 217 - protein, 25 aa, Homo sapiens
SYVLTQPPSVSVSPGQTARITCSGD

SEQ ID NO: 218 - protein, 25 aa, Homo sapiens
QSVLTQPSSVSGAPGQRVTISCTGS

SEQ ID NO: 219 - protein, 25 aa, Homo sapiens
QAGLTQPPSASGTPGQRVTISCSGS

SEQ ID NO: 220 - protein, 25 aa, Homo sapiens
QSVLTQPPSVSVSPGQTASITCSGD

SEQ ID NO: 221 - protein, 25 aa, Homo sapiens
SYELMQPPSVSVSPGQTASITCSGD

SEQ ID NO: 222 - protein, 26 aa, Homo sapiens
DIVMTQTPLSLPVTLGQPASISCRSS

SEQ ID NO: 223 - protein, 25 aa, Homo sapiens
QAGLTQPPSVSVSPGQTVTITCSAD

SEQ ID NO: 224 - protein, 25 aa, Homo sapiens
LPVLTQPPSVSVSPGQTASITCSGD

SEQ ID NO: 225 - protein, 25 aa, Homo sapiens
QPVLTQPPSVSVSPGQTASITCSGD

SEQ ID NO: 226 - protein, 25 aa, Homo sapiens
QPVLTQPPSASGTPGQRVSISCSGS

SEQ ID NO: 227 - protein, 25 aa, Homo sapiens
QAGLTQPPSVSVSPGQTAIIPCSGE

SEQ ID NO: 228 - protein, 25 aa, Homo sapiens
SYVLTQPPSVSVSPGQTATITCSGD

SEQ ID NO: 229 - protein, 25 aa, Homo sapiens
QAVLTQPPSASGTPGQRVTISCSGS

SEQ ID NO: 230 - protein, 25 aa, Homo sapiens
QPVLTQPPSASGTPGQRVTISCSGS

SEQ ID NO: 231 - protein, 25 aa, Homo sapiens
LPVLTQPPSASGTPGQRVTISCSGS

SEQ ID NO: 232 - protein, 25 aa, Homo sapiens
QSVVTQPPSVSGTPGQRVSISCSGS

SEQ ID NO: 233 - protein, 25 aa, Homo sapiens
QAGLTQPPSVSVSPGQTASITCSGE

SEQ ID NO: 234 - protein, 25 aa, Homo sapiens
SYELMQPPSLSVSPGQTASITCSGD

SEQ ID NO: 235 - protein, 25 aa, Homo sapiens
QSVLTQPPSASGTPGQRVTISCSGS

SEQ ID NO: 236 - protein, 26 aa, Homo sapiens
DIQMTQTPLSLSVTLGQPASISCRSS

SEQ ID NO: 237 - protein, 25 aa, Homo sapiens
QSVLTQPPSVSVSPGQTASITCSGN

SEQ ID NO: 238 - protein, 25 aa, Homo sapiens
QAALTQPASVSANPGETVKITCSGG

SEQ ID NO: 239 - protein, 25 aa, Homo sapiens
QAALTQPSSVSANPGETVKITCSGP

SEQ ID NO: 240 - protein, 25 aa, Homo sapiens
QAALTQPASVSANLGGTVEITCSGG

SEQ ID NO: 241 - protein, 25 aa, Homo sapiens
QAALTQPSSVSANPGETVKITCSGS

SEQ ID NO: 242 - protein, 25 aa, Homo sapiens
QAALTQPASVSANPGETVKITCSGY

SEQ ID NO: 243 - protein, 25 aa, Homo sapiens
QAALTQPASVSSNPGETVKITCSGG

SEQ ID NO: 244 - protein, 17 aa, Homo sapiens
VSWYQQKPGQSPVLVIY

SEQ ID NO: 245 - protein, 17 aa, Homo sapiens
VHWYQQVAGLAPKLLIH

SEQ ID NO: 246 - protein, 17 aa, Homo sapiens
VSWYQQHPGKAPKLMIF

SEQ ID NO: 247 - protein, 17 aa, Homo sapiens
ASWYQQKPGQAPVLVIY

SEQ ID NO: 248 - protein, 17 aa, Homo sapiens
VSWYQQHPGKAPKLILY

SEQ ID NO: 249 - protein, 17 aa, Homo sapiens
VSWYQLKPGQSPVVVIY

SEQ ID NO: 250 - protein, 17 aa, Homo sapiens
VHWYQQLPGTAPKLLIY

SEQ ID NO: 251 - protein, 17 aa, Homo sapiens
VSWYRQIPGTAPKFLLY

SEQ ID NO: 252 - protein, 17 aa, Homo sapiens
ASWYRQKPGQSPVLVIY

SEQ ID NO: 253 - protein, 17 aa, Homo sapiens
LNWFRQRPGQSPRRLIY

SEQ ID NO: 254 - protein, 17 aa, Homo sapiens
TSWYQQRPGQSPVLVIY

SEQ ID NO: 255 - protein, 17 aa, Homo sapiens
ASWYQQKPGQSPVLVIY

SEQ ID NO: 256 - protein, 17 aa, Homo sapiens
VSWFQQKPGQSPVLVIY

SEQ ID NO: 257 - protein, 17 aa, Homo sapiens
VYWYQQLPGTAPKLLIY

SEQ ID NO: 258 - protein, 17 aa, Homo sapiens
VSWYQQKPGESPLSIIY

SEQ ID NO: 259 - protein, 17 aa, Homo sapiens
ASWYQQKPGQSPVLVIF

SEQ ID NO: 260 - protein, 17 aa, Homo sapiens
ASWYQQKPGQSPVLIIY

SEQ ID NO: 261 - protein, 17 aa, Homo sapiens
VHWYQQLPGAAPKVLIY

SEQ ID NO: 262 - protein, 17 aa, Homo sapiens
VNWYQQFPGTAPKLLMY

SEQ ID NO: 263 - protein, 17 aa, Homo sapiens
VSWYQQLPGSAPKLLIY

SEQ ID NO: 264 - protein, 17 aa, Homo sapiens
VTWFQQLPGTAPKLLIY

SEQ ID NO: 265 - protein, 17 aa, Homo sapiens
VSWYQQRPGQSPVLVMY

SEQ ID NO: 266 - protein, 17 aa, Homo sapiens
VSWYQQLPGTAPKLLIY

SEQ ID NO: 267 - protein, 17 aa, Homo sapiens
LNWFQQRPGQSPRRLIY

SEQ ID NO: 268 - protein, 18 aa, Homo sapiens
YGWFQQKSPGSAPVTVIY

SEQ ID NO: 269 - protein, 18 aa, Homo sapiens
YGWYQQKSPGSAPVTVIY

SEQ ID NO: 270 - protein, 18 aa, Homo sapiens
YGWYQQKSPGSAPVTLIY

SEQ ID NO: 271 - protein, 36 aa, Homo sapiens
KRPSGIPERFSGSNSGNTATLTISGTQAMDEADYYC

SEQ ID NO: 272 - protein, 36 aa, Homo sapiens
NRPSGVPDRFSGSKSGASASLTITGLQAEDEADYYC

SEQ ID NO: 273 - protein, 36 aa, Homo sapiens
NRPSGVSNRFSGSKSGNTASLTISGLQADDEADYYC

SEQ ID NO: 274 - protein, 36 aa, Homo sapiens
NRPSGIPDRFSGSDSGKTASLTITGAQAEDEADYYC

SEQ ID NO: 275 - protein, 36 aa, Homo sapiens
KRPSGVPDRFSGSKSGNTASLTVSGLQAEDEADYYC

SEQ ID NO: 276 - protein, 36 aa, Homo sapiens
KRPSGIPERFSASNSENTGTLTISGTQAMDEADYYC

SEQ ID NO: 277 - protein, 36 aa, Homo sapiens
NRPSGVPDRFSGSKSGTSASLAITGLRAEDEADYYC

SEQ ID NO: 278 - protein, 36 aa, Homo sapiens
QRPSGVPARFSGSKSGTSASLAVDGLQSDDEADYYC

SEQ ID NO: 279 - protein, 36 aa, Homo sapiens
KRPSGIPERFSGSNSGNTATLTISGTQAMDEGDYYC

SEQ ID NO: 280 - protein, 36 aa, Homo sapiens
NRDSGVPGTFSGSGSGTDFTLKISRVEADDVGVYFC

SEQ ID NO: 281 - protein, 36 aa, Homo sapiens
KRPSGIPARFSGSNSGNTATLTISGTQAMDEADYYC

SEQ ID NO: 282 - protein, 36 aa, Homo sapiens
KRPSGIPERFSGSNSGSTATLTITGTQAMDDADYYC

SEQ ID NO: 283 - protein, 36 aa, Homo sapiens
KRPSGIPERFSGSNSGSTATLTISGTQALDEADYYC

SEQ ID NO: 284 - protein, 36 aa, Homo sapiens
QRPSGVPDRFSGSKSGTSASLAISGLRSEDEADYYC

SEQ ID NO: 285 - protein, 36 aa, Homo sapiens
ERPSGIPERFSGSNSGNTATLTIRGTQAMDEADYYC

SEQ ID NO: 286 - protein, 36 aa, Homo sapiens
KRPSGIPERFSGSNSGNTATLTISGAQAVDEADYYC

SEQ ID NO: 287 - protein, 36 aa, Homo sapiens
QRPSGVPDRFSGSKSGTSASLAISGLRSEDETDYFC

SEQ ID NO: 288 - protein, 36 aa, Homo sapiens
QRPSGVPDRFSGSKSGTSASLAISGLQSEDEADYYC

SEQ ID NO: 289 - protein, 36 aa, Homo sapiens
KRPSRVPERFSGSNSGNTATLTISETQAMDEADYYC

SEQ ID NO: 290 - protein, 36 aa, Homo sapiens
KRPSGVPERFSGSNSGNTATLNISGTQPMDEADYYC

SEQ ID NO: 291 - protein, 36 aa, Homo sapiens
KRPSGIPKRFSGSNSGNTATLTISGTQAMDEADYYC

SEQ ID NO: 292 - protein, 36 aa, Homo sapiens
KRPSGVPERFSGSKSGTSASLAISGLQSEDEADYYC

SEQ ID NO: 293 - protein, 36 aa, Homo sapiens
NRDSGVPDRFSGSGSGTDFTLKISRVEAEDVGVYYC

SEQ ID NO: 294 - protein, 36 aa, Homo sapiens
KRPSDIPSRFSGSASGSMGALIITGVQAEDEAVYFC

SEQ ID NO: 295 - protein, 36 aa, Homo sapiens
NRPSDIPSRFSGSKSGSTATLTITGVQAEDEAVYYC

SEQ ID NO: 296 - protein, 36 aa, Homo sapiens
KRPSDIPSRFSGSASGSTATLTITGVQADDEAVYYC

SEQ ID NO: 297 - protein, 36 aa, Homo sapiens
KRPSDIPSRFSGSLSGSTNTLTITGVQVEDESVYFC

SEQ ID NO: 298 - protein, 36 aa, Homo sapiens
KRPSDIPSRFSGSTSGSMGALIITGVQAEDEAVYFC

SEQ ID NO: 299 - protein, 36 aa, Homo sapiens
NRPSDIPSRFSGSLSDSTHTLTITGVRAEDEAVYFC

SEQ ID NO: 300 - protein, 36 aa, Homo sapiens
KRPSDIPSRFSGSKSGSTGTLTITGVQAEDEAVYFC

SEQ ID NO: 301 - protein, 36 aa, Homo sapiens
KRPSDIPSRFSGSTSGSTNTLTITGVQVEDEAVYFC

SEQ ID NO: 302 - protein, 36 aa, Homo sapiens
NRPSNIPSRFSGSTSGSTATLTITGVQADDEAVYYC

SEQ ID NO: 303 - protein, 10 aa, Homo sapiens
FGGGTKLTVL

SEQ ID NO: 304 - protein, 10 aa, Homo sapiens
FGTGTKVTVL

SEQ ID NO: 305 - protein, 10 aa, Homo sapiens
FGGGTKVTVL

SEQ ID NO: 306 - protein, 10 aa, Homo sapiens
FGTGTKLTVL

SEQ ID NO: 307 - protein, 10 aa, Homo sapiens
FGSGTKLTVL

SEQ ID NO: 308 - protein, 10 aa, Homo sapiens
FGQGTKVEIK

SEQ ID NO: 309 - protein, 10 aa, Homo sapiens
FGTGTQLTVL

SEQ ID NO: 310 - protein, 10 aa, Homo sapiens
FGAGTKVTVL

SEQ ID NO: 311 - protein, 10 aa, Homo sapiens
FGGGTQLTVL

SEQ ID NO: 312 - protein, 10 aa, Homo sapiens
FGAGTTLTVL

SEQ ID NO: 313 - protein, 10 aa, Homo sapiens
YGAGTTLTVL

SEQ ID NO: 314 - protein, 116 aa, Homo sapiens
QVQLLQSAAEVKKPGESLKISCKGSGYRFTSYWIGWVRQMPGKGLEWVGIIYPDDSDTRYSPSFQGQVTISADTSISTTYLQWSSLKASDTAIYYCASGEVAFDVWGQGTTVTVSS

SEQ ID NO: 315 - protein, 106 aa, Homo sapiens
QSVLTQPPSLSVSPRQTASITCSGDKLGDKFVSWYQQKPGQSPVLVIYQDTKRPSGIPERFSGSNSGNTATLTISGTQAMDEADYYCQAWDSSTGVFGGGTKLTVL

SEQ ID NO: 316 - protein, 119 aa, Homo sapiens
QVQLVQSGAEVKKPGESLRISCKGSGYSFTNNWINWVRQMPGKGLEWMGRIDPDDSYTNYSPSFQGHVIFSADKSSNTVYLQWSSLEASDTAMYYCARRAQGGGGYDYWGQGTLVTVSS

SEQ ID NO: 317 - protein, 112 aa, Homo sapiens
QSVLTQPPSVSGAPGQRVTISCTGSSSNIGAGFNVHWYQQVAGLAPKLLIHDNINRPSGVPDRFSGSKSGASASLTITGLQAEDEADYYCQSYDSSLSGWFAFGTGTKVTVL

SEQ ID NO: 318 - protein, 121 aa, Homo sapiens
QVQLQQSGPGLVKPAQTLSLTCDISGDSVSSKSAAWHWIRQSPSRGLEWLGRTYYRSKWYSDYGESVKSRVVINADTSNNQISLQLSSVTPEDTAAYYCARGIGSLNFDIWGQGTMVTVSS

SEQ ID NO: 319 - protein, 112 aa, Homo sapiens
QSVVTQPASVSGSPGQSITISCTGTSSDIGGYDYVSWYQQHPGKAPKLMIFDVSNRPSGVSNRFSGSKSGNTASLTISGLQADDEADYYCSSYRAGSTLNLLFGGGTKLTVL

SEQ ID NO: 320 - protein, 117 aa, Homo sapiens
QVQLQQSGGGVVQPGRSLRLSCAASGFTFSNYAMHWVRQAPGKGLEWVAVISYDGSNEYYADSVKGRFTISRDNSRNTLDLQMNSLRAEDTAVYYCARGTNYGMDVWGQGTMVTVSS

SEQ ID NO: 321 - protein, 108 aa, Homo sapiens
LSELTQDPAVSVALGQTVRITCQGDSLRSYYASWYQQKPGQAPVLVIYGKNNRPSGIPDRFSGSDSGKTASLTITGAQAEDEADYYCQSRDTTGNYLLFGGGTKLTVL

SEQ ID NO: 322 - protein, 121 aa, Homo sapiens
QVQLQQSGPGLVKPSQTLSVTCVISGDSVSSKSAAWHWIRQSPSRGLEWLGRTYYRSKWYSDYGESVKSRVVINADTSNNQISLQLSSVTPEDTAAYYCARGIGSLNFDIWGQGTMVTVSS

SEQ ID NO: 323 - protein, 111 aa, Homo sapiens
QSVLTQPPSASGSPGQSVTISCTGTSGDVGGYNYVSWYQQHPGKAPKLILYEVTKRPSGVPDRFSGSKSGNTASLTVSGLQAEDEADYYCSSYGGPNNLVLFGGGTKVTVL

SEQ ID NO: 324 - protein, 117 aa, Homo sapiens
QVQLVQSGAEVKKPGESLKISCTGSGYRFSHYWIGWVRQMPGKGLEWMGMIYPDDSDTRYSPSFQGQVTISADKSINTAYLQWTSLRASDSAIYYCATGVISGTDYWGQGTLVTVSS

SEQ ID NO: 325 - protein, 106 aa, Homo sapiens
SYVLTQPPSVSVSPGQTARITCSGDKLGDKYVSWYQLKPGQSPVVVIYQDNKRPSGIPERFSASNSENTGTLTISGTQAMDEADYYCQTWDSGTGVFGTGTKLTVL

SEQ ID NO: 326 - protein, 119 aa, Homo sapiens
EVQLVESGAEVKKPGESLRISCKGSGYSFTNNWINWVRQMPGKGLEWMGRIDPDDSYTNYSPSFQGHVIFSADKSSNTVYLQWSSLEASDTAMYYCARRAQGGGGYDYWGQGTLVTVSS

SEQ ID NO: 327 - protein, 112 aa, Homo sapiens
QSVLTQPSSVSGAPGQRVTISCTGSSSNIGAGYGVHWYQQLPGTAPKLLIYDDINRPSGVPDRFSGSKSGTSASLAITGLRAEDEADYYCQSYDTSRGGLYVFGSGTKLTVL

SEQ ID NO: 328 - protein, 116 aa, Homo sapiens
QVQLVESGAEVKKPGESLKISCKGSGYSFTTYWIAWVRQMPGKGLEWMGIIYPGDSDTRYSPSFQGQVTISADKSISTAYLQWSSLKASDTAMYYCARLGGGMDVWGQGTMVTVSS

SEQ ID NO: 329 - protein, 110 aa, Homo sapiens
QAGLTQPPSASGTPGQRVTISCSGSSSNIGSNTVSWYRQIPGTAPKFLLYGNNQRPSGVPARFSGSKSGTSASLAVDGLQSDDEADYYCAAWDDSLSGYVFGTGTKLTVL

SEQ ID NO: 330 - protein, 121 aa, Homo sapiens
EVQLVQSGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTENEVWPSLDVWGQGTTVTVSS

SEQ ID NO: 331 - protein, 107 aa, Homo sapiens
QSVLTQPPSVSVSPGQTASITCSGDKLGNKYASWYRQKPGQSPVLVIYQDTKRPSGIPERFSGSNSGNTATLTISGTQAMDEGDYYCQAWDSSTAVVFGGGTKLTVL

SEQ ID NO: 332 - protein, 116 aa, Homo sapiens
QVQLVQSGAEVKKPGEPLKISCKGSGYSFPTYWIAWVRQMPGKGLEWMGIIYPGDSDTRYSPSFQGQVTISADKSISTAYLQWGSLRASDTAMYYCARGGSGLDYWGQGTLVTVSS

SEQ ID NO: 333 - protein, 106 aa, Homo sapiens
SYELMQPPSVSVSPGQTASITCSGDKLGTKYVSWYQQKPGQSPVLVIYQDSKRPSGIPERFSGSNSGNTATLTISGTQAMDEADYYCQAWDSSTVVFGGGTKLTVL

SEQ ID NO: 334 - protein, 116 aa, Homo sapiens
EVQLVQSGAEVKEPGASVKVSCKASGYTFTNYAMHWVRQAPGQRLEWMGWINPNTGGTNYAQKFQGRVTMTRDTSISTAYMELSSLRSDDTAVYYCARGGGGLVFWGQGTLVTVSS

SEQ ID NO: 335 - protein, 111 aa, Homo sapiens
DIVMTQTPLSLPVTLGQPASISCRSSQSLVDRDGNTYLNWFRQRPGQSPRRLIYKVSNRDSGVPGTFSGSGSGTDFTLKISRVEADDVGVYFCMQATHWPPTFGQGTKVEI

SEQ ID NO: 336 - protein, 117 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCATPMEDGMDVWGQGTTVTVSS

SEQ ID NO: 337 - protein, 106 aa, Homo sapiens
QAGLTQPPSVSVSPGQTVTITCSADNLGDKYTSWYQQRPGQSPVLVIYQDTKRPSGIPARFSGSNSGNTATLTISGTQAMDEADYYCQAWDSSTGVFGTGTKVTVL

SEQ ID NO: 338 - protein, 115 aa, Homo sapiens
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARGGPPDYWGQGTLVTVSS

SEQ ID NO: 339 - protein, 106 aa, Homo sapiens
LPVLTQPPSVSVSPGQTASITCSGDRLGDKYASWYQQKPGQSPVLVIYQDSKRPSGIPERFSGSNSGNTATLTISGTQAMDEADYYCQAWDSSTAVFGGGTKLTVL

SEQ ID NO: 340 - protein, 115 aa, Homo sapiens
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCTRSSFNDHWGQGTLVTVSS

SEQ ID NO: 341 - protein, 106 aa, Homo sapiens
QPVLTQPPSVSVSPGQTASITCSGDKLGNKFVSWFQQKPGQSPVLVIYQDTKRPSGIPERFSGSNSGSTATLTITGTQAMDDADYYCQAWNTNTALFGGGTKLTVL

SEQ ID NO: 342 - protein, 118 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARLGGSLPIDYWGQGTLVTVSS

SEQ ID NO: 343 - protein, 106 aa, Homo sapiens
QSVLTQPPSVSVSPGQTASITCSGDKLGDKYVSWYQQKPGQSPVLVIYQDSKRPSGIPERFSGSNSGSTATLTISGTQALDEADYYCQAWDNNIGVFGGGTKLTVL

SEQ ID NO: 344 - protein, 118 aa, Homo sapiens
EVQLVQSGAEVKKPGESLKISCQGSGYTFTNYWIAWVRQMPGKGLEWMGIIYPGDSDTRYSPSFQGQVTISADKSISTAYLQWSSLKASDTAMYYCAIGYDYGAFDIWGQGTMVTVSS

SEQ ID NO: 345 - protein, 110 aa, Homo sapiens
QPVLTQPPSASGTPGQRVSISCSGSNSNIGSNSVYWYQQLPGTAPKLLIYRNNQRPSGVPDRFSGSKSGTSASLAISGLRSEDEADYYCAAWDDSLSGYVFGTGTQLTVL

SEQ ID NO: 346 - protein, 116 aa, Homo sapiens
QVQLVESGAEVKKPGEPLKISCKGSGYSFPTYWIAWVRQMPGKGLEWMGIIYPADSDTRYSPSFQGQVTISADKSISTAYLQWGSLRASDTAMYYCARGGSGLDYWGQGTLVTVSS

SEQ ID NO: 347 - protein, 106 aa, Homo sapiens
QAGLTQPPSVSVSPGQTAIIPCSGEKLGDKYVSWYQQKPGESPLSIIYQDTERPSGIPERFSGSNSGNTATLTIRGTQAMDEADYYCQAWDSNTMVFGGGTKLTVL

SEQ ID NO: 348 - protein, 120 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCASRLLFGDDAFDIWGQGTMVTVSS

SEQ ID NO: 349 - protein, 106 aa, Homo sapiens
SYVLTQPPSVSVSPGQTATITCSGDKLGDEYVSWYQQKPGQSPVLVIYQDSKRPSGIPERFSGSNSGNTATLTISGAQAVDEADYYCQAWDSNAGAFGTGTKLTVL

SEQ ID NO: 350 - protein, 118 aa, Homo sapiens
QMQLVQSGAEVKKPGESLKISCKGSGYSFSTNWIGWVRQMPGKGLEWMGIIYPGDSDTRYSPSFQGQVTISADKSISTAYLQWSSLKASDTAMYYCARFYDSSGNDYWGQGTLVTVSS

SEQ ID NO: 351 - protein, 106 aa, Homo sapiens
QSVLTQPPSVSVSPGQTASITCSGDKLGDKYASWYQQKPGQSPVLVIFQDNKRPSGIPERFSGSNSGNTATLTISGTQAMDEADYYCQTWDSSTAIFGGGTKLTVL

SEQ ID NO: 352 - protein, 120 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCVVRPAAAGTRIDYWGQGTLVTVSS

SEQ ID NO: 353 - protein, 106 aa, Homo sapiens
QSVLTQPPSVSVSPGQTASITCSGDKLGDKYASWYQQKPGQSPVLIIYQDNKRPSGIPERFSGSNSGNTATLTISGTQAMDEADYYCQAWDSSTYVFGTGTKLTVL

SEQ ID NO: 354 - protein, 117 aa, Homo sapiens
EVQLVQSGAEVKKPGESLKISCKGSRYSFTSYWIGWVRQMPGKGLEWMGIIYPGDSDTRYSPSFQGQVTISADKSISTAYLQWSSLKASDTAMYYCARLKDGAFDIWGQGTMVTVSS

SEQ ID NO: 355 - protein, 110 aa, Homo sapiens
QAVLTQPPSASGTPGQRVTISCSGSSSNIEGNYVHWYQQLPGAAPKVLIYRNNQRPSGVPDRFSGSKSGTSASLAISGLRSEDETDYFCAAWDDSLNGYVFGAGTKVTVL

SEQ ID NO: 356 - protein, 114 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCASWGMDVWGQGTLVTVSS

SEQ ID NO: 357 - protein, 110 aa, Homo sapiens
QPVLTQPPSASGTPGQRVTISCSGSSSNIGTHTVNWYQQFPGTAPKLLMYSTFQRPSGVPDRFSGSKSGTSASLAISGLQSEDEADYYCAAWDDSLNGPVFGGGTQLTVL

SEQ ID NO: 358 - protein, 116 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARHDYGMDVWGQGTTVTVSS

SEQ ID NO: 359 - protein, 106 aa, Homo sapiens
QSVLTQPPSVSVSPGQTASITCSGDKLGDKFASWYQQKPGQSPVLVIYQDNKRPSRVPERFSGSNSGNTATLTISETQAMDEADYYCQAWVNNTVVFGGGTKVTVL

SEQ ID NO: 360 - protein, 116 aa, Homo sapiens
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARHDYGMDVWGQGTMVTVSS

SEQ ID NO: 361 - protein, 110 aa, Homo sapiens
LPVLTQPPSASGTPGQRVTISCSGSRSNVGGNTVSWYQQLPGSAPKLLIYTNNQRPSGVPDRFSGSKSGTSASLAISGLQSEDEADYYCAAWDDSLNGYVFGTGTQLTVL

SEQ ID NO: 362 - protein, 119 aa, Homo sapiens
QMQLVQSGAEVKKPGESLKISCKGSGYSFTSYWIGWVRQMPGKGLEWLGIIYPGDSDTRYSPSFQGQVTISADKSISTAYLQWSSLKASDTAMYYCARLGQGSGPFDYWGQGTLVTVSS

SEQ ID NO: 363 - protein, 110 aa, Homo sapiens
QSVVTQPPSVSGTPGQRVSISCSGSSSNIGSHSVTWFQQLPGTAPKLLIYGNSQRPSGVPDRFSGSKSGTSASLAISGLQSEDEADYYCASWDDSLNGYVFGTGTKLTVL

SEQ ID NO: 364 - protein, 118 aa, Homo sapiens
QVQLVQSGAEVKKPGESLKISCKGFGYSFTNYWIGWVRQMPGKGLEWMGIIYPGDSDTRYSPSFQGQVTISADKSITTAYLQWSSLKASDTAMYYCASHTKDGTWDIWGQGTMVTVSS

SEQ ID NO: 365 - protein, 108 aa, Homo sapiens
QAGLTQPPSVSVSPGQTASITCSGERLGDKYVSWYQQKPGQSPVLVIYQDDKRPSGVPERFSGSNSGNTATLNISGTQPMDEADYYCQAWDSSTSLYVFGTGTKLTVL

SEQ ID NO: 366 - protein, 116 aa, Homo sapiens
QVQLVQSGAEVKKPGESLKISCKGSGYSFTSYWIGWVRQMPGKGLEWMGIIYPGDSDTRYSPSFQGQVTISADKSISTAYLQWSSLKASDTAIYYCARLGGAMDVWGQGTLVTVSS

SEQ ID NO: 367 - protein, 109 aa, Homo sapiens
SYELMQPPSLSVSPGQTASITCSGDKLGDKYVSWYQQRPGQSPVLVMYQDKKRPSGIPKRFSGSNSGNTATLTISGTQAMDEADYYCAAWDDSLNGYYVFGTGTQLTVL

SEQ ID NO: 368 - protein, 122 aa, Homo sapiens
QVQLQQSGAGLVKASQTLALTCAISGDSVSSNSAAWNWIRQSPSRGLEWLGRTYYRSKWYNDYAVSVKSRITINPDTSTNQFSLQLNSVTPEDTAVYYCTRGTGDKRAFDYWGQGTLVTVSS

SEQ ID NO: 369 - protein, 110 aa, Homo sapiens
QSVLTQPPSASGTPGQRVTISCSGSSSNIGSYFVSWYQQLPGTAPKLLIYRNDKRPSGVPERFSGSKSGTSASLAISGLQSEDEADYYCAAWDDSLSGRIFGGGTKLTVL

SEQ ID NO: 370 - protein, 119 aa, Homo sapiens
QVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARAFRRYDAFDIWGQGTLVTVSS

SEQ ID NO: 371 - protein, 112 aa, Homo sapiens
DIQMTQTPLSLSVTLGQPASISCRSSQGLVYSDGNTYLNWFQQRPGQSPRRLIYKVSNRDSGVPDRFSGSGSGTDFTLKISRVEAEDVGVYYCMQGTHWPPAFGQGTKVEIK

SEQ ID NO: 372 - protein, 116 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARGSGGYDYWGQGTLVTVSS

SEQ ID NO: 373 - protein, 106 aa, Homo sapiens
QSVLTQPPSVSVSPGQTASITCSGNKLGDKFVSWYHQRPGQSPVLVIYEDNKRPSGIPERFSGSNSGNTATLTISGTQAMDEGDYYCQTWDVSSYVFGTGTKVTVL

SEQ ID NO: 374 - protein, 122 aa, Gallus gallus
AVTLDESGGGLQTPGGALSLVCKASGFTFSSFPMVWVRQAPGKGLEFVAGIVRTSTPKYGAAVTGRATISRDNGQSTVRLQLNNLRAEDTGTYFCAKSATICGNCPEDIDAWGHGTEVIVSS

SEQ ID NO: 375 - protein, 105 aa, Gallus gallus
QAALTQPASVSANPGETVKITCSGGSSDYGWFQQKSPGSAPVTVIYYSDKRPSDIPSRFSGSASGSMGALIITGVQAEDEAVYFCGSRDSSNVAIFGAGTTLTVL

SEQ ID NO: 376 - protein, 122 aa, Gallus gallus; identical to SEQ ID NO: 374

SEQ ID NO: 377 - protein, 105 aa, Gallus gallus
QAALTQPASVSANPGETVKITCSGGSSDYGWFQQKSPGSAPVTVIYYSDKRPSDIPSRFSGSTSGSMGALIITGVQAEDEAVYFCGSRDSSNVAIFGAGTTLTVL

SEQ ID NO: 378 - protein, 124 aa, Gallus gallus
AVTLDESGGGLQTPGGGLSLVCKASGFTFSNHQMAWVRQAPGKGLEYVGLINAAGSWTYYGTAVKGRATISRDNGQSTVRLQLNNLRAEDTATYYCAKDVYGYCSTCGATIDTWGHGTEVIVSS

SEQ ID NO: 379 - protein, 105 aa, Gallus gallus
QAALTQPSSVSANPGETVKITCSGPTYDYGWFQQKSPGSAPVTVIYYNDNRPSDIPSRFSGSKSGSTATLTITGVQAEDEAVYYCGTYEDNNGASFGAGTTLTVL

SEQ ID NO: 380 - protein, 126 aa, Gallus gallus
AVTLDESGGGLQTPGGALSLVCKASGFTFSSFNMFWVRQAPGKGLEFVAHIKRNGGETDYGAAVKGRATISRDDGQSTVRLQLNNLRAEDTATYFCAKDAGDNCRWNYCTTNIDAWGHGTEVIVSS

SEQ ID NO: 381 - protein, 110 aa, Gallus gallus
QAALTQPASVSANPGETVKITCSGGGRWYGTYYYGWYQQKSPGSAPVTVIYSNDKRPSDIPSRFSGSASGSTATLTITGVQADDEAVYYCGSWDSTDTTAFGAGTTLTVL

SEQ ID NO: 382 - protein, 122 aa, Gallus gallus
AVTLDESGGGLQTPGGPLSLVCEGSGFTFSSYGMAWVRQAPGKRLEWVASISSTGINAYYGAAVKGRATISRDNGQSTVRLQLNNLRAEDTGIYYCSKCAYSGSCSTSIDAWGHGTEVIVSS

SEQ ID NO: 383 - protein, 107 aa, Gallus gallus
QAALTQPASVSANLGGTVEITCSGGSSGYGYGWYQQKSPGSAPVTVIYNNNNRPSDIPSRFSGSLSDSTHTLTITGVRAEDEAVYFCGAWESSATDIFGAGTTLTVL

SEQ ID NO: 384 - protein, 122 aa, Gallus gallus
AVTLDESGGGLQTPGGALSLVCKASGFDFSSNAMGWVRQAPGKGLEFIASITTDGSYTTYGAAVKGRATISRDDGQSTLRLQLNNLRAEDTATYYCAKDSSIHWNGYDIDAWGHGTEVIVSS

SEQ ID NO: 385 - protein, 106 aa, Gallus gallus
QAALTQPSSVSANPGETVKITCSGSINNYGWYQQKSPGSAPVTVIYQNDKRPSDIPSRFSGSKSGSTGTLTITGVQAEDEAVYFCGSEDSRTYLGIFGAGTTLTVL

SEQ ID NO: 386 - protein, 123 aa, Gallus gallus
AVTLDESGGGLQTPGGGLSLVCKASGFTFSSHAMFWVRQAPGKGLEYVAYINYDGITFYGAAVKGRATISRDNGQSTVRLQLNNLRAEDTATYFCAKTTCSGCWDAAALIDAWGHGTEVIVSS

SEQ ID NO: 387 - protein, 106 aa, Gallus gallus
QAALTQPASVSSNPGETVKITCSGGSSGYAYGWYQQKSPGSAPVTLIYSNDKRPSDIPSRFSGSKSGSTGTLTITGVQAEDEAVYYCGSFDSSSYIFGAGTTLTVL

SEQ ID NO: 388 - protein, 122 aa, Gallus gallus; identical to SEQ ID NO: 374

SEQ ID NO: 389 - protein, 104 aa, Gallus gallus
QAALTQPASVSANPGETVKITCSGGSSDYGWFQQKSPGSAPVTVIYYSDKRPSDIPSRFSGSTSGSTNTLTITGVQVEDEAVYFCGSGDNSYVGFGAGTTLTVL

SEQ ID NO: 390 - protein, 123 aa, Homo sapiens
AVTLDESGGGLQTPGGALSLVCKASGFTFSSSGMAWVRQAPGKGLEFVAGITSSAGNTYYAPAVKGRATISRDNGQSTVRLQLNNLRAADTGTYFCAKDSNSGSPDASEIDAWGHGTEVIVSS

SEQ ID NO: 391 - protein, 105 aa, Homo sapiens
QAALTQPASVSANPGETVKITCSGYKSYYGWYQQKSPGSAPVTVIYDNTNRPSNIPSRFSGSTSGSTATLTITGVQADDEAVYYCGNADHSYVGVFGAGTTLTVL

SEQ ID NO: 392 - protein, 25 aa, Homo sapiens
QVQLLQSAAEVKKPGESLKISCKGS

SEQ ID NO: 393 - protein, 30 aa, Homo sapiens
QVQLLQSAAEVKKPGESLKISCKGSGYRFT

SEQ ID NO: 394 - protein, 29 aa, Homo sapiens
QVQLLQSAAEVKKPGESLKISCKGSGYRF

SEQ ID NO: 395 - protein, 7 aa, Homo sapiens
GYRFTSY

SEQ ID NO: 396 - protein, 10 aa, Homo sapiens
GYRFTSYWIG

SEQ ID NO: 397 - protein, 5 aa, Homo sapiens
SYWIG

SEQ ID NO: 398 - protein, 6 aa, Homo sapiens
TSYWIG

SEQ ID NO: 399 - protein, 19 aa, Homo sapiens
WIGWVRQMPGKGLEWVGII

SEQ ID NO: 400 - protein, 14 aa, Homo sapiens
WVRQMPGKGLEWVG

SEQ ID NO: 401 - protein, 11 aa, Homo sapiens
WVRQMPGKGLE

SEQ ID NO: 402 - protein, 6 aa, Homo sapiens
YPDDSD

SEQ ID NO: 403 - protein, 10 aa, Homo sapiens
IIYPDDSDTR

SEQ ID NO: 404 - protein, 17 aa, Homo sapiens
IIYPDDSDTRYSPSFQG

SEQ ID NO: 405 - protein, 13 aa, Homo sapiens
WVGIIYPDDSDTR

SEQ ID NO: 406 - protein, 41 aa, Homo sapiens
TRYSPSFQGQVTISADTSISTTYLQWSSLKASDTAIYYCAS

SEQ ID NO: 407 - protein, 39 aa, Homo sapiens
YSPSFQGQVTISADTSISTTYLQWSSLKASDTAIYYCAS

SEQ ID NO: 408 - protein, 32 aa, Homo sapiens
QVTISADTSISTTYLQWSSLKASDTAIYYCAS

SEQ ID NO: 409 - protein, 37 aa, Homo sapiens
YSPSFQGQVTISADTSISTTYLQWSSLKASDTAIYYC

SEQ ID NO: 410 - protein, 7 aa, Homo sapiens
GEVAFDV

SEQ ID NO: 411 - protein, 8 aa, Homo sapiens
ASGEVAFD

SEQ ID NO: 412 - protein, 12 aa, Homo sapiens
VWGQGTTVTVSS

SEQ ID NO: 413 - protein, 822 aa, Homo sapiens
MSSWIRWHGPAMARLWGFCWLVVGFWRAAFACPTSCKCSASRIWCSDPSPGIVAFPRLEPNSVDPENITEIFIANQKRLEIINEDDVEAYVGLRNLTIVDSGLKFVAHKAFLKNSNLQHINFTRNKLTSLSRKHFRHLDLSELILVGNPFTCSCDIMWIKTLQEAKSSPDTQDLYCLNESSKNIPLANLQIPNCGLPSANLAAPNLTVEEGKSITLSCSVAGDPVPNMYWDVGNLVSKHMNETSHTQGSLRITNISSDDSGKQISCVAENLVGEDQDSVNLTVHFAPTITFLESPTSDHHWCIPFTVKGNPKPALQWFYNGAILNESKYICTKIHVTNHTEYHGCLQLDNPTHMNNGDYTLIAKNEYGKDEKQISAHFMGWPGIDDGANPNYPDVIYEDYGTAANDIGDTTNRSNEIPSTDVTDKTGREHLSVYAVVVIASVVGFCLLVMLFLLKLARHSKFGMKGPASVISNDDDSASPLHHISNGSNTPSSSEGGPDAVIIGMTKIPVIENPQYFGITNSQLKPDTFVQHIKRHNIVLKRELGEGAFGKVFLAECYNLCPEQDKILVAVKTLKDASDNARKDFHREAELLTNLQHEHIVKFYGVCVEGDPLIMVFEYMKHGDLNKFLRAHGPDAVLMAEGNPPTELTQSQMLHIAQQIAAGMVYLASQHFVHRDLATRNCLVGENLLVKIGDFGMSRDVYSTDYYRVGGHTMLPIRWMPPESIMYRKFTTESDVWSLGVVLWEIFTYGKQPWYQLSNNEVIECITQGRVLQRPRTCPQEVYELMLGCWQREPHMRKNIKGIHTLLQNLAKASPVYLDILG

SEQ ID NO: 414 - protein, 8 aa, Gallus gallus
GFTFSSYS

SEQ ID NO: 415 - protein, 8 aa, Gallus gallus
GFSFGSSA

SEQ ID NO: 416 - protein, 8 aa, Gallus gallus
GGTFSSYA

SEQ ID NO: 417 - protein, 8 aa, Gallus gallus
GYTFTGHY

SEQ ID NO: 418 - protein, 8 aa, Gallus gallus
GFTFNNFY

SEQ ID NO: 419 - protein, 8 aa, Gallus gallus
GFTFRGFY

SEQ ID NO: 420 - protein, 8 aa, Gallus gallus
GFTFSTYA

SEQ ID NO: 421 - protein, 8 aa, Gallus gallus
GFTFNNSY

SEQ ID NO: 422 - protein, 8 aa, Gallus gallus
RFTFSSYP

SEQ ID NO: 423 - protein, 8 aa, Gallus gallus
GFTFDDFA

SEQ ID NO: 424 - protein, 8 aa, Gallus gallus
GYTFTRYG

SEQ ID NO: 425 - protein, 8 aa, Gallus gallus
GYTFRNYY

SEQ ID NO: 426 - protein, 8 aa, Gallus gallus
GYTFTSYG

SEQ ID NO: 427 - protein, 8 aa, Gallus gallus
GFTSNTYA

SEQ ID NO: 428 - protein, 8 aa, Gallus gallus
GYTFTSYY

SEQ ID NO: 429 - protein, 8 aa, Gallus gallus
GFTFGDYA

SEQ ID NO: 430 - protein, 8 aa, Gallus gallus
GFTFRRYG

SEQ ID NO: 431 - protein, 8 aa, Gallus gallus
GFTFSRYS

SEQ ID NO: 432 - protein, 8 aa, Gallus gallus
ISGSGGST

SEQ ID NO: 433 - protein, 8 aa, Gallus gallus
ISSSSSYI

SEQ ID NO: 434 - protein, 8 aa, Gallus gallus
ISHSGSTT

SEQ ID NO: 435 - protein, 8 aa, Gallus gallus
IIPIFGTA

SEQ ID NO: 436 - protein, 8 aa, Gallus gallus
INPRGGST

SEQ ID NO: 437 - protein, 8 aa, Gallus gallus
ISRGGNSV

SEQ ID NO: 438 - protein, 8 aa, Gallus gallus
ISSSGSAV

SEQ ID NO: 439 - protein, 8 aa, Gallus gallus
ISYDGAGK

SEQ ID NO: 440 - protein, 8 aa, Gallus gallus
ITWNSAAT

SEQ ID NO: 441 - protein, 8 aa, Gallus gallus
ISAYNGNT

SEQ ID NO: 442 - protein, 8 aa, Gallus gallus
VNPNSGNA

SEQ ID NO: 443 - protein, 8 aa, Gallus gallus
ISGSGSST

SEQ ID NO: 444 - protein, 8 aa, Gallus gallus
ISPDGSDK

SEQ ID NO: 445 - protein, 8 aa, Gallus gallus
INPSGGTT

SEQ ID NO: 446 - protein, 10 aa, Gallus gallus
IRSKAYGGTT

SEQ ID NO: 447 - protein, 8 aa, Gallus gallus
ISGGGGST

SEQ ID NO: 448 - protein, 7 aa, Gallus gallus
ARDAHDY

SEQ ID NO: 449 - protein, 12 aa, Gallus gallus
ARVKDYDSSLDY

SEQ ID NO: 450 - protein, 13 aa, Gallus gallus
ARGITGTGYGMDV

SEQ ID NO: 451 - protein, 12 aa, Gallus gallus
AKIGAYGYYFHY

SEQ ID NO: 452 - protein, 15 aa, Gallus gallus
ARDAVVVADSGSMDV

SEQ ID NO: 453 - protein, 7 aa, Gallus gallus
ATWGMDV

SEQ ID NO: 454 - protein, 14 aa, Gallus gallus
ARESIAAAGPYFDY

SEQ ID NO: 455 - protein, 8 aa, Gallus gallus
VRGGDFLY

SEQ ID NO: 456 - protein, 11 aa, Gallus gallus
ARDGGTGGFDY

SEQ ID NO: 457 - protein, 14 aa, Gallus gallus
AGEHYTLTDEEFDF

SEQ ID NO: 458 - protein, 8 aa, Gallus gallus
AKGGDYLY

SEQ ID NO: 459 - protein, 7 aa, Gallus gallus
ARDPHDF

SEQ ID NO: 460 - protein, 13 aa, Gallus gallus
ARDRGGPKDPFDI

SEQ ID NO: 461 - protein, 20 aa, Gallus gallus
ARDRGYCSGGSCPGIDAFDI

SEQ ID NO: 462 - protein, 16 aa, Gallus gallus
AREPPATVAYYYGMDV

SEQ ID NO: 463 - protein, 10 aa, Gallus gallus
ARERLIGMDV

SEQ ID NO: 464 - protein, 10 aa, Gallus gallus
AREVNYGLDV

SEQ ID NO: 465 - protein, 12 aa, Gallus gallus
ARIERYSYGIDY

SEQ ID NO: 466 - protein, 12 aa, Gallus gallus
ARNRKYTNFLDI

SEQ ID NO: 467 - protein, 9 aa, Gallus gallus
ARNWASFDY

SEQ ID NO: 468 - protein, 11 aa, Gallus gallus
TRVSDIYGMDV

SEQ ID NO: 469 - protein, 12 aa, Gallus gallus
VRDGRFINGLDV

SEQ ID NO: 470 - protein, 12 aa, Gallus gallus
VRGGWYRTPFEF

SEQ ID NO: 471 - protein, 8 aa, Gallus gallus
SSNLGNNY

SEQ ID NO: 472 - protein, 8 aa, Gallus gallus
SSNIGRNH

SEQ ID NO: 473 - protein, 8 aa, Gallus gallus
GGNIGNNY

SEQ ID NO: 474 - protein, 8 aa, Gallus gallus
GGNIASNY

SEQ ID NO: 475 - protein, 8 aa, Gallus gallus
GGSIASNY

SEQ ID NO: 476 - protein, 8 aa, Gallus gallus
SGSIASKY

SEQ ID NO: 477 - protein, 8 aa, Gallus gallus
SGSIASNY

SEQ ID NO: 478 - protein, 8 aa, Gallus gallus
SSNIGSNY

SEQ ID NO: 479 - protein, 8 aa, Gallus gallus
SGRIGSNY

SEQ ID NO: 480 - protein, 8 aa, Gallus gallus
TGSIAGNY

SEQ ID NO: 481 - protein, 8 aa, Gallus gallus
SGSVASNY

SEQ ID NO: 482 - protein, 8 aa, Gallus gallus
ASNIGNNY

SEQ ID NO: 483 - protein, 8 aa, Gallus gallus
SSNIGKNY

SEQ ID NO: 484 - protein, 6 aa, Gallus gallus
NIENKY

SEQ ID NO: 485 - protein, 8 aa, Gallus gallus
SSNIGSKY

SEQ ID NO: 486 - protein, 8 aa, Gallus gallus
TGGIAANY

SEQ ID NO: 487 - protein, 6 aa, Gallus gallus
NIGLKS

SEQ ID NO: 488 - protein, 7 aa, Gallus gallus
QSVSSSY

SEQ ID NO: 489 - protein, 8 aa, Gallus gallus
SSNIGNNY

SEQ ID NO: 490 - protein, 11 aa, Gallus gallus
GTWDSSLSAGV

SEQ ID NO: 491 - protein, 11 aa, Gallus gallus
AAWDDSLSSGV

SEQ ID NO: 492 - protein, 9 aa, Gallus gallus
QSSDSGRVV

SEQ ID NO: 493 - protein, 9 aa, Gallus gallus
QSSDNANVV

SEQ ID NO: 494 - protein, 10 aa, Gallus gallus
QSSDNINHVI

SEQ ID NO: 495 - protein, 11 aa, Gallus gallus
QSFDSTNQAVV

SEQ ID NO: 496 - protein, 10 aa, Gallus gallus
QSSDDSNHLV

SEQ ID NO: 497 - protein, 11 aa, Gallus gallus
AAWDDSLNGWV

SEQ ID NO: 498 - protein, 11 aa, Gallus gallus
ASWDDSLRGPV

SEQ ID NO: 499 - protein, 11 aa, Gallus gallus
AAWDDSLSGPV

SEQ ID NO: 500 - protein, 10 aa, Gallus gallus
QSYDSGVVVV

SEQ ID NO: 501 - protein, 9 aa, Gallus gallus
QSYDSGNVI

SEQ ID NO: 502 - protein, 9 aa, Gallus gallus
QSYDGRRLI

SEQ ID NO: 503 - protein, 11 aa, Gallus gallus
GTWDTSLRGVV

SEQ ID NO: 504 - protein, 9 aa, Gallus gallus
QSYDSSNWV

SEQ ID NO: 505 - protein, 11 aa, Gallus gallus
GTWDSSLSAVV

SEQ ID NO: 506 - protein, 11 aa, Gallus gallus
QVWDSSSDRVV

SEQ ID NO: 507 - protein, 11 aa, Gallus gallus
RSYDSSNRNWV

SEQ ID NO: 508 - protein, 10 aa, Gallus gallus
QSYDSGNPRV

SEQ ID NO: 509 - protein, 11 aa, Gallus gallus
AAWDDSLSGRV

SEQ ID NO: 510 - protein, 9 aa, Gallus gallus
HSSDNNNQV

SEQ ID NO: 511 - protein, 13 aa, Gallus gallus
QVWDDPSDQGSWV

SEQ ID NO: 512 - protein, 9 aa, Gallus gallus
QQYGSSPYT

SEQ ID NO: 513 - protein, 11 aa, Gallus gallus
ATWDDSLKAGV

SEQ ID NO: 514 - protein, 10 aa, Gallus gallus
QSYDSSNHWV

SEQ ID NO: 515 - protein, 10 aa, Gallus gallus
QAWDSSTNYV

SEQ ID NO: 516 - protein, 25 aa, Gallus gallus
EVQLVESGGGVVQPGRSLRLSCAAS

SEQ ID NO: 517 - protein, 25 aa, Gallus gallus
QVQLQQSGPGLVKPSQTLSLTCAIS

SEQ ID NO: 518 - protein, 25 aa, Gallus gallus
QVQLVQSEGGLVQPGGSLRLSCAAS

SEQ ID NO: 519 - protein, 25 aa, Gallus gallus
QVQLLQSAGGLVQPGGSLRLSCAAS

SEQ ID NO: 520 - protein, 25 aa, Gallus gallus
QVQLQQSGGGLVQSGGSLRLSCAAS

SEQ ID NO: 521 - protein, 25 aa, Gallus gallus
QVQLQQSGGGLVQPGGSLRLSCAAS

SEQ ID NO: 522 - protein, 25 aa, Gallus gallus
QVQLVQSGAEGKKPGSSVKVSCKAS

SEQ ID NO: 523 - protein, 25 aa, Gallus gallus [sequence omitted]

SEQ ID NO: 524 - protein, 25 aa, Gallus gallus [sequence omitted]

SEQ ID NO: 525 - protein, 25 aa, Gallus gallus
QVTLRESGGGLVKPGGSLRLSCSAS

SEQ ID NO: 526 - protein, 25 aa, Gallus gallus
QVQLLQSAGGVVQPGRSLRLSCAAS

SEQ ID NO: 527 - protein, 24 aa, Gallus gallus
HVQLVQSGGGSAQHGGSLRLSCAA

SEQ ID NO: 528 - protein, 25 aa, Gallus gallus
AVQLVESAGGVVQLRRSLSLSSAAS

SEQ ID NO: 529 - protein, 25 aa, Gallus gallus
QVQLQQSGAEVKKPGASLKVSCKAS

SEQ ID NO: 530 - protein, 25 aa, Gallus gallus
QVQLVQSGPEVKKPGASVNISCKTS

SEQ ID NO: 531 - protein, 25 aa, Gallus gallus [sequence omitted]

SEQ ID NO: 532 - protein, 25 aa, Gallus gallus
QVQLVQSGGGVVQPARSLRLSSAAS

SEQ ID NO: 533 - protein, 25 aa, Gallus gallus
EVQLVQSGGGLVKPGRSLRLSCTAS

SEQ ID NO: 534 - protein, 25 aa, Gallus gallus
QVNLRESGGGVVQPGGSLRLSCAAS

SEQ ID NO: 535 - protein, 25 aa, Gallus gallus
QVQLVQSGGGLVQPGGSLRLSCAAS

SEQ ID NO: 536 - protein, 17 aa, Gallus gallus
MHWVRQAPGKGLEWVSA

SEQ ID NO: 537 - protein, 17 aa, Gallus gallus
MNWVRQAPGKGLEWVSS

SEQ ID NO: 538 - protein, 17 aa, Gallus gallus
MSWVRQAPGRGLEWVSG

SEQ ID NO: 539 - protein, 17 aa, Gallus gallus
ISWVRQAPGQGLEWMGG

SEQ ID NO: 540 - protein, 17 aa, Gallus gallus
MHWVRQAPGQGLEWMGI

SEQ ID NO: 541 - protein, 17 aa, Gallus gallus
MNWVRQAPGKGLEWIST

SEQ ID NO: 542 - protein, 17 aa, Gallus gallus
MAWIRQAPGKGLEWVAY

SEQ ID NO: 543 - protein, 17 aa, Gallus gallus
VHWVRQAPGKGLEWVAA

SEQ ID NO: 544 - protein, 17 aa, Gallus gallus
MNWVRQAPGKRLEWIST

SEQ ID NO: 545 - protein, 17 aa, Gallus gallus
MHWIRQAPVKGMEWVSA

SEQ ID NO: 546 - protein, 17 aa, Gallus gallus
MHWVRQAPGKGLEWVSG

SEQ ID NO: 547 - protein, 17 aa, Gallus gallus
FSWVRQAPGQGLEWMGW

SEQ ID NO: 548 - protein, 17 aa, Gallus gallus
VHWVRQAPGQGFEYMGR

SEQ ID NO: 549 - protein, 17 aa, Gallus gallus
IHWVRQAPGKGLEWVSA

SEQ ID NO: 550 - protein, 17 aa, Gallus gallus
ISWVRQAPGQGLEWMGW

SEQ ID NO: 551 - protein, 17 aa, Gallus gallus
MSWVRQAPGKGLEWVST

SEQ ID NO: 552 - protein, 17 aa, Gallus gallus
MHWVRQAPGKRLEWVAV

SEQ ID NO: 553 - protein, 17 aa, Gallus gallus
MSWFRQAPGKGLEWVGF

SEQ ID NO: 554 - protein, 17 aa, Gallus gallus
MNWIRQAPGKGLEWVSS

SEQ ID NO: 555 - protein, 17 aa, Gallus gallus
MSWVRQAPGKGLEWVSS

SEQ ID NO: 556 - protein, 38 aa, Gallus gallus
YYADSVKGRFTISRDNSKNTLYLQMNSLRAEDTAVYYC

SEQ ID NO: 557 - protein, 40 aa, Gallus gallus
YNDYAVSVKSRITINPDTSKNQFSLQLNSVTPEDTAVYYC

SEQ ID NO: 558 - protein, 38 aa, Gallus gallus
YYADSVKGRFTISRDNSKNTLYLQMNSLRVEDTAVYYC

SEQ ID NO: 559 - protein, 38 aa, Gallus gallus
NYAQKFQGRVTITADESTSTAYMELRSLRSDDTAVYYC

SEQ ID NO: 560 - protein, 38 aa, Gallus gallus
NYAQKFQGRVTITADESTSTAYMELSSLRSEDTAVYYC

SEQ ID NO: 561 - protein, 40 aa, Gallus gallus
YNDYAVSVKSRITINPDTSKNQFSLHLNSVTPEDTAVYYC

SEQ ID NO: 562 - protein, 38 aa, Gallus gallus
SYAQKFQGRVTMTRDTSTSTVYMELSSLRSEDTAVYYC

SEQ ID NO: 563 - protein, 38 aa, Gallus gallus
YYADSVKGRFTVSRDNAKNSLFLQMDSLRDEDTAVYYC

SEQ ID NO: 564 - protein, 38 aa, Gallus gallus
YHADSVKGRFTVSRDNAKNLMYLEMNSLRADDTAVYYC

SEQ ID NO: 565 - protein, 38 aa, Gallus gallus
YYGDSVRGRFTISRDDSKNTVYLQMNSLRAEDTAVYYC

SEQ ID NO: 566 - protein, 38 aa, Gallus gallus
YYADSVKGRFTVSRDNTKNLTFLQMDSLRDEDTAVYYC

SEQ ID NO: 567 - protein, 38 aa, Gallus gallus
YYADSVKGRFTISRHNSKNTLYLQMNSLRAEDTGVYWG

SEQ ID NO: 568 - protein, 38 aa, Gallus gallus
IYADSVKGRFTISRDNAKNSLYLQMNSLRPEDTAVYYC

SEQ ID NO: 569 - protein, 38 aa, Gallus gallus
NYAQKFQGRVTMTTDTSTSTAYMEMRSLRSDDTAVYYC

SEQ ID NO: 570 - protein, 38 aa, Gallus gallus
RYAQKFQGRVAMTSDTSTSTAYMELSSLRSEDTAVYYC

SEQ ID NO: 571 - protein, 38 aa, Gallus gallus
YYADSVKGRFTISRDNAKNSLYLQMNSLRDEDTAVYYC

SEQ ID NO: 572 - protein, 38 aa, Gallus gallus
NYAQKLQGRVTMTTDTPTSTAYMEVRSLRSDDTAVYYC

SEQ ID NO: 573 - protein, 38 aa, Gallus gallus
NYADSVKGRFTISRDSSKNTLYLQMNSLRAEDTAVYYC

SEQ ID NO: 574 - protein, 38 aa, Gallus gallus
YYADSVKGRFSVSRGNSKNMLYLQMTGLTTEDTAVYYC

SEQ ID NO: 575 - protein, 38 aa, Gallus gallus
EYAASVKGRFTISRDDSKSIAYLQMNSLKTEDTAVYYC

SEQ ID NO: 576 - protein, 38 aa, Gallus gallus
YYADSVKGRFTISRDNAKNSLYLQMNSLRAEDTAMYYC

SEQ ID NO: 577 - protein, 38 aa, Gallus gallus
YYADSVKGRFTISRDNSDGSVYLKMNSLAAGDTAAYYC

SEQ ID NO: 578 - protein, 11 aa, Gallus gallus
WGRGTLVTVSS

SEQ ID NO: 579 - protein, 25 aa, Gallus gallus
QSVLTQPPSVSAAPGQKATISCSGG

SEQ ID NO: 580 - protein, 25 aa, Gallus gallus
SYELMQPPSVSVSPGQTARITCSGF

SEQ ID NO: 581 - protein, 25 aa, Gallus gallus
NFMLTQPHSVSESPGKTVTISCTGS

SEQ ID NO: 582 - protein, 25 aa, Gallus gallus
NFMLTQPHSVSESPGKTVTISCTGN

SEQ ID NO: 583 - protein, 25 aa, Gallus gallus
NFMLTQPHSVSESPGKTVTISCTRS

SEQ ID NO: 584 - protein, 25 aa, Gallus gallus
NFMLTQPHSLSESPGKTVTISCTRS

SEQ ID NO: 585 - protein, 25 aa, Gallus gallus
NFMLTQPHSVSESPGRTVTISCTRS

SEQ ID NO: 586 - protein, 25 aa, Gallus gallus
QPVLTQSPSASGTPGQRVTISCSGS

SEQ ID NO: 587 - protein, 25 aa, Gallus gallus
QPVLTQPPSASGTPEQRVTISCSGS

SEQ ID NO: 588 - protein, 25 aa, Gallus gallus
QSALTQPHSVSESPGKTVTISCTRT

SEQ ID NO: 589 - protein, 25 aa, Gallus gallus
NFMLTQPHSVSESPGKTVAISCSRS

SEQ ID NO: 590 - protein, 25 aa, Gallus gallus
QSVLTQPPSVSAAPGQKVTISCSGS

SEQ ID NO: 591 - protein, 25 aa, Gallus gallus
NFMLTQPHSVSESPGKTVTLSCTRS

SEQ ID NO: 592 - protein, 25 aa, Gallus gallus
QSVLTQPPSVSAAPGKKVTISCSGG

SEQ ID NO: 593 - protein, 25 aa, Gallus gallus
SYVLTQPPSVSVAPGQTAKITCGGD

SEQ ID NO: 594 - protein, 25 aa, Gallus gallus
NFMLTQPHSVAESPGKTVTISCTRS

SEQ ID NO: 595 - protein, 25 aa, Gallus gallus
NFMLTQPHSVSESPGKTVTISCTRN

SEQ ID NO: 596 - protein, 25 aa, Gallus gallus
SYVLTQPPSVSVAPGQTARITCGGD

SEQ ID NO: 597 - protein, 26 aa, Gallus gallus [sequence omitted]

SEQ ID NO: 598 - protein, 25 aa, Gallus gallus
SYVLTQPPSVSASPGQTASITCSGH

SEQ ID NO: 599 - protein, 17 aa, Gallus gallus
VQWYQQRPDSPPSTVIY

SEQ ID NO: 600 - protein, 17 aa, Gallus gallus
VQWYQHRPDSAPIAVIY

SEQ ID NO: 601 - protein, 17 aa, Gallus gallus
VQWYQQRPGSSPTTVIY

SEQ ID NO: 602 - protein, 17 aa, Gallus gallus
VQWYQQRPGSAPTTVIY

SEQ ID NO: 603 - protein, 17 aa, Gallus gallus
VNWYQQLPGTAPKLLIY

SEQ ID NO: 604 - protein, 17 aa, Gallus gallus
VQWYQQRPGSAPTVVIY

SEQ ID NO: 605 - protein, 17 aa, Gallus gallus
VQWYQQRPGSSPTTVIL

SEQ ID NO: 606 - protein, 17 aa, Gallus gallus
VQWYQQRPGNVPTTIIY

SEQ ID NO: 607 - protein, 17 aa, Gallus gallus
VQWYQQRPGSAPTIVIF

SEQ ID NO: 608 - protein, 17 aa, Gallus gallus
VHWYQQKPGQAPVVVVY

SEQ ID NO: 609 - protein, 17 aa, Gallus gallus
VHWYQQRPGSAPTTVIY

SEQ ID NO: 610 - protein, 17 aa, Gallus gallus
VQWYHQRPGSAPTTVIY

SEQ ID NO: 611 - protein, 17 aa, Gallus gallus
VHWYQQKPGQAPVLVVY

SEQ ID NO: 612 - protein, 17 aa, Gallus gallus
LAWYQQKPGQAPRLLIY

SEQ ID NO: 613 - protein, 17 aa, Gallus gallus
TSWYQQKPGQSPVLVIY

SEQ ID NO: 614 - protein, 36 aa, Gallus gallus
KRPSGIPDRFSGSKSGTSAALAITGLQTGDEADYYC

SEQ ID NO: 615 - protein, 36 aa, Gallus gallus
QRPSGVPDRFSGSKSGTSGSLTISGLQAEDEADYYC

SEQ ID NO: 616 - protein, 38 aa, Gallus gallus
QRPSGVPDRFSGSIDRSSNSASLTISGLRTEDEADYYC

SEQ ID NO: 617 - protein, 38 aa, Gallus gallus
QRPSGIPDRFSGSIDRSSNSASLTISGLRPEDEADYYC

SEQ ID NO: 618 - protein, 38 aa, Gallus gallus
QRPSGVPDRFSGSIDSSSNSASLTISGLKTEDEADYYC

SEQ ID NO: 619 - protein, 38 aa, Gallus gallus
QRPSGVPDRFSGSFDSSSNSASLTISGLKTEDEADYYC

SEQ ID NO: 620 - protein, 38 aa, Gallus gallus
QRLSGVPDRFSGSIDRSSNSASLTISGLKTEDEADYYC

SEQ ID NO: 621 - protein, 36 aa, Gallus gallus
QRPSGVPDRFSASKSGTSASLAISGLRSEDEADYYC

SEQ ID NO: 622 - protein, 38 aa, Gallus gallus
QRPSGVPDRFSGSIDSSTNSASLTISRLKTEDEADYYC

SEQ ID NO: 623 - protein, 38 aa, Gallus gallus
QRPSGVPDRFSGSIDTSSNSATLTISGLKTEDEADYYC

SEQ ID NO: 624 - protein, 38 aa, Gallus gallus
QRPSGVPDRFSGSIDKSSNSASLTISGLKTEDEADYYC

SEQ ID NO: 625 - protein, 36 aa, Gallus gallus
QRPSGIPDRFSGSKSGTSATLGITGLQTGDEADYYC

SEQ ID NO: 626 - protein, 38 aa, Gallus gallus
RRPSGVPDRFSGSIDRSSNSASLTIFGMKTEDEADYYC

SEQ ID NO: 627 - protein, 36 aa, Gallus gallus
KRPSGIPDRFSGSKSGTSASLAITGLQTGDEADYYC

SEQ ID NO: 628 - protein, 36 aa, Gallus gallus
KRPSGIPDRFSGSKSGTSATLGITGLQTGDEADYYC

SEQ ID NO: 629 - protein, 36 aa, Gallus gallus
DRPSGIPDRFSGSNSGNTATLTITRVEAGDEADYYC

SEQ ID NO: 630 - protein, 35 aa, Gallus gallus
QRPSGVPDRFSGSKSGTSASLVISGLRSEDEADYH

SEQ ID NO: 631 - protein, 38 aa, Gallus gallus
QRPSGVPGRFSGSIDGSSNSASLTISGLQAEDEGDYYC

SEQ ID NO: 632 - protein, 36 aa, Gallus gallus
DRPSDIPERFSGSNSGNTATLTISRVEAGDEADYYC

SEQ ID NO: 633 - protein, 36 aa, Gallus gallus [sequence omitted]

SEQ ID NO: 634 - protein, 38 aa, Gallus gallus
KRPSGVPDRFSGSIDSSTNSASLTISGLKTEDEADYYC

SEQ ID NO: 635 - protein, 10 aa, Gallus gallus
FGQGTKVDIK

SEQ ID NO: 636 - protein, 114 aa, Gallus gallus
EVQLVESGGGVVQPGRSLRLSCAASGFTFSSYAMHWVRQAPGKGLEWVSAISGSGGSTYYADSVKGRFTISRDNSKNTLYLQMNSLRAEDTAVYYCARDAHDYWGQGTLVTVSS

SEQ ID NO: 637 - protein, 110 aa, Gallus gallus
QSVLTQPPSVSAAPGQKATISCSGGSSNLGNNYVSWYQQLPGTAPKLLIYDNNKRPSGIPDRFSGSKSGTSAALAITGLQTGDEADYYCGTWDSSLSAGVFGGGTKLTVL

SEQ ID NO: 638 - protein, 119 aa, Gallus gallus
EVQLVQSGGGLVKPGGSLRLSCAASGFTFSSYSMNWVRQAPGKGLEWVSSISSSSSYIYYADSVKGRFTISRDNSKNTLYLQMNSLRAEDTAVYYCARVKDYDSSLDYWGQGTLVTVSS

SEQ ID NO: 639 - protein, 106 aa, Gallus gallus
SYELMQPPSVSVSPGQTARITCSGFKLGDKYVSWYQQKPGQSPVLVIYQDSKRPSGIPERFSGSNSGNTATLTISGTQAMDEADYYCQAWDSSTVVFGGGTKLTVL

SEQ ID NO: 640 - protein, 123 aa, Gallus gallus
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARGITGTGYGMDVWGQGTLVTVSS

SEQ ID NO: 641 - protein, 110 aa, Gallus gallus
QSVLTQPPSASGTPGQRVTISCSGSSSNIGRNHVYWYQQLPGTAPKLLIYNNDQRPSGVPDRFSGSKSGTSGSLTISGLQAEDEADYYCAAWDDSLSSGVFGGGTKVTVL

SEQ ID NO: 642 - protein, 119 aa, Gallus gallus
QVQLVQSEGGLVQPGGSLRLSCAASGFSFGSSAMSWVRQAPGRGLEWVSGISHSGSTTYYADSVKGRFTISRDNSKNTLYLQMNSLRVEDTAVYYCAKIGAYGYYFHYWGQGTLVTVSS

SEQ ID NO: 643 - protein, 109 aa, Gallus gallus
NFMLTQPHSVSESPGKTVTISCTGSGGNIGNNYVQWYQQRPDSPPSTVIYDDSQRPSGVPDRFSGSIDRSSNSASLTISGLRTEDEADYYCQSSDSGRVVFGGGTKLTV

SEQ ID NO: 644 - protein, 119 aa, Gallus gallus
QVQLLQSAGGLVQPGGSLRLSCAASGYSFGSSAMSWVRQAPGRGLEWVSGISHSGSTTYYADSVKGRFTISRDNSKNTLYLQMNSLRAEDTAVYYCAKIGAYGYYFHYWGQGTLVTVSS

SEQ ID NO: 645 - protein, 110 aa, Gallus gallus
NFMLTQPHSVSESPGKTVTISCTGNGGNIASNYVQWYQHRPDSAPIAVIYDDSQRPSGIPDRFSGSIDRSSNSASLTISGLRPEDEADYYCQSSDNANVVFGGGTKLTVL

SEQ ID NO: 646 - protein, 119 aa, Gallus gallus
QVQLQQSGGGLVQSGGSLRLSCAASGFSFGSSAMSWVRQAPGRGLEWVSGISHSGSTTYYADSVKGRFTISRDNSKNTLYLQMNSLRVEDTAVYYCAKIGAYGYYFHYWGQGTLVTVSS

SEQ ID NO: 647 - protein, 111 aa, Gallus gallus
NFMLTQPHSVSESPGKTVTISCTRSGGSIASNYVQWYQQRPGSSPTTVIYDDNQRPSGVPDRFSGSIDSSSNSASLTISGLKTEDEADYYCQSSDNINHVIFGGGTKLTVL

SEQ ID NO: 648 - protein, 119 aa, Gallus gallus
QVQLQQSGGGLVQPGGSLRLSCAASGFSFGSSAMSWVRQAPGRGLEWVSGISHSGSTTYYADSVKGRFTISRDNSKNTLYLQMNSLRVEDTAVYYCAKIGAYGYYFHYWGQGTLVTVSS

SEQ ID NO: 649 - protein, 112 aa, Gallus gallus
NFMLTQPHSLSESPGKTVTISCTRSSGSIASKYVQWYQQRPGSAPTTVIYDDKQRPSGVPDRFSGSFDSSSNSASLTISGLKTEDEADYYCQSFDSTNQAVVFGGGTKLTVL

SEQ ID NO: 650 - protein, 119 aa, Gallus gallus; identical to SEQ ID NO: 648

SEQ ID NO: 651 - protein, 111 aa, Gallus gallus
NFMLTQPHSVSESPGRTVTISCTRSSGSIASNYVQWYQQRPGSAPTTVIYEDNQRLSGVPDRFSGSIDRSSNSASLTISGLKTEDEADYYCQSSDDSNHLVFGGGTKLTVL

SEQ ID NO: 652 - protein, 122 aa, Gallus gallus
QVQLVQSGAEGKKPGSSVKVSCKASGGTFSSYAISWVRQAPGQGLEWMGGIIPIFGTANYAQKFQGRVTITADESTSTAYMELRSLRSDDTAVYYCARDAVVVADSGSMDVWGQGTTVTVSS

SEQ ID NO: 653 - protein, 110 aa, Gallus gallus
QPVLTQSPSASGTPGQRVTISCSGSSSNIGSNTVNWYQQLPGTAPKLLIYSNNQRPSGVPDRFSGSKSGTSASLAISGLQSEDEADYYCAAWDDSLNGWVFGGGTKVTVL

SEQ ID NO: 654 - protein, 122 aa, Gallus gallus
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDAVVVADSGSMDVWGQGTTVTVSS

SEQ ID NO: 655 - protein, 110 aa, Gallus gallus
QAGLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGWVFGGGTKLTVL

SEQ ID NO: 656 - protein, 117 aa, Gallus gallus
QVQLQQSGPGLVKPSQTLSLTCAISGDSVSSNSAAWNWIRQSPSRGLEWLGRTYYRSKWYNDYAVSVKSRITINPDTSKNQFSLHLNSVTPEDTAVYYCATWGMDVWGQGTTVTVSS

SEQ ID NO: 657 - protein, 110 aa, Gallus gallus
QSVLTQPPSASGTPGQRVTISCSGSSSNIGSNYVYWYQQLPGTAPKLLIYRNNQRPSGVPDRFSASKSGTSASLAISGLRSEDEADYYCASWDDSLRGPVFGGGTKLTVL

SEQ ID NO: 658 - protein, 117 aa, Gallus gallus
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCATWGMDVWGQGTTVTVSS

SEQ ID NO: 659 - protein, 110 aa, Gallus gallus
QPVLTQPPSASGTPGQRVTISCSGSSSNIGSNYVYWYQQLPGTAPKLLIYRNNQRPSGVPDRFSGSKSGTSASLAISGLQSEDEADYYCAAWDDSLNGPVFGGGTKLTVL

SEQ ID NO: 660 - protein, 117 aa, Gallus gallus
QVQLQQSGPGLVKPSQTLSLTCAISGDSVSSNSAAWNWIRQSPSRGLEWLGRTYYRSKWYNDYAVSVKSRITINPDTSKNQFSLHLNSVTPEDTAVYYCATWGMDVWGQGTMVTVSS

SEQ ID NO: 661 - protein, 110 aa, Gallus gallus
QPVLTQPPSASGTPEQRVTISCSGSSSNIGSNYVYWYQQLPGTAPKLLIYRNNQRPSGVPDRFSGSKSGTSASLAISGLRSEDEADYYCAAWDDSLSGPVFGGGTKLTVL

SEQ ID NO: 662 - protein, 121 aa, Gallus gallus
QVQLLQSAAEVKKPGASVKVPCKASGYTFTGHYMHWVRQAPGQGLEWMGIINPRGGSTSYAQKFQGRVTMTRDTSTSTVYMELSSLRSEDTAVYYCARESIAAAGPYFDYWGQGTLVTVSS

SEQ ID NO: 663 - protein, 110 aa, Gallus gallus
QSALTQPHSVSESPGKTVTISCTRTSGRIGSNYVQWYQQRPGSAPTVVIYEDNQRPSGVPDRFSGSIDSSTNSASLTISRLKTEDEADYYCQSYDSGVVVVFGGGTKLTV

SEQ ID NO: 664 - protein, 115 aa, Gallus gallus
EVQLVESGGGLVQPGGSLRLSCAASGFTFNNFYMNWVRQAPGKGLEWISTISRGGNSVYYADSVKGRFTVSRDNAKNSLFLQMDSLRDEDTAVYYCVRGGDFLYWGQGTLVTVSS

SEQ ID NO: 665 - protein, 110 aa, Gallus gallus
NFMLTQPHSVSESPGKTVTISCTRSTGSIAGNYVQWYQQRPGSSPTTVILEDKQRPSGVPDRFSGSIDTSSNSATLTISGLKTEDEADYYCQSYDSGNVIFGGGTKLTVL

SEQ ID NO: 666 - protein, 118 aa, Gallus gallus
QVTLRESGGGLVKPGGSLRLSCSASGFTFRGFYMAWIRQAPGKGLEWVAYISSSGSAVYHADSVKGRFTVSRDNAKNLMYLEMNSLRADDTAVYYCARDGGTGGFDYWGQGTLVTVSS

SEQ ID NO: 667 - protein, 110 aa, Gallus gallus
NFMLTQPHSVSESPGKTVAISCSRSSGSVASNYVQWYQQRPGNVPTTIIYQDNQRPSGVPDRFSGSIDKSSNSASLTISGLKTEDEADYYCQSYDGRRLIFGGGTKLTVL

SEQ ID NO: 668 - protein, 121 aa, Gallus gallus
QVQLLQSAGGVVQPGRSLRLSCAASGFTFSTYAVHWVRQAPGKGLEWVAAISYDGAGKYYGDSVRGRFTISRDDSKNTVYLQMNSLRAEDTAVYYCAGEHYTLTDEEFDFWGQGTLVTVSS

SEQ ID NO: 669 - protein, 110 aa, Gallus gallus
QSVLTQPPSVSAAPGQKVTISCSGSASNIGNNYVSWYQQLPGTAPKLLIYDNNQRPSGIPDRFSGSKSGTSATLGITGLQTGDEADYYCGTWDTSLRGVVFGGGTKVTVL

SEQ ID NO: 670 - protein, 115 aa, Gallus gallus
HVQLVQSGGGSAQHGGSLRLSCAASGFTFNNSYMNWVRQAPGKRLEWISTISRGGNSVYYADSVKGRFTVSRDNTKNLTFLQMDSLRDEDTAVYYCAKGGDYLYWGQGTLVTVSS

SEQ ID NO: 671 - protein, 110 aa, Gallus gallus
NFMLTQPHSVSESPGKTVTLSCTRSSGSIASKYVQWYQQRPGSAPTIVIFDDDRRPSGVPDRFSGSIDRSSNSASLTIFGMKTEDEADYYCQSYDSSNWVFGGGTKLTVL

SEQ ID NO: 672 - protein, 114 aa, Gallus gallus
AVQLVESAGGVVQLRRSLSLSSAASRFTFSSYPMHWIRQAPVKGMEWVSAISGSGGSTYYADSVKGRFTISRHNSKNTLYLQMNSLRAEDTGVYWGARDPHDFWGQGTLVTVSS

SEQ ID NO: 673 - protein, 110 aa, Gallus gallus
QSVLTQPPSVSAAPGQKATISCSGGSSNLGNNYVSWYQQLPGTAPKLLIYDNNKRPSGIPDRFSGSKSGTSASLAITGLQTGDEADYYCGTWDSSLSAGVFGGGTKLTVL

SEQ ID NO: 674 - protein, 120 aa, Gallus gallus
QVQLVQSGGGLVQPGRSLRLSCAASGFTFDDFAMHWVRQAPGKGLEWVSGITWNSAATIYADSVKGRFTISRDNAKNSLYLQMNSLRPEDTAVYYCARDRGGPKDPFDIWGQGTMVTVSS

SEQ ID NO: 675 - protein, 110 aa, Gallus gallus
QSVLTQPPSVSAAPGKKVTISCSGGSSNIGKNYVSWYQQLPGTAPKLLIYDNNKRPSGIPDRFSGSKSGTSATLGITGLQTGDEADYYCGTWDSSLSAVVFGGGTKLTVL

SEQ ID NO: 676 - protein, 127 aa, Gallus gallus
QVQLQQSGAEVKKPGASLKVSCKASGYTFTRYGFSWVRQAPGQGLEWMGWISAYNGNTNYAQKFQGRVTMTTDTSTSTAYMEMRSLRSDDTAVYYCARDRGYCSGGSCPGIDAFDIWGQGTMVTVSS

SEQ ID NO: 677 - protein, 108 aa, Gallus gallus
SYVLTQPPSVSVAPGQTAKITCGGDNIENKYVHWYQQKPGQAPVVVVYDDSDRPSGIPDRFSGSNSGNTATLTITRVEAGDEADYYCQVWDSSSDRVVFGGGTKLTVL

SEQ ID NO: 678 - protein, 123 aa, Gallus gallus
QVQLVQSGPEVKKPGASVNISCKTSGYTFRNYYVHWVRQAPGQGFEYMGRVNPNSGNARYAQKFQGRVAMTSDTSTSTAYMELSSLRSEDTAVYYCAREPPATVAYYYGMDVWGQGTTVTVSS

SEQ ID NO: 679 - protein, 112 aa, Gallus gallus
NFMLTQPHSVSESPGKTVTISCTGSSGSIASNYVHWYQQRPGSAPTTVIYEDNQRPSGVPDRFSGSIDSSSNSASLTISGLKTEDEADYYCRSYDSSNRNWVFGGGTKLTVL

SEQ ID NO: 680 - protein, 117 aa, Gallus gallus
EVQLVESGGGVVQPGRSLRLSCAASGFTFSSYGIHWVRQAPGKGLEWVSAISGSGGSTYYADSVKGRFTISRDNAKNSLYLQMNSLRDEDTAVYYCARERLIGMDVWGQGTTVTVSS

SEQ ID NO: 681 - protein, 111 aa, Gallus gallus
NFMLTQPHSVAESPGKTVTISCTRSSGSIASNYVQWYQQRPGSAPTTVIYEDNQRPSGVPDRFSGSIDSSSNSASLTISGLKTEDEADYYCQSYDSGNPRVFGGGTKLTVL

SEQ ID NO: 682 - protein, 117 aa, Gallus gallus
QVQLVQSGAEVKKPGASVKVSCKASGYTFTSYGISWVRQAPGQGLEWMGWISAYNGNTNYAQKLQGRVTMTTDTPTSTAYMEVRSLRSDDTAVYYCAREVNYGLDVWGQGTLVTVSS

SEQ ID NO: 683 - protein, 110 aa, Gallus gallus
QPVLTQPPSASGTPGQRVTISCSGSSSNIGSKYVYWYQQLPGTAPKLLIYRNNQRPSGVPDRFSGSKSGTSASLVISGLRSEDEADYHCAAWDDSLSGRVFGGGTKLTVL

SEQ ID NO: 684 - protein, 119 aa, Gallus gallus
EVQLVESGGGLVQPGGSLRLSCAASGFTSNTYAMSWVRQAPGKGLEWVSTISGSGSSTNYADSVKGRFTISRDSSKNTLYLQMNSLRAEDTAVYYCARIERYSYGIDYWGQGTLVTVSS

SEQ ID NO: 685 - protein, 110 aa, Gallus gallus
NFMLTQPHSVSESPGKTVTISCTRNTGGIAANYVQWYHQRPGSAPTTVIYEDKQRPSGVPGRFSGSIDGSSNSASLTISGLQAEDEGDYYCHSSDNNNQVFGGGTKLTVL

SEQ ID NO: 686 - protein, 119 aa, Gallus gallus
QVQLVQSGGGVVQPARSLRLSSAASGFTFSSYGMHWVRQAPGKRLEWVAVISPDGSDKYYADSVKGRFSVSRGNSKNMLYLQMTGLTTEDTAVYYCARNRKYTNFLDIWGQGTMVTVSS

SEQ ID NO: 687 - protein, 110 aa, Gallus gallus
SYVLTQPPSVSVAPGQTARITCGGDNIGLKSVHWYQQKPGQAPVLVVYDDVDRPSDIPERFSGSNSGNTATLTISRVEAGDEADYYCQVWDDPSDQGSWVFGGGTKLTVL

SEQ ID NO: 688 - protein, 116 aa, Gallus gallus
QVQLVQSGAEVKKPGASVKVSCKASGYTFTSYYMHWVRQAPGQGLEWMGIINPSGGTTSYAQKFQGRVTMTRDTSTSTVYMELSSLRSEDTAVYYCARNWASFDYWGQGTLVTVSS

SEQ ID NO: 689 - protein, 108 aa, Gallus gallus
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPYTFGQGTKVDIK

SEQ ID NO: 690 - protein, 120 aa, Gallus gallus
EVQLVQSGGGLVKPGRSLRLSCTASGFTFGDYAMSWFRQAPGKGLEWVGFIRSKAYGGTTEYAASVKGRFTISRDDSKSIAYLQMNSLKTEDTAVYYCTRVSDIYGMDVWGQGTTVTVSS

SEQ ID NO: 691 - protein, 110 aa, Gallus gallus
QSVLTQPPSVSAAPGQKVTISCSGSSSNIGNNYVSWYQQLPGTAPKLLIYDNNKRPSGIPDRFSGSKSGTSATLGITGLQTGDEADYYCATWDDSLKAGVFGGGTKLTVL

SEQ ID NO: 692 - protein, 119 aa, Gallus gallus
QVNLRESGGGVVQPGGSLRLSCAASGFTFRRYGMNWIRQAPGKGLEWVSSISSSSSYIYYADSVKGRFTISRDNAKNSLYLQMNSLRAEDTAMYYCVRDGRFINGLDVWGQGTLVTVSS

SEQ ID NO: 693 - protein, 111 aa, Gallus gallus
NFMLTQPHSVSESPGRTVTISCTRSSGSIASNYVQWYQQRPGSSPTTVIYDDKKRPSGVPDRFSGSIDSSTNSASLTISGLKTEDEADYYCQSYDSSNHWVFGGGTKVTVL

SEQ ID NO: 694 - protein, 119 aa, Gallus gallus
QVQLVQSGGGLVQPGGSLRLSCAASGFTFSRYSMSWVRQAPGKGLEWVSSISGGGGSTYYADSVKGRFTISRDNSDGSVYLKMNSLAAGDTAAYYCVRGGWYRTPFEFWGRGTLVTVSS

SEQ ID NO: 695 - protein, 107 aa, Gallus gallus
SYVLTQPPSVSASPGQTASITCSGHKLGDKYTSWYQQKPGQSPVLVIYQDNKRPSGIPERFSGSNSGNTATLTISGTQAMDEADYYCQAWDSSTNYVFGTGTKLTVL

SEQ ID NO: 696 - protein, 9 aa, Homo sapiens
ARGRNGMDV

SEQ ID NO: 697 - protein, 8 aa, Homo sapiens
SSNIGHKY

SEQ ID NO: 698 - protein, 11 aa, Homo sapiens
ATWDDSLNGVV

SEQ ID NO: 699 - protein, 40 aa, Homo sapiens
YTDFAVSVKSRITINPDTSKNQFSLQLNSVTPEDTAVYYC

SEQ ID NO: 700 - protein, 17 aa, Homo sapiens
VYWYQQLPGAAPKLLIY

SEQ ID NO: 701 - protein, 119 aa, Homo sapiens
QVQLQQSGPGLVKPSQTLSLTCAISGDSVSSNSAVWNWIRQSPSRGLEWLGRTYYRSKWYTDFAVSVKSRITINPDTSKNQFSLQLNSVTPEDTAVYYCARGRNGMDVWGQGTMVTVST

SEQ ID NO: 702 - protein, 110 aa, Homo sapiens
LPVLTQPPSASGTPGQRVTISCSGSSSNIGHKYVYWYQQLPGAAPKLLIYRNNQRPSGVPDRFSASKSGTSASLAISGLRSEDEADYYCATWDDSLNGVVFGGGTKVTVL

SEQ ID NO: 703 - protein, 8 aa, Gallus gallus
GYSFGSSA

SEQ ID NO: 704 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 705 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 706 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 707 - protein, 17 aa, synthetic construct
SSGGGGSGGGGSGGSAL

SEQ ID NO: 708 - protein, 121 aa, Homo sapiens
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVE

SEQ ID NO: 709 - protein, 110 aa, Homo sapiens
GGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 710 - protein, 8 aa, Gallus gallus
GFTFSSYT

SEQ ID NO: 711 - protein, 8 aa, Gallus gallus
ICNDGTTT

SEQ ID NO: 712 - protein, 16 aa, Gallus gallus
TRGVDICWNSGNNIDA

SEQ ID NO: 713 - protein, 123 aa, Gallus gallus
AVTLDESGGGLQTPGGGLSLVCKASGFTFSSYTMQWVRQAPGKGLEWVAGICNDGTTTNYGSAVKGRATISRDNGQSTLRLQLNNLRAEDTGIYFCTRGVDICWNSGNNIDAWGHGTEVIVSS

SEQ ID NO: 714 - protein, 110 aa, Gallus gallus
QAALTQPASVSANPGETVKITCSGGGSYYGSYYYGWYQQKSPGSAPVTVIYNNDKRPSDIPSRFSGSLSGSTNTLTITGVQVEDESVYFCGSKDSSGAGIYGAGTTLTVL